(19) United States
(12) Patent Application Publication
(10) Pub. No.: US 2026/0234236 A1
(43) Pub. Date: Aug. 13, 2026

(54) CANINE ANTIBODIES TO CANINE IL-13

(71) Applicant: INTERVET INC., Rahway, NJ (US)

(72) Inventors: Mohamad MORSEY, Omaha, NE (US); Zhisong QIAO, Omaha, NE (US); Seth D. STAUFFER, New Palestine, IN (US); Yuanzheng ZHANG, Somerset, NJ (US)

(73) Assignee: INTERVET INC., Rahway, NJ (US)

(21) Appl. No.: 19/153,746

(22) PCT Filed: Feb. 12, 2024
(86) PCT No.: PCT/EP2024/053457

§ 371 Date: Aug. 5, 2025

Related U.S. Application Data
(60) Provisional application No. 63/484,622, filed on Feb. 13, 2023.

(51) Int. Cl. C07K 16/24; A61K 39/00; A61P 17/00; A61P 17/04; A61P 29/00

(52) U.S. Cl. CPC C07K 16/244; A61P 17/00; A61P 17/04; A61P 29/00; A61K 2039/505; C07K 2317/20; C07K 2317/34; C07K 2317/565; C07K 2317/622; C07K 2317/76; C07K 2317/92; C07K 2317/94

(57) ABSTRACT

The present invention provides canine antibodies that bind to canine interleukin-13 (canine IL-13) that have a high binding affinity for canine IL-13 and that can block the binding of canine IL-13 to the canine Type II heterodimer IL-4 receptor. The present invention also provides methods of using the antibodies of the present invention in the treatment of atopic dermatitis in dogs.

### FIELD OF THE INVENTION

The present invention relates to canine antibodies that bind to canine interleukin-13 (cIL-13) that have a high binding affinity for canine IL-13 and that can block the binding of canine IL-13 to the canine Type II heterodimer IL-4 receptor. The present invention also relates to the use of the antibodies of the present invention in the treatment of atopic dermatitis in dogs.

### BACKGROUND OF THE INVENTION

The immune system comprises a network of resident and recirculating specialized cells that function collaboratively to protect the host against infectious diseases and cancer. The ability of the immune system to perform this function depends to a large extent on the biological activities of a group of signaling proteins secreted by leukocytes and collectively referred to as interleukins. Two such interleukins are interleukin-4 (IL-4) and interleukin-13 (IL-13). IL-4 and IL-13 are closely related proteins that can be secreted by many cell types including CD4⁺ Th2 cells, natural killer T cells (NKT), macrophages, mast cells, and basophils. IL-4 and IL-13 display many overlapping functions and are critical to the development of T cell-dependent humoral immune responses. Both IL-4 and IL-13 also are part of a signaling pathway involved in atopic dermatitis.

It is known that IL-4 binds with high affinity to two heterodimer receptors i.e., the Type I and Type II IL-4 receptors. The Type I heterodimer IL-4 receptor consists of the IL-4 receptor alpha (IL-4Rα) chain and the common gamma C (γc) chain. The Type II heterodimer IL-4 receptor consists of the IL-4Rα chain and the IL-13 receptor alpha 1 (IL-13R alpha 1) chain. IL-13 binds to the IL-13R alpha 1 chain of the IL-4 Type II receptor. IL-13 also binds to a unique receptor designated as the IL-13 receptor alpha 2 (IL-13R alpha 2), which is secreted in a soluble form. The binding of IL-13 to IL-13R alpha 2 is not currently thought to transduce a signal. Accordingly, IL-13R alpha 2 has often been referred to as a decoy receptor.

Genes encoding the IL-4 protein from various species have been cloned and expressed in bacterial and mammalian cells. For example, the cDNA encoding human IL-4 (hIL-4) shows that the mature human IL-4 is a secreted polypeptide of 129 amino acids with a predicted molecular weight of 15 Kd [Yokota et al., Proc Natl Acad Sci USA. 83(16): 5894-5898 (1986)]. The cDNA encoding canine IL-4 (cIL-4) also has been identified and shown to encode a 132 amino acid polypeptide that shares 40% sequence identity with human IL-4 [van der Kaaij et al., Immunogenetics 49:142-143 (1999)].

The gene encoding human IL-13 (hIL-13) has been cloned and expressed in a variety of host systems [Minty et al., Nature 362:248-50 (1993)]. A cDNA encoding hIL-13 shows that the mature hIL-13 is a secreted polypeptide with a 12.4 Kd molecular weight. A cDNA encoding canine IL-13 (cIL-13) also has been identified [Yang et al., J. Interferon and Cytokine Research 20:779-785 (2000)]. The predicted cIL-13 mature polypeptide consists of 111 amino acids and shares 61.8% sequence identity with hIL-13. Although IL-4 and IL-13 are cytokines that are involved in the development of immune responses that are required for protection against extracellular pathogens (e.g., tissue or lumen dwelling parasites), these cytokines also have been implicated in the pathogenesis of allergic diseases in humans and animals, including atopic dermatitis (AD).

The therapeutic use of monoclonal antibodies to block signal transduction in specific pathways by binding to either a protein ligand or its protein receptor has proven to be widely successful. Indeed, such monoclonal antibodies play a critical role in the rapid growth of human biopharmaceuticals and as of 2017, claimed over 25% of the human biopharmaceutical market. Among the 20 drugs with the highest sales in 2014, six were monoclonal antibodies [Chung, Experimental & Molecular Medicine 49:e304; doi: 10.1038/emm.2017.46 (2017)]. This trend continues to grow. Monoclonal antibodies raised against human IL-4Rα have been developed and some of these antibodies have been extensively tested for their therapeutic effects for treating atopic dermatitis in humans [see, e.g., US2015/0017176 A1]. One such antibody (dupilumab) was produced by the immunization of transgenic mice in which the mouse antibody genes were replaced with human antibody genes and therefore, the resulting antibody is a human antibody as opposed to e.g., a humanized murine antibody.

Although initially limited to human biopharmaceuticals due to the high cost of monoclonal antibody therapeutics, canine monoclonal products have recently become available because of significant reductions in production costs. Early indications suggest that such monoclonal antibodies also are likely to become major therapeutics in the companion animal market as well. For example, an antibody against human interleukin-31 receptor alpha (hIL-31RA) has been tested and found to have a significant effect on pruritus associated with atopic dermatitis in humans [Ruzicka, et al., New England Journal of Medicine, 376(9), 826-835 (2017)]. In addition, antibodies against canine IL-31 (cIL-31) have been shown to have a significant effect on pruritus associated with atopic dermatitis in dogs [U.S. Pat. Nos. 8,790,651 B2; 10,093,731 B2]. This caninized antibody blocks the binding of cIL-31 to the canine IL-31 receptor alpha (cIL-31RA), thereby blocking the cIL-31/cIL-31RA signaling pathway. Accordingly, blocking cIL-31 binding to its receptor cIL-31RA results in the relief of pruritus associated with atopic dermatitis. However, merely blocking the cIL-31/cIL-31R signaling pathway only ameliorates the pruritic effect of atopic dermatitis but does nothing to stop the concomitant skin inflammation caused by (cIL-4 or cIL-13)/canine Type II IL-4 receptor signaling pathways.

More recently, caninized antibodies to canine IL-4Rα (that cIL-4Rα) also have been disclosed [US2018/0346580A1, hereby incorporated by reference in its entirety] that block the binding of cIL-4 to cIL-4Rα. These antibodies were produced by immunization of conventional, i.e., non-transgenic mice, with the cIL-4Rα extra-cellular domain (ECD). Because the Type II cIL-4 receptor consists of the cIL-4Rα chain and the cIL-13R alpha 1 chain, antibodies to canine IL-4 Rα have been obtained that can block both cIL-4 and cIL-13 from binding the Type II cIL-4 receptor, thereby serving to help block the inflammation associated with atopic dermatitis. Recently therapies based on monoclonal antibodies that target hIL-4 and/or hIL-13 signaling pathways have been approved for the treatment of AD in humans.

However, despite recent successes in treating pruritus associated with atopic dermatitis, and recent encouraging disclosures on the treatment of the associated inflammation, many subjects suffering from this condition still do not experience a rapid onset of antipruritic action concomitant with a significant effect on the skin inflammation. Therefore, there is a need to design alternative therapies to address this unmet medical need.

Atopic dermatitis is also a common disease in companion animals, especially dogs, where its prevalence has been estimated to be approximately 10-15% of the canine population. The pathogenesis of atopic dermatitis in dogs and cats [reviewed in Nuttall et al., Veterinary Records 172(8):201-207 (2013)] bears significant similarities to that of atopic dermatitis in man including skin infiltration by a variety of immune cells and CD4 Th2 polarized cytokine milieu including preponderance of IL-4 and IL-13 cytokines. Thus, there is an unmet medical need for a safe and effective treatment option for atopic dermatitis in companion animals. Such treatment could interfere with the underlying mechanism of disease including correction of apparent Th2 immune dysregulation resulting from aberrant expression of IL-4 and IL-13 cytokines in diseased animals.

### SUMMARY OF THE INVENTION

In one aspect, the present invention relates to canine antibodies and scFvs that bind to canine interleukin-13 (cIL-13). The present invention also relates to antigen binding fragments of the canine antibodies, which bind to cIL-13. The present invention further provides these canine antibodies, and the antigen binding fragments thereof, that both bind cIL-13 and block the binding of cIL-13 to the canine Type II heterodimer interleukin-4 receptor (Type II cIL-4R). In certain embodiments, a canine antibody or an antigen binding fragment thereof binds cIL-13 and blocks the binding of cIL-13 to the canine Type II cIL-4R, but does not block the binding of CIL-13 to cIL-13R alpha 2.

Accordingly, the present invention provides canine antibodies, including isolated canine antibodies, that bind cIL-13 and comprise a heavy chain and a light chain that together comprise a set of six complementary determining regions (CDRs), three of which are heavy chain CDRs: CDR heavy 1 (HCDR1), CDR heavy 2 (HCDR2) and CDR heavy 3 (HCDR3) and three of which are light chain CDRs: CDR light 1 (LCDR1), CDR light 2 (LCDR2), and CDR light 3 (LCDR3), the canine heavy chain variable regions of the heavy chain of the canine antibodies, the light chain variable regions of the light chain of the canine antibodies, the antigen binding fragments of the heavy and light chains of the canine antibodies, and related scFvs.

The present invention further provides nucleic acids, including isolated nucleic acids, that encode: any of the sets of three heavy chain CDRs (HCDR1, HCDR2, and HCDR3), canine heavy chain variable regions of the heavy chains of the canine antibodies and/or the heavy chains of the canine antibodies or antigen binding fragments thereof, any of the sets of three light chain CDRs (LCDR1, LCDR2, and LCDR3), the light chain variable regions of the canine antibodies and/or the light chains of the canine antibodies or antigen binding fragments of the present invention, and related scFvs.

The canine antibodies that bind cIL-13, comprise a heavy chain and a light chain that together comprise a set of six CDRs, three of which are heavy chain CDRs and three of which are light chain CDRs. In particular embodiments, the canine antibodies or antigen binding fragment thereof comprise the HCDR1 that comprises the amino acid sequence of SEQ ID NO: 114, the HCDR2 comprises the amino acid sequence of SEQ ID NO: 115, the HCDR3 comprises the amino acid sequence of SEQ ID NO: 116, the LCDR1 comprises the amino acid sequence of SEQ ID NO: 117, the LCDR2 comprises the amino acid sequence of SEQ ID NO: 118, and the LCDR3 comprises the amino acid sequence of SEQ ID NO: 119. In certain embodiments of this type, the antibody is a canine antibody or antigen binding fragment of the canine antibody. In specific embodiments, the antibody is a canine antibody.

In alternative embodiments of the canine antibodies or antigen binding fragment thereof, the HCDR1 comprises the amino acid sequence of SEQ ID NO: 120, the HCDR2 comprises the amino acid sequence of SEQ ID NO: 121, the HCDR3 comprises the amino acid sequence of SEQ ID NO: 122, the LCDR1 comprises the amino acid sequence of SEQ ID NO: 123, the LCDR2 comprises the amino acid sequence of SEQ ID NO: 124, and the LCDR3 comprises the amino acid sequence of SEQ ID NO: 125. In certain embodiments of this type, the antibody is a canine antibody or antigen binding fragment of the canine antibody. In specific embodiments, the antibody is a canine antibody.

In other embodiments of the canine antibodies or antigen binding fragment thereof, the HCDR1 comprises the amino acid sequence of SEQ ID NO: 126, the HCDR2 comprises the amino acid sequence of SEQ ID NO: 127, the HCDR3 comprises the amino acid sequence of SEQ ID NO: 128, the LCDR1 comprises the amino acid sequence of SEQ ID NO: 129, the LCDR2 comprises the amino acid sequence of SEQ ID NO: 130, and the LCDR3 comprises the amino acid sequence of SEQ ID NO: 131. In certain embodiments of this type, the antibody is a canine antibody or antigen binding fragment of the canine antibody. In specific embodiments, the antibody is a canine antibody.

In yet other embodiments of the canine antibodies or antigen binding fragment thereof, the HCDR1 comprises the amino acid sequence of SEQ ID NO: 132, the HCDR2 comprises the amino acid sequence of SEQ ID NO: 133, the HCDR3 comprises the amino acid sequence of SEQ ID NO: 134, the LCDR1 comprises the amino acid sequence of SEQ ID NO: 135, the LCDR2 comprises the amino acid sequence of SEQ ID NO: 136, and the LCDR3 comprises the amino acid sequence of SEQ ID NO: 137. In certain embodiments of this type, the antibody is a canine antibody or antigen binding fragment of the canine antibody. In specific embodiments, the antibody is a canine antibody.

In still other embodiments of the canine antibodies or antigen binding fragment thereof, the HCDR1 comprises the amino acid sequence of SEQ ID NO: 138, the HCDR2 comprises the amino acid sequence of SEQ ID NO: 139, the HCDR3 comprises the amino acid sequence of SEQ ID NO: 140, the LCDR1 comprises the amino acid sequence of SEQ ID NO: 141, the LCDR2 comprises the amino acid sequence of SEQ ID NO: 142, and the LCDR3 comprises the amino acid sequence of SEQ ID NO: 143. In certain embodiments of this type, the antibody is a canine antibody or antigen binding fragment of the canine antibody. In specific embodiments, the antibody is a canine antibody.

In yet other embodiments of the canine antibodies or antigen binding fragment thereof, the HCDR1 comprises the amino acid sequence of SEQ ID NO: 144, the HCDR2 comprises the amino acid sequence of SEQ ID NO: 145, the HCDR3 comprises the amino acid sequence of SEQ ID NO: 146, the LCDR1 comprises the amino acid sequence of SEQ ID NO: 147, the LCDR2 comprises the amino acid sequence of SEQ ID NO: 148, and the LCDR3 comprises the amino acid sequence of SEQ ID NO: 149. In certain embodiments of this type, the antibody is a canine antibody or antigen binding fragment of the canine antibody. In specific embodiments, the antibody is a canine antibody.

In still other embodiments of the canine antibodies or antigen binding fragment thereof, the HCDR1 comprises the amino acid sequence of SEQ ID NO: 150, the HCDR2 comprises the amino acid sequence of SEQ ID NO: 151, the HCDR3 comprises the amino acid sequence of SEQ ID NO: 152, the LCDR1 comprises the amino acid sequence of SEQ ID NO: 153, the LCDR2 comprises the amino acid sequence of SEQ ID NO: 154, and the LCDR3 comprises the amino acid sequence of SEQ ID NO: 155. In certain embodiments of this type, the antibody is a canine antibody or antigen binding fragment of the canine antibody. In specific embodiments, the antibody is a canine antibody.

In yet other embodiments of the canine antibodies or antigen binding fragment thereof, the HCDR1 comprises the amino acid sequence of SEQ ID NO: 150, the HCDR2 comprises the amino acid sequence of SEQ ID NO: 151, the HCDR3 comprises the amino acid sequence of SEQ ID NO: 152, the LCDR1 comprises the amino acid sequence of SEQ ID NO: 153, the LCDR2 comprises the amino acid sequence of SEQ ID NO: 235, and the LCDR3 comprises the amino acid sequence of SEQ ID NO: 155. In certain embodiments of this type, the antibody is a canine antibody or antigen binding fragment of the canine antibody. In specific embodiments, the antibody is a canine antibody.

In yet other embodiments of the canine antibodies or antigen binding fragment thereof, the HCDR1 comprises the amino acid sequence of SEQ ID NO: 248, the HCDR2 comprises the amino acid sequence of SEQ ID NO: 249, the HCDR3 comprises the amino acid sequence of SEQ ID NO: 250, the LCDR1 comprises the amino acid sequence of SEQ ID NO: 251, the LCDR2 comprises the amino acid sequence of SEQ ID NO: 252, and the LCDR3 comprises the amino acid sequence of SEQ ID NO: 253. In certain embodiments of this type, the antibody is a canine antibody or antigen binding fragment of the canine antibody. In specific embodiments, the antibody is a canine antibody.

In particular embodiments of the canine antibodies or antigen binding fragments thereof that comprise the aforementioned sets of six CDRs, (including when the CDRs are in presently disclosed heavy chain variable regions and light chain variable regions, and heavy chains and light chains), when bound to cIL-13, the canine antibodies or antigen binding fragments thereof (i) bind to an epitope comprised by the amino acid sequence of SEQ ID NO: 95, SEQ ID NO: 96, SEQ ID NO: 239, SEQ ID NO: 93, SEQ ID NO: 94, SEQ ID NO: 98, or any combination thereof, (ii) have an IC50 of lower than 50 nM for canine IL-13 mediated STAT-6 phosphorylation in DH82 cells, or (iii) both bind to an epitope comprised by the amino acid sequence of SEQ ID NO: 95, SEQ ID NO: 96, SEQ ID NO: 239, SEQ ID NO: 93, SEQ ID NO: 94, SEQ ID NO: 98, or any combination thereof, and have an IC50 of lower than 50 nM for canine IL-13 mediated STAT-6 phosphorylation in DH82 cells. In more particular embodiments, the canine antibody or antigen binding fragment thereof, when bound to cIL-13 binds to an epitope comprised by the amino acid sequence of SEQ ID NO: 95, SEQ ID NO: 96, SEQ ID NO: 239, or any combination thereof. In even more particular embodiments, the canine antibody or antigen binding fragment thereof binds to one or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In related embodiments, the antibody or antigen binding fragment thereof binds to two or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In other embodiments, the antibody or antigen binding fragment thereof binds to three or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In yet other embodiments, the antibody or antigen binding fragment thereof binds to four or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In still other embodiments, the antibody or antigen binding fragment thereof binds to five or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In yet other embodiments, the antibody binds to all six of the serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2.

In yet other embodiments, the canine antibodies or antigen binding fragments thereof bind cIL-13 and have an IC50 of lower than 25 nM for canine IL-13 mediated STAT-6 phosphorylation in DH82 cells. In still other embodiments, the canine antibodies or antigen binding fragments thereof bind cIL-13 and have an IC50 of between 0.5 to 50 nM for canine IL-13 mediated STAT-6 phosphorylation in DH82 cells. In yet other embodiments, the canine antibodies or antigen binding fragments thereof bind cIL-13 and have an IC50 of between 2 to 25 nM for canine IL-13 mediated STAT-6 phosphorylation in DH82 cells. In still other embodiments, the canine antibodies or antigen binding fragments thereof bind cIL-13 and have an IC50 of between 4 to 25 nM for canine IL-13 mediated STAT-6 phosphorylation in DH82 cells.

In even more particular embodiments, the canine antibody or antigen binding fragment thereof, binds to an epitope of cIL-13 comprised by the amino acid sequence of SEQ ID NO: 95, SEQ ID NO: 96, SEQ ID NO: 239 or any combination thereof, binds to one, two, three, four, five, or six serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2, and has an IC50 of lower than 50 nM, lower than 25 nM, and/or between 2 and/or 4 to 25 nM for canine IL-13 mediated STAT-6 phosphorylation in DH82 cells.

The present invention further provides nucleic acids that comprise nucleotide sequences that encode the specific sets of three heavy chain CDRs of the present invention and nucleic acids that comprise nucleotide sequences that encode the specific sets of three light chain CDRs of the present invention. In a particular embodiment, the nucleic acid encoding a set of three heavy chain CDRs comprises a nucleotide sequence that encodes an HCDR1 comprising the amino acid sequence of SEQ ID NO: 114, a nucleotide sequence that encodes an HCDR2 comprising the amino acid sequence of SEQ ID NO: 115, and a nucleotide sequence that encodes an HCDR3 comprising the amino acid sequence of SEQ ID NO: 116. In a companion embodiment, a nucleic acid encoding a set of three light chain CDRs comprises a nucleotide sequence that encodes an LCDR1 comprising the amino acid sequence of SEQ ID NO: 117, a nucleotide sequence that encodes an LCDR2 comprising the amino acid sequence of SEQ ID NO: 118, and a nucleotide sequence that encodes an LCDR3 comprising the amino acid sequence of SEQ ID NO: 119. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

chain CDRs encodes the light chain of that canine antibody or antigen binding fragment thereof.

In yet another embodiment, the nucleic acid encoding a set of three heavy chain CDRs comprises a nucleotide sequence that encodes an HCDR1 comprising the amino acid sequence of SEQ ID NO: 120, a nucleotide sequence that encodes an HCDR2 comprising the amino acid sequence of SEQ ID NO: 121, and a nucleotide sequence that encodes an HCDR3 comprising the amino acid sequence of SEQ ID NO: 122. In a companion embodiment, the nucleic acid encoding a set of three light chain CDRs comprises a nucleotide sequence that encodes an LCDR1 comprising the amino acid sequence of SEQ ID NO: 123, a nucleotide sequence that encodes an LCDR2 comprising the amino acid sequence of SEQ ID NO: 124, and a nucleotide sequence that encodes an LCDR3 comprising the amino acid sequence of SEQ ID NO: 125. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In still another embodiment, the nucleic acid encoding a set of three heavy chain CDRs comprises a nucleotide sequence that encodes an HCDR1 comprising the amino acid sequence of SEQ ID NO: 126, a nucleotide sequence that encodes an HCDR2 comprising the amino acid sequence of SEQ ID NO: 127, and a nucleotide sequence that encodes an HCDR3 comprising the amino acid sequence of SEQ ID NO: 128. In a companion embodiment, the nucleic acid encoding a set of three light chain CDRs comprises a nucleotide sequence that encodes an LCDR1 comprising the amino acid sequence of SEQ ID NO: 129, a nucleotide sequence that encodes an LCDR2 comprising the amino acid sequence of SEQ ID NO: 130, and a nucleotide sequence that encodes an LCDR3 comprising the amino acid sequence of SEQ ID NO: 131. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In yet another embodiment, the nucleic acid encoding a set of three heavy chain CDRs comprises a nucleotide sequence that encodes an HCDR1 comprising the amino acid sequence of SEQ ID NO: 132, a nucleotide sequence that encodes an HCDR2 comprising the amino acid sequence of SEQ ID NO: 133, and a nucleotide sequence that encodes an HCDR3 comprising the amino acid sequence of SEQ ID NO: 134. In a companion embodiment, the nucleic acid encoding a set of three light chain CDRs comprises a nucleotide sequence that encodes an LCDR1 comprising the amino acid sequence of SEQ ID NO: 135, a nucleotide sequence that encodes an LCDR2 comprising the amino acid sequence of SEQ ID NO: 136, and a nucleotide sequence that encodes an LCDR3 comprising the amino acid sequence of SEQ ID NO: 137. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In still another embodiment, the nucleic acid encoding a set of three heavy chain CDRs comprises a nucleotide sequence that encodes an HCDR1 comprising the amino acid sequence of SEQ ID NO: 138, a nucleotide sequence that encodes an HCDR2 comprising the amino acid sequence of SEQ ID NO: 139, and a nucleotide sequence that encodes an HCDR3 comprising the amino acid sequence of SEQ ID NO: 140. In a companion embodiment, the nucleic acid encoding a set of three light chain CDRs comprises a nucleotide sequence that encodes an LCDR1 comprising the amino acid sequence of SEQ ID NO: 141, a nucleotide sequence that encodes an LCDR2 comprising the amino acid sequence of SEQ ID NO: 142, and a nucleotide sequence that encodes an LCDR3 comprising the amino acid sequence of SEQ ID NO: 143. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In still another embodiment, the nucleic acid encoding a set of three heavy chain CDRs comprises a nucleotide sequence that encodes an HCDR1 comprising the amino acid sequence of SEQ ID NO: 144, a nucleotide sequence that encodes an HCDR2 comprising the amino acid sequence of SEQ ID NO: 145, and a nucleotide sequence that encodes an HCDR3 comprising the amino acid sequence of SEQ ID NO: 146. In a companion embodiment, a nucleic acid encoding a set of three light chain CDRs comprises a nucleotide sequence that encodes an LCDR1 comprising the amino acid sequence of SEQ ID NO: 147, a nucleotide sequence that encodes an LCDR2 comprising the amino acid sequence of SEQ ID NO: 148, and a nucleotide sequence that encodes an LCDR3 comprising the amino acid sequence of SEQ ID NO: 149. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In yet another embodiment, the nucleic acid encoding a set of three heavy chain CDRs comprises a nucleotide sequence that encodes an HCDR1 comprising the amino acid sequence of SEQ ID NO: 150, a nucleotide sequence that encodes an HCDR2 comprising the amino acid sequence of SEQ ID NO: 151, and a nucleotide sequence that encodes an HCDR3 comprising the amino acid sequence of SEQ ID NO: 152. In a companion embodiment, the nucleic acid encoding a set of three light chain CDRs comprises a nucleotide sequence that encodes an LCDR1 comprising the amino acid sequence of SEQ ID NO: 153, a nucleotide sequence that encodes an LCDR2 comprising the amino acid sequence of SEQ ID NO: 154, and a nucleotide sequence that encodes an LCDR3 comprising the amino acid sequence of SEQ ID NO: 155. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In still another embodiment, the nucleic acid encoding a set of three heavy chain CDRs comprises a nucleotide sequence that encodes an HCDR1 comprising the amino acid sequence of SEQ ID NO: 150, a nucleotide sequence that encodes an HCDR2 comprising the amino acid sequence of SEQ ID NO: 151, and a nucleotide sequence that encodes an HCDR3 comprising the amino acid sequence of SEQ ID NO: 152. In a companion embodiment, the nucleic acid encoding a set of three light chain CDRs comprises a nucleotide sequence that encodes an LCDR1 comprising the amino acid sequence of SEQ ID NO: 153, a nucleotide sequence that encodes an LCDR2 comprising the amino acid sequence of SEQ ID NO: 235, and a nucleotide sequence that encodes an LCDR3 comprising the amino acid sequence of SEQ ID NO: 155. In still another embodiment, the nucleic acid encoding a set of three heavy chain CDRs comprises a nucleotide sequence that encodes an HCDR1 comprising the amino acid sequence of SEQ ID NO: 248, a nucleotide sequence that encodes an HCDR2 comprising the amino acid sequence of SEQ ID NO: 249, and a nucleotide sequence that encodes an HCDR3 comprising the amino acid sequence of SEQ ID NO: 250. In a companion embodiment, the nucleic acid encoding a set of three light chain CDRs comprises a nucleotide sequence that encodes an LCDR1 comprising the amino acid sequence of SEQ ID NO: 251, a nucleotide sequence that encodes an LCDR2 comprising the amino acid sequence of SEQ ID NO: 252, and a nucleotide sequence that encodes an LCDR3 comprising the amino acid sequence of SEQ ID NO: 253. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

The canine antibody or antigen binding fragment thereof, can further comprise a heavy chain variable region that comprises at least 90%, 95%, 98%, 99%, or 100% sequence identity with the amino acid sequence of SEQ ID NO: 62. Alternatively, the canine antibody or antigen binding fragment thereof, can further comprise a heavy chain variable region that comprises at least 90%, 95%, 98%, 99%, or 100% sequence identity with the amino acid sequence of SEQ ID NO: 65. In yet other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 68. In yet other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 71. In still other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 74. In yet other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 77. In still other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 80. In still other embodiments, the canine antibody or antigen binding fragment thereof, can further comprise a heavy chain variable region comprising at least 90%, 95%, 98%, 99%, or 100% sequence identity with the amino acid sequence of SEQ ID NO: 244. In certain embodiments of this type, the antibody is a canine antibody or antigen binding fragment of the canine antibody. In specific embodiments, the antibody is a canine antibody.

The canine antibody or antigen binding fragment thereof, also can further comprise a light chain variable region comprising at least 90%, 95%, 98%, 99%, or 100% sequence identity with the amino acid sequence of SEQ ID NO: 63. Alternatively, the canine antibody or antigen binding fragment thereof, also can further comprise a light chain variable region comprising at least 90%, 95%, 98%, 99%, or 100% sequence identity with the amino acid sequence of SEQ ID NO: 66. In yet other embodiments, the canine antibody or antigen binding fragment thereof, can further comprise a light chain variable region comprising at least 90%, 95%, 98%, 99%, or 100% sequence identity with the amino acid sequence of SEQ ID NO: 69. In still other embodiments, the canine antibody or antigen binding fragment thereof, can further comprise a light chain variable region comprising at least 90%, 95%, 98%, 99%, or 100% sequence identity with the amino acid sequence of SEQ ID NO: 72. In yet other embodiments, the canine antibody or antigen binding fragment thereof, can further comprise a light chain variable region comprising at least 90%, 95%, 98%, 99%, or 100% sequence identity with the amino acid sequence of SEQ ID NO: 75. In still other embodiments, the canine antibody or antigen binding fragment thereof, can further comprise a light chain variable region comprising at least 90%, 95%, 98%, 99%, or 100% sequence identity with the amino acid sequence of SEQ ID NO: 78. In yet other embodiments, the canine antibody or antigen binding fragment thereof, can further comprise a light chain variable region comprising at least 90%, 95%, 98%, 99%, or 100% sequence identity with the amino acid sequence of SEQ ID NO: 81. In still other embodiments, the canine antibody or antigen binding fragment thereof, can further comprise a light chain variable region comprising at least 90%, 95%, 98%, 99%, or 100% sequence identity with the amino acid sequence of SEQ ID NO: 234. In still other embodiments, the canine antibody or antigen binding fragment thereof, can further comprise a light chain variable region comprising at least 90%, 95%, 98%, 99%, or 100% sequence identity with the amino acid sequence of SEQ ID NO: 245. In certain embodiments of this type, the antibody is a canine antibody or antigen binding fragment of the canine antibody. In specific embodiments, the antibody is a canine antibody.

The canine antibody or antigen binding fragment thereof can further comprise a heavy chain variable region that comprises at least 90%, 95%, 98%, 99% or 100% sequence identity with the amino acid sequence of SEQ ID NO: 62, while still retaining an HCDR1 that comprises the amino acid sequence of SEQ ID NO: 114, an HCDR2 that comprises the amino acid sequence of SEQ ID NO: 115, and an HCDR3 that comprises the amino acid sequence of SEQ ID NO: 116, and a light chain variable region that comprises at least 90%, 95%, 98%, 99% or 100% sequence identity with the amino acid sequence of SEQ ID NO: 63, while still retaining an LCDR1 that comprises the amino acid sequence of SEQ ID NO: 117, an LCDR2 that comprises the amino acid sequence of SEQ ID NO: 118, and an LCDR3 that comprises the amino acid sequence of SEQ ID NO: 119. In certain embodiments of this type, the antibody is a canine antibody or antigen binding fragment of the canine antibody. In yet other embodiments, the antibody is a canine antibody. In specific embodiments, the canine antibody or antigen binding fragment thereof comprises a heavy chain variable region that comprises the amino acid sequence of SEQ ID NO: 62 and a light chain variable region that comprises the amino acid sequence of SEQ ID NO: 63.

The canine antibody or antigen binding fragment thereof can further comprise a heavy chain variable region that comprises at least 90%, 95%, 98%, 99% or 100% sequence identity with the amino acid sequence of SEQ ID NO: 65, while still retaining an HCDR1 that comprises the amino acid sequence of SEQ ID NO: 120, an HCDR2 that comprises the amino acid sequence of SEQ ID NO: 121, and an HCDR3 that comprises the amino acid sequence of SEQ ID NO: 122, and a light chain variable region that comprises at least 90%, 95%, 98%, 99% or 100%, sequence identity with the amino acid sequence of SEQ ID NO: 66, while still retaining an LCDR1 that comprises the amino acid sequence of SEQ ID NO: 123, an LCDR2 that comprises the amino acid sequence of SEQ ID NO: 124, and an LCDR3 that comprises the amino acid sequence of SEQ ID NO: 125. In certain embodiments of this type, the antibody is a canine antibody or antigen binding fragment of the canine antibody. In still other embodiments, the antibody is a canine antibody. In specific embodiments, the canine antibody or antigen binding fragment thereof comprises a heavy chain variable region that comprises the amino acid sequence of SEQ ID NO: 65 and a light chain variable region that comprises the amino acid sequence of SEQ ID NO: 66.

The canine antibody or antigen binding fragment thereof can further comprise a heavy chain variable region that comprises at least 90%, 95%, 98%, 99% or 100% sequence identity with the amino acid sequence of SEQ ID NO: 68, while still retaining an HCDR1 that comprises the amino acid sequence of SEQ ID NO: 126, an HCDR2 that comprises the amino acid sequence of SEQ ID NO: 127, and an HCDR3 that comprises the amino acid sequence of SEQ ID NO: 128, and a light chain variable region that comprises at least 90%, 95%, 98%, 99% or 100% sequence identity with the amino acid sequence of SEQ ID NO: 69, while still retaining an LCDR1 that comprises the amino acid sequence of SEQ ID NO: 129, an LCDR2 that comprises the amino acid sequence of SEQ ID NO: 130, and an LCDR3 that comprises the amino acid sequence of SEQ ID NO: 131. In certain embodiments of this type, the antibody is a canine antibody or antigen binding fragment of the canine antibody. In still other embodiments, the antibody is a canine antibody. In specific embodiments, the canine antibody or antigen binding fragment thereof comprises a heavy chain variable region that comprises the amino acid sequence of SEQ ID NO: 68 and a light chain variable region that comprises the amino acid sequence of SEQ ID NO: 69.

The canine antibody or antigen binding fragment thereof can further comprise a heavy chain variable region that comprises at least 90%, 95%, 98%, 99% or 100% sequence identity with the amino acid sequence of SEQ ID NO: 71, while still retaining an HCDR1 that comprises the amino acid sequence of SEQ ID NO: 132, an HCDR2 that comprises the amino acid sequence of SEQ ID NO: 133, and an HCDR3 that comprises the amino acid sequence of SEQ ID NO: 134, and a light chain variable region that comprises at least 90%, 95%, 98%, 99% or 100% sequence identity with the amino acid sequence of SEQ ID NO: 72, while still retaining an LCDR1 that comprises the amino acid sequence of SEQ ID NO: 135, an LCDR2 that comprises the amino acid sequence of SEQ ID NO: 136, and an LCDR3 that comprises the amino acid sequence of SEQ ID NO: 137. In certain embodiments of this type, the antibody is a canine antibody or antigen binding fragment of the canine antibody. In still other embodiments, the antibody is a canine antibody. In specific embodiments, the canine antibody or antigen binding fragment thereof comprises a heavy chain variable region that comprises the amino acid sequence of SEQ ID NO: 71 and a light chain variable region that comprises the amino acid sequence of SEQ ID NO: 72.

The canine antibody or antigen binding fragment thereof can further comprise a heavy chain variable region that comprises at least 90%, 95%, 98%, 99% or 100% sequence identity with the amino acid sequence of SEQ ID NO: 74, while still retaining an HCDR1 that comprises the amino acid sequence of SEQ ID NO: 138, an HCDR2 that comprises the amino acid sequence of SEQ ID NO: 139, and an HCDR3 that comprises the amino acid sequence of SEQ ID NO: 140, and a light chain variable region that comprises at least 90%, 95%, 98%, 99% or 100% sequence identity with the amino acid sequence of SEQ ID NO: 75, while still retaining an LCDR1 that comprises the amino acid sequence of SEQ ID NO: 141, an LCDR2 that comprises the amino acid sequence of SEQ ID NO: 142, and an LCDR3 that comprises the amino acid sequence of SEQ ID NO: 143. In certain embodiments of this type, the antibody is a canine antibody or antigen binding fragment of the canine antibody. In still other embodiments, the antibody is a canine antibody. In specific embodiments, the canine antibody or antigen binding fragment thereof comprises a heavy chain variable region that comprises the amino acid sequence of SEQ ID NO: 74 and a light chain variable region that comprises the amino acid sequence of SEQ ID NO: 75.

The canine antibody or antigen binding fragment thereof can further comprise a heavy chain variable region that comprises at least 90%, 95%, 98%, 99% or 100% sequence identity with the amino acid sequence of SEQ ID NO: 77, while still retaining an HCDR1 that comprises the amino acid sequence of SEQ ID NO: 144, an HCDR2 that comprises the amino acid sequence of SEQ ID NO: 145, and an HCDR3 that comprises the amino acid sequence of SEQ ID NO: 146, and a light chain variable region that comprises at least 90%, 95%, 98%, 99% or 100% sequence identity with the amino acid sequence of SEQ ID NO: 78, while still retaining an LCDR1 that comprises the amino acid sequence of SEQ ID NO: 147, an LCDR2 that comprises the amino acid sequence of SEQ ID NO: 148, and an LCDR3 that comprises the amino acid sequence of SEQ ID NO: 149. In certain embodiments of this type, the antibody is a canine antibody or antigen binding fragment of the canine antibody. In still other embodiments, the antibody is a canine antibody. In specific embodiments, the canine antibody or antigen binding fragment thereof comprises a heavy chain variable region that comprises the amino acid sequence of SEQ ID NO: 77 and a light chain variable region that comprises the amino acid sequence of SEQ ID NO: 78.

The canine antibody or antigen binding fragment thereof can further comprise a heavy chain variable region that comprises at least 90%, 95%, 98%, 99% or 100% sequence identity with the amino acid sequence of SEQ ID NO: 80, while still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152, and a light chain variable region that comprises at least 90%, 95%, 98%, 99% or 100% sequence identity with the amino acid sequence of SEQ ID NO: 81, while still retaining an LCDR1 that comprises the amino acid sequence of SEQ ID NO: 153, an LCDR2 that comprises the amino acid sequence of SEQ ID NO: 154, and an LCDR3 that comprises the amino acid sequence of SEQ ID NO: 155. In certain embodiments of this type, the antibody is a canine antibody or antigen binding fragment of the canine antibody. In still other embodiments, the antibody is a canine antibody. In specific embodiments, the canine antibody or antigen binding fragment thereof comprises a heavy chain variable region that comprises the amino acid sequence of SEQ ID NO: 80 and a light chain variable region that comprises the amino acid sequence of SEQ ID NO: 81.

The canine antibody or antigen binding fragment thereof can further comprise a heavy chain variable region that comprises at least 90%, 95%, 98%, 99% or 100% sequence identity with the amino acid sequence of SEQ ID NO: 80, while still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152, and a light chain variable region that comprises at least 90%, 95%, 98%, 99% or 100% sequence identity with the amino acid sequence of SEQ ID NO: 234, while still retaining an LCDR1 that comprises the amino acid sequence of SEQ ID NO: 153, an LCDR2 that comprises the amino acid sequence of SEQ ID NO: 235, and an LCDR3 that comprises the amino acid sequence of SEQ ID NO: 155. In certain embodiments, the antibody is a canine antibody or antigen binding fragment of the canine antibody. In still other embodiments, the antibody is a canine antibody.

In particular embodiments of the canine antibodies or antigen binding fragments thereof that comprise the aforementioned heavy chain variable regions and light chain variable regions, when bound to cIL-13, the canine antibodies or antigen binding fragments thereof (i) bind to an epitope comprised by the amino acid sequence of SEQ ID NO: 95, SEQ ID NO: 96, SEQ ID NO: 239, SEQ ID NO: 93, SEQ ID NO: 94, SEQ ID NO: 98, or any combination thereof, (ii) have an IC50 of lower than 50 nM for canine IL-13 mediated STAT-6 phosphorylation in DH82 cells, or (iii) both bind to an epitope comprised by the amino acid sequence of SEQ ID NO: 95, SEQ ID NO: 96, SEQ ID NO: 239, SEQ ID NO: 93, SEQ ID NO: 94, SEQ ID NO: 98, or any combination thereof, and have an IC50 of lower than 50 nM for canine IL-13 mediated STAT-6 phosphorylation in DH82 cells. In more particular embodiments, the canine antibody or antigen binding fragment thereof, when bound to cIL-13 binds to an epitope comprised by the amino acid sequence of SEQ ID NO: 95, SEQ ID NO: 96, SEQ ID NO: 239, or any combination thereof. In even more particular embodiments, the canine antibody or antigen binding fragment thereof binds to one or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In related embodiments, the antibody or antigen binding fragment thereof binds to two or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In other embodiments, the antibody or antigen binding fragment thereof binds to three or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In yet other embodiments, the antibody or antigen binding fragment thereof binds to four or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In still other embodiments, the antibody or antigen binding fragment thereof binds to five or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In yet other embodiments, the antibody binds to all six of the serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2.

In yet other embodiments, the canine antibodies or antigen binding fragments thereof bind cIL-13 and have an IC50 of lower than 25 nM for canine IL-13 mediated STAT-6 phosphorylation in DH82 cells. In still other embodiments, the canine antibodies or antigen binding fragments thereof bind cIL-13 and have an IC50 of between 0.5 to 50 nM for canine IL-13 mediated STAT-6 phosphorylation in DH82 cells. In yet other embodiments, the canine antibodies or antigen binding fragments thereof bind cIL-13 and have an IC50 of between 2 to 25 nM for canine IL-13 mediated STAT-6 phosphorylation in DH82 cells. In still other embodiments, the canine antibodies or antigen binding fragments thereof bind cIL-13 and have an IC50 of between 4 to 25 nM for canine IL-13 mediated STAT-6 phosphorylation in DH82 cells.

In even more particular embodiments, the canine antibody or antigen binding fragment thereof, binds to an epitope of cIL-13 comprised by the amino acid sequence of SEQ ID NO: 95, SEQ ID NO: 96, SEQ ID NO: 239 or any combination thereof, binds to one, two, three, four, five, or six serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2, and has an IC50 of lower than 50 nM, lower than 25 nM, and/or between 2 and/or 4 to 25 nM for canine IL-13 mediated STAT-6 phosphorylation in DH82 cells.

The present invention further provides nucleic acids that individually encode one of the heavy chain variable regions of the present invention and nucleic acids that individually encode one of the corresponding light chain variable regions of the present invention. The present invention further provides as a pair, a nucleic acid encoding the heavy chain variable region of a canine antibody or antigen binding fragment thereof and a nucleic acid that encodes the corresponding light chain variable region of that canine antibody or antigen binding fragment thereof. The present invention also provides a kit containing this pair of two nucleic acids. In certain embodiments, a nucleic acid encoding the heavy chain variable region encodes the heavy chain of a canine antibody or antigen binding fragment thereof and the corresponding nucleic acid encoding the light chain variable region encodes the light chain of a canine antibody or antigen binding fragment thereof.

In specific embodiments, one nucleic acid encodes a heavy chain variable region that comprises at least 90%, 95%, 98%, 99% or 100% sequence identity with the amino acid sequence of SEQ ID NO: 80, while still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152, and another nucleic acid encodes a corresponding light chain variable region that comprises at least 90%, 95%, 98%, 99% or 100% sequence identity with the amino acid sequence of SEQ ID NO: 234, while still retaining an LCDR1 that comprises the amino acid sequence of SEQ ID NO: 153, an LCDR2 that comprises the amino acid sequence of SEQ ID NO: 235, and an LCDR3 that comprises the amino acid sequence of SEQ ID NO: 155. The present invention further provides as a pair, a nucleic acid encoding the heavy chain variable region of an antibody and a nucleic acid that encodes the corresponding light chain variable region of that antibody. The present invention also provides a kit containing this pair of two nucleic acids. In certain embodiments, a nucleic acid encoding the heavy chain variable region encodes the heavy chain of a canine antibody or antigen binding fragment thereof and the corresponding nucleic acid encoding the light chain variable region encodes the light chain of that canine antibody or antigen binding fragment thereof.

In a more specific embodiment, a nucleic acid encodes a heavy chain variable region of a canine antibody or antigen binding fragment thereof that comprises the amino acid sequence of SEQ ID NO: 80 and in a related embodiment a nucleic acid encodes the corresponding light chain variable region that comprises the amino acid sequence of SEQ ID NO: 234. The present invention further provides as a pair, a nucleic acid encoding the heavy chain variable region that comprises the amino acid sequence of SEQ ID NO: 80 and a nucleic acid encoding the light chain variable region comprising the amino acid sequence of SEQ ID NO: 234. The present invention also provides a kit containing this pair of two nucleic acids. In certain embodiments, a nucleic acid encoding the heavy chain variable region encodes the heavy chain of a canine antibody or antigen binding fragment of thereof and the corresponding nucleic acid encoding the light chain variable region encodes the light chain of that canine antibody or antigen binding fragment thereof.

The canine antibodies of the present invention comprise a canine hinge region. In particular embodiments, the hinge region comprises the amino acid sequence that comprises at least 90%, 95%, or 100% sequence identity with the amino acid sequence of SEQ ID NO: 109. In other embodiments, the hinge region comprises the amino acid sequence that comprises at least 90%, 95%, or 100% sequence identity with the amino acid sequence of SEQ ID NO: 110. In yet other embodiments, the hinge region comprises the amino acid sequence that comprises at least 90%, 95%, or 100% sequence identity with the amino acid sequence of SEQ ID NO: 111. In still other embodiments, the hinge region comprises the amino acid sequence that comprises at least 90%, 95%, or 100% sequence identity with the amino acid sequence of SEQ ID NO: 112. In yet other embodiments, the hinge region comprises the amino acid sequence that comprises at least 90%, 95%, or 100% sequence identity with the amino acid sequence of SEQ ID NO: 113. In certain embodiments of this type, the antibody is a canine antibody or antigen binding fragment of the canine antibody. In specific embodiments, the antibody is a canine antibody.

The canine antibodies of the present invention comprise a canine fragment crystallizable region (cFc). In particular embodiments, the cFc comprises an amino acid sequence that comprises at least 90%, 95%, 98%, 99%, or 100% sequence identity with the amino acid sequence of SEQ ID NO: 99. In other embodiments, the cFc comprises an amino acid sequence that comprises at least 90%, 95%, 98%, 99%, or 100% sequence identity with the amino acid sequence of SEQ ID NO: 100. In still other embodiments, the cFc comprises an amino acid sequence that comprises at least 90%, 95%, 98%, 99%, or 100% sequence identity with the amino acid sequence of SEQ ID NO: 101. In yet other embodiments, the cFc comprises an amino acid sequence that comprises at least 90%, 95%, 98%, 99%, or 100% sequence identity with the amino acid sequence of SEQ ID NO: 102. In still other embodiments, the cFc comprises an amino acid sequence that comprises at least 90%, 95%, 98%, 99%, or 100% sequence identity with the amino acid sequence of SEQ ID NO: 103. In yet other embodiments, the cFc comprises an amino acid sequence that comprises at least 90%, 95%, 98%, 99%, or 100% sequence identity with the amino acid sequence of SEQ ID NO: 104. In still other embodiments, the cFc comprises an amino acid sequence that comprises at least 90%, 95%, 98%, 99%, or 100% sequence identity with the amino acid sequence of SEQ ID NO: 105. In yet other embodiments, the cFc comprises an amino acid sequence that comprises at least 90%, 95%, 98%, 99%, or 100% sequence identity with the amino acid sequence of SEQ ID NO: 106. In certain embodiments of this type, the antibody is a canine antibody or antigen binding fragment of the canine antibody. In specific embodiments, the antibody is a canine antibody.

In yet other embodiments, the cFc comprises an amino acid sequence that comprises at least 90%, 95%, 98%, 99%, or 100% sequence identity with the amino acid sequence of SEQ ID NO: 255. In certain embodiments of this type, the antibody is a canine antibody or antigen binding fragment of the canine antibody. In specific embodiments, the antibody is a canine antibody.

In yet other, preferred, embodiments, the cFc comprises an amino acid sequence that comprises at least 90%, 95%, 98%, 99%, or 100% sequence identity with the amino acid sequence of SEQ ID NO: 256. In certain embodiments of this type, the antibody is a canine antibody or antigen binding fragment of the canine antibody. In specific embodiments, the antibody is a canine antibody.

In yet other embodiments, the cFc comprises an amino acid sequence that comprises at least 90%, 95%, 98%, 99%, or 100% sequence identity with the amino acid sequence of SEQ ID NO: 257. In certain embodiments of this type, the antibody is a canine antibody or antigen binding fragment of the canine antibody. In specific embodiments, the antibody is a canine antibody.

In yet other embodiments, the cFc comprises an amino acid sequence that comprises at least 90%, 95%, 98%, 99%, or 100% sequence identity with the amino acid sequence of SEQ ID NO: 258. In certain embodiments of this type, the antibody is a canine antibody or antigen binding fragment of the canine antibody. In specific embodiments, the antibody is a canine antibody.

In certain embodiments, the canine antibodies comprise a heavy chain that comprises at least 90%, 95%, 98%, 99% or 100% sequence identity with the amino acid sequence of SEQ ID NO: 82, while still retaining an HCDR1 that comprises the amino acid sequence of SEQ ID NO: 144, an HCDR2 that comprises the amino acid sequence of SEQ ID NO: 145, and an HCDR3 that comprises the amino acid sequence of SEQ ID NO: 146 and a light chain that comprises at least 90%, 95%, 98%, 99% or 100% sequence identity with the amino acid sequence of SEQ ID NO: 83, while still retaining an LCDR1 that comprises the amino acid sequence of SEQ ID NO: 147, an LCDR2 that comprises the amino acid sequence of SEQ ID NO: 148, and an LCDR3 that comprises the amino acid sequence of SEQ ID NO: 149. In other embodiments, the canine antibody comprises a heavy chain that comprises the amino acid sequence of SEQ ID NO: 82 and a light chain that comprises the amino acid sequence of SEQ ID NO: 83. The present invention further provides antigen binding fragments of these canine antibodies.

In certain embodiments, the canine antibodies comprise a heavy chain that comprises at least 90%, 95%, 98%, 99% or 100% sequence identity with the amino acid sequence of SEQ ID NO: 82, wherein the amino acid sequence at least comprises a tyrosine residue at amino acid residue position 252 and an aspartic acid residue at amino acid residue position 256, as numbered according to the EU index as in Kabat, and while still retaining an HCDR1 that comprises the amino acid sequence of SEQ ID NO: 144, an HCDR2 that comprises the amino acid sequence of SEQ ID NO: 145, and an HCDR3 that comprises the amino acid sequence of SEQ ID NO: 146 and a light chain that comprises at least 90%, 95%, 98%, 99% or 100% sequence identity with the amino acid sequence of SEQ ID NO: 83, while still retaining an LCDR1 that comprises the amino acid sequence of SEQ ID NO: 147, an LCDR2 that comprises the amino acid sequence of SEQ ID NO: 148, and an LCDR3 that comprises the amino acid sequence of SEQ ID NO: 149. The present invention further provides antigen binding fragments of these canine antibodies.

In certain embodiments, the canine antibodies comprise a heavy chain that comprises at least 90%, 95%, 98%, 99% or 100% sequence identity with the amino acid sequence of SEQ ID NO: 246, while still retaining an HCDR1 that comprises the amino acid sequence of SEQ ID NO: 248, an HCDR2 that comprises the amino acid sequence of SEQ ID NO: 249, and an HCDR3 that comprises the amino acid sequence of SEQ ID NO: 250 and a light chain that comprises at least 90%, 95%, 98%, 99% or 100% sequence identity with the amino acid sequence of SEQ ID NO: 247, while still retaining an LCDR1 that comprises the amino acid sequence of SEQ ID NO: 251, an LCDR2 that comprises the amino acid sequence of SEQ ID NO: 252, and an LCDR3 that comprises the amino acid sequence of SEQ ID NO: 253. In other embodiments, the canine antibody comprises a heavy chain that comprises the amino acid sequence of SEQ ID NO: 246 and a light chain that comprises the amino acid sequence of SEQ ID NO: 247. The present invention further provides antigen binding fragments of these canine antibodies.

In certain embodiments, the canine antibodies comprise a heavy chain that comprises at least 90%, 95%, 98%, 99% or 100% sequence identity with the amino acid sequence of SEQ ID NO: 246, wherein the amino acid sequence at least comprises a tyrosine residue at amino acid residue position 252 and an aspartic acid residue at amino acid residue position 256, as numbered according to the EU index as in Kabat, and while still retaining an HCDR1 that comprises the amino acid sequence of SEQ ID NO: 248, an HCDR2 that comprises the amino acid sequence of SEQ ID NO: 249, and an HCDR3 that comprises the amino acid sequence of SEQ ID NO: 250 and a light chain that comprises at least 90%, 95%, 98%, 99% or 100% sequence identity with the amino acid sequence of SEQ ID NO: 247, while still retaining an LCDR1 that comprises the amino acid sequence of SEQ ID NO: 251, an LCDR2 that comprises the amino acid sequence of SEQ ID NO: 252, and an LCDR3 that comprises the amino acid sequence of SEQ ID NO: 253. The present invention further provides antigen binding fragments of these canine antibodies.

In other embodiments, the canine antibodies comprise a heavy chain that comprises at least 90%, 95%, 98%, 99% or 100% sequence identity with the amino acid sequence of SEQ ID NO: 87, while still retaining an HCDR1 that comprises the amino acid sequence of SEQ ID NO: 114, an HCDR2 that comprises the amino acid sequence of SEQ ID NO: 115, and an HCDR3 that comprises the amino acid sequence of SEQ ID NO: 116 and a light chain that comprises at least 90%, 95%, 98%, 99% or 100% sequence identity with the amino acid sequence of SEQ ID NO: 88, while still retaining an LCDR1 that comprises the amino acid sequence of SEQ ID NO: 117, an LCDR2 that comprises the amino acid sequence of SEQ ID NO: 118, and an LCDR3 that comprises the amino acid sequence of SEQ ID NO: 119. In more particular embodiments, the canine antibody comprises a heavy chain that comprises the amino acid sequence of SEQ ID NO: 87 and a light chain that comprises the amino acid sequence of SEQ ID NO: 88. The present invention further provides antigen binding fragments of these canine antibodies.

In other embodiments, the canine antibodies comprise a heavy chain that comprises at least 90%, 95%, 98%, 99% or 100% sequence identity with the amino acid sequence of SEQ ID NO: 87, wherein the amino acid sequence at least comprises a tyrosine residue at amino acid residue position 252 and an aspartic acid residue at amino acid residue position 256, as numbered according to the EU index as in Kabat, and while still retaining an HCDR1 that comprises the amino acid sequence of SEQ ID NO: 114, an HCDR2 that comprises the amino acid sequence of SEQ ID NO: 115, and an HCDR3 that comprises the amino acid sequence of SEQ ID NO: 116 and a light chain that comprises at least 90%, 95%, 98%, 99% or 100% sequence identity with the amino acid sequence of SEQ ID NO: 88, while still retaining an LCDR1 that comprises the amino acid sequence of SEQ ID NO: 117, an LCDR2 that comprises the amino acid sequence of SEQ ID NO: 118, and an LCDR3 that comprises the amino acid sequence of SEQ ID NO: 119. The present invention further provides antigen binding fragments of these canine antibodies.

In still other embodiments, the canine antibodies comprise a heavy chain that comprises at least 90%, 95%, 98%, 99% or 100% sequence identity with the amino acid sequence of SEQ ID NO: 84, while still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152 and a light chain that comprises at least 90%, 95%, 98%, 99% or 100% sequence identity with the amino acid sequence of SEQ ID NO: 86, while still retaining an LCDR1 that comprises the amino acid sequence of SEQ ID NO: 153, an LCDR2 that comprises the amino acid sequence of SEQ ID NO: 154, and an LCDR3 that comprises the amino acid sequence of SEQ ID NO: 155. In more particular embodiments, the canine antibody comprises a heavy chain that comprises the amino acid sequence of SEQ ID NO: 84 and a light chain that comprises the amino acid sequence of SEQ ID NO: 86. The present invention further provides antigen binding fragments of these canine antibodies.

In yet other embodiments, the canine antibodies comprise a heavy chain that comprises at least 90%, 95%, 98%, 99% or 100% sequence identity with the amino acid sequence of SEQ ID NO: 84, while still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152, and a light chain that comprises at least 90%, 95%, 98%, 99% or 100% sequence identity with the amino acid sequence of SEQ ID NO: 85, while still retaining an LCDR1 that comprises the amino acid sequence of SEQ ID NO: 153, an LCDR2 that comprises the amino acid sequence of SEQ ID NO: 235, and an LCDR3 that comprises the amino acid sequence of SEQ ID NO: 155. In more particular embodiments of this type, the canine antibody or antigen binding fragment thereof, when bound to cIL-13 binds to an epitope comprised by the amino acid sequence of SEQ ID NO: 95, SEQ ID NO: 96, SEQ ID NO: 239, or any combination thereof. In specific embodiments, the canine antibody comprises a heavy chain that comprises the amino acid sequence of SEQ ID NO: 84 and a light chain that comprises the amino acid sequence of SEQ ID NO: 85. The present invention further provides antigen binding fragments of these canine antibodies.

In some preferred embodiments, the canine antibodies provided herein comprise a heavy chain comprising an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152 and a light chain comprising an LCDR1 that comprises the amino acid sequence of SEQ ID NO: 153, an LCDR2 that comprises the amino acid sequence of SEQ ID NO: 235, and an LCDR3 that comprises the amino acid sequence of SEQ ID NO: 155 and that comprises a cFc having an amino acid sequence that comprises at least 90%, 95%, 98%, 99%, or 100% sequence identity with any one of the amino acid sequences selected from the group consisting of: SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 255, SEQ ID NO: 256, SEQ ID NO: 257 and SEQ ID NO: 258. In certain embodiments of this type, the antibody is a canine antibody or antigen binding fragment of the canine antibody. In specific embodiments, the antibody is a canine antibody.

In still other embodiments, the canine antibodies comprise a heavy chain that comprises at least 90%, 95%, 98%, 99% or 100% sequence identity with the amino acid sequence of SEQ ID NO: 240, while still retaining the tyrosine residue at amino acid residue position 252 and the aspartic acid residue at amino acid residue position 256, as numbered according to the EU index as in Kabat, while also still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152 and a light chain that comprises at least 90%, 95%, 98%, 99% or 100% sequence identity with the amino acid sequence of SEQ ID NO: 86, and while still retaining an LCDR1 that comprises the amino acid sequence of SEQ ID NO: 153, an LCDR2 that comprises the amino acid sequence of SEQ ID NO: 154, and an LCDR3 that comprises the amino acid sequence of SEQ ID NO: 155. In more particular embodiments, the canine antibody comprises a heavy chain that comprises the amino acid sequence of SEQ ID NO: 240 and a light chain that comprises the amino acid sequence of SEQ ID NO: 86. The present invention further provides antigen binding fragments of these canine antibodies.

In yet other embodiments, the canine antibodies comprise a heavy chain that comprises at least 90%, 95%, 98%, 99% or 100% sequence identity with the amino acid sequence of SEQ ID NO: 240, while still retaining the tyrosine residue at amino acid residue position 252 and the aspartic acid residue at amino acid residue position 256, as numbered according to the EU index as in Kabat, and while also still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152, and a light chain that comprises at least 90%, 95%, 98%, 99% or 100% sequence identity with the amino acid sequence of SEQ ID NO: 85, while still retaining an LCDR1 that comprises the amino acid sequence of SEQ ID NO: 153, an LCDR2 that comprises the amino acid sequence of SEQ ID NO: 235, and an LCDR3 that comprises the amino acid sequence of SEQ ID NO: 155. In more particular embodiments of this type, the canine antibody or antigen binding fragment thereof, when bound to cIL-13 binds to an epitope comprised by the amino acid sequence of SEQ ID NO: 95, SEQ ID NO: 96, SEQ ID NO: 239, or any combination thereof. In specific embodiments, the canine antibody comprises a heavy chain that comprises the amino acid sequence of SEQ ID NO: 240 and a light chain that comprises the amino acid sequence of SEQ ID NO: 85. The present invention further provides antigen binding fragments of these canine antibodies.

It was found that the canine antibodies comprise a heavy chain that comprises at least 90%, 95%, 98%, 99% or 100%, preferably 100%, sequence identity with the amino acid sequence of SEQ ID NO: 240, while still retaining the tyrosine residue (Y) at amino acid residue position 252 and the aspartic acid (D) residue at amino acid residue position 256, as numbered according to the EU index as in Kabat, and while also still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152, and a light chain that comprises at least 90%, 95%, 98%, 99% or 100%, preferably 100%, sequence identity with the amino acid sequence of SEQ ID NO: 85, while still retaining an LCDR1 that comprises the amino acid sequence of SEQ ID NO: 153, an LCDR2 that comprises the amino acid sequence of SEQ ID NO: 235, and an LCDR3 that comprises the amino acid sequence of SEQ ID NO: 155 has an improved half-life.

The cFc of the heavy chain of the canine antibodies of the present invention or antigen binding fragments thereof, can further comprise one or more amino acid residue substitutions, wherein the amino acid residue positions are numbered according to the EU index as in Kabat. In certain embodiments, one amino acid residue substitution is at amino acid residue position 252. In a particular embodiment of this type, the substitution at amino acid residue position 252 is with a tyrosine residue. In other embodiments, one amino acid residue substitution is at amino acid residue position 254. In a particular embodiment of this type, the substitution at amino acid residue position 254 is with a threonine residue. In still other embodiments, one amino acid residue substitution is at amino acid residue position 256. In a particular embodiment of this type, the substitution at amino acid residue position 256 is with an aspartic acid residue or a glutamic acid residue. In yet other embodiments, one amino acid residue substitution is at amino acid residue position 308. In a particular embodiment of this type, the substitution at amino acid residue position 308 is with a proline residue. In still other embodiments, an amino acid residue substitution is at amino acid residue position 433. In a particular embodiment of this type, the substitution at amino acid residue position 433 is with a lysine residue or a leucine residue. In yet other embodiments, an amino acid residue substitution is at amino acid residue position 434. In a particular embodiment of this type, the substitution at amino acid residue position 434 is with a phenylalanine residue, a histidine residue, or a tyrosine residue. In a more particular embodiment of this type, the substitution at amino acid residue position 434 is with a histidine residue. In still other embodiments, an amino acid residue substitution is at amino acid residue position 436. In a particular embodiment of this type, the substitution at amino acid residue position 436 is with a threonine residue.

In some embodiments, there is provided for a canine IL13 antibody according to the present invention, said antibody comprising a modified Fc region having an amino acid sequence of at least 90%, 95%, 98%, 99%, or 100% sequence identity to SEQ ID NO: 101 or SEQ ID NO: 102, said amino acid sequence comprising a least a substitution at amino acid residue position 434 as numbered according to the EU index as in Kabat, wherein said substitution is with a histidine (H) residue.

In some embodiments, there is provided for a canine IL13 antibody comprising a modified Fc region having an amino acid sequence of at least 95%, preferably 100%, sequence identity to SEQ ID NO: 257 or SEQ ID NO: 258, wherein the substitution at amino acid residue position 434 as numbered according to the EU index as in Kabat that is with a histidine residue is retained, and comprising a heavy chain comprising an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152, and comprising a light chain comprising an LCDR1 that comprises the amino acid sequence of SEQ ID NO: 153, an LCDR2 that comprises the amino acid sequence of SEQ ID NO: 235, and an LCDR3 that comprises the amino acid sequence of SEQ ID NO: 155.

In related embodiments, it was found that canine IL13 antibodies comprising such a modified Fc region, and preferably comprising said HCDRs and/or LCDRs, have an extended half-life compared to canine IL13 antibodies with an unmodified cFc.

The cFc of the heavy chain of the canine antibodies of the present invention or antigen binding fragments thereof, can further comprise two or more amino acid residue substitutions, wherein the amino acid residue positions are numbered according to the EU index as in Kabat. In certain embodiments, one amino acid residue substitution is at amino acid residue position 252 and a second amino acid residue substitution is at amino acid residue position 256. In a particular embodiment of this type, the substitution at amino acid residue position 252 is with a tyrosine residue and the substitution at amino acid residue position 256 is with an aspartic acid residue.

As described and provided herein, it was found that the substitution at amino acid residue position 252 with a tyrosine residue and the substitution at amino acid residue position 256 with an aspartic acid residue extend the antibody half-life, preferably of any one or more of the canine antibodies disclosed herein. For example, the substitution at amino acid residue position 252 with a tyrosine residue and the substitution at amino acid residue position 256 with an aspartic acid residue may extend the half-life of a canine IL13 antibody provided herein.

In preferred embodiments, the substitution at amino acid residue position 252 with a tyrosine residue and the substitution at amino acid residue position 256 with an aspartic acid residue is in the amino acid sequence of a canine IL13 antibody with a modified Fc region, represented by heavy chain SEQ ID NO: 240 and light chain SEQ ID NO: 85.

In one non-limiting example, a canine IL13 antibody with a modified Fc region had an extended antibody half-life in vivo compared to a canine IL13 antibody with an unmodified Fc region.

In a further non-limiting example, see, Example 8, a canine IL13 antibody with a modified Fc region had an extended antibody half-life in vivo compared to a canine IL13 antibody with an unmodified Fc region. In one non-limiting example, a canine IL13 antibody with a modified Fc region having an amino acid sequence according to SEQ ID NO: 255 or SEQ ID NO: 256 and comprising a heavy chain comprising an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152, and comprising a light chain comprising an LCDR1 that comprises the amino acid sequence of SEQ ID NO: 153, an LCDR2 that comprises the amino acid sequence of SEQ ID NO: 235, and an LCDR3 that comprises the amino acid sequence of SEQ ID NO: 155 was found to exhibit an extended antibody half-life in vivo compared to a canine IL13 antibody with an unmodified Fc region having an amino acid sequence according to SEQ ID NO: 101 or SEQ ID NO: 102 and comprising a heavy chain comprising an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152, and comprising a light chain comprising an LCDR1 that comprises the amino acid sequence of SEQ ID NO: 153, an LCDR2 that comprises the amino acid sequence of SEQ ID NO: 235, and an LCDR3 that comprises the amino acid sequence of SEQ ID NO: 155.

Hence, in some embodiments, there is provided for a canine IL13 antibody comprising a modified Fc region having an amino acid sequence comprising at least 90%, 95%, 98%, 99%, or 100% sequence identity according to SEQ ID NO: 255 or SEQ ID NO: 256, wherein the substitution comprising a tyrosine residue at amino acid residue position 252 and an aspartic acid residue at amino acid residue position 256 is retained, and comprising a heavy chain comprising an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152, and comprising a light chain comprising an LCDR1 that comprises the amino acid sequence of SEQ ID NO: 153, an LCDR2 that comprises the amino acid sequence of SEQ ID NO: 235, and an LCDR3 that comprises the amino acid sequence of SEQ ID NO: 155.

In other embodiments, one amino acid residue substitution is at amino acid residue position 256 and a second amino acid residue substitution is at amino acid residue position 434. In a particular embodiment of this type, the substitution at amino acid residue position 256 is with an aspartic acid residue and the substitution at amino acid residue position 434 is with a tyrosine residue. In an alternative embodiment of this type, the substitution at amino acid residue position 256 is with an aspartic acid residue and the substitution at amino acid residue position 434 is with a histidine residue. In yet other embodiments, one amino acid residue substitution is at amino acid residue position 308 and a second amino acid residue substitution is at amino acid residue position 434. In a particular embodiment of this type, the substitution at amino acid residue position 308 is with a proline residue and the substitution at amino acid residue position 434 is with a tyrosine residue. In an alternative embodiment of this type, the substitution at amino acid residue position 308 is with a proline residue and the substitution at amino acid residue position 434 is with a histidine residue. In still other embodiments, one amino acid residue substitution is at amino acid residue position 433 and a second amino acid residue substitution is at amino acid residue position 434. In a particular embodiment of this type, the substitution at amino acid residue position 433 is with a lysine residue and the substitution at amino acid residue position 434 is with a tyrosine residue. In an alternative embodiment of this type, the substitution at amino acid residue position 433 is with a lysine residue and the substitution at amino acid residue position 434 is with a histidine residue. In yet another embodiment of this type, the substitution at amino acid residue position 433 is with a lysine residue and the substitution at amino acid residue position 434 is with a phenylalanine residue. In still another embodiment of this type, the substitution at amino acid residue position 433 is with a leucine residue and the substitution at amino acid residue position 434 is with a phenylalanine residue. In yet another embodiment of this type, the substitution at amino acid residue position 433 is with a leucine residue and the substitution at amino acid residue position 434 is with a tyrosine residue. In still another embodiment of this type, the substitution at amino acid residue position 433 is with a leucine residue and the substitution at amino acid residue position 434 is with a histidine residue. In yet other embodiments, one amino acid residue substitution is at amino acid residue position 434 and a second amino acid residue substitution is at amino acid residue position 436. In a particular embodiment of this type, the substitution is at amino acid residue position 434 with a tyrosine residue and the substitution at amino acid residue position 436 is with a threonine residue. In another embodiment of this type, the substitution at amino acid residue position 434 is with a phenylalanine residue and the substitution at amino acid residue position 436 is with a threonine residue. In an alternative embodiment of this type, the substitution at amino acid residue position 434 is with a histidine residue and the substitution at amino acid residue position 436 is with a threonine residue.

The cFc of the heavy chain of the canine antibodies of the present invention or antigen binding fragments thereof, can further comprise three or more amino acid residue substitutions, wherein the amino acid residue positions are numbered according to the EU index as in Kabat. In certain embodiments, one amino acid residue substitution is at amino acid residue position 252, a second amino acid residue substitution is at amino acid residue position 254, and a third amino acid residue substitution is at amino acid residue position 256. In a particular embodiment of this type, the substitution at amino acid residue position 252 is with a tyrosine residue, the substitution at amino acid residue position 254 is with a with a threonine residue, and the substitution at amino acid residue position 256 is with a glutamic acid residue. In other embodiments, one amino acid residue substitution is at amino acid residue position 256, a second amino acid residue substitution is at amino acid residue position 308, and a third amino acid residue substitution is at amino acid residue position 434. In a particular embodiment of this type, the substitution at amino acid residue position 256 is with an aspartic acid residue, the substitution at amino acid residue position 308 is with a proline residue, and the substitution at amino acid residue position 434 is with a tyrosine residue. In yet other embodiments, one amino acid residue substitution is at amino acid residue position 433, a second amino acid residue substitution is at amino acid residue position 434, and a third amino acid residue substitution is at amino acid residue position 436. In a particular embodiment of this type, the substitution at amino acid residue position 433 is with a lysine residue, the substitution at amino acid residue position 434 is with a phenylalanine residue, and the substitution at amino acid residue position 436 is with a threonine residue. In another embodiment of this type, the substitution at amino acid residue position 433 is with a lysine residue, the substitution at amino acid residue position 434 is with a tyrosine residue, and the substitution at amino acid residue position 436 is with a threonine residue. In yet another embodiment of this type, the substitution at amino acid residue position 433 is with a lysine residue, the substitution at amino acid residue position 434 is with a histidine residue, and the substitution at amino acid residue position 436 is with a threonine residue. In still another embodiment of this type, the substitution at amino acid residue position 433 is with a leucine residue, the substitution at amino acid residue position 434 is with a phenylalanine residue, and the substitution at amino acid residue position 436 is with a threonine residue. In yet another embodiment of this type, the substitution at amino acid residue position 433 is with a leucine residue, the substitution at amino acid residue position 434 is with a tyrosine residue, and the substitution at amino acid residue position 436 is with a threonine residue. In still another embodiment of this type, the substitution at amino acid residue position 433 is with a leucine residue, the substitution at amino acid residue position 434 is with a histidine residue, and the substitution at amino acid residue position 436 is with a threonine residue.

In particular embodiments of the canine antibodies or antigen binding fragments thereof that comprise the aforementioned heavy chains and light chains, when bound to cIL-13, the canine antibodies or antigen binding fragments thereof (i) bind to an epitope comprised by the amino acid sequence of SEQ ID NO: 95, SEQ ID NO: 96, SEQ ID NO: 239, SEQ ID NO: 93, SEQ ID NO: 94, SEQ ID NO: 98, or any combination thereof, (ii) have an IC50 of lower than 50 nM for canine IL-13 mediated STAT-6 phosphorylation in DH82 cells, or (iii) both bind to an epitope comprised by the amino acid sequence of SEQ ID NO: 95, SEQ ID NO: 96, SEQ ID NO: 239, SEQ ID NO: 93, SEQ ID NO: 94, SEQ ID NO: 98, or any combination thereof, and have an IC50 of lower than 50 nM for canine IL-13 mediated STAT-6 phosphorylation in DH82 cells. In more particular embodiments, the canine antibody or antigen binding fragment thereof, when bound to cIL-13 binds to an epitope comprised by the amino acid sequence of SEQ ID NO: 95, SEQ ID NO: 96, SEQ ID NO: 239, or any combination thereof. In even more particular embodiments, the canine antibody or antigen binding fragment thereof binds to one or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In related embodiments, the antibody or antigen binding fragment thereof binds to two or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In other embodiments, the antibody or antigen binding fragment thereof binds to three or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In yet other embodiments, the antibody or antigen binding fragment thereof binds to four or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In still other embodiments, the antibody or antigen binding fragment thereof binds to five or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In yet other embodiments, the antibody binds to all six of the serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2.

In yet other embodiments, the canine antibodies or antigen binding fragments thereof bind cIL-13 and have an IC50 of lower than 25 nM for canine IL-13 mediated STAT-6 phosphorylation in DH82 cells. In still other embodiments, the canine antibodies or antigen binding fragments thereof bind cIL-13 and have an IC50 of between 0.5 to 50 nM for canine IL-13 mediated STAT-6 phosphorylation in DH82 cells. In yet other embodiments, the canine antibodies or antigen binding fragments thereof bind cIL-13 and have an IC50 of between 2 to 25 nM for canine IL-13 mediated STAT-6 phosphorylation in DH82 cells. In still other embodiments, the canine antibodies or antigen binding fragments thereof bind cIL-13 and have an IC50 of between 4 to 25 nM for canine IL-13 mediated STAT-6 phosphorylation in DH82 cells.

In even more particular embodiments, the canine antibody or antigen binding fragment thereof, binds to an epitope of cIL-13 comprised by the amino acid sequence of SEQ ID NO: 95, SEQ ID NO: 96, SEQ ID NO: 239 or any combination thereof, binds to one, two, three, four, five, or six serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2, and has an IC50 of lower than 50 nM, lower than 25 nM, and/or between 2 and/or 4 to 25 nM for canine IL-13 mediated STAT-6 phosphorylation in DH82 cells.

The present invention further provides nucleic acids that individually encode one of the heavy chains of the antibodies of the present invention and nucleic acids that individually encode one of the corresponding light chains of the antibodies of the present invention or an antigen binding fragment thereof. The present invention further provides as a pair, a nucleic acid encoding the heavy chain or an antigen binding fragment thereof of an antibody of the present invention and a nucleic acid that encodes the corresponding light chain or an antigen binding fragment thereof of that antibody of the present invention. The present invention also provides a kit containing this pair of two nucleic acids.

In certain embodiments, the nucleic acid encodes a heavy chain of a canine antibody or an antigen binding fragment of the heavy chain that comprises at least 90%, 95%, 98%, 99% or 100% sequence identity with the amino acid sequence of SEQ ID NO: 87, while still retaining an HCDR1 that comprises the amino acid sequence of SEQ ID NO: 114, an HCDR2 that comprises the amino acid sequence of SEQ ID NO: 115, and an HCDR3 that comprises the amino acid sequence of SEQ ID NO: 116, and another nucleic acid that encodes a corresponding light chain of that antibody that comprises at least 90%, 95%, 98%, 99% or 100% sequence identity with the amino acid sequence of SEQ ID NO: 88, while still retaining an LCDR1 that comprises the amino acid sequence of SEQ ID NO: 117, an LCDR2 that comprises the amino acid sequence of SEQ ID NO: 118, and an LCDR3 that comprises the amino acid sequence of SEQ ID NO: 119. The present invention further provides as a pair, the nucleic acid encoding the heavy chain of the canine antibody and the nucleic acid that encodes the corresponding light chain of that canine antibody. The present invention also provides a kit containing this pair of two nucleic acids.

In other embodiments, a nucleic acid encodes a heavy chain of a canine antibody or antigen binding fragment thereof that comprises the amino acid sequence of SEQ ID NO: 87 and another nucleic acid that encodes a corresponding light chain of that antibody that comprises the amino acid sequence of SEQ ID NO: 88. The present invention further provides as a pair, a nucleic acid encoding the heavy chain that comprises the amino acid sequence of SEQ ID NO: 87 and a nucleic acid that encodes the corresponding light chain that comprises the amino acid sequence of SEQ ID NO: 88. The present invention also provides a kit containing this pair of two nucleic acids.

In yet other embodiments, the nucleic acid encodes a heavy chain of a canine antibody or antigen binding fragment thereof that comprises at least 90%, 95%, 98%, 99% or 100% sequence identity with the amino acid sequence of SEQ ID NO: 82, while still retaining an HCDR1 that comprises the amino acid sequence of SEQ ID NO: 144, an HCDR2 that comprises the amino acid sequence of SEQ ID NO: 145, and an HCDR3 that comprises the amino acid sequence of SEQ ID NO: 146, and another nucleic acid that encodes a corresponding light chain of that antibody that comprises at least 90%, 95%, 98%, 99% or 100% sequence identity with the amino acid sequence of SEQ ID NO: 83, while still retaining an LCDR1 that comprises the amino acid sequence of SEQ ID NO: 147, an LCDR2 that comprises the amino acid sequence of SEQ ID NO: 148, and an LCDR3 that comprises the amino acid sequence of SEQ ID NO: 149. The present invention further provides as a pair, a nucleic acid encoding the heavy chain of the canine antibody and a nucleic acid that encodes the corresponding light chain of that canine antibody. The present invention also provides a kit containing this pair of two nucleic acids.

In still other embodiments, a nucleic acid encodes a heavy chain of a canine antibody or antigen binding fragment thereof that comprises the amino acid sequence of SEQ ID NO: 82 and the corresponding light chain of that antibody that comprises the amino acid sequence of SEQ ID NO: 83. The present invention further provides as a pair, a nucleic acid encoding the heavy chain that comprises the amino acid sequence of SEQ ID NO: 82 and a nucleic acid that encodes the corresponding light chain that comprises the amino acid sequence of SEQ ID NO: 83. The present invention also provides a kit containing this pair of two nucleic acids.

In particular embodiments, the nucleic acid encodes a heavy chain of a canine antibody or antigen binding fragment thereof that comprises at least 90%, 95%, 98%, 99% or 100% sequence identity with the amino acid sequence of SEQ ID NO: 84, while still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152, and another nucleic acid that encodes a corresponding light chain of that antibody that comprises at least 90%, 95%, 98%, 99% or 100% sequence identity with the amino acid sequence of SEQ ID NO: 86, while still retaining an LCDR1 that comprises the amino acid sequence of SEQ ID NO: 153, an LCDR2 that comprises the amino acid sequence of SEQ ID NO: 154, and an LCDR3 that comprises the amino acid sequence of SEQ ID NO: 155. The present invention further provides as a pair, a nucleic acid encoding the heavy chain of the canine antibody and a nucleic acid that encodes the corresponding light chain of that canine antibody. The present invention also provides a kit containing this pair of two nucleic acids.

In a more particular embodiment, a nucleic acid encodes a heavy chain of a canine antibody that comprises the amino acid sequence of SEQ ID NO: 84 and another nucleic acid that encodes a corresponding light chain of that antibody that comprises the amino acid sequence of SEQ ID NO: 86. The present invention further provides as a pair, a nucleic acid encoding the heavy chain that comprises the amino acid sequence of SEQ ID NO: 84 and a nucleic acid that encodes the corresponding light chain that comprises the amino acid sequence of SEQ ID NO: 86. The present invention also provides a kit containing this pair of two nucleic acids.

In specific embodiments, the nucleic acid encodes a heavy chain of a canine antibody or antigen binding fragment thereof that comprises at least 90%, 95%, 98%, 99% or 100% sequence identity with the amino acid sequence of SEQ ID NO: 84, while still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152, and another nucleic acid that encodes a corresponding light chain of that antibody that comprises at least 90%, 95%, 98%, 99% or 100% sequence identity with the amino acid sequence of SEQ ID NO: 85, while still retaining an LCDR1 that comprises the amino acid sequence of SEQ ID NO: 153, an LCDR2 that comprises the amino acid sequence of SEQ ID NO: 235, and an LCDR3 that comprises the amino acid sequence of SEQ ID NO: 155. The present invention further provides as a pair, a nucleic acid encoding the heavy chain of the canine antibody and a nucleic acid that encodes the corresponding light chain of that canine antibody. The present invention also provides a kit containing this pair of two nucleic acids.

In a more specific embodiment, a nucleic acid encodes a heavy chain of a canine antibody or antigen binding fragment thereof that comprises the amino acid sequence of SEQ ID NO: 84 and another nucleic acid that encodes a corresponding light chain of that antibody that comprises the amino acid sequence of SEQ ID NO: 85. The present invention further provides as a pair, a nucleic acid encoding the heavy chain that comprises the amino acid sequence of SEQ ID NO: 84 and a nucleic acid that encodes the corresponding light chain that comprises the amino acid sequence of SEQ ID NO: 85. The present invention also provides a kit containing this pair of two nucleic acids.

In related particular embodiments, the nucleic acid encodes a heavy chain of a canine antibody or antigen binding fragment thereof that comprises at least 90%, 95%, 98%, 99% or 100% sequence identity with the amino acid sequence of SEQ ID NO: 240, while still retaining the tyrosine residue at amino acid residue position 252 and the aspartic acid residue at amino acid residue position 256, as numbered according to the EU index as in Kabat, while also still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152, and another nucleic acid that encodes a corresponding light chain of that antibody that comprises at least 90%, 95%, 98%, 99% or 100% sequence identity with the amino acid sequence of SEQ ID NO: 86, while still retaining an LCDR1 that comprises the amino acid sequence of SEQ ID NO: 153, an LCDR2 that comprises the amino acid sequence of SEQ ID NO: 154, and an LCDR3 that comprises the amino acid sequence of SEQ ID NO: 155. The present invention further provides as a pair, a nucleic acid encoding the heavy chain of the canine antibody and a nucleic acid that encodes the corresponding light chain of that canine antibody. The present invention also provides a kit containing this pair of two nucleic acids.

In a more particular embodiment, a nucleic acid encodes a heavy chain of a canine antibody that comprises the amino acid sequence of SEQ ID NO: 240 and another nucleic acid that encodes a corresponding light chain of that antibody that comprises the amino acid sequence of SEQ ID NO: 86. The present invention further provides as a pair, a nucleic acid encoding the heavy chain that comprises the amino acid sequence of SEQ ID NO: 240 and a nucleic acid that encodes the corresponding light chain that comprises the amino acid sequence of SEQ ID NO: 86. The present invention also provides a kit containing this pair of two nucleic acids.

In specific embodiments, the nucleic acid encodes a heavy chain of a canine antibody or antigen binding fragment thereof that comprises at least 90%, 95%, 98%, 99% or 100% sequence identity with the amino acid sequence of SEQ ID NO: 240, while still retaining the tyrosine residue at amino acid residue position 252 and the aspartic acid residue at amino acid residue position 256, as numbered according to the EU index as in Kabat, while also still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152, and another nucleic acid that encodes a corresponding light chain of that antibody that comprises at least 90%, 95%, 98%, 99% or 100% sequence identity with the amino acid sequence of SEQ ID NO: 85, while still retaining an LCDR1 that comprises the amino acid sequence of SEQ ID NO: 153, an LCDR2 that comprises the amino acid sequence of SEQ ID NO: 235, and an LCDR3 that comprises the amino acid sequence of SEQ ID NO: 155. The present invention further provides as a pair, a nucleic acid encoding the heavy chain of the canine antibody and a nucleic acid that encodes the corresponding light chain of that canine antibody. The present invention also provides a kit containing this pair of two nucleic acids.

In a more specific embodiment, a nucleic acid encodes a heavy chain of a canine antibody or antigen binding fragment thereof that comprises the amino acid sequence of SEQ ID NO: 240 and another nucleic acid that encodes a corresponding light chain of that antibody that comprises the amino acid sequence of SEQ ID NO: 85. The present invention further provides as a pair, a nucleic acid encoding the heavy chain that comprises the amino acid sequence of SEQ ID NO: 240 and a nucleic acid that encodes the corresponding light chain that comprises the amino acid sequence of SEQ ID NO: 85. The present invention also provides a kit containing this pair of two nucleic acids.

In yet other embodiments, the nucleic acid encodes a heavy chain of a canine antibody or antigen binding fragment thereof that comprises at least 90%, 95%, 98%, 99% or 100% sequence identity with the amino acid sequence of SEQ ID NO: 246, while still retaining an HCDR1 that comprises the amino acid sequence of SEQ ID NO: 248, an HCDR2 that comprises the amino acid sequence of SEQ ID NO: 249, and an HCDR3 that comprises the amino acid sequence of SEQ ID NO: 250, and another nucleic acid that encodes a corresponding light chain of that antibody that comprises at least 90%, 95%, 98%, 99% or 100% sequence identity with the amino acid sequence of SEQ ID NO: 247, while still retaining an LCDR1 that comprises the amino acid sequence of SEQ ID NO: 251, an LCDR2 that comprises the amino acid sequence of SEQ ID NO: 252, and an LCDR3 that comprises the amino acid sequence of SEQ ID NO: 253. The present invention further provides as a pair, a nucleic acid encoding the heavy chain of the canine antibody and a nucleic acid that encodes the corresponding light chain of that canine antibody. The present invention also provides a kit containing this pair of two nucleic acids.

In still other embodiments, a nucleic acid encodes a heavy chain of a canine antibody or antigen binding fragment thereof that comprises the amino acid sequence of SEQ ID NO: 246 and the corresponding light chain of that antibody that comprises the amino acid sequence of SEQ ID NO: 247. The present invention further provides as a pair, a nucleic acid encoding the heavy chain that comprises the amino acid sequence of SEQ ID NO: 246 and a nucleic acid that encodes the corresponding light chain that comprises the amino acid sequence of SEQ ID NO: 247. The present invention also provides a kit containing this pair of two nucleic acids.

The present invention further provides canine antibodies or antigen binding fragments thereof, that when bound to cIL-13 (i) bind to an epitope comprised by the amino acid sequence of SEQ ID NO: 95, SEQ ID NO: 96, SEQ ID NO: 239, SEQ ID NO: 93, SEQ ID NO: 94, SEQ ID NO: 98, or any combination thereof, (ii) have an IC50 of lower than 50 nM for canine IL-13 mediated STAT-6 phosphorylation in DH82 cells, or (iii) both bind to an epitope comprised by the amino acid sequence of SEQ ID NO: 95, SEQ ID NO: 96, SEQ ID NO: 239, SEQ ID NO: 93, SEQ ID NO: 94, SEQ ID NO: 98, or any combination thereof, and have an IC50 of lower than 50 nM for canine IL-13 mediated STAT-6 phosphorylation in DH82 cells. In particular embodiments, the canine antibody or antigen binding fragment thereof, when bound to cIL-13 binds to an epitope comprised by the amino acid sequence of SEQ ID NO: 95, SEQ ID NO: 96, SEQ ID NO: 239, or any combination thereof. In more particular embodiments, the canine antibody or antigen binding fragment thereof binds to one or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In related embodiments, the antibody or antigen binding fragment thereof binds to two or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In other embodiments, the antibody or antigen binding fragment thereof binds to three or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In yet other embodiments, the antibody or antigen binding fragment thereof binds to four or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In still other embodiments, the antibody or antigen binding fragment thereof binds to five or more serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2. In yet other embodiments, the antibody binds to all six of the serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2.

In yet other embodiments, the canine antibodies or antigen binding fragments thereof bind cIL-13 and have an IC50 of lower than 25 nM for canine IL-13 mediated STAT-6 phosphorylation in DH82 cells. In still other embodiments, the canine antibodies or antigen binding fragments thereof bind cIL-13 and have an IC50 of between 0.5 to 50 nM for canine IL-13 mediated STAT-6 phosphorylation in DH82 cells. In yet other embodiments, the canine antibodies or antigen binding fragments thereof bind cIL-13 and have an IC50 of between 2 to 25 nM for canine IL-13 mediated STAT-6 phosphorylation in DH82 cells. In still other embodiments, the canine antibodies or antigen binding fragments thereof bind cIL-13 and have an IC50 of between 4 to 25 nM for canine IL-13 mediated STAT-6 phosphorylation in DH82 cells.

In even more particular embodiments, the canine antibody or antigen binding fragment thereof, binds to an epitope of cIL-13 comprised by the amino acid sequence of SEQ ID NO: 95, SEQ ID NO: 96, SEQ ID NO: 239 or any combination thereof, binds to one, two, three, four, five, or six serine residues of cIL-13 at the following amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2, and has an IC50 of lower than 50 nM, lower than 25 nM, and/or between 2 and/or 4 to 25 nM for canine IL-13 mediated STAT-6 phosphorylation in DH82 cells.

The present invention further provides expression vectors that comprise and express one or more of the nucleic acids of the present invention. In particular embodiments, the expression vector comprises and expresses a nucleic acid encoding a heavy chain of a canine antibody of the present invention. In other embodiments, the expression vector comprises and expresses a nucleic acid encoding a light chain of that canine antibody. In more specific embodiments, the expression vector comprises a pair of nucleic acids, wherein one of the pair of nucleic acids comprises a nucleotide sequence that encodes the light chain of a specific canine antibody whereas the other of the pair of nucleic acids comprises a nucleotide sequence that encodes the heavy chain of said specific canine antibody. In related embodiments, the present invention provides a pair of expression vectors, one expression vector comprising the one of a pair of nucleic acids that comprises a nucleotide sequence that encodes the heavy chain of the specific canine antibody, whereas the other expression vector comprises the other of the pair of nucleic acids that comprises the nucleotide sequence that encodes the light chain of that specific canine antibody. The present invention also provides host cells that comprise one or more expression vectors of the present invention or one or more pairs of expression vectors. The present invention further provides pairs of host cells, with one of the host cell pairs comprising one of a pair of expression vectors, which comprises one of a pair of nucleic acids that comprises a nucleotide sequence encoding the heavy chain of the specific canine antibody, whereas the other one of the pair of host cells comprises the other one of the pair of expression vectors, which comprises the other one of the pair of nucleic acids that comprises a nucleotide sequence encoding the light chain of that specific canine antibody.

The present invention also provides pharmaceutical compositions and/or methods of treating atopic dermatitis with the pharmaceutical compositions comprising the canine antibodies and/or antigen binding fragments of the antibodies and a pharmaceutically acceptable carrier or diluent. In certain embodiments, the pharmaceutical compositions comprise canine antibodies that comprise a heavy chain variable region that comprises at least 90%, 95%, 98%, 99% or 100% sequence identity with the amino acid sequence of SEQ ID NO: 80, while still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152 and a light chain variable region that comprises at least 90%, 95%, 98%, 99% or 100% sequence identity with the amino acid sequence of SEQ ID NO: 81, while still retaining an LCDR1 that comprises the amino acid sequence of SEQ ID NO: 153, an LCDR2 that comprises the amino acid sequence of SEQ ID NO: 154, and an LCDR3 that comprises the amino acid sequence of SEQ ID NO: 155. In more particular embodiments, the pharmaceutical compositions comprise canine antibodies that comprise a heavy chain variable region that comprises the amino acid sequence of SEQ ID NO: 80 and a light chain variable region that comprises the amino acid sequence of SEQ ID NO: 81. In yet other embodiments, the pharmaceutical compositions comprise canine antibodies that comprise a heavy chain variable region that comprises at least 90%, 95%, 98%, 99% or 100% sequence identity with the amino acid sequence of SEQ ID NO: 80, while still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152, and a light chain variable region that comprises at least 90%, 95%, 98%, 99% or 100% sequence identity with the amino acid sequence of SEQ ID NO: 234, while still retaining an LCDR1 that comprises the amino acid sequence of SEQ ID NO: 153, an LCDR2 that comprises the amino acid sequence of SEQ ID NO: 235, and an LCDR3 that comprises the amino acid sequence of SEQ ID NO: 155. In specific embodiments, the pharmaceutical compositions comprise canine antibodies that comprise a heavy chain variable region that comprises the amino acid sequence of SEQ ID NO: 80 and a light chain variable region that comprises the amino acid sequence of SEQ ID NO: 234.

In particular embodiments, the pharmaceutical compositions comprise canine antibodies that comprise a heavy chain that comprises at least 90%, 95%, 98%, 99% or 100% sequence identity with the amino acid sequence of SEQ ID NO: 84, while still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152 and a light chain that comprises at least 90%, 95%, 98%, 99% or 100% sequence identity with the amino acid sequence of SEQ ID NO: 86, while still retaining an LCDR1 that comprises the amino acid sequence of SEQ ID NO: 153, an LCDR2 that comprises the amino acid sequence of SEQ ID NO: 154, and an LCDR3 that comprises the amino acid sequence of SEQ ID NO: 155. In more particular embodiments, the pharmaceutical compositions comprise canine antibodies that comprise a heavy chain that comprises the amino acid sequence of SEQ ID NO: 84 and a light chain that comprises the amino acid sequence of SEQ ID NO: 86. In yet other embodiments, the pharmaceutical compositions comprise canine antibodies that comprise a heavy chain that comprises at least 90%, 95%, 98%, 99% or 100% sequence identity with the amino acid sequence of SEQ ID NO: 84, while still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152, and a light chain that comprises at least 90%, 95%, 98%, 99% or 100% sequence identity with the amino acid sequence of SEQ ID NO: 85, while still retaining an LCDR1 that comprises the amino acid sequence of SEQ ID NO: 153, an LCDR2 that comprises the amino acid sequence of SEQ ID NO: 235, and an LCDR3 that comprises the amino acid sequence of SEQ ID NO: 155. In specific embodiments, the pharmaceutical compositions comprise canine antibodies that comprise a heavy chain that comprises the amino acid sequence of SEQ ID NO: 84 and a light chain that comprises the amino acid sequence of SEQ ID NO: 85.

The present invention further provides pharmaceutical compositions and/or methods of treating atopic dermatitis with the pharmaceutical compositions comprising canine antibodies that comprise a heavy chain that comprises at least 90%, 95%, 98%, 99% or 100% sequence identity with the amino acid sequence of SEQ ID NO: 240, while still retaining the tyrosine residue at amino acid residue position 252 and the aspartic acid residue at amino acid residue position 256, as numbered according to the EU index as in Kabat, while also still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152 and a light chain that comprises at least 90%, 95%, 98%, 99% or 100% sequence identity with the amino acid sequence of SEQ ID NO: 86, and while still retaining an LCDR1 that comprises the amino acid sequence of SEQ ID NO: 153, an LCDR2 that comprises the amino acid sequence of SEQ ID NO: 154, and an LCDR3 that comprises the amino acid sequence of SEQ ID NO: 155. In more particular embodiments, the pharmaceutical compositions comprise canine antibodies that comprise a heavy chain that comprises the amino acid sequence of SEQ ID NO: 240 and a light chain that comprises the amino acid sequence of SEQ ID NO: 86. In yet other embodiments, the pharmaceutical compositions comprise canine antibodies that comprise a heavy chain that comprises at least 90%, 95%, 98%, 99% or 100% sequence identity with the amino acid sequence of SEQ ID NO: 240, while still retaining the tyrosine residue at amino acid residue position 252 and the aspartic acid residue at amino acid residue position 256, as numbered according to the EU index as in Kabat, and while also still retaining an HCDR1 that comprises the amino acid sequence of SEQ ID NO: 150, an HCDR2 that comprises the amino acid sequence of SEQ ID NO: 151, and an HCDR3 that comprises the amino acid sequence of SEQ ID NO: 152, and a light chain that comprises at least 90%, 95%, 98%, 99% or 100% sequence identity with the amino acid sequence of SEQ ID NO: 85, while still retaining an LCDR1 that comprises the amino acid sequence of SEQ ID NO: 153, an LCDR2 that comprises the amino acid sequence of SEQ ID NO: 235, and an LCDR3 that comprises the amino acid sequence of SEQ ID NO: 155. In specific embodiments, the pharmaceutical compositions comprise canine antibodies that comprise a heavy chain that comprises the amino acid sequence of SEQ ID NO: 240 and a light chain that comprises the amino acid sequence of SEQ ID NO: 85.

In addition, pharmaceutical compositions are provided that comprise an expression vector comprising both a nucleic acid encoding a heavy chain of a canine antibody of the present invention and a nucleic acid encoding the light chain of that canine antibody, and a pharmaceutically acceptable carrier or diluent. In other embodiments, the pharmaceutical composition comprises a pharmaceutically acceptable carrier or diluent and two expression vectors, one comprising a nucleic acid encoding a heavy chain of a canine antibody of the present invention and the other expression vector comprises a nucleic acid encoding the light chain of that canine antibody. Such pharmaceutical compositions are capable of expressing the canine antibody and/or antigen binding fragments of the antibody of the present invention, in vivo. Any of the pharmaceutical compositions of the present invention can further comprise a canine or caninized antibody that binds canine interleukin-4 (cIL-4), a canine or caninized antibody that binds a canine interleukin-4 receptor alpha (cIL-4Rα), a canine or caninized antibody that binds canine interleukin-31 receptor alpha (cIL-31RA), or a canine or caninized antibody that binds canine interleukin-31 (cIL-31) or any combination thereof. In yet another embodiment, a pharmaceutical composition is provided for use in a method of decreasing the skin inflammation associated with atopic dermatitis in a canine, comprising administering to the canine in need thereof a therapeutically effective amount of the canine antibody that binds cIL-13 of the present invention, or antigen binding fragment thereof, or an expression vector of the present invention, or a pair of expression vectors of the present invention, or any combination thereof, and a pharmaceutically acceptable carrier or diluent; wherein said method is used for the treatment of atopic dermatitis. In yet further embodiments, the present invention provides for a pharmaceutical composition for use in the treatment of atopic dermatitis, preferably atopic dermatitis in a canine, wherein the treatment comprises administering to the canine in need thereof a therapeutically effective amount of the canine antibody that binds cIL-13 of the present invention, or antigen binding fragment thereof, or an expression vector of the present invention, or a pair of expression vectors of the present invention, or any combination thereof, and a pharmaceutically acceptable carrier or diluent. The present invention further provides a pharmaceutical composition for use in the treatment of atopic dermatitis, said treatment comprising the administering one or more of the herein provided pharmaceutical compositions to an animal subject that has atopic dermatitis. Preferably, the animal subject is a canine.

The present invention further provides additional methods of treating atopic dermatitis comprising administering one of the aforesaid pharmaceutical compositions to an animal subject that has atopic dermatitis. In particular embodiments, the animal subject is a canine. The present invention also provides methods of aiding in the blocking of the skin inflammation associated with atopic dermatitis, comprising administering to an animal subject in need thereof of a therapeutically effective amount of a pharmaceutical composition of the present invention. In particular embodiments, the animal subject is a canine.

In specific embodiments, methods of aiding in the blocking of the skin inflammation associated with atopic dermatitis are provided comprising administering to a subject in need thereof a therapeutically effective amount of a pharmaceutical composition of the present invention. In particular embodiments, the animal subject is a canine. In other embodiments, methods of aiding in the blocking of the skin inflammation, pruritus, or both skin inflammation and pruritus associated with atopic dermatitis, comprises administering to a subject in need thereof a therapeutically effective amount of the pharmaceutical composition of the present invention comprising a canine antibody of the present invention that binds cIL-13 and blocks the binding of cIL-13 with the IL-13 receptor alpha 1 (cIL-13R alpha 1), together with a canine or caninized antibody that binds canine interleukin-4 (cIL-4), a canine or caninized antibody that binds canine interleukin-4 receptor alpha (cIL-4Rα), a canine or caninized antibody that binds canine interleukin-31 receptor alpha (cIL-31RA), a canine or caninized antibody that binds canine interleukin-31 (cIL-31) or any combination thereof. In particular embodiments, the animal subject is a canine.

In addition, the present invention provides methods of producing a canine antibody or antigen binding fragment thereof that binds canine IL-13. In particular embodiments, the method includes culturing host cells comprising one or more expression vectors that encode and express the light chain of a canine antibody of the present invention or the heavy chain of that canine antibody, or both the light chain of a canine antibody of the present invention and the heavy chain of that canine antibody in a culture medium (or culture media) under conditions in which the nucleic acid(s) is expressed, thereby producing a polypeptide comprising the light chain of a canine antibody of the present invention, the heavy chain of that canine antibody or both the light chain of a canine antibody of the present invention and the heavy chain of that canine antibody. The polypeptides are then recovered from the host cells or culture medium (or culture media). In certain embodiments, the polypeptides comprising the light chain of a canine antibody of the present invention and the polypeptides comprising the heavy chain of that canine antibody are combined with each under conditions that are conducive to form a canine antibody.

The present invention further provides single-chain Fv (scFv) antibodies that bind cIL-13, and comprise a set of six complementary determining regions, three of which are heavy chain CDRs (HCDR1, HCDR2, and HCDR3) and three of which are light chain CDRs (LCDR1, LCDR2, and LCDR3). In particular embodiments of the scFvs, the HCDR1 comprises the amino acid sequence of SEQ ID NO: 114, the HCDR2 comprises the amino acid sequence of SEQ ID NO: 115, the HCDR3 comprises the amino acid sequence of SEQ ID NO: 116, the LCDR1 comprises the amino acid sequence of SEQ ID NO: 117, the LCDR2 comprises the amino acid sequence of SEQ ID NO: 118, and the LCDR3 comprises the amino acid sequence of SEQ ID NO: 119.

In alternative embodiments of the scFvs, the HCDR1 comprises the amino acid sequence of SEQ ID NO: 120, the HCDR2 comprises the amino acid sequence of SEQ ID NO: 121, the HCDR3 comprises the amino acid sequence of SEQ ID NO: 122, the LCDR1 comprises the amino acid sequence of SEQ ID NO: 123, the LCDR2 comprises the amino acid sequence of SEQ ID NO: 124, and the LCDR3 comprises the amino acid sequence of SEQ ID NO: 125.

In other embodiments of the scFvs, the HCDR1 comprises the amino acid sequence of SEQ ID NO: 126, the HCDR2 comprises the amino acid sequence of SEQ ID NO: 127, the HCDR3 comprises the amino acid sequence of SEQ ID NO: 128, the LCDR1 comprises the amino acid sequence of SEQ ID NO: 129, the LCDR2 comprises the amino acid sequence of SEQ ID NO: 130, and the LCDR3 comprises the amino acid sequence of SEQ ID NO: 131.

In yet other embodiments of the scFvs, the HCDR1 comprises the amino acid sequence of SEQ ID NO: 132, the HCDR2 comprises the amino acid sequence of SEQ ID NO: 133, the HCDR3 comprises the amino acid sequence of SEQ ID NO: 134, the LCDR1 comprises the amino acid sequence of SEQ ID NO: 135, the LCDR2 comprises the amino acid sequence of SEQ ID NO: 136, and the LCDR3 comprises the amino acid sequence of SEQ ID NO: 137.

In still other embodiments of the scFvs, the HCDR1 comprises the amino acid sequence of SEQ ID NO: 138, the HCDR2 comprises the amino acid sequence of SEQ ID NO: 139, the HCDR3 comprises the amino acid sequence of SEQ ID NO: 140, the LCDR1 comprises the amino acid sequence of SEQ ID NO: 141, the LCDR2 comprises the amino acid sequence of SEQ ID NO: 142, and the LCDR3 comprises the amino acid sequence of SEQ ID NO: 143.

In yet other embodiments of the scFvs, the HCDR1 comprises the amino acid sequence of SEQ ID NO: 144, the HCDR2 comprises the amino acid sequence of SEQ ID NO: 145, the HCDR3 comprises the amino acid sequence of SEQ ID NO: 146, the LCDR1 comprises the amino acid sequence of SEQ ID NO: 147, the LCDR2 comprises the amino acid sequence of SEQ ID NO: 148, and the LCDR3 comprises the amino acid sequence of SEQ ID NO: 149.

In still other embodiments of the scFvs, the HCDR1 comprises the amino acid sequence of SEQ ID NO: 150, the HCDR2 comprises the amino acid sequence of SEQ ID NO: 151, the HCDR3 comprises the amino acid sequence of SEQ ID NO: 152, the LCDR1 comprises the amino acid sequence of SEQ ID NO: 153, the LCDR2 comprises the amino acid sequence of SEQ ID NO: 154, and the LCDR3 comprises the amino acid sequence of SEQ ID NO: 155.

In yet other embodiments of the scFvs, the HCDR1 comprises the amino acid sequence of SEQ ID NO: 150, the HCDR2 comprises the amino acid sequence of SEQ ID NO: 151, the HCDR3 comprises the amino acid sequence of SEQ ID NO: 152, the LCDR1 comprises the amino acid sequence of SEQ ID NO: 153, the LCDR2 comprises the amino acid sequence of SEQ ID NO: 235, and the LCDR3 comprises the amino acid sequence of SEQ ID NO: 155.

In yet other embodiments of the scFvs, the HCDR1 comprises the amino acid sequence of SEQ ID NO: 248, the HCDR2 comprises the amino acid sequence of SEQ ID NO: 249, the HCDR3 comprises the amino acid sequence of SEQ ID NO: 250, the LCDR1 comprises the amino acid sequence of SEQ ID NO: 251, the LCDR2 comprises the amino acid sequence of SEQ ID NO: 252, and the LCDR3 comprises the amino acid sequence of SEQ ID NO: 253.

The present invention further provides single-chain Fv (scFv) antibodies that bind cIL-13 and comprise a heavy chain variable region that comprises at least 90%, 95%, 98%, 99%, or 100% sequence identity with the amino acid sequence of SEQ ID NO: 62 and the light chain variable region comprises at least 90%, 95%, 98%, 99%, or 100% sequence identity with the amino acid sequence of SEQ ID NO: 63. In other embodiments of the scFV, the heavy chain variable region comprises at least 90%, 95%, 98%, 99%, or 100% sequence identity with the amino acid sequence of SEQ ID NO: 65 and the light chain variable region comprises at least 90%, 95%, 98%, 99%, or 100% sequence identity with the amino acid sequence of SEQ ID NO: 66. In still other embodiments of the scFV, the heavy chain variable region comprises at least 90%, 95%, 98%, 99%, or 100% sequence identity with the amino acid sequence of SEQ ID NO: 68 and the light chain variable region comprises at least 90%, 95%, 98%, 99%, or 100% sequence identity with the amino acid sequence of SEQ ID NO: 69.

In yet other embodiments of the scFV, the heavy chain variable region comprises at least 90%, 95%, 98%, 99%, or 100% sequence identity with the amino acid sequence of SEQ ID NO: 71 and the light chain variable region comprises at least 90%, 95%, 98%, 99%, or 100% sequence identity with the amino acid sequence of SEQ ID NO: 72. In still other embodiments of the scFV, the heavy chain variable region comprises at least 90%, 95%, 98%, 99%, or 100% sequence identity with the amino acid sequence of SEQ ID NO: 74 and the light chain variable region comprises at least 90%, 95%, 98%, 99%, or 100% sequence identity with the amino acid sequence of SEQ ID NO: 75. In yet other embodiments of the scFV, the heavy chain variable region comprises at least 90%, 95%, 98%, 99%, or 100% sequence identity with the amino acid sequence of SEQ ID NO: 77 and the light chain variable region comprises at least 90%, 95%, 98%, 99%, or 100% sequence identity with the amino acid sequence of SEQ ID NO: 78.

In still other embodiments of the scFV, the heavy chain variable region comprises at least 90%, 95%, 98%, 99%, or 100% sequence identity with the amino acid sequence of SEQ ID NO: 80 and the light chain variable region comprises at least 90%, 95%, 98%, 99%, or 100% sequence identity with the amino acid sequence of SEQ ID NO: 81. In yet other embodiments of the scFV, the heavy chain variable region comprises at least 90%, 95%, 98%, 99%, or 100% sequence identity with the amino acid sequence of SEQ ID NO: 80 and the light chain variable region comprises at least 90%, 95%, 98%, 99%, or 100% sequence identity with the amino acid sequence of SEQ ID NO: 234.

In yet other embodiments of the scFV, the heavy chain variable region comprises at least 90%, 95%, 98%, 99%, or 100% sequence identity with the amino acid sequence of SEQ ID NO: 244 and the light chain variable region comprises at least 90%, 95%, 98%, 99%, or 100% sequence identity with the amino acid sequence of SEQ ID NO: 245.

The present invention further provides a single-chain Fv (scFv) antibody that binds cIL-13 and comprises the amino acid sequence of SEQ ID NO: 61. In still other embodiments, the scFV comprises the amino acid sequence of SEQ ID NO: 64. In yet other embodiments, the scFV, comprises the amino acid sequence of SEQ ID NO: 67. In still other embodiments, the scFV, comprises the amino acid sequence of SEQ ID NO: 70. In yet other embodiments, the scFV, comprises the amino acid sequence of SEQ ID NO: 73. In still other embodiments, the scFV, comprises the amino acid sequence of SEQ ID NO: 76. In yet other embodiments, the scFV, comprises the amino acid sequence of SEQ ID NO: 79. In yet other embodiments, the scFV comprises the amino acid sequence of SEQ ID NO: 243. In yet other embodiments, the scFV comprises the amino acid sequence of SEQ ID NO: 254.

The present invention further provides individual nucleic acids that individually encode each of the scFvs of the present invention that bind cIL-13.

In another aspect, the present invention further relates to canine antibodies and scFvs that bind to canine interleukin-4 (cIL-4). The present invention also relates to antigen binding fragments of the canine antibodies, which bind to cIL-4. The present invention further provides these canine antibodies, and the antigen binding fragments thereof, that both bind cIL-4 and block the binding of cIL-4 to the canine Type II heterodimer interleukin-4 receptor (Type II cIL-4R) and more particularly to canine interleukin-4 receptor alpha (cIL-4Rα). In certain embodiments, a canine antibody or an antigen binding fragment thereof binds cIL-4 and blocks the binding of cIL-4 to the Type II cIL-4R and more particularly blocks the binding of cIL-4 to CIL-4Rα.

Accordingly, the present invention provides canine antibodies, including isolated canine antibodies, that bind cIL-4 and comprise a heavy chain and a light chain that together comprise a set of six complementary determining regions (CDRs), three of which are heavy chain CDRs: CDR heavy 1 (HCDR1), CDR heavy 2 (HCDR2) and CDR heavy 3 (HCDR3) and three of which are light chain CDRs: CDR light 1 (LCDR1), CDR light 2 (LCDR2), and CDR light 3 (LCDR3), the canine heavy chain variable regions of the heavy chains of the canine antibodies, the light chain variable regions of the canine antibodies, and antigen binding fragments of the heavy and light chains of the canine antibodies, and related scFvs.

The present invention further provides nucleic acids, including isolated nucleic acids, that encode: any of the sets of three heavy chain CDRs (HCDR1, HCDR2, and HCDR3), canine heavy chain variable regions of the heavy chains of the canine antibodies and/or the heavy chains of the canine antibodies or antigen binding fragments thereof, any of the sets of three light chain CDRs (LCDR1, LCDR2, and LCDR3), the light chain variable regions of the canine antibodies and/or the light chains of the canine antibodies or antigen binding fragments of the present invention, and related scFvs.

The canine antibodies that bind cIL-4, comprise a heavy chain and a light chain that together comprise a set of six CDRs, three of which are heavy chain CDRs and three of which are light chain CDRs. In particular embodiments, the canine antibodies or antigen binding fragment thereof comprise the HCDR1 that comprises the amino acid sequence of SEQ ID NO: 156, the HCDR2 comprises the amino acid sequence of SEQ ID NO: 157, the HCDR3 comprises the amino acid sequence of SEQ ID NO: 158, the LCDR1 comprises the amino acid sequence of SEQ ID NO: 159, the LCDR2 comprises the amino acid sequence of SEQ ID NO: 160, and the LCDR3 comprises the amino acid sequence of SEQ ID NO: 161. In certain embodiments of this type, the antibody is a canine antibody or antigen binding fragment of the canine antibody. In specific embodiments, the antibody is a canine antibody.

In alternative embodiments of the canine antibodies or antigen binding fragment thereof, the HCDR1 comprises the amino acid sequence of SEQ ID NO: 162, the HCDR2 comprises the amino acid sequence of SEQ ID NO: 163, the HCDR3 comprises the amino acid sequence of SEQ ID NO: 164, the LCDR1 comprises the amino acid sequence of SEQ ID NO: 165, the LCDR2 comprises the amino acid sequence of SEQ ID NO: 166, and the LCDR3 comprises the amino acid sequence of SEQ ID NO: 167. In certain embodiments of this type, the antibody is a canine antibody or antigen binding fragment of the canine antibody. In specific embodiments, the antibody is a canine antibody.

In related embodiments of the canine antibodies or antigen binding fragment thereof, the HCDR1 comprises the amino acid sequence of SEQ ID NO: 162, the HCDR2 comprises the amino acid sequence of SEQ ID NO: 237, the HCDR3 comprises the amino acid sequence of SEQ ID NO: 164, the LCDR1 comprises the amino acid sequence of SEQ ID NO: 165, the LCDR2 comprises the amino acid sequence of SEQ ID NO: 166, and the LCDR3 comprises the amino acid sequence of SEQ ID NO: 167. In certain embodiments of this type, the antibody is a canine antibody or antigen binding fragment of the canine antibody. In specific embodiments, the antibody is a canine antibody.

In other embodiments of the canine antibodies or antigen binding fragment thereof, the HCDR1 comprises the amino acid sequence of SEQ ID NO: 168, the HCDR2 comprises the amino acid sequence of SEQ ID NO: 169, the HCDR3 comprises the amino acid sequence of SEQ ID NO: 170, the LCDR1 comprises the amino acid sequence of SEQ ID NO: 171, the LCDR2 comprises the amino acid sequence of SEQ ID NO: 172, and the LCDR3 comprises the amino acid sequence of SEQ ID NO: 173. In certain embodiments of this type, the antibody is a canine antibody or antigen binding fragment of the canine antibody. In specific embodiments, the antibody is a canine antibody.

In yet other embodiments of the canine antibodies or antigen binding fragment thereof, the HCDR1 comprises the amino acid sequence of SEQ ID NO: 174, the HCDR2 comprises the amino acid sequence of SEQ ID NO: 175, the HCDR3 comprises the amino acid sequence of SEQ ID NO: 176, the LCDR1 comprises the amino acid sequence of SEQ ID NO: 177, the LCDR2 comprises the amino acid sequence of SEQ ID NO: 178, and the LCDR3 comprises the amino acid sequence of SEQ ID NO: 179. In certain embodiments of this type, the antibody is a canine antibody or antigen binding fragment of the canine antibody. In specific embodiments, the antibody is a canine antibody.

In still other embodiments of the canine antibodies or antigen binding fragment thereof, the HCDR1 comprises the amino acid sequence of SEQ ID NO: 180, the HCDR2 comprises the amino acid sequence of SEQ ID NO: 181, the HCDR3 comprises the amino acid sequence of SEQ ID NO: 182, the LCDR1 comprises the amino acid sequence of SEQ ID NO: 183, the LCDR2 comprises the amino acid sequence of SEQ ID NO: 184, and the LCDR3 comprises the amino acid sequence of SEQ ID NO: 185. In certain embodiments of this type, the antibody is a canine antibody or antigen binding fragment of the canine antibody. In specific embodiments, the antibody is a canine antibody.

In yet other embodiments of the canine antibodies or antigen binding fragment thereof, the HCDR1 comprises the amino acid sequence of SEQ ID NO: 186, the HCDR2 comprises the amino acid sequence of SEQ ID NO: 187, the HCDR3 comprises the amino acid sequence of SEQ ID NO: 188, the LCDR1 comprises the amino acid sequence of SEQ ID NO: 189, the LCDR2 comprises the amino acid sequence of SEQ ID NO: 190, and the LCDR3 comprises the amino acid sequence of SEQ ID NO: 191. In certain embodiments of this type, the antibody is a canine antibody or antigen binding fragment of the canine antibody. In specific embodiments, the antibody is a canine antibody.

In still other embodiments of the canine antibodies or antigen binding fragment thereof, the HCDR1 comprises the amino acid sequence of SEQ ID NO: 192, the HCDR2 comprises the amino acid sequence of SEQ ID NO: 193, the HCDR3 comprises the amino acid sequence of SEQ ID NO: 194, the LCDR1 comprises the amino acid sequence of SEQ ID NO: 195, the LCDR2 comprises the amino acid sequence of SEQ ID NO: 196, and the LCDR3 comprises the amino acid sequence of SEQ ID NO: 197. In certain embodiments of this type, the antibody is a canine antibody or antigen binding fragment of the canine antibody. In specific embodiments, the antibody is a canine antibody.

In yet other embodiments of the canine antibodies or antigen binding fragment thereof, the HCDR1 comprises the amino acid sequence of SEQ ID NO: 198, the HCDR2 comprises the amino acid sequence of SEQ ID NO: 199, the HCDR3 comprises the amino acid sequence of SEQ ID NO: 200, the LCDR1 comprises the amino acid sequence of SEQ ID NO: 201, the LCDR2 comprises the amino acid sequence of SEQ ID NO: 202, and the LCDR3 comprises the amino acid sequence of SEQ ID NO: 203. In certain embodiments of this type, the antibody is a canine antibody or antigen binding fragment of the canine antibody. In specific embodiments, the antibody is a canine antibody.

In still other embodiments of the canine antibodies or antigen binding fragment thereof, the HCDR1 comprises the amino acid sequence of SEQ ID NO: 204, the HCDR2 comprises the amino acid sequence of SEQ ID NO: 205, the HCDR3 comprises the amino acid sequence of SEQ ID NO: 206, the LCDR1 comprises the amino acid sequence of SEQ ID NO: 207, the LCDR2 comprises the amino acid sequence of SEQ ID NO: 208, and the LCDR3 comprises the amino acid sequence of SEQ ID NO: 209. In certain embodiments of this type, the antibody is a canine antibody or antigen binding fragment of the canine antibody. In specific embodiments, the antibody is a canine antibody.

In yet other embodiments of the canine antibodies or antigen binding fragment thereof, the HCDR1 comprises the amino acid sequence of SEQ ID NO: 210, the HCDR2 comprises the amino acid sequence of SEQ ID NO: 211, the HCDR3 comprises the amino acid sequence of SEQ ID NO: 212, the LCDR1 comprises the amino acid sequence of SEQ ID NO: 213, the LCDR2 comprises the amino acid sequence of SEQ ID NO: 214, and the LCDR3 comprises the amino acid sequence of SEQ ID NO: 215. In certain embodiments of this type, the antibody is a canine antibody or antigen binding fragment of the canine antibody. In specific embodiments, the antibody is a canine antibody.

In still other embodiments of the canine antibodies or antigen binding fragment thereof, the HCDR1 comprises the amino acid sequence of SEQ ID NO: 216, the HCDR2 comprises the amino acid sequence of SEQ ID NO: 217, the HCDR3 comprises the amino acid sequence of SEQ ID NO: 218, the LCDR1 comprises the amino acid sequence of SEQ ID NO: 219, the LCDR2 comprises the amino acid sequence of SEQ ID NO: 220, and the LCDR3 comprises the amino acid sequence of SEQ ID NO: 221. In certain embodiments of this type, the antibody is a canine antibody or antigen binding fragment of the canine antibody. In specific embodiments, the antibody is a canine antibody.

In yet other embodiments of the canine antibodies or antigen binding fragment thereof, the HCDR1 comprises the amino acid sequence of SEQ ID NO: 222, the HCDR2 comprises the amino acid sequence of SEQ ID NO: 223, the HCDR3 comprises the amino acid sequence of SEQ ID NO: 224, the LCDR1 comprises the amino acid sequence of SEQ ID NO: 225, the LCDR2 comprises the amino acid sequence of SEQ ID NO: 226, and the LCDR3 comprises the amino acid sequence of SEQ ID NO: 227. In certain embodiments of this type, the antibody is a canine antibody or antigen binding fragment of the canine antibody. In specific embodiments, the antibody is a canine antibody.

In yet other embodiments of the canine antibodies or antigen binding fragment thereof, the HCDR1 comprises the amino acid sequence of SEQ ID NO: 228, the HCDR2 comprises the amino acid sequence of SEQ ID NO: 229, the HCDR3 comprises the amino acid sequence of SEQ ID NO: 230, the LCDR1 comprises the amino acid sequence of SEQ ID NO: 231, the LCDR2 comprises the amino acid sequence of SEQ ID NO: 232, and the LCDR3 comprises the amino acid sequence of SEQ ID NO: 233. In certain embodiments of this type, the antibody is a canine antibody or antigen binding fragment of the canine antibody. In specific embodiments, the antibody is a canine antibody.

In particular embodiments of the canine antibodies or antigen binding fragments thereof that comprise the aforementioned sets of six CDRs, (including when the CDRs are in presently disclosed heavy chain variable regions and light chain variable regions, and heavy chains and light chains), when bound to cIL-4, the canine antibodies or antigen binding fragments thereof (i) bind to an epitope comprised by the amino acid sequence of SEQ ID NO: 89, SEQ ID NO: 90, SEQ ID NO: 91, SEQ ID NO: 92, SEQ ID NO: 238, or any combination thereof, (ii) have an IC50 of lower than 40 nM for canine IL-4 mediated STAT-6 phosphorylation in DH82 cells, or (iii) both bind to an epitope comprised by the amino acid sequence of SEQ ID NO: 89, SEQ ID NO: 90, SEQ ID NO: 91, SEQ ID NO: 92, SEQ ID NO: 238, or any combination thereof, and have an IC50 of lower than 40 nM for canine IL-4 mediated STAT-6 phosphorylation in DH82 cells. In more particular embodiments, the canine antibody or antigen binding fragment thereof, when bound to cIL-4 binds to an epitope comprised by the amino acid sequence of SEQ ID NO: 89, SEQ ID NO: 90, SEQ ID NO: 91, SEQ ID NO: 92, SEQ ID NO: 238, or any combination thereof. In certain embodiments, the canine antibody or antigen binding fragment thereof binds to one or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In other embodiments, the canine antibody or antigen binding fragment thereof binds to two or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In yet other embodiments, the canine antibody or antigen binding fragment thereof binds to three or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In still other embodiments, the canine antibody or antigen binding fragment thereof binds to four or more amino acid residues of cIL-4 at the following amino acid residue positions: 6 (T₆), 8 (Kg), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In yet other embodiments, the canine antibody or antigen binding fragment thereof binds to five or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In still other embodiments, the canine antibody or antigen binding fragment thereof binds to six or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In particular embodiments, the canine antibody or antigen binding fragment thereof binds to three to five amino acid residues of cIL-4 at the following amino acid residue positions: 6 (T₆), 8 (K₈), 12 (K₁₂), 74 (S₇₄), and 79 (T₇₉) of the amino acid sequence of SEQ ID NO: 1. In other particular embodiments, the canine antibody or antigen binding fragment thereof binds to three to five amino acid residues or more of cIL-4 at the following amino acid residue positions: 12 (K₁₂), 20 (R₂₀), 23 (S₂₃), 28 (T₂₈), 37 (K₃₇), 39 (T₃₉), 86 (K₈₆), 87 (K₈₇), 88 (S₈₈), 89 (T₈₉), 91 (K₉₁), and 96 (R₉₆) of the amino acid sequence of SEQ ID NO: 1.

The present invention further provides canine antibodies or antigen binding fragments thereof that bind cIL-4 and have an IC50 of lower than 40 nM for canine IL-4 mediated STAT-6 phosphorylation in DH82 cells. The present invention further provides canine antibodies or antigen binding fragments thereof that bind cIL-4 and have an IC50 of lower than 20 nM for canine IL-4 mediated STAT-6 phosphorylation in DH82 cells. In particular embodiments, the canine antibodies or antigen binding fragments thereof bind cIL-4 and have an IC50 of lower than 10 nM for canine IL-4 mediated STAT-6 phosphorylation in DH82 cells. In still other embodiments, the canine antibodies or antigen binding fragments thereof bind cIL-4 and have an IC50 of between 0.5 to 50 nM for canine IL-4 mediated STAT-6 phosphorylation in DH82 cells. In yet other embodiments, the canine antibodies or antigen binding fragments thereof bind cIL-4 and have an IC50 of between 2 to 25 nM for canine IL-4 mediated STAT-6 phosphorylation in DH82 cells. In still other embodiments, the canine antibodies or antigen binding fragments thereof bind cIL-4 and have an IC50 of between 4 to 20 nM for canine IL-4 mediated STAT-6 phosphorylation in DH82 cells.

In even more particular embodiments, the canine antibody or antigen binding fragment thereof, binds to an epitope of cIL-4 comprised by the amino acid sequence of SEQ ID NO: 89, SEQ ID NO: 90, SEQ ID NO: 91, SEQ ID NO: 92, and SEQ ID NO: 238, or any combination thereof, binds to one, two, three, four, five, or more amino acid residues of cIL-4 at the following amino acid residue positions: 12 (K₁₂), 20 (R₂₀), 23 (S₂₃), 28 (T₂₈), 37 (K₃₇), 39 (T₃₉), 86 (K₈₆), 87 (K₈₇), 88 (S₈₈), 89 (T₈₉), 91 (K₉₁), and 96 (R₉₆) of the amino acid sequence of SEQ ID NO: 1, and has an IC50 of lower than 50 nM, lower than 25 nM, and/or between 2 and/or 4 to 20 nM for canine IL-4 mediated STAT-6 phosphorylation in DH82 cells.

The present invention further provides nucleic acids that comprise nucleotide sequences that encode the specific sets of three heavy chain CDRs of the present invention and nucleic acids that comprise nucleotide sequences that encode the specific sets of three light chain CDRs of the present invention. In a particular embodiment, the nucleic acid encoding a set of three heavy chain CDRs comprises a nucleotide sequence that encodes an HCDR1 comprising the amino acid sequence of SEQ ID NO: 156, a nucleotide sequence that encodes an HCDR2 comprising the amino acid sequence of SEQ ID NO: 157, and a nucleotide sequence that encodes an HCDR3 comprising the amino acid sequence of SEQ ID NO: 158. In a companion embodiment, the nucleic acid encoding a set of three light chain CDRs comprises a nucleotide sequence that encodes an LCDR1 comprising the amino acid sequence of SEQ ID NO: 159, a nucleotide sequence that encodes an LCDR2 comprising the amino acid sequence of SEQ ID NO: 160, and a nucleotide sequence that encodes an LCDR3 comprising the amino acid sequence of SEQ ID NO: 161. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In another embodiment, the nucleic acid encoding a set of three heavy chain CDRs comprises a nucleotide sequence that encodes an HCDR1 comprising the amino acid sequence of SEQ ID NO: 162, a nucleotide sequence that encodes an HCDR2 comprising the amino acid sequence of SEQ ID NO: 163, and a nucleotide sequence that encodes an HCDR3 comprising the amino acid sequence of SEQ ID NO: 164. In a companion embodiment, the nucleic acid encoding a set of three light chain CDRs comprises a nucleotide sequence that encodes an LCDR1 comprising the amino acid sequence of SEQ ID NO: 165, a nucleotide sequence that encodes an LCDR2 comprising the amino acid sequence of SEQ ID NO: 166, and a nucleotide sequence that encodes an LCDR3 comprising the amino acid sequence of SEQ ID NO: 167. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In yet another embodiment, the nucleic acid encoding a set of three heavy chain CDRs comprises a nucleotide sequence that encodes an HCDR1 comprising the amino acid sequence of SEQ ID NO: 162, a nucleotide sequence that encodes an HCDR2 comprising the amino acid sequence of SEQ ID NO: 237, and a nucleotide sequence that encodes an HCDR3 comprising the amino acid sequence of SEQ ID NO: 164. In a companion embodiment, the nucleic acid encoding a set of three light chain CDRs comprises a nucleotide sequence that encodes an LCDR1 comprising the amino acid sequence of SEQ ID NO: 165, a nucleotide sequence that encodes an LCDR2 comprising the amino acid sequence of SEQ ID NO: 166, and a nucleotide sequence that encodes an LCDR3 comprising the amino acid sequence of SEQ ID NO: 167. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In still another embodiment, the nucleic acid encoding a set of three heavy chain CDRs comprises a nucleotide sequence that encodes an HCDR1 comprising the amino acid sequence of SEQ ID NO: 168, a nucleotide sequence that encodes an HCDR2 comprising the amino acid sequence of SEQ ID NO: 169, and a nucleotide sequence that encodes an HCDR3 comprising the amino acid sequence of SEQ ID NO: 170. In a companion embodiment, the nucleic acid encoding a set of three light chain CDRs comprises a nucleotide sequence that encodes an LCDR1 comprising the amino acid sequence of SEQ ID NO: 171, a nucleotide sequence that encodes an LCDR2 comprising the amino acid sequence of SEQ ID NO: 172, and a nucleotide sequence that encodes an LCDR3 comprising the amino acid sequence of SEQ ID NO: 173. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In yet another embodiment, the nucleic acid encoding a set of three heavy chain CDRs comprises a nucleotide sequence that encodes an HCDR1 comprising the amino acid sequence of SEQ ID NO: 174, a nucleotide sequence that encodes an HCDR2 comprising the amino acid sequence of SEQ ID NO: 175, and a nucleotide sequence that encodes an HCDR3 comprising the amino acid sequence of SEQ ID NO: 176. In a companion embodiment, the nucleic acid encoding a set of three light chain CDRs comprises a nucleotide sequence that encodes an LCDR1 comprising the amino acid sequence of SEQ ID NO: 177, a nucleotide sequence that encodes an LCDR2 comprising the amino acid sequence of SEQ ID NO: 178, and a nucleotide sequence that encodes an LCDR3 comprising the amino acid sequence of SEQ ID NO: 179. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In still another embodiment, the nucleic acid encoding a set of three heavy chain CDRs comprises a nucleotide sequence that encodes an HCDR1 comprising the amino acid sequence of SEQ ID NO: 180, a nucleotide sequence that encodes an HCDR2 comprising the amino acid sequence of SEQ ID NO: 181, and a nucleotide sequence that encodes an HCDR3 comprising the amino acid sequence of SEQ ID NO: 182. In a companion embodiment, the nucleic acid encoding a set of three light chain CDRs comprises a nucleotide sequence that encodes an LCDR1 comprising the amino acid sequence of SEQ ID NO: 183, a nucleotide sequence that encodes an LCDR2 comprising the amino acid sequence of SEQ ID NO: 184, and a nucleotide sequence that encodes an LCDR3 comprising the amino acid sequence of SEQ ID NO: 185. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In still another embodiment, the nucleic acid encoding a set of three heavy chain CDRs comprises a nucleotide sequence that encodes an HCDR1 comprising the amino acid sequence of SEQ ID NO: 186, a nucleotide sequence that encodes an HCDR2 comprising the amino acid sequence of SEQ ID NO: 187, and a nucleotide sequence that encodes an HCDR3 comprising the amino acid sequence of SEQ ID NO: 188. In a companion embodiment, a nucleic acid encoding a set of three light chain CDRs comprises a nucleotide sequence that encodes an LCDR1 comprising the amino acid sequence of SEQ ID NO: 189, a nucleotide sequence that encodes an LCDR2 comprising the amino acid sequence of SEQ ID NO: 190, and a nucleotide sequence that encodes an LCDR3 comprising the amino acid sequence of SEQ ID NO: 191. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In yet another embodiment, the nucleic acid encoding a set of three heavy chain CDRs comprises a nucleotide sequence that encodes an HCDR1 comprising the amino acid sequence of SEQ ID NO: 192, a nucleotide sequence that encodes an HCDR2 comprising the amino acid sequence of SEQ ID NO: 193, and a nucleotide sequence that encodes an HCDR3 comprising the amino acid sequence of SEQ ID NO: 194. In a companion embodiment, the nucleic acid encoding a set of three light chain CDRs comprises a nucleotide sequence that encodes an LCDR1 comprising the amino acid sequence of SEQ ID NO: 195, a nucleotide sequence that encodes an LCDR2 comprising the amino acid sequence of SEQ ID NO: 196, and a nucleotide sequence that encodes an LCDR3 comprising the amino acid sequence of SEQ ID NO: 197. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In still another embodiment, the nucleic acid encoding a set of three heavy chain CDRs comprises a nucleotide sequence that encodes an HCDR1 comprising the amino acid sequence of SEQ ID NO: 198, a nucleotide sequence that encodes an HCDR2 comprising the amino acid sequence of SEQ ID NO: 199, and a nucleotide sequence that encodes an HCDR3 comprising the amino acid sequence of SEQ ID NO: 200. In a companion embodiment, the nucleic acid encoding a set of three light chain CDRs comprises a nucleotide sequence that encodes an LCDR1 comprising the amino acid sequence of SEQ ID NO: 201, a nucleotide sequence that encodes an LCDR2 comprising the amino acid sequence of SEQ ID NO: 202, and a nucleotide sequence that encodes an LCDR3 comprising the amino acid sequence of SEQ ID NO: 203. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In still another embodiment, the nucleic acid encoding a set of three heavy chain CDRs comprises a nucleotide sequence that encodes an HCDR1 comprising the amino acid sequence of SEQ ID NO: 204, a nucleotide sequence that encodes an HCDR2 comprising the amino acid sequence of SEQ ID NO: 205, and a nucleotide sequence that encodes an HCDR3 comprising the amino acid sequence of SEQ ID NO: 206. In a companion embodiment, a nucleic acid encoding a set of three light chain CDRs comprises a nucleotide sequence that encodes an LCDR1 comprising the amino acid sequence of SEQ ID NO: 207, a nucleotide sequence that encodes an LCDR2 comprising the amino acid sequence of SEQ ID NO: 208, and a nucleotide sequence that encodes an LCDR3 comprising the amino acid sequence of SEQ ID NO: 209. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In yet another embodiment, the nucleic acid encoding a set of three heavy chain CDRs comprises a nucleotide sequence that encodes an HCDR1 comprising the amino acid sequence of SEQ ID NO: 210, a nucleotide sequence that encodes an HCDR2 comprising the amino acid sequence of SEQ ID NO: 211, and a nucleotide sequence that encodes an HCDR3 comprising the amino acid sequence of SEQ ID NO: 212. In a companion embodiment, the nucleic acid encoding a set of three light chain CDRs comprises a nucleotide sequence that encodes an LCDR1 comprising the amino acid sequence of SEQ ID NO: 213, a nucleotide sequence that encodes an LCDR2 comprising the amino acid sequence of SEQ ID NO: 214, and a nucleotide sequence that encodes an LCDR3 comprising the amino acid sequence of SEQ ID NO: 215. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In still another embodiment, the nucleic acid encoding a set of three heavy chain CDRs comprises a nucleotide sequence that encodes an HCDR1 comprising the amino acid sequence of SEQ ID NO: 216, a nucleotide sequence that encodes an HCDR2 comprising the amino acid sequence of SEQ ID NO: 217, and a nucleotide sequence that encodes an HCDR3 comprising the amino acid sequence of SEQ ID NO: 218. In a companion embodiment, the nucleic acid encoding a set of three light chain CDRs comprises a nucleotide sequence that encodes an LCDR1 comprising the amino acid sequence of SEQ ID NO: 219, a nucleotide sequence that encodes an LCDR2 comprising the amino acid sequence of SEQ ID NO: 220, and a nucleotide sequence that encodes an LCDR3 comprising the amino acid sequence of SEQ ID NO: 221. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In yet another embodiment, the nucleic acid encoding a set of three heavy chain CDRs comprises a nucleotide sequence that encodes an HCDR1 comprising the amino acid sequence of SEQ ID NO: 222, a nucleotide sequence that encodes an HCDR2 comprising the amino acid sequence of SEQ ID NO: 223, and a nucleotide sequence that encodes an HCDR3 comprising the amino acid sequence of SEQ ID NO: 224. In a companion embodiment, the nucleic acid encoding a set of three light chain CDRs comprises a nucleotide sequence that encodes an LCDR1 comprising the amino acid sequence of SEQ ID NO: 225, a nucleotide sequence that encodes an LCDR2 comprising the amino acid sequence of SEQ ID NO: 226, and a nucleotide sequence that encodes an LCDR3 comprising the amino acid sequence of SEQ ID NO: 227. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

In still another embodiment, the nucleic acid encoding a set of three heavy chain CDRs comprises a nucleotide sequence that encodes an HCDR1 comprising the amino acid sequence of SEQ ID NO: 228, a nucleotide sequence that encodes an HCDR2 comprising the amino acid sequence of SEQ ID NO: 229, and a nucleotide sequence that encodes an HCDR3 comprising the amino acid sequence of SEQ ID NO: 230. In a companion embodiment, the nucleic acid encoding a set of three light chain CDRs comprises a nucleotide sequence that encodes an LCDR1 comprising the amino acid sequence of SEQ ID NO: 231, a nucleotide sequence that encodes an LCDR2 comprising the amino acid sequence of SEQ ID NO: 232, and a nucleotide sequence that encodes an LCDR3 comprising the amino acid sequence of SEQ ID NO: 233. The present invention further provides as a pair, a nucleic acid encoding the set of the three heavy chain CDRs and a nucleic acid that encodes the set of the three light chain CDRs. The present invention also provides a kit containing this pair of two nucleic acids. In certain embodiments, a nucleic acid encoding the set of the three heavy chain CDRs encodes the heavy chain variable region of a canine antibody and the corresponding nucleic acid encoding the set of the three light chain CDRs encodes the light chain variable region of that canine antibody. In related embodiments, a nucleic acid encoding the set of the three heavy chain CDRs encodes the heavy chain of a canine antibody or antigen binding fragment thereof and the corresponding nucleic acid encoding the set of the three light chain CDRs encodes the light chain of that canine antibody or antigen binding fragment thereof.

The canine antibody or antigen binding fragment thereof, can further comprise a heavy chain variable region that comprises at least 90%, 95%, 98%, 99%, or 100% sequence identity with the amino acid sequence of SEQ ID NO: 4. Alternatively, the canine antibody or antigen binding fragment thereof, can further comprise a heavy chain variable region that comprises at least 90%, 95%, 98%, 99%, or 100% sequence identity with the amino acid sequence of SEQ ID NO: 7. In yet other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 10. In still other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 236. In yet other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 13. In still other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 16. In yet other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 19. In still other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 22. In yet other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 25. In still other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 28.

In yet other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 31. In still other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 34. In yet other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 37. In still other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 40. In certain embodiments of this type, the antibody is a canine antibody or antigen binding fragment of the canine antibody. In specific embodiments, the antibody is a canine antibody.

The canine antibody or antigen binding fragment thereof, also can further comprise a light chain variable region comprising at least 90%, 95%, 98%, 99%, or 100% sequence identity with the amino acid sequence of SEQ ID NO: 5. Alternatively, the canine antibody or antigen binding fragment thereof, also can further comprise a light chain variable region comprising at least 90%, 95%, 98%, 99%, or 100% sequence identity with the amino acid sequence of SEQ ID NO: 8. In yet other embodiments, the canine antibody or antigen binding fragment thereof, can further comprise a light chain variable region comprising at least 90%, 95%, 98%, 99%, or 100% sequence identity with the amino acid sequence of SEQ ID NO: 11. In still other embodiments, the canine antibody or antigen binding fragment thereof, can further comprise a light chain variable region comprising at least 90%, 95%, 98%, 99%, or 100% sequence identity with the amino acid sequence of SEQ ID NO: 14. In yet other embodiments, the canine antibody or antigen binding fragment thereof, can further comprise a light chain variable region comprising at least 90%, 95%, 98%, 99%, or 100% sequence identity with the amino acid sequence of SEQ ID NO: 17.

In still other embodiments, the canine antibody or antigen binding fragment thereof, can further comprise a light chain variable region comprising at least 90%, 95%, 98%, 99%, or 100% sequence identity with the amino acid sequence of SEQ ID NO: 20. In yet other embodiments, the canine antibody or antigen binding fragment thereof, can further comprise a light chain variable region comprising at least 90%, 95%, 98%, 99%, or 100% sequence identity with the amino acid sequence of SEQ ID NO: 23. In still other embodiments, the canine antibody or antigen binding fragment thereof, can further comprise a light chain variable region comprising at least 90%, 95%, 98%, 99%, or 100% sequence identity with the amino acid sequence of SEQ ID NO: 26. In certain embodiments of this type, the antibody is a canine antibody or antigen binding fragment of the canine antibody. In specific embodiments, the antibody is a canine antibody.

In yet other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 29. In still other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 32. In yet other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 35. In still other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 38. In yet other embodiments, the canine antibody or antigen binding fragment thereof, can further comprise a heavy chain variable region that comprises at least 90%, 95%, 98%, 99%, or 100% sequence identity with the amino acid sequence of SEQ ID NO: 41.

The canine antibody or antigen binding fragment thereof can further comprise a heavy chain variable region that comprises at least 90%, 95%, 98%, 99% or 100% sequence identity with the amino acid sequence of SEQ ID NO: 4, while still retaining an HCDR1 that comprises the amino acid sequence of SEQ ID NO: 156, an HCDR2 that comprises the amino acid sequence of SEQ ID NO: 157, and an HCDR3 that comprises the amino acid sequence of SEQ ID NO: 158, and a light chain variable region that comprises at least 90%, 95%, 98%, 99% or 100% sequence identity with the amino acid sequence of SEQ ID NO: 5, while still retaining an LCDR1 that comprises the amino acid sequence of SEQ ID NO: 159, an LCDR2 that comprises the amino acid sequence of SEQ ID NO: 160, and an LCDR3 that comprises the amino acid sequence of SEQ ID NO: 161. In certain embodiments of this type, the antibody is a canine antibody or antigen binding fragment of the canine antibody. In yet other embodiments, the antibody is a canine antibody. In specific embodiments, the canine antibody or antigen binding fragment thereof comprises a heavy chain variable region that comprises the amino acid sequence of SEQ ID NO: 4 and a light chain variable region that comprises the amino acid sequence of SEQ ID NO: 5.

The canine antibody or antigen binding fragment thereof can further comprise a heavy chain variable region that comprises at least 90%, 95%, 98%, 99% or 100% sequence identity with the amino acid sequence of SEQ ID NO: 7, while still retaining an HCDR1 that comprises the amino acid sequence of SEQ ID NO: 162, an HCDR2 that comprises the amino acid sequence of SEQ ID NO: 163, and an HCDR3 that comprises the amino acid sequence of SEQ ID NO: 164, and a light chain variable region that comprises at least 90%, 95%, 98%, 99% or 100%, sequence identity with the amino acid sequence of SEQ ID NO: 8, while still retaining an LCDR1 that comprises the amino acid sequence of SEQ ID NO: 165, an LCDR2 that comprises the amino acid sequence of SEQ ID NO: 166, and an LCDR3 that comprises the amino acid sequence of SEQ ID NO: 167. In certain embodiments of this type, the antibody is a canine antibody or antigen binding fragment of the canine antibody. In still other embodiments, the antibody is a canine antibody. In specific embodiments, the canine antibody or antigen binding fragment thereof comprises a heavy chain variable region that comprises the amino acid sequence of SEQ ID NO: 7 and a light chain variable region that comprises the amino acid sequence of SEQ ID NO: 8.

The canine antibody or antigen binding fragment thereof can further comprise a heavy chain variable region that comprises at least 90%, 95%, 98%, 99% or 100% sequence identity with the amino acid sequence of SEQ ID NO: 236, while still retaining an HCDR1 that comprises the amino acid sequence of SEQ ID NO: 162, an HCDR2 that comprises the amino acid sequence of SEQ ID NO: 237, and an HCDR3 that comprises the amino acid sequence of SEQ ID NO: 164, and a light chain variable region that comprises at least 90%, 95%, 98%, 99% or 100%, sequence identity with the amino acid sequence of SEQ ID NO: 8, while still retaining an LCDR1 that comprises the amino acid sequence of SEQ ID NO: 165, an LCDR2 that comprises the amino acid sequence of SEQ ID NO: 166, and an LCDR3 that comprises the amino acid sequence of SEQ ID NO: 167. In certain embodiments of this type, the antibody is a canine antibody or antigen binding fragment of the canine antibody. In still other embodiments, the antibody is a canine antibody. In specific embodiments, the canine antibody or antigen binding fragment thereof comprises a heavy chain variable region that comprises the amino acid sequence of SEQ ID NO: 236 and a light chain variable region that comprises the amino acid sequence of SEQ ID NO: 8.

The canine antibody or antigen binding fragment thereof can further comprise a heavy chain variable region that comprises at least 90%, 95%, 98%, 99% or 100% sequence identity with the amino acid sequence of SEQ ID NO: 10, while still retaining an HCDR1 that comprises the amino acid sequence of SEQ ID NO: 168, an HCDR2 that comprises the amino acid sequence of SEQ ID NO: 169, and an HCDR3 that comprises the amino acid sequence of SEQ ID NO: 170, and a light chain variable region that comprises at least 90%, 95%, 98%, 99% or 100% sequence identity with the amino acid sequence of SEQ ID NO: 11, while still retaining an LCDR1 that comprises the amino acid sequence of SEQ ID NO: 171, an LCDR2 that comprises the amino acid sequence of SEQ ID NO: 172, and an LCDR3 that comprises the amino acid sequence of SEQ ID NO: 173. In certain embodiments of this type, the antibody is a canine antibody or antigen binding fragment of the canine antibody. In still other embodiments, the antibody is a canine antibody. In specific embodiments, the canine antibody or antigen binding fragment thereof comprises a heavy chain variable region that comprises the amino acid sequence of SEQ ID NO: 10 and a light chain variable region that comprises the amino acid sequence of SEQ ID NO: 11.

The canine antibody or antigen binding fragment thereof can further comprise a heavy chain variable region that comprises at least 90%, 95%, 98%, 99% or 100% sequence identity with the amino acid sequence of SEQ ID NO: 13, while still retaining an HCDR1 that comprises the amino acid sequence of SEQ ID NO: 174, an HCDR2 that comprises the amino acid sequence of SEQ ID NO: 175, and an HCDR3 that comprises the amino acid sequence of SEQ ID NO: 176, and a light chain variable region that comprises at least 90%, 95%, 98%, 99% or 100% sequence identity with the amino acid sequence of SEQ ID NO: 14, while still retaining an LCDR1 that comprises the amino acid sequence of SEQ ID NO: 177, an LCDR2 that comprises the amino acid sequence of SEQ ID NO: 178, and an LCDR3 that comprises the amino acid sequence of SEQ ID NO: 179. In certain embodiments of this type, the antibody is a canine antibody or antigen binding fragment of the canine antibody. In still other embodiments, the antibody is a canine antibody. In specific embodiments, the canine antibody or antigen binding fragment thereof comprises a heavy chain variable region that comprises the amino acid sequence of SEQ ID NO: 13 and a light chain variable region that comprises the amino acid sequence of SEQ ID NO: 14.

The canine antibody or antigen binding fragment thereof can further comprise a heavy chain variable region that comprises at least 90%, 95%, 98%, 99% or 100% sequence identity with the amino acid sequence of SEQ ID NO: 16, while still retaining an HCDR1 that comprises the amino acid sequence of SEQ ID NO: 180, an HCDR2 that comprises the amino acid sequence of SEQ ID NO: 181, and an HCDR3 that comprises the amino acid sequence of SEQ ID NO: 182, and a light chain variable region that comprises at least 90%, 95%, 98%, 99% or 100% sequence identity with the amino acid sequence of SEQ ID NO: 17, while still retaining an LCDR1 that comprises the amino acid sequence of SEQ ID NO: 183, an LCDR2 that comprises the amino acid sequence of SEQ ID NO: 184, and an LCDR3 that comprises the amino acid sequence of SEQ ID NO: 185. In certain embodiments of this type, the antibody is a canine antibody or antigen binding fragment of the canine antibody. In still other embodiments, the antibody is a canine antibody. In specific embodiments, the canine antibody or antigen binding fragment thereof comprises a heavy chain variable region that comprises the amino acid sequence of SEQ ID NO: 16 and a light chain variable region that comprises the amino acid sequence of SEQ ID NO: 17.

The canine antibody or antigen binding fragment thereof can further comprise a heavy chain variable region that comprises at least 90%, 95%, 98%, 99% or 100% sequence identity with the amino acid sequence of SEQ ID NO: 19, while still retaining an HCDR1 that comprises the amino acid sequence of SEQ ID NO: 186, an HCDR2 that comprises the amino acid sequence of SEQ ID NO: 187, and an HCDR3 that comprises the amino acid sequence of SEQ ID NO: 188, and a light chain variable region that comprises at least 90%, 95%, 98%, 99% or 100% sequence identity with the amino acid sequence of SEQ ID NO: 20, while still retaining an LCDR1 that comprises the amino acid sequence of SEQ ID NO: 189, an LCDR2 that comprises the amino acid sequence of SEQ ID NO: 190, and an LCDR3 that comprises the amino acid sequence of SEQ ID NO: 191. In certain embodiments of this type, the antibody is a canine antibody or antigen binding fragment of the canine antibody. In still other embodiments, the antibody is a canine antibody. In specific embodiments, the canine antibody or antigen binding fragment thereof comprises a heavy chain variable region that comprises the amino acid sequence of SEQ ID NO: 19 and a light chain variable region that comprises the amino acid sequence of SEQ ID NO: 20.

The canine antibody or antigen binding fragment thereof can further comprise a heavy chain variable region that comprises at least 90%, 95%, 98%, 99% or 100% sequence identity with the amino acid sequence of SEQ ID NO: 22, while still retaining an HCDR1 that comprises the amino acid sequence of SEQ ID NO: 192, an HCDR2 that comprises the amino acid sequence of SEQ ID NO: 193, and an HCDR3 that comprises the amino acid sequence of SEQ ID NO: 194, and a light chain variable region that comprises at least 90%, 95%, 98%, 99% or 100% sequence identity with the amino acid sequence of SEQ ID NO: 23, while still retaining an LCDR1 that comprises the amino acid sequence of SEQ ID NO: 195, an LCDR2 that comprises the amino acid sequence of SEQ ID NO: 196, and an LCDR3 that comprises the amino acid sequence of SEQ ID NO: 197. In certain embodiments of this type, the antibody is a canine antibody or antigen binding fragment of the canine antibody. In still other embodiments, the antibody is a canine antibody. In specific embodiments, the canine antibody or antigen binding fragment thereof comprises a heavy chain variable region that comprises the amino acid sequence of SEQ ID NO: 22 and a light chain variable region that comprises the amino acid sequence of SEQ ID NO: 23.

The canine antibody or antigen binding fragment thereof can further comprise a heavy chain variable region that comprises at least 90%, 95%, 98%, 99% or 100% sequence identity with the amino acid sequence of SEQ ID NO: 25, while still retaining an HCDR1 that comprises the amino acid sequence of SEQ ID NO: 198, an HCDR2 that comprises the amino acid sequence of SEQ ID NO: 199, and an HCDR3 that comprises the amino acid sequence of SEQ ID NO: 200, and a light chain variable region that comprises at least 90%, 95%, 98%, 99% or 100% sequence identity with the amino acid sequence of SEQ ID NO: 26, while still retaining an LCDR1 that comprises the amino acid sequence of SEQ ID NO: 201, an LCDR2 that comprises the amino acid sequence of SEQ ID NO: 202, and an LCDR3 that comprises the amino acid sequence of SEQ ID NO: 203. In certain embodiments of this type, the antibody is a canine antibody or antigen binding fragment of the canine antibody. In yet other embodiments, the antibody is a canine antibody. In specific embodiments, the canine antibody or antigen binding fragment thereof comprises a heavy chain variable region that comprises the amino acid sequence of SEQ ID NO: 25 and a light chain variable region that comprises the amino acid sequence of SEQ ID NO: 26.

The canine antibody or antigen binding fragment thereof can further comprise a heavy chain variable region that comprises at least 90%, 95%, 98%, 99% or 100% sequence identity with the amino acid sequence of SEQ ID NO: 28, while still retaining an HCDR1 that comprises the amino acid sequence of SEQ ID NO: 204, an HCDR2 that comprises the amino acid sequence of SEQ ID NO: 205, and an HCDR3 that comprises the amino acid sequence of SEQ ID NO: 206, and a light chain variable region that comprises at least 90%, 95%, 98%, 99% or 100% sequence identity with the amino acid sequence of SEQ ID NO: 29, while still retaining an LCDR1 that comprises the amino acid sequence of SEQ ID NO: 207, an LCDR2 that comprises the amino acid sequence of SEQ ID NO: 208, and an LCDR3 that comprises the amino acid sequence of SEQ ID NO: 209. In certain embodiments of this type, the antibody is a canine antibody or antigen binding fragment of the canine antibody. In yet other embodiments, the antibody is a canine antibody. In specific embodiments, the canine antibody or antigen binding fragment thereof comprises a heavy chain variable region that comprises the amino acid sequence of SEQ ID NO: 28 and a light chain variable region that comprises the amino acid sequence of SEQ ID NO: 29.

The canine antibody or antigen binding fragment thereof can further comprise a heavy chain variable region that comprises at least 90%, 95%, 98%, 99% or 100% sequence identity with the amino acid sequence of SEQ ID NO: 31, while still retaining an HCDR1 that comprises the amino acid sequence of SEQ ID NO: 210, an HCDR2 that comprises the amino acid sequence of SEQ ID NO: 211, and an HCDR3 that comprises the amino acid sequence of SEQ ID NO: 212, and a light chain variable region that comprises at least 90%, 95%, 98%, 99% or 100%, sequence identity with the amino acid sequence of SEQ ID NO: 32, while still retaining an LCDR1 that comprises the amino acid sequence of SEQ ID NO: 213, an LCDR2 that comprises the amino acid sequence of SEQ ID NO: 214, and an LCDR3 that comprises the amino acid sequence of SEQ ID NO: 215. In certain embodiments of this type, the antibody is a canine antibody or antigen binding fragment of the canine antibody. In still other embodiments, the antibody is a canine antibody. In specific embodiments, the canine antibody or antigen binding fragment thereof comprises a heavy chain variable region that comprises the amino acid sequence of SEQ ID NO: 31 and a light chain variable region that comprises the amino acid sequence of SEQ ID NO: 32.

The canine antibody or antigen binding fragment thereof can further comprise a heavy chain variable region that comprises at least 90%, 95%, 98%, 99% or 100% sequence identity with the amino acid sequence of SEQ ID NO: 34, while still retaining an HCDR1 that comprises the amino acid sequence of SEQ ID NO: 216, an HCDR2 that comprises the amino acid sequence of SEQ ID NO: 217, and an HCDR3 that comprises the amino acid sequence of SEQ ID NO: 218, and a light chain variable region that comprises at least 90%, 95%, 98%, 99% or 100% sequence identity with the amino acid sequence of SEQ ID NO: 35, while still retaining an LCDR1 that comprises the amino acid sequence of SEQ ID NO: 219, an LCDR2 that comprises the amino acid sequence of SEQ ID NO: 220, and an LCDR3 that comprises the amino acid sequence of SEQ ID NO: 221. In certain embodiments of this type, the antibody is a canine antibody or antigen binding fragment of the canine antibody. In still other embodiments, the antibody is a canine antibody. In specific embodiments, the canine antibody or antigen binding fragment thereof comprises a heavy chain variable region that comprises the amino acid sequence of SEQ ID NO: 34 and a light chain variable region that comprises the amino acid sequence of SEQ ID NO: 35.

The canine antibody or antigen binding fragment thereof can further comprise a heavy chain variable region that comprises at least 90%, 95%, 98%, 99% or 100% sequence identity with the amino acid sequence of SEQ ID NO: 37, while still retaining an HCDR1 that comprises the amino acid sequence of SEQ ID NO: 222, an HCDR2 that comprises the amino acid sequence of SEQ ID NO: 223, and an HCDR3 that comprises the amino acid sequence of SEQ ID NO: 224, and a light chain variable region that comprises at least 90%, 95%, 98%, 99% or 100% sequence identity with the amino acid sequence of SEQ ID NO: 38, while still retaining an LCDR1 that comprises the amino acid sequence of SEQ ID NO: 225, an LCDR2 that comprises the amino acid sequence of SEQ ID NO: 226, and an LCDR3 that comprises the amino acid sequence of SEQ ID NO: 227. In certain embodiments of this type, the antibody is a canine antibody or antigen binding fragment of the canine antibody. In still other embodiments, the antibody is a canine antibody. In specific embodiments, the canine antibody or antigen binding fragment thereof comprises a heavy chain variable region that comprises the amino acid sequence of SEQ ID NO: 37 and a light chain variable region that comprises the amino acid sequence of SEQ ID NO: 38.

The canine antibody or antigen binding fragment thereof can further comprise a heavy chain variable region that comprises at least 90%, 95%, 98%, 99% or 100% sequence identity with the amino acid sequence of SEQ ID NO: 40, while still retaining an HCDR1 that comprises the amino acid sequence of SEQ ID NO: 228, an HCDR2 that comprises the amino acid sequence of SEQ ID NO: 229, and an HCDR3 that comprises the amino acid sequence of SEQ ID NO: 230, and a light chain variable region that comprises at least 90%, 95%, 98%, 99% or 100% sequence identity with the amino acid sequence of SEQ ID NO: 41, while still retaining an LCDR1 that comprises the amino acid sequence of SEQ ID NO: 231, an LCDR2 that comprises the amino acid sequence of SEQ ID NO: 232, and an LCDR3 that comprises the amino acid sequence of SEQ ID NO: 233. In certain embodiments of this type, the antibody is a canine antibody or antigen binding fragment of the canine antibody. In still other embodiments, the antibody is a canine antibody. In specific embodiments, the canine antibody or antigen binding fragment thereof comprises a heavy chain variable region that comprises the amino acid sequence of SEQ ID NO: 40 and a light chain variable region that comprises the amino acid sequence of SEQ ID NO: 41.

In particular embodiments of the canine antibodies or antigen binding fragments thereof that comprise the aforementioned heavy chain variable regions and light chain variable regions, when bound to cIL-4, the canine antibodies or antigen binding fragments thereof (i) bind to an epitope comprised by the amino acid sequence of SEQ ID NO: 89, SEQ ID NO: 90, SEQ ID NO: 91, SEQ ID NO: 92, SEQ ID NO: 238, or any combination thereof, (ii) have an IC50 of lower than 40 nM for canine IL-4 mediated STAT-6 phosphorylation in DH82 cells, or (iii) both bind to an epitope comprised by the amino acid sequence of SEQ ID NO: 89, SEQ ID NO: 90, SEQ ID NO: 91, SEQ ID NO: 92, SEQ ID NO: 238, or any combination thereof, and have an IC50 of lower than 40 nM for canine IL-4 mediated STAT-6 phosphorylation in DH82 cells. In more particular embodiments, the canine antibody or antigen binding fragment thereof, when bound to cIL-4 binds to an epitope comprised by the amino acid sequence of SEQ ID NO: 89, SEQ ID NO: 90, SEQ ID NO: 91, SEQ ID NO: 92, SEQ ID NO: 238, or any combination thereof. In certain embodiments, the canine antibody or antigen binding fragment thereof binds to one or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In other embodiments, the canine antibody or antigen binding fragment thereof binds to two or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In yet other embodiments, the canine antibody or antigen binding fragment thereof binds to three or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In still other embodiments, the canine antibody or antigen binding fragment thereof binds to four or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In yet other embodiments, the canine antibody or antigen binding fragment thereof binds to five or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In still other embodiments, the canine antibody or antigen binding fragment thereof binds to six or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In particular embodiments, the canine antibody or antigen binding fragment thereof binds to three to five amino acid residues of cIL-4 at the following amino acid residue positions: 6 (T₆), 8 (K₈), 12 (K₁₂), 74 (S₇₄), and 79 (T₇₉) of the amino acid sequence of SEQ ID NO: 1. In other particular embodiments, the canine antibody or antigen binding fragment thereof binds to three to five amino acid residues or more of cIL-4 at the following amino acid residue positions: 12 (K₁₂), 20 (R₂₀), 23 (S₂₃), 28 (T₂₈), 37 (K₃₇), 39 (T₃₉), 86 (K₈₆), 87 (K₈₇), 88 (S₈₈), 89 (T₈₉), 91 (K₉₁), and 96 (R₉₆) of the amino acid sequence of SEQ ID NO: 1.

The present invention further provides canine antibodies or antigen binding fragments thereof that bind cIL-4 and have an IC50 of lower than 40 nM for canine IL-4 mediated STAT-6 phosphorylation in DH82 cells. The present invention further provides canine antibodies or antigen binding fragments thereof that bind cIL-4 and have an IC50 of lower than 20 nM for canine IL-4 mediated STAT-6 phosphorylation in DH82 cells. In particular embodiments, the canine antibodies or antigen binding fragments thereof bind cIL-4 and have an IC50 of lower than 10 nM for canine IL-4 mediated STAT-6 phosphorylation in DH82 cells. In still other embodiments, the canine antibodies or antigen binding fragments thereof bind cIL-4 and have an IC50 of between 0.5 to 50 nM for canine IL-4 mediated STAT-6 phosphorylation in DH82 cells. In yet other embodiments, the canine antibodies or antigen binding fragments thereof bind cIL-4 and have an IC50 of between 2 to 25 nM for canine IL-4 mediated STAT-6 phosphorylation in DH82 cells. In still other embodiments, the canine antibodies or antigen binding fragments thereof bind cIL-4 and have an IC50 of between 4 to 20 nM for canine IL-4 mediated STAT-6 phosphorylation in DH82 cells.

In even more particular embodiments, the canine antibody or antigen binding fragment thereof, binds to an epitope of cIL-4 comprised by the amino acid sequence of SEQ ID NO: 89, SEQ ID NO: 90, SEQ ID NO: 91, SEQ ID NO: 92, and SEQ ID NO: 238, or any combination thereof, binds to one, two, three, four, five, or more amino acid residues of cIL-4 at the following amino acid residue positions: 12 (K₁₂), 20 (R₂₀), 23 (S₂₃), 28 (T₂₈), 37 (K₃₇), 39 (T₃₉), 86 (K₈₆), 87 (K₈₇), 88 (S₈₈), 89 (T₈₉), 91 (K₉₁), and 96 (R₉₆) of the amino acid sequence of SEQ ID NO: 1, and has an IC50 of lower than 50 nM, lower than 25 nM, and/or between 2 and/or 4 to 20 nM for canine IL-4 mediated STAT-6 phosphorylation in DH82 cells.

The present invention further provides nucleic acids that individually encode one of the heavy chain variable regions of the present invention and nucleic acids that individually encode one of the corresponding light chain variable regions of the present invention. The present invention further provides as a pair, a nucleic acid encoding the heavy chain variable region of a canine antibody or antigen binding fragment thereof and a nucleic acid that encodes the corresponding light chain variable region of that canine antibody or antigen binding fragment thereof. The present invention also provides a kit containing this pair of two nucleic acids. In certain embodiments, a nucleic acid encoding the heavy chain variable region encodes the heavy chain of a canine antibody or antigen binding fragment thereof and the corresponding nucleic acid encoding the light chain variable region encodes the light chain of a canine antibody or antigen binding fragment thereof.

In specific embodiments, one nucleic acid encodes a heavy chain variable region that comprises at least 90%, 95%, 98%, 99% or 100% sequence identity with the amino acid sequence of SEQ ID NO: 25, while still retaining an HCDR1 that comprises the amino acid sequence of SEQ ID NO: 198, an HCDR2 that comprises the amino acid sequence of SEQ ID NO: 199, and an HCDR3 that comprises the amino acid sequence of SEQ ID NO: 200, and another nucleic acid encodes a corresponding light chain variable region that comprises at least 90%, 95%, 98%, 99% or 100% sequence identity with the amino acid sequence of SEQ ID NO: 26, while still retaining an LCDR1 that comprises the amino acid sequence of SEQ ID NO: 201, an LCDR2 that comprises the amino acid sequence of SEQ ID NO: 202, and an LCDR3 that comprises the amino acid sequence of SEQ ID NO: 203. The present invention further provides as a pair, a nucleic acid encoding the heavy chain variable region of an antibody and a nucleic acid that encodes the corresponding light chain variable region of that antibody. The present invention also provides a kit containing this pair of two nucleic acids. In certain embodiments, a nucleic acid encoding the heavy chain variable region encodes the heavy chain of a canine antibody or antigen binding fragment thereof and the corresponding nucleic acid encoding the light chain variable region encodes the light chain of that canine antibody or antigen binding fragment thereof.

In a more specific embodiment, a nucleic acid encodes a heavy chain variable region of a canine antibody or antigen binding fragment thereof that comprises the amino acid sequence of SEQ ID NO: 25 and in a related embodiment a nucleic acid encodes the corresponding light chain variable region that comprises the amino acid sequence of SEQ ID NO: 26. The present invention further provides as a pair, a nucleic acid encoding the heavy chain variable region that comprises the amino acid sequence of SEQ ID NO: 25 and a nucleic acid encoding the light chain variable region comprising the amino acid sequence of SEQ ID NO: 26. The present invention also provides a kit containing this pair of two nucleic acids. In certain embodiments, a nucleic acid encoding the heavy chain variable region encodes the heavy chain of a canine antibody or antigen binding fragment of thereof and the corresponding nucleic acid encoding the light chain variable region encodes the light chain of that canine antibody or antigen binding fragment thereof.

In other specific embodiments, one nucleic acid encodes a heavy chain variable region that comprises at least 90%, 95%, 98%, 99% or 100% sequence identity with the amino acid sequence of SEQ ID NO: 28, while still retaining an HCDR1 that comprises the amino acid sequence of SEQ ID NO: 204, an HCDR2 that comprises the amino acid sequence of SEQ ID NO: 205, and an HCDR3 that comprises the amino acid sequence of SEQ ID NO: 206, and another nucleic acid encodes a corresponding light chain variable region that comprises at least 90%, 95%, 98%, 99% or 100% sequence identity with the amino acid sequence of SEQ ID NO: 29, while still retaining an LCDR1 that comprises the amino acid sequence of SEQ ID NO: 207, an LCDR2 that comprises the amino acid sequence of SEQ ID NO: 208, and an LCDR3 that comprises the amino acid sequence of SEQ ID NO: 209. The present invention further provides as a pair, a nucleic acid encoding the heavy chain variable region of an antibody and a nucleic acid that encodes the corresponding light chain variable region of that antibody. The present invention also provides a kit containing this pair of two nucleic acids. In certain embodiments, a nucleic acid encoding the heavy chain variable region encodes the heavy chain of a canine antibody or antigen binding fragment thereof and the corresponding nucleic acid encoding the light chain variable region encodes the light chain of that canine antibody or antigen binding fragment thereof.

In a more specific embodiment, a nucleic acid encodes a heavy chain variable region of a canine antibody or antigen binding fragment thereof that comprises the amino acid sequence of SEQ ID NO: 28 and in a related embodiment a nucleic acid encodes the corresponding light chain variable region that comprises the amino acid sequence of SEQ ID NO: 29. The present invention further provides as a pair, a nucleic acid encoding the heavy chain variable region that comprises the amino acid sequence of SEQ ID NO: 28 and a nucleic acid encoding the light chain variable region comprising the amino acid sequence of SEQ ID NO: 29. The present invention also provides a kit containing this pair of two nucleic acids. In certain embodiments, a nucleic acid encoding the heavy chain variable region encodes the heavy chain of a canine antibody or antigen binding fragment of thereof and the corresponding nucleic acid encoding the light chain variable region encodes the light chain of that canine antibody or antigen binding fragment thereof.

The canine antibodies of the present invention comprise a canine hinge region. In particular embodiments, the hinge region comprises the amino acid sequence that comprises at least 90%, 95%, or 100% sequence identity with the amino acid sequence of SEQ ID NO: 109. In other embodiments, the hinge region comprises the amino acid sequence that comprises at least 90%, 95%, or 100% sequence identity with the amino acid sequence of SEQ ID NO: 110. In yet other embodiments, the hinge region comprises the amino acid sequence that comprises at least 90%, 95%, or 100% sequence identity with the amino acid sequence of SEQ ID NO: 111. In still other embodiments, the hinge region comprises the amino acid sequence that comprises at least 90%, 95%, or 100% sequence identity with the amino acid sequence of SEQ ID NO: 112. In yet other embodiments, the hinge region comprises the amino acid sequence that comprises at least 90%, 95%, or 100% sequence identity with the amino acid sequence of SEQ ID NO: 113. In certain embodiments of this type, the antibody is a canine antibody or antigen binding fragment of the canine antibody. In specific embodiments, the antibody is a canine antibody.

The canine antibodies of the present invention comprise a canine fragment crystallizable region (cFc). In particular embodiments, the cFc comprises an amino acid sequence that comprises at least 90%, 95%, 98%, 99%, or 100% sequence identity with the amino acid sequence of SEQ ID NO: 99. In other embodiments, the cFc comprises an amino acid sequence that comprises at least 90%, 95%, 98%, 99%, or 100% sequence identity with the amino acid sequence of SEQ ID NO: 100. In still other embodiments, the cFc comprises an amino acid sequence that comprises at least 90%, 95%, 98%, 99%, or 100% sequence identity with the amino acid sequence of SEQ ID NO: 101. In yet other embodiments, the cFc comprises an amino acid sequence that comprises at least 90%, 95%, 98%, 99%, or 100% sequence identity with the amino acid sequence of SEQ ID NO: 102. In still other embodiments, the cFc comprises an amino acid sequence that comprises at least 90%, 95%, 98%, 99%, or 100% sequence identity with the amino acid sequence of SEQ ID NO: 103. In yet other embodiments, the cFc comprises an amino acid sequence that comprises at least 90%, 95%, 98%, 99%, or 100% sequence identity with the amino acid sequence of SEQ ID NO: 104. In still other embodiments, the cFc comprises an amino acid sequence that comprises at least 90%, 95%, 98%, 99%, or 100% sequence identity with the amino acid sequence of SEQ ID NO: 105. In yet other embodiments, the cFc comprises an amino acid sequence that comprises at least 90%, 95%, 98%, 99%, or 100% sequence identity with the amino acid sequence of SEQ ID NO: 106. In yet other embodiments, the cFc comprises an amino acid sequence that comprises at least 90%, 95%, 98%, 99%, or 100% sequence identity with the amino acid sequence of SEQ ID NO: 255. In yet other embodiments, the cFc comprises an amino acid sequence that comprises at least 90%, 95%, 98%, 99%, or 100% sequence identity with the amino acid sequence of SEQ ID NO: 256. In yet other embodiments, the cFc comprises an amino acid sequence that comprises at least 90%, 95%, 98%, 99%, or 100% sequence identity with the amino acid sequence of SEQ ID NO: 257. In yet other embodiments, the cFc comprises an amino acid sequence that comprises at least 90%, 95%, 98%, 99%, or 100% sequence identity with the amino acid sequence of SEQ ID NO: 258. In certain embodiments of this type, the antibody is a canine antibody or antigen binding fragment of the canine antibody. In specific embodiments, the antibody is a canine antibody.

In particular embodiments, the canine antibodies comprise a heavy chain that comprises at least 90%, 95%, 98%, 99% or 100% sequence identity with the amino acid sequence of SEQ ID NO: 42, while still retaining an HCDR1 that comprises the amino acid sequence of SEQ ID NO: 204, an HCDR2 that comprises the amino acid sequence of SEQ ID NO: 205, and an HCDR3 that comprises the amino acid sequence of SEQ ID NO: 206 and a light chain that comprises at least 90%, 95%, 98%, 99% or 100% sequence identity with the amino acid sequence of SEQ ID NO: 43, while still retaining an LCDR1 that comprises the amino acid sequence of SEQ ID NO: 207, an LCDR2 that comprises the amino acid sequence of SEQ ID NO: 208, and an LCDR3 that comprises the amino acid sequence of SEQ ID NO: 209. In other embodiments, the canine antibody comprises a heavy chain that comprises the amino acid sequence of SEQ ID NO: 42 and a light chain that comprises the amino acid sequence of SEQ ID NO: 43. The present invention further provides antigen binding fragments of these canine antibodies.

In specific embodiments, the canine antibodies comprise a heavy chain that comprises at least 90%, 95%, 98%, 99% or 100% sequence identity with the amino acid sequence of SEQ ID NO: 44, while still retaining an HCDR1 that comprises the amino acid sequence of SEQ ID NO: 198, an HCDR2 that comprises the amino acid sequence of SEQ ID NO: 199, and an HCDR3 that comprises the amino acid sequence of SEQ ID NO: 200 and a light chain that comprises at least 90%, 95%, 98%, 99% or 100% sequence identity with the amino acid sequence of SEQ ID NO: 45, while still retaining an LCDR1 that comprises the amino acid sequence of SEQ ID NO: 201, an LCDR2 that comprises the amino acid sequence of SEQ ID NO: 202, and an LCDR3 that comprises the amino acid sequence of SEQ ID NO: 203. In more particular embodiments, the canine antibody comprises a heavy chain that comprises the amino acid sequence of SEQ ID NO: 44 and a light chain that comprises the amino acid sequence of SEQ ID NO: 45. The present invention further provides antigen binding fragments of these canine antibodies.

In other specific embodiments, the canine antibodies comprise a heavy chain that comprises at least 90%, 95%, 98%, 99% or 100% sequence identity with the amino acid sequence of SEQ ID NO: 241, while still retaining an HCDR1 that comprises the amino acid sequence of SEQ ID NO: 198, an HCDR2 that comprises the amino acid sequence of SEQ ID NO: 199, and an HCDR3 that comprises the amino acid sequence of SEQ ID NO: 200 and a light chain that comprises at least 90%, 95%, 98%, 99% or 100% sequence identity with the amino acid sequence of SEQ ID NO: 45, while still retaining an LCDR1 that comprises the amino acid sequence of SEQ ID NO: 201, an LCDR2 that comprises the amino acid sequence of SEQ ID NO: 202, and an LCDR3 that comprises the amino acid sequence of SEQ ID NO: 203. In more particular embodiments, the canine antibody comprises a heavy chain that comprises the amino acid sequence of SEQ ID NO: 241 and a light chain that comprises the amino acid sequence of SEQ ID NO: 45. The present invention further provides antigen binding fragments of these canine antibodies.

In yet other embodiments, the canine antibodies comprise a heavy chain that comprises at least 90%, 95%, 98%, 99% or 100% sequence identity with the amino acid sequence of SEQ ID NO: 241, while still retaining the tyrosine residue at amino acid residue position 252 and the aspartic acid residue at amino acid residue position 256, as numbered according to the EU index as in Kabat, while still retaining an HCDR1 that comprises the amino acid sequence of SEQ ID NO: 198, an HCDR2 that comprises the amino acid sequence of SEQ ID NO: 199, and an HCDR3 that comprises the amino acid sequence of SEQ ID NO: 200 and a light chain that comprises at least 90%, 95%, 98%, 99% or 100% sequence identity with the amino acid sequence of SEQ ID NO: 45, while still retaining an LCDR1 that comprises the amino acid sequence of SEQ ID NO: 201, an LCDR2 that comprises the amino acid sequence of SEQ ID NO: 202, and an LCDR3 that comprises the amino acid sequence of SEQ ID NO: 203. In more particular embodiments, the canine antibody comprises a heavy chain that comprises the amino acid sequence of SEQ ID NO: 241 and a light chain that comprises the amino acid sequence of SEQ ID NO: 45. The present invention further provides antigen binding fragments of these canine antibodies.

In still other embodiments, the canine antibodies comprise a heavy chain that comprises at least 90%, 95%, 98%, 99% or 100% sequence identity with the amino acid sequence of SEQ ID NO: 46, while still retaining an HCDR1 that comprises the amino acid sequence of SEQ ID NO: 192, an HCDR2 that comprises the amino acid sequence of SEQ ID NO: 193, and an HCDR3 that comprises the amino acid sequence of SEQ ID NO: 194 and a light chain that comprises at least 90%, 95%, 98%, 99% or 100% sequence identity with the amino acid sequence of SEQ ID NO: 47, while still retaining an LCDR1 that comprises the amino acid sequence of SEQ ID NO: 195, an LCDR2 that comprises the amino acid sequence of SEQ ID NO: 196, and an LCDR3 that comprises the amino acid sequence of SEQ ID NO: 197. In more particular embodiments, the canine antibody comprises a heavy chain that comprises the amino acid sequence of SEQ ID NO: 46 and a light chain that comprises the amino acid sequence of SEQ ID NO: 47. The present invention further provides antigen binding fragments of these canine antibodies.

In yet other embodiments, the canine antibodies comprise a heavy chain that comprises at least 90%, 95%, 98%, 99% or 100% sequence identity with the amino acid sequence of SEQ ID NO: 48, while still retaining an HCDR1 that comprises the amino acid sequence of SEQ ID NO: 186, an HCDR2 that comprises the amino acid sequence of SEQ ID NO: 187, and an HCDR3 that comprises the amino acid sequence of SEQ ID NO: 188, and a light chain that comprises at least 90%, 95%, 98%, 99% or 100% sequence identity with the amino acid sequence of SEQ ID NO: 49, while still retaining an LCDR1 that comprises the amino acid sequence of SEQ ID NO: 189, an LCDR2 that comprises the amino acid sequence of SEQ ID NO: 190, and an LCDR3 that comprises the amino acid sequence of SEQ ID NO: 191. In more particular embodiments, the canine antibody comprises a heavy chain that comprises the amino acid sequence of SEQ ID NO: 48 and a light chain that comprises the amino acid sequence of SEQ ID NO: 49. The present invention further provides antigen binding fragments of these canine antibodies.

In still other embodiments, the canine antibodies comprise a heavy chain that comprises at least 90%, 95%, 98%, 99% or 100% sequence identity with the amino acid sequence of SEQ ID NO: 50, while still retaining an HCDR1 that comprises the amino acid sequence of SEQ ID NO: 180, an HCDR2 that comprises the amino acid sequence of SEQ ID NO: 181, and an HCDR3 that comprises the amino acid sequence of SEQ ID NO: 182 and a light chain that comprises at least 90%, 95%, 98%, 99% or 100% sequence identity with the amino acid sequence of SEQ ID NO: 51, while still retaining an LCDR1 that comprises the amino acid sequence of SEQ ID NO: 183, an LCDR2 that comprises the amino acid sequence of SEQ ID NO: 184, and an LCDR3 that comprises the amino acid sequence of SEQ ID NO: 185. In more particular embodiments, the canine antibody comprises a heavy chain that comprises the amino acid sequence of SEQ ID NO: 50 and a light chain that comprises the amino acid sequence of SEQ ID NO: 51. The present invention further provides antigen binding fragments of these canine antibodies.

In yet other embodiments, the canine antibodies comprise a heavy chain that comprises at least 90%, 95%, 98%, 99% or 100% sequence identity with the amino acid sequence of SEQ ID NO: 52, while still retaining an HCDR1 that comprises the amino acid sequence of SEQ ID NO: 156, an HCDR2 that comprises the amino acid sequence of SEQ ID NO: 157, and an HCDR3 that comprises the amino acid sequence of SEQ ID NO: 158 and a light chain that comprises at least 90%, 95%, 98%, 99% or 100% sequence identity with the amino acid sequence of SEQ ID NO: 53, while still retaining an LCDR1 that comprises the amino acid sequence of SEQ ID NO: 159, an LCDR2 that comprises the amino acid sequence of SEQ ID NO: 160, and an LCDR3 that comprises the amino acid sequence of SEQ ID NO: 161. In more particular embodiments, the canine antibody comprises a heavy chain that comprises the amino acid sequence of SEQ ID NO: 52 and a light chain that comprises the amino acid sequence of SEQ ID NO: 53. The present invention further provides antigen binding fragments of these canine antibodies.

In still other embodiments, the canine antibodies comprise a heavy chain that comprises at least 90%, 95%, 98%, 99% or 100% sequence identity with the amino acid sequence of SEQ ID NO: 54, while still retaining an HCDR1 that comprises the amino acid sequence of SEQ ID NO: 162, an HCDR2 that comprises the amino acid sequence of SEQ ID NO: 163, and an HCDR3 that comprises the amino acid sequence of SEQ ID NO: 164, and a light chain that comprises at least 90%, 95%, 98%, 99% or 100% sequence identity with the amino acid sequence of SEQ ID NO: 55, while still retaining an LCDR1 that comprises the amino acid sequence of SEQ ID NO: 165, an LCDR2 that comprises the amino acid sequence of SEQ ID NO: 166, and an LCDR3 that comprises the amino acid sequence of SEQ ID NO: 167. In more particular embodiments, the canine antibody comprises a heavy chain that comprises the amino acid sequence of SEQ ID NO: 54 and a light chain that comprises the amino acid sequence of SEQ ID NO: 55. The present invention further provides antigen binding fragments of these canine antibodies.

In yet other embodiments, the canine antibodies comprise a heavy chain that comprises at least 90%, 95%, 98%, 99% or 100% sequence identity with the amino acid sequence of SEQ ID NO: 56, while still retaining an HCDR1 that comprises the amino acid sequence of SEQ ID NO: 162, an HCDR2 that comprises the amino acid sequence of SEQ ID NO: 237, and an HCDR3 that comprises the amino acid sequence of SEQ ID NO: 164, and a light chain that comprises at least 90%, 95%, 98%, 99% or 100% sequence identity with the amino acid sequence of SEQ ID NO: 55, while still retaining an LCDR1 that comprises the amino acid sequence of SEQ ID NO: 165, an LCDR2 that comprises the amino acid sequence of SEQ ID NO: 166, and an LCDR3 that comprises the amino acid sequence of SEQ ID NO: 167. In more particular embodiments, the canine antibody comprises a heavy chain that comprises the amino acid sequence of SEQ ID NO: 56 and a light chain that comprises the amino acid sequence of SEQ ID NO: 55. The present invention further provides antigen binding fragments of these canine antibodies.

In still other embodiments, the canine antibodies comprise a heavy chain that comprises at least 90%, 95%, 98%, 99% or 100% sequence identity with the amino acid sequence of SEQ ID NO: 57, while still retaining an HCDR1 that comprises the amino acid sequence of SEQ ID NO: 168, an HCDR2 that comprises the amino acid sequence of SEQ ID NO: 169, and an HCDR3 that comprises the amino acid sequence of SEQ ID NO: 170 and a light chain that comprises at least 90%, 95%, 98%, 99% or 100% sequence identity with the amino acid sequence of SEQ ID NO: 58, while still retaining an LCDR1 that comprises the amino acid sequence of SEQ ID NO: 171, an LCDR2 that comprises the amino acid sequence of SEQ ID NO: 172, and an LCDR3 that comprises the amino acid sequence of SEQ ID NO: 173. In more particular embodiments, the canine antibody comprises a heavy chain that comprises the amino acid sequence of SEQ ID NO: 57 and a light chain that comprises the amino acid sequence of SEQ ID NO: 58. The present invention further provides antigen binding fragments of these canine antibodies.

In yet other embodiments, the canine antibodies comprise a heavy chain that comprises at least 90%, 95%, 98%, 99% or 100% sequence identity with the amino acid sequence of SEQ ID NO: 59 while still retaining an HCDR1 that comprises the amino acid sequence of SEQ ID NO: 174 an HCDR2 that comprises the amino acid sequence of SEQ ID NO: 175, and an HCDR3 that comprises the amino acid sequence of SEQ ID NO: 176, and a light chain that comprises at least 90%, 95%, 98%, 99% or 100% sequence identity with the amino acid sequence of SEQ ID NO: 60, while still retaining an LCDR1 that comprises the amino acid sequence of SEQ ID NO: 177, an LCDR2 that comprises the amino acid sequence of SEQ ID NO: 178, and an LCDR3 that comprises the amino acid sequence of SEQ ID NO: 179. In more particular embodiments, the canine antibody comprises a heavy chain that comprises the amino acid sequence of SEQ ID NO: 59 and a light chain that comprises the amino acid sequence of SEQ ID NO: 60. The present invention further provides antigen binding fragments of these canine antibodies.

The cFc of the heavy chain of the canine antibodies of the present invention or antigen binding fragments thereof, can further comprise one or more amino acid residue substitutions, wherein the amino acid residue positions are numbered according to the EU index as in Kabat. In certain embodiments, one amino acid residue substitution is at amino acid residue position 252. In a particular embodiment of this type, the substitution at amino acid residue position 252 is with a tyrosine residue. In other embodiments, one amino acid residue substitution is at amino acid residue position 254. In a particular embodiment of this type, the substitution at amino acid residue position 254 is with a threonine residue. In still other embodiments, one amino acid residue substitution is at amino acid residue position 256. In a particular embodiment of this type, the substitution at amino acid residue position 256 is with an aspartic acid residue or a glutamic acid residue. In yet other embodiments, one amino acid residue substitution is at amino acid residue position 308. In a particular embodiment of this type, the substitution at amino acid residue position 308 is with a proline residue. In still other embodiments, an amino acid residue substitution is at amino acid residue position 433. In a particular embodiment of this type, the substitution at amino acid residue position 433 is with a lysine residue or a leucine residue. In yet other embodiments, an amino acid residue substitution is at amino acid residue position 434. In a particular embodiment of this type, the substitution at amino acid residue position 434 is with a phenylalanine residue, a histidine residue, or a tyrosine residue. In still other embodiments, an amino acid residue substitution is at amino acid residue position 436.

In a particular embodiment of this type, the substitution at amino acid residue position 436 is with a threonine residue.

The cFc of the heavy chain of the canine antibodies of the present invention or antigen binding fragments thereof, can further comprise two or more amino acid residue substitutions, wherein the amino acid residue positions are numbered according to the EU index as in Kabat. In certain embodiments, one amino acid residue substitution is at amino acid residue position 252 and a second amino acid residue substitution is at amino acid residue position 256. In a particular embodiment of this type, the substitution at amino acid residue position 252 is with a tyrosine residue and the substitution at amino acid residue position 256 is with an aspartic acid residue. In other embodiments, one amino acid residue substitution is at amino acid residue position 256 and a second amino acid residue substitution is at amino acid residue position 434. In a particular embodiment of this type, the substitution at amino acid residue position 256 is with an aspartic acid residue and the substitution at amino acid residue position 434 is with a tyrosine residue. In an alternative embodiment of this type, the substitution at amino acid residue position 256 is with an aspartic acid residue and the substitution at amino acid residue position 434 is with a histidine residue. In yet other embodiments, one amino acid residue substitution is at amino acid residue position 308 and a second amino acid residue substitution is at amino acid residue position 434. In a particular embodiment of this type, the substitution at amino acid residue position 308 is with a proline residue and the substitution at amino acid residue position 434 is with a tyrosine residue. In an alternative embodiment of this type, the substitution at amino acid residue position 308 is with a proline residue and the substitution at amino acid residue position 434 is with a histidine residue. In still other embodiments, one amino acid residue substitution is at amino acid residue position 433 and a second amino acid residue substitution is at amino acid residue position 434. In a particular embodiment of this type, the substitution at amino acid residue position 433 is with a lysine residue and the substitution at amino acid residue position 434 is with a tyrosine residue. In an alternative embodiment of this type, the substitution at amino acid residue position 433 is with a lysine residue and the substitution at amino acid residue position 434 is with a histidine residue. In yet another embodiment of this type, the substitution at amino acid residue position 433 is with a lysine residue and the substitution at amino acid residue position 434 is with a phenylalanine residue. In still another embodiment of this type, the substitution at amino acid residue position 433 is with a leucine residue and the substitution at amino acid residue position 434 is with a phenylalanine residue. In yet another embodiment of this type, the substitution at amino acid residue position 433 is with a leucine residue and the substitution at amino acid residue position 434 is with a tyrosine residue. In still another embodiment of this type, the substitution at amino acid residue position 433 is with a leucine residue and the substitution at amino acid residue position 434 is with a histidine residue. In yet other embodiments, one amino acid residue substitution is at amino acid residue position 434 and a second amino acid residue substitution is at amino acid residue position 436. In a particular embodiment of this type, the substitution is at amino acid residue position 434 with a tyrosine residue and the substitution at amino acid residue position 436 is with a threonine residue. In another embodiment of this type, the substitution at amino acid residue position 434 is with a phenylalanine residue and the substitution at amino acid residue position 436 is with a threonine residue. In an alternative embodiment of this type, the substitution at amino acid residue position 434 is with a histidine residue and the substitution at amino acid residue position 436 is with a threonine residue.

The cFc of the heavy chain of the canine antibodies of the present invention or antigen binding fragments thereof, can further comprise three or more amino acid residue substitutions, wherein the amino acid residue positions are numbered according to the EU index as in Kabat. In certain embodiments, one amino acid residue substitution is at amino acid residue position 252, a second amino acid residue substitution is at amino acid residue position 254, and a third amino acid residue substitution is at amino acid residue position 256. In a particular embodiment of this type, the substitution at amino acid residue position 252 is with a tyrosine residue, the substitution at amino acid residue position 254 is with a with a threonine residue, and the substitution at amino acid residue position 256 is with a glutamic acid residue. In other embodiments, one amino acid residue substitution is at amino acid residue position 256, a second amino acid residue substitution is at amino acid residue position 308, and a third amino acid residue substitution is at amino acid residue position 434. In a particular embodiment of this type, the substitution at amino acid residue position 256 is with an aspartic acid residue, the substitution at amino acid residue position 308 is with a proline residue, and the substitution at amino acid residue position 434 is with a tyrosine residue. In yet other embodiments, one amino acid residue substitution is at amino acid residue position 433, a second amino acid residue substitution is at amino acid residue position 434, and a third amino acid residue substitution is at amino acid residue position 436. In a particular embodiment of this type, the substitution at amino acid residue position 433 is with a lysine residue, the substitution at amino acid residue position 434 is with a phenylalanine residue, and the substitution at amino acid residue position 436 is with a threonine residue. In another embodiment of this type, the substitution at amino acid residue position 433 is with a lysine residue, the substitution at amino acid residue position 434 is with a tyrosine residue, and the substitution at amino acid residue position 436 is with a threonine residue. In yet another embodiment of this type, the substitution at amino acid residue position 433 is with a lysine residue, the substitution at amino acid residue position 434 is with a histidine residue, and the substitution at amino acid residue position 436 is with a threonine residue. In still another embodiment of this type, the substitution at amino acid residue position 433 is with a leucine residue, the substitution at amino acid residue position 434 is with a phenylalanine residue, and the substitution at amino acid residue position 436 is with a threonine residue. In yet another embodiment of this type, the substitution at amino acid residue position 433 is with a leucine residue, the substitution at amino acid residue position 434 is with a tyrosine residue, and the substitution at amino acid residue position 436 is with a threonine residue. In still another embodiment of this type, the substitution at amino acid residue position 433 is with a leucine residue, the substitution at amino acid residue position 434 is with a histidine residue, and the substitution at amino acid residue position 436 is with a threonine residue.

In particular embodiments of the canine antibodies or antigen binding fragments thereof that comprise the aforementioned heavy chains and light chains, when bound to cIL-4, the canine antibodies or antigen binding fragments thereof (i) bind to an epitope comprised by the amino acid sequence of SEQ ID NO: 89, SEQ ID NO: 90, SEQ ID NO: 91, SEQ ID NO: 92, SEQ ID NO: 238, or any combination thereof, (ii) have an IC50 of lower than 40 nM for canine IL-4 mediated STAT-6 phosphorylation in DH82 cells, or (iii) both bind to an epitope comprised by the amino acid sequence of SEQ ID NO: 89, SEQ ID NO: 90, SEQ ID NO: 91, SEQ ID NO: 92, SEQ ID NO: 238, or any combination thereof, and have an IC50 of lower than 40 nM for canine IL-4 mediated STAT-6 phosphorylation in DH82 cells. In more particular embodiments, the canine antibody or antigen binding fragment thereof, when bound to cIL-4 binds to an epitope comprised by the amino acid sequence of SEQ ID NO: 89, SEQ ID NO: 90, SEQ ID NO: 91, SEQ ID NO: 92, SEQ ID NO: 238, or any combination thereof. In certain embodiments, the canine antibody or antigen binding fragment thereof binds to one or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In other embodiments, the canine antibody or antigen binding fragment thereof binds to two or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In yet other embodiments, the canine antibody or antigen binding fragment thereof binds to three or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In still other embodiments, the canine antibody or antigen binding fragment thereof binds to four or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In yet other embodiments, the canine antibody or antigen binding fragment thereof binds to five or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In still other embodiments, the canine antibody or antigen binding fragment thereof binds to six or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In particular embodiments, the canine antibody or antigen binding fragment thereof binds to three to five amino acid residues of cIL-4 at the following amino acid residue positions: 6 (T₆), 8 (K₈), 12 (K₁₂), 74 (S₇₄), and 79 (T₇₉) of the amino acid sequence of SEQ ID NO: 1. In other particular embodiments, the canine antibody or antigen binding fragment thereof binds to three to five amino acid residues or more of cIL-4 at the following amino acid residue positions: 12 (K₁₂), 20 (R₂₀), 23 (S₂₃), 28 (T₂₈), 37 (K₃₇), 39 (T₃₉), 86 (K₈₆), 87 (K₈₇), 88 (S₈₈), 89 (T₈₉), 91 (K₉₁), and 96 (R₉₆) of the amino acid sequence of SEQ ID NO: 1.

The present invention further provides canine antibodies or antigen binding fragments thereof that bind cIL-4 and have an IC50 of lower than 40 nM for canine IL-4 mediated STAT-6 phosphorylation in DH82 cells. The present invention further provides canine antibodies or antigen binding fragments thereof that bind cIL-4 and have an IC50 of lower than 20 nM for canine IL-4 mediated STAT-6 phosphorylation in DH82 cells. In particular embodiments, the canine antibodies or antigen binding fragments thereof bind cIL-4 and have an IC50 of lower than 10 nM for canine IL-4 mediated STAT-6 phosphorylation in DH82 cells. In still other embodiments, the canine antibodies or antigen binding fragments thereof bind cIL-4 and have an IC50 of between 0.5 to 50 nM for canine IL-4 mediated STAT-6 phosphorylation in DH82 cells. In yet other embodiments, the canine antibodies or antigen binding fragments thereof bind cIL-4 and have an IC50 of between 2 to 25 nM for canine IL-4 mediated STAT-6 phosphorylation in DH82 cells. In still other embodiments, the canine antibodies or antigen binding fragments thereof bind cIL-4 and have an IC50 of between 4 to 20 nM for canine IL-4 mediated STAT-6 phosphorylation in DH82 cells.

In even more particular embodiments, the canine antibody or antigen binding fragment thereof, binds to an epitope of cIL-4 comprised by the amino acid sequence of SEQ ID NO: 89, SEQ ID NO: 90, SEQ ID NO: 91, SEQ ID NO: 92, and SEQ ID NO: 238, or any combination thereof, binds to one, two, three, four, five, or more amino acid residues of cIL-4 at the following amino acid residue positions: 12 (K₁₂), 20 (R₂₀), 23 (S₂₃), 28 (T₂₈), 37 (K₃₇), 39 (T₃₉), 86 (K₈₆), 87 (K₈₇), 88 (S₈₈), 89 (T₈₉), 91 (K₉₁), and 96 (R₉₆) of the amino acid sequence of SEQ ID NO: 1, and has an IC50 of lower than 50 nM, lower than 25 nM, and/or between 2 and/or 4 to 20 nM for canine IL-4 mediated STAT-6 phosphorylation in DH82 cells.

The present invention further provides nucleic acids that individually encode one of the heavy chains of the antibodies of the present invention and nucleic acids that individually encode one of the corresponding light chains of the antibodies of the present invention or an antigen binding fragment thereof. The present invention further provides as a pair, a nucleic acid encoding the heavy chain or an antigen binding fragment thereof of an antibody of the present invention and a nucleic acid that encodes the corresponding light chain or an antigen binding fragment thereof of that antibody of the present invention. The present invention also provides a kit containing this pair of two nucleic acids.

In particular embodiments, the nucleic acid encodes a heavy chain that comprises at least 90%, 95%, 98%, 99% or 100% sequence identity with the amino acid sequence of SEQ ID NO: 42, while still retaining an HCDR1 that comprises the amino acid sequence of SEQ ID NO: 204, an HCDR2 that comprises the amino acid sequence of SEQ ID NO: 205, and an HCDR3 that comprises the amino acid sequence of SEQ ID NO: 206, and another nucleic acid that encodes a corresponding a light chain that comprises at least 90%, 95%, 98%, 99% or 100% sequence identity with the amino acid sequence of SEQ ID NO: 43, while still retaining an LCDR1 that comprises the amino acid sequence of SEQ ID NO: 207, an LCDR2 that comprises the amino acid sequence of SEQ ID NO: 208, and an LCDR3 that comprises the amino acid sequence of SEQ ID NO: 209. The present invention further provides as a pair, the nucleic acid encoding the heavy chain of the canine antibody and the nucleic acid that encodes the corresponding light chain of that canine antibody. The present invention also provides a kit containing this pair of two nucleic acids.

In more particular embodiments, a nucleic acid encodes a heavy chain of a canine antibody or antigen binding fragment thereof that comprises the amino acid sequence of SEQ ID NO: 42 and another nucleic acid that encodes a corresponding light chain of that antibody that comprises the amino acid sequence of SEQ ID NO: 43. The present invention further provides as a pair, a nucleic acid encoding the heavy chain that comprises the amino acid sequence of SEQ ID NO: 42 and a nucleic acid that encodes the corresponding light chain that comprises the amino acid sequence of SEQ ID NO: 43. The present invention also provides a kit containing this pair of two nucleic acids.

In specific embodiments, the nucleic acid encodes a heavy chain that comprises at least 90%, 95%, 98%, 99% or 100% sequence identity with the amino acid sequence of SEQ ID NO: 44, while still retaining an HCDR1 that comprises the amino acid sequence of SEQ ID NO: 198, an HCDR2 that comprises the amino acid sequence of SEQ ID NO: 199, and an HCDR3 that comprises the amino acid sequence of SEQ ID NO: 200 and another nucleic acid that encodes a corresponding a light chain that comprises at least 90%, 95%, 98%, 99% or 100% sequence identity with the amino acid sequence of SEQ ID NO: 45, while still retaining an LCDR1 that comprises the amino acid sequence of SEQ ID NO: 201, an LCDR2 that comprises the amino acid sequence of SEQ ID NO: 202, and an LCDR3 that comprises the amino acid sequence of SEQ ID NO: 203. The present invention further provides as a pair, a nucleic acid encoding the heavy chain of the canine antibody and a nucleic acid that encodes the corresponding light chain of that canine antibody. The present invention also provides a kit containing this pair of two nucleic acids.

In more specific embodiments, a nucleic acid encodes a heavy chain of a canine antibody or antigen binding fragment thereof that comprises the amino acid sequence of SEQ ID NO: 44 and the corresponding light chain of that antibody that comprises the amino acid sequence of SEQ ID NO: 45. The present invention further provides as a pair, a nucleic acid encoding the heavy chain that comprises the amino acid sequence of SEQ ID NO: 44 and a nucleic acid that encodes the corresponding light chain that comprises the amino acid sequence of SEQ ID NO: 45. The present invention also provides a kit containing this pair of two nucleic acids.

In other specific embodiments, the nucleic acid encodes a heavy chain that comprises at least 90%, 95%, 98%, 99% or 100% sequence identity with the amino acid sequence of SEQ ID NO: 241, while still retaining an HCDR1 that comprises the amino acid sequence of SEQ ID NO: 198, an HCDR2 that comprises the amino acid sequence of SEQ ID NO: 199, and an HCDR3 that comprises the amino acid sequence of SEQ ID NO: 200 and another nucleic acid that encodes a corresponding a light chain that comprises at least 90%, 95%, 98%, 99% or 100% sequence identity with the amino acid sequence of SEQ ID NO: 45, while still retaining an LCDR1 that comprises the amino acid sequence of SEQ ID NO: 201, an LCDR2 that comprises the amino acid sequence of SEQ ID NO: 202, and an LCDR3 that comprises the amino acid sequence of SEQ ID NO: 203. The present invention further provides as a pair, a nucleic acid encoding the heavy chain of the canine antibody and a nucleic acid that encodes the corresponding light chain of that canine antibody. The present invention also provides a kit containing this pair of two nucleic acids.

In yet other embodiments the nucleic acid encodes a heavy chain that comprises at least 90%, 95%, 98%, 99% or 100% sequence identity with the amino acid sequence of SEQ ID NO: 241, while still retaining the tyrosine residue at amino acid residue position 252 and the aspartic acid residue at amino acid residue position 256, as numbered according to the EU index as in Kabat, while still retaining an HCDR1 that comprises the amino acid sequence of SEQ ID NO: 198, an HCDR2 that comprises the amino acid sequence of SEQ ID NO: 199, and an HCDR3 that comprises the amino acid sequence of SEQ ID NO: 200 and a light chain that comprises at least 90%, 95%, 98%, 99% or 100% sequence identity with the amino acid sequence of SEQ ID NO: 45, while still retaining an LCDR1 that comprises the amino acid sequence of SEQ ID NO: 201, an LCDR2 that comprises the amino acid sequence of SEQ ID NO: 202, and an LCDR3 that comprises the amino acid sequence of SEQ ID NO: 203. The present invention further provides as a pair, a nucleic acid encoding the heavy chain of the canine antibody and a nucleic acid that encodes the corresponding light chain of that canine antibody. The present invention also provides a kit containing this pair of two nucleic acids.

In more specific embodiments, a nucleic acid encodes a heavy chain of a canine antibody or antigen binding fragment thereof that comprises the amino acid sequence of SEQ ID NO: 241 and the corresponding light chain of that antibody that comprises the amino acid sequence of SEQ ID NO: 45. The present invention further provides as a pair, a nucleic acid encoding the heavy chain that comprises the amino acid sequence of SEQ ID NO: 241 and a nucleic acid that encodes the corresponding light chain that comprises the amino acid sequence of SEQ ID NO: 45. The present invention also provides a kit containing this pair of two nucleic acids.

In particular embodiments, the nucleic acid encodes a heavy chain of a canine antibody or antigen binding fragment thereof that comprises at least 90%, 95%, 98%, 99% or 100% sequence identity with the amino acid sequence of SEQ ID NO: 46, while still retaining an HCDR1 that comprises the amino acid sequence of SEQ ID NO: 192, an HCDR2 that comprises the amino acid sequence of SEQ ID NO: 193, and an HCDR3 that comprises the amino acid sequence of SEQ ID NO: 194, and another nucleic acid that encodes a corresponding light chain of that antibody that comprises at least 90%, 95%, 98%, 99% or 100% sequence identity with the amino acid sequence of SEQ ID NO: 47, while still retaining an LCDR1 that comprises the amino acid sequence of SEQ ID NO: 195, an LCDR2 that comprises the amino acid sequence of SEQ ID NO: 196, and an LCDR3 that comprises the amino acid sequence of SEQ ID NO: 197. The present invention further provides as a pair, a nucleic acid encoding the heavy chain of the canine antibody and a nucleic acid that encodes the corresponding light chain of that canine antibody. The present invention also provides a kit containing this pair of two nucleic acids.

In a more particular embodiment, a nucleic acid encodes a heavy chain of a canine antibody that comprises the amino acid sequence of SEQ ID NO: 46 and another nucleic acid that encodes a corresponding light chain of that antibody that comprises the amino acid sequence of SEQ ID NO: 47. The present invention further provides as a pair, a nucleic acid encoding the heavy chain that comprises the amino acid sequence of SEQ ID NO: 46 and a nucleic acid that encodes the corresponding light chain that comprises the amino acid sequence of SEQ ID NO: 47. The present invention also provides a kit containing this pair of two nucleic acids.

In other particular embodiments, the nucleic acid encodes a heavy chain of a canine antibody or antigen binding fragment thereof that comprises at least 90%, 95%, 98%, 99% or 100% sequence identity with the amino acid sequence of SEQ ID NO: 48, while still retaining an HCDR1 that comprises the amino acid sequence of SEQ ID NO: 186, an HCDR2 that comprises the amino acid sequence of SEQ ID NO: 187, and an HCDR3 that comprises the amino acid sequence of SEQ ID NO: 188, and another nucleic acid that encodes a corresponding light chain of that antibody that comprises at least 90%, 95%, 98%, 99% or 100% sequence identity with the amino acid sequence of SEQ ID NO: 49, while still retaining an LCDR1 that comprises the amino acid sequence of SEQ ID NO: 189, an LCDR2 that comprises the amino acid sequence of SEQ ID NO: 190, and an LCDR3 that comprises the amino acid sequence of SEQ ID NO: 191. The present invention further provides as a pair, a nucleic acid encoding the heavy chain of the canine antibody and a nucleic acid that encodes the corresponding light chain of that canine antibody. The present invention also provides a kit containing this pair of two nucleic acids.

In still more particular embodiments, a nucleic acid encodes a heavy chain of a canine antibody or antigen binding fragment thereof that comprises the amino acid sequence of SEQ ID NO: 48 and another nucleic acid that encodes a corresponding light chain of that antibody that comprises the amino acid sequence of SEQ ID NO: 49. The present invention further provides as a pair, a nucleic acid encoding the heavy chain that comprises the amino acid sequence of SEQ ID NO: 48 and a nucleic acid that encodes the corresponding light chain that comprises the amino acid sequence of SEQ ID NO: 49. The present invention also provides a kit containing this pair of two nucleic acids.

In yet other particular embodiments, the nucleic acid encodes a heavy chain of a canine antibody or antigen binding fragment thereof that comprises at least 90%, 95%, 98%, 99% or 100% sequence identity with the amino acid sequence of SEQ ID NO: 52, while still retaining an HCDR1 that comprises the amino acid sequence of SEQ ID NO: 156, an HCDR2 that comprises the amino acid sequence of SEQ ID NO: 157, and an HCDR3 that comprises the amino acid sequence of SEQ ID NO: 158, and another nucleic acid that encodes a corresponding light chain of that antibody that comprises at least 90%, 95%, 98%, 99% or 100% sequence identity with the amino acid sequence of SEQ ID NO: 53, while still retaining an LCDR1 that comprises the amino acid sequence of SEQ ID NO: 159, an LCDR2 that comprises the amino acid sequence of SEQ ID NO: 160, and an LCDR3 that comprises the amino acid sequence of SEQ ID NO: 161. The present invention further provides as a pair, a nucleic acid encoding the heavy chain of the canine antibody and a nucleic acid that encodes the corresponding light chain of that canine antibody. The present invention also provides a kit containing this pair of two nucleic acids.

In yet more particular embodiments, a nucleic acid encodes a heavy chain of a canine antibody or antigen binding fragment thereof that comprises the amino acid sequence of SEQ ID NO: 52 and another nucleic acid that encodes a corresponding light chain of that antibody that comprises the amino acid sequence of SEQ ID NO: 53. The present invention further provides as a pair, a nucleic acid encoding the heavy chain that comprises the amino acid sequence of SEQ ID NO: 52 and a nucleic acid that encodes the corresponding light chain that comprises the amino acid sequence of SEQ ID NO: 53. The present invention also provides a kit containing this pair of two nucleic acids.

The present invention further provides canine antibodies or antigen binding fragments thereof, that when bound to cIL-4, (i) bind to an epitope comprised by the amino acid sequence of SEQ ID NO: 89, SEQ ID NO: 90, SEQ ID NO: 91, SEQ ID NO: 92, SEQ ID NO: 238, or any combination thereof, (ii) have an IC50 of lower than 40 nM for canine IL-4 mediated STAT-6 phosphorylation in DH82 cells, or (iii) both bind to an epitope comprised by the amino acid sequence of SEQ ID NO: 89, SEQ ID NO: 90, SEQ ID NO: 91, SEQ ID NO: 92, SEQ ID NO: 238, or any combination thereof, and have an IC50 of lower than 40 nM for canine IL-4 mediated STAT-6 phosphorylation in DH82 cells. In more particular embodiments, the canine antibody or antigen binding fragment thereof, when bound to cIL-4 binds to an epitope comprised by the amino acid sequence of SEQ ID NO: 89, SEQ ID NO: 90, SEQ ID NO: 91, SEQ ID NO: 92, SEQ ID NO: 238, or any combination thereof. In certain embodiments, the canine antibody or antigen binding fragment thereof binds to one or more amino acid residues of cIL-4 at the following amino acid residue positions: 6 (T₆), 8 (Kg), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In other embodiments, the canine antibody or antigen binding fragment thereof binds to two or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In yet other embodiments, the canine antibody or antigen binding fragment thereof binds to three or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In still other embodiments, the canine antibody or antigen binding fragment thereof binds to four or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In yet other embodiments, the canine antibody or antigen binding fragment thereof binds to five or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In still other embodiments, the canine antibody or antigen binding fragment thereof binds to six or more amino acid residues of cIL-4 at the following amino acid residue positions: 6 (T₆), 8 (K₈), 12 (K₁₂), 20 (R₂₀), 23 (S₂₃), 28 (T₂₈), 37 (K₃₇), 39 (T₃₉), 74 (S₇₄), 79 (T₇₉), 86 (K₈₆), 87 (K₈₇), 88 (S₈₈), 89 (T₈₉), 91 (K₉₁), and 96 (R₉₆) of the amino acid sequence of SEQ ID NO: 1. In particular embodiments, the canine antibody or antigen binding fragment thereof binds to three to five amino acid residues of cIL-4 at the following amino acid residue positions: 6 (T₆), 8 (K₈), 12 (K₁₂), 74 (S₇₄), and 79 (T₇₉) of the amino acid sequence of SEQ ID NO: 1. In other particular embodiments, the canine antibody or antigen binding fragment thereof binds to three to five amino acid residues or more of cIL-4 at the following amino acid residue positions: 12 (K₁₂), 20 (R₂₀), 23 (S₂₃), 28 (T₂₈), 37 (K₃₇), 39 (T₃₉), 86 (K₈₆), 87 (K₈₇), 88 (S₈₈), 89 (T₈₉), 91 (K₉₁), and 96 (R₉₆) of the amino acid sequence of SEQ ID NO: 1.

The present invention further provides canine antibodies or antigen binding fragments thereof that bind cIL-4 and have an IC50 of lower than 40 nM for canine IL-4 mediated STAT-6 phosphorylation in DH82 cells. The present invention further provides canine antibodies or antigen binding fragments thereof that bind cIL-4 and have an IC50 of lower than 20 nM for canine IL-4 mediated STAT-6 phosphorylation in DH82 cells. In particular embodiments, the canine antibodies or antigen binding fragments thereof bind cIL-4 and have an IC50 of lower than 10 nM for canine IL-4 mediated STAT-6 phosphorylation in DH82 cells. In still other embodiments, the canine antibodies or antigen binding fragments thereof bind cIL-4 and have an IC50 of between 0.5 to 50 nM for canine IL-4 mediated STAT-6 phosphorylation in DH82 cells. In yet other embodiments, the canine antibodies or antigen binding fragments thereof bind cIL-4 and have an IC50 of between 2 to 25 nM for canine IL-4 mediated STAT-6 phosphorylation in DH82 cells. In still other embodiments, the canine antibodies or antigen binding fragments thereof bind cIL-4 and have an IC50 of between 4 to 20 nM for canine IL-4 mediated STAT-6 phosphorylation in DH82 cells.

In even more particular embodiments, the canine antibody or antigen binding fragment thereof, binds to an epitope of cIL-4 comprised by the amino acid sequence of SEQ ID NO: 89, SEQ ID NO: 90, SEQ ID NO: 91, SEQ ID NO: 92, and SEQ ID NO: 238, or any combination thereof, binds to one, two, three, four, five, or more amino acid residues of cIL-4 at the following amino acid residue positions: 12 (K₁₂), 20 (R₂₀), 23 (S₂₃), 28 (T₂₈), 37 (K₃₇), 39 (T₃₉), 86 (K₈₆), 87 (K₈₇), 88 (S₈₈), 89 (T₈₉), 91 (K₉₁), and 96 (R₉₆) of the amino acid sequence of SEQ ID NO: 1, and has an IC50 of lower than 50 nM, lower than 25 nM, and/or between 2 and/or 4 to 20 nM for canine IL-4 mediated STAT-6 phosphorylation in DH82 cells.

The present invention further provides expression vectors that comprise and express one or more of the nucleic acids of the present invention. In particular embodiments, the expression vector comprises and expresses a nucleic acid encoding a heavy chain of a canine antibody of the present invention. In other embodiments, the expression vector comprises and expresses a nucleic acid encoding a light chain of that canine antibody. In more specific embodiments, the expression vector comprises a pair of nucleic acids, wherein one of the pair of nucleic acids comprises a nucleotide sequence that encodes the light chain of a specific canine antibody whereas the other of the pair of nucleic acids comprises a nucleotide sequence that encodes the heavy chain of said specific canine antibody. In related embodiments, the present invention provides a pair of expression vectors, one expression vector comprising the one of a pair of nucleic acids that comprises a nucleotide sequence that encodes the heavy chain of the specific canine antibody, whereas the other expression vector comprises the other of the pair of nucleic acids that comprises the nucleotide sequence that encodes the light chain of that specific canine antibody. The present invention also provides host cells that comprise one or more expression vectors of the present invention or one or more pairs of expression vectors. The present invention further provides pairs of host cells, with one of the host cell pairs comprising one of a pair of expression vectors, which comprises one of a pair of nucleic acids that comprises a nucleotide sequence encoding the heavy chain of the specific canine antibody, whereas the other one of the pair of host cells comprises the other one of the pair of expression vectors, which comprises the other one of the pair of nucleic acids that comprises a nucleotide sequence encoding the light chain of that specific canine antibody.

The present invention also provides pharmaceutical compositions and/or methods of treating atopic dermatitis with the pharmaceutical compositions comprising the canine antibodies and/or antigen binding fragments of the antibodies and a pharmaceutically acceptable carrier or diluent. In certain embodiments, the pharmaceutical compositions comprise canine antibodies that comprise a heavy chain variable region that comprises at least 90%, 95%, 98%, 99% or 100% sequence identity with the amino acid sequence of SEQ ID NO: 25, while still retaining an HCDR1 that comprises the amino acid sequence of SEQ ID NO: 198, an HCDR2 that comprises the amino acid sequence of SEQ ID NO: 199, and an HCDR3 that comprises the amino acid sequence of SEQ ID NO: 200, and a light chain variable region that comprises at least 90%, 95%, 98%, 99% or 100% sequence identity with the amino acid sequence of SEQ ID NO: 26, while still retaining an LCDR1 that comprises the amino acid sequence of SEQ ID NO: 201, an LCDR2 that comprises the amino acid sequence of SEQ ID NO: 202, and an LCDR3 that comprises the amino acid sequence of SEQ ID NO: 203. In specific embodiments, the pharmaceutical compositions comprise canine antibodies that comprise a heavy chain variable region that comprises the amino acid sequence of SEQ ID NO: 25 and a light chain variable region that comprises the amino acid sequence of SEQ ID NO: 26. In yet other embodiments, the pharmaceutical compositions comprise canine antibodies that comprise a heavy chain variable region that comprises at least 90%, 95%, 98%, 99% or 100% sequence identity with the amino acid sequence of SEQ ID NO: 28, while still retaining an HCDR1 that comprises the amino acid sequence of SEQ ID NO: 204, an HCDR2 that comprises the amino acid sequence of SEQ ID NO: 205, and an HCDR3 that comprises the amino acid sequence of SEQ ID NO: 206, and a light chain variable region that comprises at least 90%, 95%, 98%, 99% or 100% sequence identity with the amino acid sequence of SEQ ID NO: 29, while still retaining an LCDR1 that comprises the amino acid sequence of SEQ ID NO: 207, an LCDR2 that comprises the amino acid sequence of SEQ ID NO: 208, and an LCDR3 that comprises the amino acid sequence of SEQ ID NO: 209. In specific embodiments, the pharmaceutical compositions comprise canine antibodies that comprises a heavy chain variable region that comprises the amino acid sequence of SEQ ID NO: 28 and a light chain variable region that comprises the amino acid sequence of SEQ ID NO: 29.

In particular embodiments, the pharmaceutical compositions comprise canine antibodies that comprise a heavy chain that comprises at least 90%, 95%, 98%, 99% or 100% sequence identity with the amino acid sequence of SEQ ID NO: 44, while still retaining an HCDR1 that comprises the amino acid sequence of SEQ ID NO: 198, an HCDR2 that comprises the amino acid sequence of SEQ ID NO: 199, and an HCDR3 that comprises the amino acid sequence of SEQ ID NO: 200 and a light chain that comprises at least 90%, 95%, 98%, 99% or 100% sequence identity with the amino acid sequence of SEQ ID NO: 45, while still retaining an LCDR1 that comprises the amino acid sequence of SEQ ID NO: 201, an LCDR2 that comprises the amino acid sequence of SEQ ID NO: 202, and an LCDR3 that comprises the amino acid sequence of SEQ ID NO: 203. In more particular embodiments, the pharmaceutical compositions comprise canine antibodies that comprises a heavy chain that comprises the amino acid sequence of SEQ ID NO: 44 and a light chain that comprises the amino acid sequence of SEQ ID NO: 45.

In other embodiments, the pharmaceutical compositions comprise canine antibodies that comprise a heavy chain that comprises at least 90%, 95%, 98%, 99% or 100% sequence identity with the amino acid sequence of SEQ ID NO: 241, while still retaining an HCDR1 that comprises the amino acid sequence of SEQ ID NO: 198, an HCDR2 that comprises the amino acid sequence of SEQ ID NO: 199, and an HCDR3 that comprises the amino acid sequence of SEQ ID NO: 200 and a light chain that comprises at least 90%, 95%, 98%, 99% or 100% sequence identity with the amino acid sequence of SEQ ID NO: 45, while still retaining an LCDR1 that comprises the amino acid sequence of SEQ ID NO: 201, an LCDR2 that comprises the amino acid sequence of SEQ ID NO: 202, and an LCDR3 that comprises the amino acid sequence of SEQ ID NO: 203. In more particular embodiments, the pharmaceutical compositions comprise canine antibodies that comprise a heavy chain that comprises the amino acid sequence of SEQ ID NO: 241 and a light chain that comprises the amino acid sequence of SEQ ID NO: 45.

In related embodiments, the pharmaceutical compositions comprise canine antibodies that comprise a heavy chain that comprises at least 90%, 95%, 98%, 99% or 100% sequence identity with the amino acid sequence of SEQ ID NO: 241, while still retaining the tyrosine residue at amino acid residue position 252 and the aspartic acid residue at amino acid residue position 256, as numbered according to the EU index as in Kabat, while still retaining an HCDR1 that comprises the amino acid sequence of SEQ ID NO: 198, an HCDR2 that comprises the amino acid sequence of SEQ ID NO: 199, and an HCDR3 that comprises the amino acid sequence of SEQ ID NO: 200 and a light chain that comprises at least 90%, 95%, 98%, 99% or 100% sequence identity with the amino acid sequence of SEQ ID NO: 45, while still retaining an LCDR1 that comprises the amino acid sequence of SEQ ID NO: 201, an LCDR2 that comprises the amino acid sequence of SEQ ID NO: 202, and an LCDR3 that comprises the amino acid sequence of SEQ ID NO: 203.

In yet other embodiments, the pharmaceutical compositions comprise canine antibodies that comprise a heavy chain that comprises at least 90%, 95%, 98%, 99% or 100% sequence identity with the amino acid sequence of SEQ ID NO: 42, while still retaining an HCDR1 that comprises the amino acid sequence of SEQ ID NO: 204, an HCDR2 that comprises the amino acid sequence of SEQ ID NO: 205, and an HCDR3 that comprises the amino acid sequence of SEQ ID NO: 206 and a light chain that comprises at least 90%, 95%, 98%, 99% or 100% sequence identity with the amino acid sequence of SEQ ID NO: 43, while still retaining an LCDR1 that comprises the amino acid sequence of SEQ ID NO: 207, an LCDR2 that comprises the amino acid sequence of SEQ ID NO: 208, and an LCDR3 that comprises the amino acid sequence of SEQ ID NO: 209. In still other embodiments, the pharmaceutical compositions comprise canine antibodies that comprise a heavy chain that comprises the amino acid sequence of SEQ ID NO: 42 and a light chain that comprises the amino acid sequence of SEQ ID NO: 43.

In addition, pharmaceutical compositions are provided that comprise an expression vector comprising both a nucleic acid encoding a heavy chain of a canine antibody of the present invention and a nucleic acid encoding the light chain of that canine antibody, and a pharmaceutically acceptable carrier or diluent. In other embodiments, the pharmaceutical composition comprises a pharmaceutically acceptable carrier or diluent and two expression vectors, one comprising a nucleic acid encoding a heavy chain of a canine antibody of the present invention and the other expression vector comprises a nucleic acid encoding the light chain of that canine antibody. Such pharmaceutical compositions are capable of expressing the canine antibody and/or antigen binding fragments of the antibody of the present invention, in vivo. Any of the pharmaceutical compositions of the present invention can further comprise a canine or caninized antibody that binds canine interleukin-13 (cIL-13), a canine or caninized antibody that binds a canine interleukin-13 receptor alpha 1 (cIL-13R alpha 1), a canine or caninized antibody that binds canine interleukin-31 receptor alpha (cIL-31RA), or a canine or caninized antibody that binds canine interleukin-31 (cIL-31) or any combination thereof. In yet another embodiment, a pharmaceutical composition is provided for use in a method of decreasing the skin inflammation associated with atopic dermatitis in a canine, comprising administering to the canine in need thereof a therapeutically effective amount of the canine antibody that binds cIL-4 of the present invention, or antigen binding fragment thereof, or an expression vector of the present invention, or a pair of expression vectors of the present invention, or any combination thereof, and a pharmaceutically acceptable carrier or diluent; wherein said method is used for the treatment of atopic dermatitis.

In yet another embodiment, the present invention provides for a pharmaceutical composition for use in the treatment of atopic dermatitis, preferably atopic dermatitis in a canine, wherein the treatment comprises administering to the canine in need thereof a therapeutically effective amount of the canine antibody that binds cIL-13 of the present invention, or antigen binding fragment thereof, or an expression vector of the present invention, or a pair of expression vectors of the present invention, or any combination thereof, and a pharmaceutically acceptable carrier or diluent. Preferably, the treatment has the effect of decreasing the skin inflammation and/or pruritus associated with atopic dermatitis in a canine.

The present invention further provides additional methods of treating atopic dermatitis comprising administering one of the aforesaid pharmaceutical compositions to an animal subject that has atopic dermatitis. In particular embodiments, the animal subject is a canine. The present invention also provides methods of aiding in the blocking of the skin inflammation associated with atopic dermatitis, comprising administering to an animal subject in need thereof of a therapeutically effective amount of a pharmaceutical composition of the present invention. In particular embodiments, the animal subject is a canine.

The present invention further provides a pharmaceutical composition for use in the treatment of atopic dermatitis, said treatment comprising the administering one of the aforesaid pharmaceutical compositions to an animal subject that has atopic dermatitis. Preferably, the animal subject is a canine. Preferably, the treatment has the effect of decreasing the skin inflammation and/or pruritus associated with atopic dermatitis in a canine. In specific embodiments, methods of aiding in the blocking of the skin inflammation associated with atopic dermatitis are provided comprising administering to a subject in need thereof a therapeutically effective amount of the pharmaceutical composition of the present invention. In particular embodiments, the animal subject is a canine. In certain embodiments, methods of aiding in the blocking of the skin inflammation, pruritus, or both skin inflammation and pruritus associated with atopic dermatitis, comprises administering to a subject in need thereof a therapeutically effective amount of the pharmaceutical composition of the present invention comprising a canine antibody of the present invention that binds cIL-4 and blocks the binding of cIL-4 with the interleukin-4 receptor alpha (cIL-4Rα), together with a canine or caninized antibody that binds canine interleukin-13 (cIL-13), a canine or caninized antibody that binds canine IL-13 receptor alpha 1 (cIL-13R alpha 1), a canine or caninized antibody that binds canine interleukin-31 receptor alpha (cIL-31RA), a canine or caninized antibody that binds canine interleukin-31 (cIL-31) or any combination thereof. In particular embodiments, the animal subject is a canine.

In addition, the present invention provides methods of producing a canine antibody or antigen binding fragment thereof that binds canine IL-4. In particular embodiments, the method includes culturing host cells comprising one or more expression vectors that encode and express the light chain of a canine antibody of the present invention or the heavy chain of that canine antibody, or both the light chain of a canine antibody of the present invention and the heavy chain of that canine antibody in a culture medium (or culture media) under conditions in which the nucleic acid(s) is expressed, thereby producing a polypeptide comprising the light chain of a canine antibody of the present invention, the heavy chain of that canine antibody or both the light chain of a canine antibody of the present invention and the heavy chain of that canine antibody. The polypeptides are then recovered from the host cells or culture medium (or culture media). In certain embodiments, the polypeptides comprising the light chain of a canine antibody of the present invention and the polypeptides comprising the heavy chain of that canine antibody are combined with each under conditions that are conducive to form a canine antibody.

The present invention further provides single-chain Fv (scFv) antibodies that bind cIL-4, and comprise a set of six complementary determining regions, three of which are heavy chain CDRs (HCDR1, HCDR2, and HCDR3) and three of which are light chain CDRs (LCDR1, LCDR2, and LCDR3).

In particular embodiments of the scFvs, the HCDR1 that comprises the amino acid sequence of SEQ ID NO: 156, the HCDR2 comprises the amino acid sequence of SEQ ID NO: 157, the HCDR3 comprises the amino acid sequence of SEQ ID NO: 158, the LCDR1 comprises the amino acid sequence of SEQ ID NO: 159, the LCDR2 comprises the amino acid sequence of SEQ ID NO: 160, and the LCDR3 comprises the amino acid sequence of SEQ ID NO: 161.

In alternative embodiments of the scFvs, the HCDR1 comprises the amino acid sequence of SEQ ID NO: 162, the HCDR2 comprises the amino acid sequence of SEQ ID NO: 163, the HCDR3 comprises the amino acid sequence of SEQ ID NO: 164, the LCDR1 comprises the amino acid sequence of SEQ ID NO: 165, the LCDR2 comprises the amino acid sequence of SEQ ID NO: 166, and the LCDR3 comprises the amino acid sequence of SEQ ID NO: 167.

In related embodiments of the scFvs, the HCDR1 comprises the amino acid sequence of SEQ ID NO: 162, the HCDR2 comprises the amino acid sequence of SEQ ID NO: 237, the HCDR3 comprises the amino acid sequence of SEQ ID NO: 164, the LCDR1 comprises the amino acid sequence of SEQ ID NO: 165, the LCDR2 comprises the amino acid sequence of SEQ ID NO: 166, and the LCDR3 comprises the amino acid sequence of SEQ ID NO: 167.

In other embodiments of the scFvs, the HCDR1 comprises the amino acid sequence of SEQ ID NO: 168, the HCDR2 comprises the amino acid sequence of SEQ ID NO: 169, the HCDR3 comprises the amino acid sequence of SEQ ID NO: 170, the LCDR1 comprises the amino acid sequence of SEQ ID NO: 171, the LCDR2 comprises the amino acid sequence of SEQ ID NO: 172, and the LCDR3 comprises the amino acid sequence of SEQ ID NO: 173.

In yet other embodiments of the scFvs, the HCDR1 comprises the amino acid sequence of SEQ ID NO: 174, the HCDR2 comprises the amino acid sequence of SEQ ID NO: 175, the HCDR3 comprises the amino acid sequence of SEQ ID NO: 176, the LCDR1 comprises the amino acid sequence of SEQ ID NO: 177, the LCDR2 comprises the amino acid sequence of SEQ ID NO: 178, and the LCDR3 comprises the amino acid sequence of SEQ ID NO: 179.

In still other embodiments of the scFvs, the HCDR1 comprises the amino acid sequence of SEQ ID NO: 180, the HCDR2 comprises the amino acid sequence of SEQ ID NO: 181, the HCDR3 comprises the amino acid sequence of SEQ ID NO: 182, the LCDR1 comprises the amino acid sequence of SEQ ID NO: 183, the LCDR2 comprises the amino acid sequence of SEQ ID NO: 184, and the LCDR3 comprises the amino acid sequence of SEQ ID NO: 185.

In yet other embodiments of the scFvs, the HCDR1 comprises the amino acid sequence of SEQ ID NO: 186, the HCDR2 comprises the amino acid sequence of SEQ ID NO: 187, the HCDR3 comprises the amino acid sequence of SEQ ID NO: 188, the LCDR1 comprises the amino acid sequence of SEQ ID NO: 189, the LCDR2 comprises the amino acid sequence of SEQ ID NO: 190, and the LCDR3 comprises the amino acid sequence of SEQ ID NO: 191.

In still other embodiments of the scFvs, the HCDR1 comprises the amino acid sequence of SEQ ID NO: 192, the HCDR2 comprises the amino acid sequence of SEQ ID NO: 193, the HCDR3 comprises the amino acid sequence of SEQ ID NO: 194, the LCDR1 comprises the amino acid sequence of SEQ ID NO: 195, the LCDR2 comprises the amino acid sequence of SEQ ID NO: 196, and the LCDR3 comprises the amino acid sequence of SEQ ID NO: 197.

In yet other embodiments of the scFvs, the HCDR1 comprises the amino acid sequence of SEQ ID NO: 198, the HCDR2 comprises the amino acid sequence of SEQ ID NO: 199, the HCDR3 comprises the amino acid sequence of SEQ ID NO: 200, the LCDR1 comprises the amino acid sequence of SEQ ID NO: 201, the LCDR2 comprises the amino acid sequence of SEQ ID NO: 202, and the LCDR3 comprises the amino acid sequence of SEQ ID NO: 203.

In still other embodiments of the scFvs, the HCDR1 comprises the amino acid sequence of SEQ ID NO: 204, the HCDR2 comprises the amino acid sequence of SEQ ID NO: 205, the HCDR3 comprises the amino acid sequence of SEQ ID NO: 206, the LCDR1 comprises the amino acid sequence of SEQ ID NO: 207, the LCDR2 comprises the amino acid sequence of SEQ ID NO: 208, and the LCDR3 comprises the amino acid sequence of SEQ ID NO: 209.

In yet other embodiments of the scFvs, the HCDR1 comprises the amino acid sequence of SEQ ID NO: 210, the HCDR2 comprises the amino acid sequence of SEQ ID NO: 211, the HCDR3 comprises the amino acid sequence of SEQ ID NO: 212, the LCDR1 comprises the amino acid sequence of SEQ ID NO: 213, the LCDR2 comprises the amino acid sequence of SEQ ID NO: 214, and the LCDR3 comprises the amino acid sequence of SEQ ID NO: 215.

In still other embodiments of the scFvs, the HCDR1 comprises the amino acid sequence of SEQ ID NO: 216, the HCDR2 comprises the amino acid sequence of SEQ ID NO: 217, the HCDR3 comprises the amino acid sequence of SEQ ID NO: 218, the LCDR1 comprises the amino acid sequence of SEQ ID NO: 219, the LCDR2 comprises the amino acid sequence of SEQ ID NO: 220, and the LCDR3 comprises the amino acid sequence of SEQ ID NO: 221.

In yet other embodiments of the scFvs, the HCDR1 comprises the amino acid sequence of SEQ ID NO: 222, the HCDR2 comprises the amino acid sequence of SEQ ID NO: 223, the HCDR3 comprises the amino acid sequence of SEQ ID NO: 224, the LCDR1 comprises the amino acid sequence of SEQ ID NO: 225, the LCDR2 comprises the amino acid sequence of SEQ ID NO: 226, and the LCDR3 comprises the amino acid sequence of SEQ ID NO: 227.

In still other embodiments of the scFvs, the HCDR1 comprises the amino acid sequence of SEQ ID NO: 228, the HCDR2 comprises the amino acid sequence of SEQ ID NO: 229, the HCDR3 comprises the amino acid sequence of SEQ ID NO: 230, the LCDR1 comprises the amino acid sequence of SEQ ID NO: 231, the LCDR2 comprises the amino acid sequence of SEQ ID NO: 232, and the LCDR3 comprises the amino acid sequence of SEQ ID NO: 233.

The present invention further provides single-chain Fv (scFv) antibodies that bind cIL-4 and comprise a heavy chain variable region that comprises at least 90%, 95%, 98%, 99%, or 100% sequence identity with the amino acid sequence of SEQ ID NO: 4 and the light chain variable region comprises at least 90%, 95%, 98%, 99%, or 100% sequence identity with the amino acid sequence of SEQ ID NO: 5. In other embodiments of the scFV, the heavy chain variable region comprises at least 90%, 95%, 98%, 99%, or 100% sequence identity with the amino acid sequence of SEQ ID NO: 7 and the light chain variable region comprises at least 90%, 95%, 98%, 99%, or 100% sequence identity with the amino acid sequence of SEQ ID NO: 8. In still other embodiments of the scFV, the heavy chain variable region comprises at least 90%, 95%, 98%, 99%, or 100% sequence identity with the amino acid sequence of SEQ ID NO: 236 and the light chain variable region comprises at least 90%, 95%, 98%, 99%, or 100% sequence identity with the amino acid sequence of SEQ ID NO: 8.

In yet other embodiments of the scFV, the heavy chain variable region comprises at least 90%, 95%, 98%, 99%, or 100% sequence identity with the amino acid sequence of SEQ ID NO: 10 and the light chain variable region comprises at least 90%, 95%, 98%, 99%, or 100% sequence identity with the amino acid sequence of SEQ ID NO: 11. In still other embodiments of the scFV, the heavy chain variable region comprises at least 90%, 95%, 98%, 99%, or 100% sequence identity with the amino acid sequence of SEQ ID NO: 13 and the light chain variable region comprises at least 90%, 95%, 98%, 99%, or 100% sequence identity with the amino acid sequence of SEQ ID NO: 14. In yet other embodiments of the scFV, the heavy chain variable region comprises at least 90%, 95%, 98%, 99%, or 100% sequence identity with the amino acid sequence of SEQ ID NO: 16 and the light chain variable region comprises at least 90%, 95%, 98%, 99%, or 100% sequence identity with the amino acid sequence of SEQ ID NO: 17.

In still other embodiments of the scFV, the heavy chain variable region comprises at least 90%, 95%, 98%, 99%, or 100% sequence identity with the amino acid sequence of SEQ ID NO: 19 and the light chain variable region comprises at least 90%, 95%, 98%, 99%, or 100% sequence identity with the amino acid sequence of SEQ ID NO: 20. In yet other embodiments of the scFV, the heavy chain variable region comprises at least 90%, 95%, 98%, 99%, or 100% sequence identity with the amino acid sequence of SEQ ID NO: 22 and the light chain variable region comprises at least 90%, 95%, 98%, 99%, or 100% sequence identity with the amino acid sequence of SEQ ID NO: 23.

In yet other embodiments of the scFV, the heavy chain variable region comprises at least 90%, 95%, 98%, 99%, or 100% sequence identity with the amino acid sequence of SEQ ID NO: 25 and the light chain variable region comprises at least 90%, 95%, 98%, 99%, or 100% sequence identity with the amino acid sequence of SEQ ID NO: 26. In still other embodiments of the scFV, the heavy chain variable region comprises at least 90%, 95%, 98%, 99%, or 100% sequence identity with the amino acid sequence of SEQ ID NO: 28 and the light chain variable region comprises at least 90%, 95%, 98%, 99%, or 100% sequence identity with the amino acid sequence of SEQ ID NO: 29.

In still other embodiments of the scFV, the heavy chain variable region comprises at least 90%, 95%, 98%, 99%, or 100% sequence identity with the amino acid sequence of SEQ ID NO: 31 and the light chain variable region comprises at least 90%, 95%, 98%, 99%, or 100% sequence identity with the amino acid sequence of SEQ ID NO: 32. In yet other embodiments of the scFV, the heavy chain variable region comprises at least 90%, 95%, 98%, 99%, or 100% sequence identity with the amino acid sequence of SEQ ID NO: 34 and the light chain variable region comprises at least 90%, 95%, 98%, 99%, or 100% sequence identity with the amino acid sequence of SEQ ID NO: 35.

In still other embodiments of the scFV, the heavy chain variable region comprises at least 90%, 95%, 98%, 99%, or 100% sequence identity with the amino acid sequence of SEQ ID NO: 37 and the light chain variable region comprises at least 90%, 95%, 98%, 99%, or 100% sequence identity with the amino acid sequence of SEQ ID NO: 38. In yet other embodiments of the scFV, the heavy chain variable region comprises at least 90%, 95%, 98%, 99%, or 100% sequence identity with the amino acid sequence of SEQ ID NO: 40 and the light chain variable region comprises at least 90%, 95%, 98%, 99%, or 100% sequence identity with the amino acid sequence of SEQ ID NO: 41.

The present invention further provides a single-chain Fv (scFv) antibody that binds cIL-4 and comprises the amino acid sequence of SEQ ID NO: 3. In still other embodiments, the scFV comprises the amino acid sequence of SEQ ID NO: 6. In yet other embodiments, the scFV, comprises the amino acid sequence of SEQ ID NO: 9. In still other embodiments, the scFV, comprises the amino acid sequence of SEQ ID NO: 12. In yet other embodiments, the scFV, comprises the amino acid sequence of SEQ ID NO: 15. In still other embodiments, the scFV, comprises the amino acid sequence of SEQ ID NO: 18. In yet other embodiments, the scFV, comprises the amino acid sequence of SEQ ID NO: 21. In still other embodiments, the scFV, comprises the amino acid sequence of SEQ ID NO: 24. In yet other embodiments, the scFV, comprises the amino acid sequence of SEQ ID NO: 27. In still other embodiments, the scFV, comprises the amino acid sequence of SEQ ID NO: 30. In yet other embodiments, the scFV, comprises the amino acid sequence of SEQ ID NO: 33. In still other embodiments, the scFV, comprises the amino acid sequence of SEQ ID NO: 36. In yet other embodiments, the scFV, comprises the amino acid sequence of SEQ ID NO: 39.

The present invention further provides individual nucleic acids that individually encode each of the scFvs of the present invention that bind cIL-4.

These and other aspects of the present invention will be better appreciated by reference to the following Brief Description of the Drawings and the Detailed Description.

### BRIEF DESCRIPTION OF THE DRAWINGS

FIGS. 1 and 2 depict the dose response curve for inhibition of canine IL-4 mediated STAT-6 phosphorylation in DH82 cells. The cells were treated with either canine IL-4 alone or with canine IL-4 mixed with various concentrations of anti-canine IL-4 antibodies. The results shows that the addition of anti-canine IL-4 antibodies result in a dose dependent inhibition of STAT-6 phosphorylation. For FIG. 1: The IL-4 control is depicted by just the lone bigger dot (●) at the upper right-hand corner of the plot.
IL4-P3-1B9VH-cIgGB/IL4-P3-1B9VL-cCL (●)
IL4-P3-1B10VH-cIgGB/IL4-P3-1B10VL-cCL (▪)
IL4-P3-1B10VHm-cIgGB/IL4-P3-1B10VL-cCL (▴)
IL4-P3-1D7VH-cIgGB/IL4-P3-D7VL-cCL (▾)
IL4-P4-1G3VH-cIgGB/IL4-P4-1G3VL-cCl (♦)

FIG. 2 depicts the IL-4 control by just the lone bigger square (▪) at the upper right-hand corner of the plot.
IL4-P4A-2D11VH-cIgGB/IL4-P4A-2D11VL-cCL (●)
IL4-P4A-1F10VH-cIgGB/IL4-P4A-1F10VL-cCL (▪)
IL4-P4A-1A10VH-cIgGB/IL4-P4-1A10VL-cCL (▴)
IL4-P3-1A6VH-cIgGB/IL4-P3-1A6VL-cCL (▾)
IL4-P3-1C4VH-cIgGB/IL4-P3-1C4VL-cCl (♦)

FIG. 3 depicts the dose response curve for inhibition of canine IL-13 mediated STAT-6 phosphorylation in DH82 cells. The cells were treated with either canine IL-13 alone or with canine IL-13 mixed with various concentrations of anti-canine IL-13 antibodies. The results show that the addition of anti-canine IL-13 antibodies result in a dose dependent inhibition of STAT-6 phosphorylation. The canine IL-13 control is depicted by just the lone upside-down triangle (▾) at the upper right-hand corner of the plot.
IL13-P3-1G5VH-cIgGB/IL13-P3-1G5VL-cCL (●)
IL13-P3-05VH-cIgGB/IL13-P3-05VL-cCL (▪)
IL13-P3-05VH-cIgGB/IL13-P3-05VLm-cCL (▴)

FIG. 4 depicts the dose response curve for inhibition of canine IL-4 mediated STAT-6 phosphorylation in DH82 cells. The cells were treated with either canine IL-4 alone or with canine IL-4 mixed with various concentrations of anti-canine IL-4 antibodies. The results show that the addition of anti-canine IL-4 antibodies result in a dose dependent inhibition of STAT-6 phosphorylation. The canine IL-4 control is depicted by just the lone bigger square (▪) at the upper right-hand corner of the plot.
IL4-P4A-1A4VH-cIgGB/IL4-P4A-1A4VL-cCL (▾)
IL4-P4A-1D9VH-cIgGB/IL4-P4A-1D9VL-cCl (♦)

FIG. 5 depicts an alignment of the amino acid sequences of the Fc regions from human IgG1 Fc [SEQ ID NO: 107], canine IgG-A [SEQ ID NO: 99], canine IgG-B [SEQ ID NO: 101], canine IgG-C [SEQ ID NO: 103], canine IgG-D [SEQ ID NO: 105], along with a consensus sequence [SEQ ID NO: 242]. The solid arrows denote particular amino acid residue positions that reflect the EU numbering scheme according to Sequences of Proteins of Immunological Interest, 5th ed., Kabat et al., National Institutes of Health, Bethesda, Md. (1991). The “X” reflects the lack of uniformity at positions 252 and 254 in the consensus sequence for the five amino acid sequences being compared.

FIGS. 6A-6B shows the characterization of the interface between canine IL-4 and two antibodies depicted as IL4-2D11 (i.e., IL4-P4A-2D11VH-cIgGB/IL4-P4A-2D11VL-cCL) and IL4-1F10 (i.e., IL4-P4A-1F10VH-cIgGB/IL4-P4A-1F10VL-cCL). The amino acid residue positions shown are with respect to the numbering of amino acid sequence of SEQ ID NO: 1. The determinations were performed by chemical cross-linking, High-Mass MALDI mass spectrometry and nLC-Orbitrap mass spectrometry. (See, Example 7 below).

FIG. 6A shows the characterization of the interface between canine IL-4 and the canine antibody IL4-P4A-2D11VH-cIgGB/IL4-P4A-2D11VL-cCL identified as having an epitope with two closely positioned interaction sites (labeled as two separate epitopes in the figure) comprised by the amino acid sequence of SEQ ID NO: 89, and another epitope, which is comprised by the amino acid sequence SEQ ID NO: 90 (see, Table 7 below). The particular amino acid residues identified as binding to the IL-4 antigen of the two interaction sites of the first epitope are the lysine residue at position 12 (K₁₂), the arginine residue at position 20 (R₂₀), and the serine residue at position 23 (S₂₃) at the first interaction site, and the threonine residue at position 28 (T₂₈), the lysine residue at position 37 (K₃₇), the threonine residue at position 39 (T₃₉) at the second interaction site. The particular amino acid residues identified as binding to the IL-4 antigen of the lone interaction site of the second epitope are the pair of lysine residues at positions 86 and 87 (K₈₆ and K₈₇), the serine residue at position 88 (S₈₈), the threonine residue at position 89 (T₈₉), the lysine residue at position 91 (K₉₁), and the arginine at position at position 96 (R₉₆).

FIG. 6B shows the characterization of the interface between canine IL-4 and the canine antibody IL4-P4A-1F10VH-cIgGB/IL4-P4A-1F10VL-cCL identified has having two epitopes, the first comprised by the amino acid sequence of SEQ ID NO: 91 and the second comprised by the amino acid sequence of SEQ ID NO: 92 (see, Table 7 below). The particular amino acid residues identified as binding to the IL-4 antigen of the first epitope are the threonine residue at position 6 (T₆), the lysine residue at position 8 (K₈), and the lysine residue at position 12 (K₁₂). K₁₂ is identified as binding to the IL-4 antigen by both the IL4-2D11 and IL4-1F10 antibodies. The particular amino acid residues identified as binding to the IL-4 antigen of the second epitope are the serine residue at position 74 (S₇₄) and the threonine residue at position 79 (T₇₉).

FIG. 7 shows the characterization of the interface between canine IL-13 and the canine antibody IL13-P3-05VH-cIgGB/IL13-P3-05VLm-cCL. The amino acid residue positions shown are with respect to the numbering of amino acid sequence of SEQ ID NO: 2. The determination was performed by chemical cross-linking, High-Mass MALDI mass spectrometry and nLC-Orbitrap mass spectrometry. (See, Example 7 below). The particular amino acid residues identified as binding to the IL-13 antigen of the first epitope are the three serine residues at: positions 27 (S₂₇), 32 (S₃₂), and 36 (S₃₆), respectively. The particular amino acid residues identified as binding to the IL-13 antigen of the second epitope are the serine residues at: positions 50 (S₅₀), 55 (S₅₅), and 58 (S₅₈), respectively.

### DETAILED DESCRIPTION OF THE INVENTION

It is contemplated that any product, method, use or composition described herein can be implemented with respect to any other product, method, use or composition described herein. Embodiments disclosed in the context of products, methods, uses or compositions of the invention may be employed with respect to any other product, method, use, or composition described herein. Thus, an embodiment pertaining to one product, method, use or composition may be applied to other products, methods, uses or compositions of the invention as well.

The present invention provides antibodies that bind to either canine IL-4 or canine IL-13. These antibodies include canine and scFv antibodies. In particular embodiments, the antibodies are canine antibodies, as specifically exemplified below.

It is known that IL-4 binds with high affinity to two heterodimer receptors i.e., the Type I and Type II IL-4 receptors. The Type I heterodimer IL-4 receptor consists of the IL-4 receptor alpha chain and the common gamma C (γc) chain. The Type II heterodimer IL-4 receptor consists of the IL-4 receptor alpha chain and the IL-13 receptor alpha 1 (IL-13R alpha 1) chain. IL-13 is known to bind to the IL-13R alpha 1 chain of the IL-4 Type II receptor, and to a unique receptor designated as the IL-13 receptor alpha 2.

The canine antibodies of the present invention comprise canine IgG-A, IgG-B, IgG-C, or IgG-D heavy chains together with canine kappa or lambda light chains of the present invention. In particular embodiments, the canine antibodies comprise a canine IgG-B Fc, as specifically exemplified below. Specific CDR sets for the canine IL-4 or canine IL-13 antibodies are provided, as are corresponding variable heavy chain regions and variable light chain regions.

Accordingly, the present invention provides canine antibodies that bind to either canine IL-13 or canine IL-4, respectively, wherein the canine antibodies or antigen binding fragments thereof comprise specific sets of six CDRS, three in the heavy chain variable region of the heavy chain and three in the light chain variable region of the light chain. The canine antibodies or antigen binding fragments thereof can also comprise specific heavy chain variable regions of the heavy chains and specific light chain variable regions of the light chain. The canine antibodies or antigen binding fragments thereof can further comprise specific heavy chains and light chains of the canine antibodies.

The present invention further provides nucleic acids that encode any one of the canine antibodies of the present invention, as well as any one of the antigen binding fragments of the antibodies of the present invention. The present invention further provides nucleic acids encoding the heavy chain variable regions of the canine antibodies or antigen binding fragments thereof; the heavy chains of the canine antibodies or antigen binding fragments thereof, the light chain variable regions of the canine antibodies or antigen binding fragments thereof, and/or the light chains of the canine antibodies or antigen binding fragments thereof.

Therefore, the present invention provides nucleic acids that encode the heavy chain variable region of a canine antibody or an antigen binding fragment thereof of the present invention. The present invention further provides nucleic acids that encode the heavy chain of a canine antibody or an antigen binding fragment thereof of the present invention. The present invention also provides nucleic acids that encode the light chain variable region of a canine antibody or an antigen binding fragment thereof of the present invention. The present invention also provides nucleic acids that encode the light chain of a canine antibody or an antigen binding fragment thereof of the present invention. In certain embodiments, the nucleic acid encoding the heavy chain variable region encodes the heavy chain of a canine antibody and the corresponding nucleic acid encoding the light chain variable region encodes the light chain of that canine antibody.

The present invention also provides pairs of nucleic acids, wherein one of the pair of nucleic acids comprises a nucleotide sequence that encodes the light chain of a specific canine antibody of any one of the antibodies of the present invention and the other of the pair of nucleic acids comprises a nucleotide sequence that encodes the heavy chain of that (said) specific canine antibody. The present invention also provides expression vectors that comprise such pairs of nucleic acids, or alternatively individual nucleic acids of the present invention. In addition, the present invention provides pairs of expression vectors, wherein one of the pair of expression vectors comprises a nucleic acid comprising a nucleotide sequence that encodes the light chain of a specific canine antibody of any one of the canine antibodies of the present invention, and the other of the pair of expression vectors comprises a nucleic acid comprising a nucleotide sequence that encodes the heavy chain of that (said) specific canine antibody.

### Abbreviations

Throughout the detailed description and examples of the invention the following abbreviations will be used:

| | |
|---|---|
| ADCC | Antibody-dependent cellular cytotoxicity |
| CDC | Complement-dependent cyotoxicity |
| CDR | Complementarity determining region in the immunoglobulin variable regions, |
| | defined using the Kabat numbering system |
| EC50 | concentration resulting in 50% efficacy or binding |
| ELISA | Enzyme-linked immunosorbant assay |
| FR | Antibody framework region: the immunoglobulin variable regions excluding |
| | the CDR regions. |
| IC50 | concentration resulting in 50% inhibition |
| IgG | Immunoglobulin G |
| Kabat | An immunoglobulin alignment and numbering system pioneered by Elvin A. |
| | Kabat [Sequences of Proteins of Immunological Interest, 5th Ed. Public Health |
| | Service, National Institutes of Health, Bethesda, Md. (1991)]. |
| KD | Dissociation constant, which is the concentration of ligand (e.g., a specific |
| | antibody), in which half the ligand binding sites on the protein (e.g., protein |
| | antigen, such as cIL-4 or cIL-13) for that specific ligand are occupied in the |
| | system equilibrium. It can be calculated by dividing the koff value (k₋₁) by the |
| | kon value (k₁), e.g., KD = k₋₁/k₁. |
| mAb | Monoclonal antibody (also Mab or MAb) |
| V region | The segment of IgG chains which is variable in sequence between different |
| | antibodies. |
| VH | Immunoglobulin heavy chain variable region |
| VL | Immunoglobulin light chain variable region |
| Vk | Immunoglobulin kappa light chain variable region |
| Vl | Immunoglobulin lambda light chain variable region |

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Activity” may refer to modulation of components of the innate or the adaptive immune systems.

“Administration” and “treatment”, as it applies to an animal, e.g., a canine subject, cell, tissue, organ, or biological fluid, refers to contact of an exogenous pharmaceutical, therapeutic, diagnostic agent, or composition to the animal e.g., a canine subject, cell, tissue, organ, or biological fluid. Treatment of a cell encompasses contact of a reagent to the cell, as well as contact of a reagent to a fluid, where the fluid is in contact with the cell.

“Administration” and “treatment” also mean in vitro and ex vivo treatments, e.g., of a cell, by a reagent, diagnostic, binding compound, or by another cell. The term “subject” includes any organism, preferably a non-human animal, more preferably a mammal (e.g., canine or feline) and most preferably a canine.

“Treat” or “treating” means to administer a therapeutic agent, such as a composition containing any of the antibodies of the present invention, internally or externally to e.g., a canine subject or patient having one or more signs/symptoms, or being suspected of having a condition, for which the agent has therapeutic activity. Typically, the agent is administered in an amount effective to alleviate and/or ameliorate one or more disease/condition signs/symptoms in the treated subject or population, whether by inducing the regression of or inhibiting the progression of such sign(s)/symptom(s) by any clinically measurable degree. The amount of a therapeutic agent that is effective to alleviate any particular disease/condition sign/symptom (also referred to as the “therapeutically effective amount”) may vary according to factors such as the disease/condition state, age, and weight of the patient (e.g., canine), and the ability of the pharmaceutical composition to elicit a desired response in the subject. Whether a disease/condition sign/symptom has been alleviated or ameliorated can be assessed by any clinical measurement typically used by veterinarians or other skilled healthcare providers to assess the severity or progression status of that sign/symptom. While an embodiment of the present invention (e.g., a treatment method or article of manufacture) may not be effective in alleviating the target disease/condition sign(s)/symptom(s) in every subject, it should alleviate the target disease/condition sign(s)/symptom(s) in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Treatment,” as it applies to a veterinary (e.g., canine) or research subject, refers to therapeutic treatment, as well as research and diagnostic applications. “Treatment” as it applies to a veterinary (e.g., canine), or research subject, or cell, tissue, or organ, encompasses contact of the antibodies of the present invention to e.g., a canine or other animal subject (e.g., feline), a cell, tissue, physiological compartment, or physiological fluid.

As used herein, the term “canine” includes all domestic dogs, Canis lupus familiaris or Canis familiaris, unless otherwise indicated.

As used herein, the term “feline” refers to any member of the Felidae family. Members of this family include wild, zoo, and domestic members, including domestic cats, pure-bred and/or mongrel companion cats, show cats, laboratory cats, cloned cats, and wild or feral cats.

As used herein, the term “antibody” refers to any form of antibody that exhibits the desired biological activity. An antibody can be a monomer, dimer, or larger multimer. Thus, it is used in the broadest sense and specifically covers, but is not limited to, monoclonal antibodies (including full length monoclonal antibodies), polyclonal antibodies, multi-specific antibodies (e.g., bispecific antibodies), caninized antibodies, fully canine antibodies, chimeric antibodies and camelized single domain antibodies. “Parental antibodies” are antibodies obtained by exposure of an immune system to an antigen prior to modification of the antibodies for an intended use, such as caninization of an antibody for use as a canine therapeutic antibody.

In preferred embodiments of the present invention an antibody is a homodimer of two identical pairs of a canine light and heavy chain (H2L2), i.e., a canine IgG antibody. Canine antibodies can be obtained in a number of ways, including (i) by injecting an immunogen into a dog and recovering the canine antibodies, (ii) by injecting an immunogen into a rodent that has been genetically engineered to comprise a canine immune system in place of its natural rodent immune system and then obtaining the canine antibodies from the genetically engineered rodent, and (iii) from a phage display library that has been genetically engineered to produce canine variable heavy and light regions. Canine antibodies can further comprise one or more specific substitution(s) of an amino acid residue(s) in their constant regions (e.g., their cFc regions and/or hinge regions) to, for example, significantly diminish the antibody-dependent cytotoxicity (ADCC) and complement-dependent cytotoxicity (CDC) of the naturally occurring canine antibody as described below, and/or to increase/extend their half-life in vivo, as noted below.

Each of the two heavy chains of an IgG antibody consists of one variable domain (VH) and three constant domains referred to as CH-1, CH-2, and CH-3. The CH-1 domain is connected to the CH-2 domain via an amino acid sequence referred to as the “hinge” or alternatively as the “hinge region”. The light chain has one constant domain (CL) and one variable domain (VH). The variable regions of each light/heavy chain pair form the antibody binding site. Thus, in general, an intact antibody has two binding sites. Except in bifunctional or bispecific antibodies, the two binding sites are, in general, the same. Typically, the variable domains of both the heavy and light chains comprise three hypervariable regions, also called complementarity determining regions (CDRs), located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat, Adv. Prot. Chem. 32:1-75 (1978); Kabat, et al., J. Biol. Chem. 252:6609-6616 (1977); Chothia, et al., J. Mol. Biol. 196:901-917 (1987) or Chothia, et al., Nature 342:878-883 (1989)].

As used herein, the term “hypervariable region” refers to the amino acid residues of an antibody that are responsible for antigen-binding. The hypervariable region comprises amino acid residues from a “complementarity determining region” or “CDR” (i.e., LCDR1, LCDR2 and LCDR3 in the light chain variable domain and HCDR1, HCDR2 and HCDR3 in the heavy chain variable domain). Accordingly, there are six CDRs in each monomer of a cIL-4 or cIL-13 dimeric antibody: three CDRs in each light chain and three CDRs in each heavy chain. [See, Kabat et al. Sequences of Proteins of Immunological Interest, 5th Ed. Public Health Service, National Institutes of Health, Bethesda, Md. (1991), defining the CDR regions of an antibody by sequence; see also Chothia and Lesk, J. Mol. Biol. 196:901-917 (1987) defining the CDR regions of an antibody by structure]. As used herein, the term “framework” or “FR” residues refers to those variable domain residues other than the hypervariable region residues defined herein as CDR residues.

The “Fragment crystallizable region” abbreviated as “Fc” or used interchangeably with “Fc region” corresponds to the CH3-CH2 portion of an antibody that interacts with cell surface receptors called Fc receptors. The canine fragment crystallizable region (cFc region) of each of the four canine IgGs were first described by Tang et al. [Vet. Immunol. Immunopathol. 80: 259-270 (2001); see also, Bergeron et al., Vet. Immunol. Immunopathol. 157: 31-41 (2014) and U.S. Pat. No. 10,106,607 B2] and their amino acid sequences are provided below.

As used herein the canine Fc (cFc) “IgG-Bm” is canine IgG-B Fc comprising two (2) amino acid residue substitutions, D31A and N63A, as in the amino acid sequence of SEQ ID NO: 102 of IgG-B (see below). Accordingly, both the aspartic acid residue (D) at position 31 and the asparagine residue (N) at position 63 of SEQ ID NO: 101, are substituted by an alanine residue (A) in IgG-Bm. These two amino acid residue substitutions serve to significantly diminish the antibody-dependent cytotoxicity (ADCC) and complement-dependent cytotoxicity (CDC) of the naturally occurring canine IgG-B [see, U.S. Pat. No. 10,106,607 B2, the contents of which are hereby incorporated by reference in their entirety].

As used herein, a “substitution of an amino acid residue” with another amino acid residue in an amino acid sequence of an antibody for example, is equivalent to “replacing an amino acid residue” with another amino acid residue and denotes that a particular amino acid residue at a specific position in the amino acid sequence has been replaced by (or substituted for) by a different amino acid residue. Such substitutions can be particularly designed i.e., purposefully replacing an alanine with a serine at a specific position in the amino acid sequence by e.g., recombinant DNA technology. Alternatively, a particular amino acid residue or string of amino acid residues of an antibody can be replaced by one or more amino acid residues through more natural selection processes e.g., based on the ability of the antibody produced by a cell to bind to a given region on that antigen, e.g., one containing an epitope or a portion thereof, and/or for the antibody to comprise a particular CDR that retains the same canonical structure as the CDR it is replacing. Such substitutions/replacements can lead to “variant” CDRs and/or variant antibodies.

As used herein the term “canine frame” refers to the amino acid sequence of the heavy chain and light chain of a canine antibody other than the hypervariable region residues defined herein as CDR residues. With regard to a caninized antibody, in the majority of embodiments the amino acid sequences of the native canine CDRs are replaced with the corresponding foreign CDRs (e.g., those from a mouse or human) in both chains. Optionally the heavy and/or light chains of the canine antibody may contain some foreign non-CDR residues, e.g., so as to preserve the conformation of the foreign CDRs within the canine antibody, and/or to modify the Fc region function, as described above and/or disclosed in U.S. Pat. No. 10,106,607 B2, hereby incorporated by reference herein in its entirety.

As used herein, a “chimeric antibody” is an antibody having the variable domain from a first antibody and the constant domain from a second antibody, where the first and second antibodies are from different species. [U.S. Pat. No. 4,816,567; and Morrison et al., Proc. Natl. Acad. Sci. USA 81: 6851-6855 (1984)]. Typically the variable domains are obtained from an antibody from an experimental animal (the “parental antibody”), such as a rodent (or a rodent that comprises a human immune system) and the constant domain sequences are obtained from the animal subject antibodies, e.g., canine so that the resulting chimeric antibody will be less likely to elicit an adverse immune response in a canine subject respectively, than the parental (e.g., rodent) antibody.

As used herein, the term “canine antibody” refers to an antibody that has an amino acid sequence, including the six CDRs of the canine antibody, that originates from a canine source. Three examples of a canine source include (i) a canine cell, e.g., an antibody raised in a canine in response to the injection of an antigen, which can be a canine antigen coupled to a foreign (non-canine) protein to help break self-tolerance, (ii) a cell from a non-canine mammal genetically engineered to have a canine immune system (such as a caninized mouse or rat), e.g., an antibody raised in a caninized mouse in response to the injection of an antigen, which can be a canine antigen coupled to a foreign (non-canine) protein to help break self-tolerance, or (iii) a canine phage display library (cPDL), such as the canine IgM/IgG/λ/κ scFv phage display library exemplified below. In the case of the canine source being a cPDL, the canine antibody is genetically engineered by combining the amino acid sequences of the variable heavy (VH) region and the variable light (VL) region obtained from the cPDL together with the corresponding amino acid sequences of the remaining regions of a canine heavy chain and a canine light chain, respectively, and as exemplified below. These canine amino acid sequences can be further genetically engineered to modify and/or optimize a particular property of the antibody, such as increasing the half-life of the antibody in vivo, or eliminating a putative glycosylation site in a CDR, as described below.

As used herein, the term “caninized antibody” refers to forms of antibodies that contain amino acid sequences from both canine and non-canine (e.g., mouse or human) antibodies. In general, the caninized antibody will comprise substantially all of at least one or more typically, two variable domains in which all or substantially all of the hypervariable loops correspond to those of a non-canine immunoglobulin (e.g., comprising 6 CDRs), and all or substantially all of the framework (FR) regions (and typically all or substantially all of the remaining canine frame) are those of a canine immunoglobulin sequence. A caninized antibody can comprise both the three heavy chain CDRs and the three light chain CDRS from e.g., a murine anti-canine IL-4Rα or IL-31RA antibody together with a canine frame or a modified canine frame. A modified canine frame comprises one or more amino acids changes as exemplified herein that further optimize the effectiveness of the caninized antibody, e.g., to increase its binding to its canine antigen and/or its ability to block the binding of that canine antigen to the canine antigen's natural binding partner. Caninized antibodies are an alternative to canine antibodies, but they are more likely to lead to deleterious antidrug antibodies (ADA) in a treated canine host.

As used herein, antibodies of the present invention that “block” or an antibody that is “blocking” or is “blocking the binding” of two or more proteins e.g., a canine receptor to its binding partner (ligand), is an antibody that blocks (partially or fully) the binding of the canine receptor to its canine ligand and vice versa, as can be determined in standard binding assays (e.g., BIACore®, ELISA, or flow cytometry). When an antibody blocks a protein-protein interaction involved in a signal transduction pathway in situ or in vivo, such blocking can lead to partially or fully blocking the signal transduction pathway, which in vivo, may lead to attenuation or even prevention of the physiological consequence of that signaling.

Typically, an antibody or antigen binding fragment of the invention retains at least 10% of its canine antigen binding activity (when compared to the parental antibody) when that activity is expressed on a molar basis. Preferably, an antibody or antigen binding fragment of the invention retains at least 20%, 50%, 70%, 80%, 90%, 95% or 100% or more of the canine antigen binding affinity as the parental antibody. It is also intended that an antibody or antigen binding fragment of the invention can include conservative or non-conservative amino acid substitutions (referred to as “conservative variants” or “function conserved variants” of the antibody) that do not substantially alter its biologic activity.

An “isolated antibody” refers to the purification status and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

As used herein, an antibody is said to bind specifically to a polypeptide comprising a given antigen sequence (e.g., a portion of the amino acid sequence of cIL-4 or cIL-13) if it binds to polypeptides comprising the portion of the amino acid sequence of cIL-4 or cIL-13, but does not bind to other canine proteins lacking that portion of the sequence of cIL-4 or cIL-13. For example, an antibody that specifically binds to a polypeptide comprising cIL-4 or cIL-13, may bind to a FLAG®-tagged form of cIL-4 or cIL-13, but will not bind to other FLAG®-tagged canine proteins.

As used herein, unless otherwise indicated, “antibody fragment” or “antigen binding fragment” refers to antigen binding fragments of antibodies, i.e., antibody fragments that retain the ability to bind specifically to the antigen (e.g., cIL-4 or cIL-13) bound by the full-length antibody, e.g., fragments that retain one or more CDR regions. Examples of antigen binding fragments include, but are not limited to, Fab, Fab′, F(ab′) 2, and Fv fragments; diabodies; linear antibodies; single-chain antibody molecules, e.g., scFv; nanobodies and multispecific antibodies formed from antibody fragments.

The term “single-chain Fv” or “scFv” antibody refers to antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen binding. [See, Pluckthun, THE PHARMACOLOGY OF MONOCLONAL ANTIBODIES, vol. 113 Rosenburg and Moore eds., Springer-Verlag, New York, pp. 269-315 (1994); WO 88/01649; and U.S. Pat. Nos. 4,946,778 and 5,260,203.] Accordingly, a single-chain variable fragment (scFv) is not actually a fragment of an antibody, but instead is a fusion protein of the variable regions of the heavy (VH) and light chains (VL) of immunoglobulins, connected with a short linker peptide, e.g., of about ten to about 25 amino acids. scFvs can have valuable research, diagnostic, and clinical applications [see e.g., Ahmad et al., Clin Dev Immunol. 2012; 2012:980250. doi: 10.1155/2012/980250. Epub 2012 Mar. 15. PMID: 22474489; PMCID: PMC3312285].

An antibody, or binding compound derived from the antigen-binding site of an antibody, binds to its canine antigen, or a variant or mutein thereof, “with specificity” when it has an affinity for that canine antigen or a variant or mutein thereof which is at least ten-times greater, more preferably at least 20-times greater, and even more preferably at least 100-times greater than its affinity for any other canine antigen tested. An antibody that binds cIL-4 or cIL-13 “with specificity” may still bind an IL-4 or IL-13 respectively, from another species (e.g., feline IL-4 or IL-13 and/or human IL-4 or IL-13).

“Homology”, as used herein, refers to sequence similarity between two polynucleotide sequences or between two polypeptide sequences when they are optimally aligned. When a position in both of the two compared sequences is occupied by the same base or amino acid residue, e.g., if a position in each of two DNA molecules is occupied by adenine, then the molecules are homologous at that position. The percent of homology is the number of homologous positions shared by the two sequences divided by the total number of positions compared×100. For example, if 6 of 10 of the positions in two sequences are matched or homologous when the sequences are optimally aligned then the two sequences are 60% homologous. Generally, the comparison is made when two sequences are aligned to give maximum percent homology.

As used herein, “sequence identity” refers to the degree to which the amino acid residues of two polypeptides or nucleic acids are the same (i.e., identical) at the equivalent positions when a query sequence is optimally aligned over the length of a reference sequence, introducing gaps as necessary if the reference and query sequence do not have the same length to achieve a maximum percent identity (called a “global alignment”). For this disclosure, a reference sequence is always an amino acid or nucleic acid sequence specifically disclosed herein, and a query sequence is any other sequence aligned to the reference sequence. Sequence identity is expressed as a percent sequence identity (% sequence identity or % identity). The global alignment and calculation of the percent identity is performed over a contiguous block of residue positions for the entire reference sequence. If the query sequence is longer than the reference sequence, then the percent identity is determined only using the residue positions of the reference sequence. Any residues of the query sequence with positions beyond the first and last positions of the reference sequence after an initial global alignment are removed and the reference and query sequences are re-aligned to determine percent identity. A query sequence has 100% identity to a reference sequence when the residues of both sequences are identical at the equivalent positions over the length of the reference sequence. Similarly, a query sequence has 50% identity to a reference sequence when 50% of the residues of the two amino acid sequences are identical at the equivalent positions over the length of the reference sequence.

In particular embodiments in which the reference sequence of the present invention comprises the amino acid sequences of the CDRs in a given heavy chain or light chain, and/or corresponding heavy chain variable region or light chain variable region as provided below; and/or comprises defined amino acid residue substitutions specifically placed in the amino acid sequence of a given cFc of the heavy chain to increase/extend the antibody half-life and/or to decrease effector functions such as antibody-dependent cytotoxicity (ADCC) and complement-dependent cytotoxicity (CDC) as discussed below, it is understood that such defined amino acid residues and/or defined amino acid residue substitutions themselves are, (and/or remain) identical at the equivalent positions in the two amino acid sequences being compared.

A global alignment can be created using the global alignment tool “Needle” from the online European Molecular Biology Open Software Suite (EMBOSS) (www.ebi.ac.uk/Tools/psa/emboss_needle/) or the global alignment tool “BLAST®»Global Alignment” from the National Center for Biotechnology Information (NCBI) (blast.ncbi.nlm.nih.gov/Blast.cgiPROGRAM=blastn&PAGE_TYPE=BlastSearch&PROG_DEF AULTS=on&BLAST_INIT=GlobalAln&BLAST_SPEC-GlobalAln&BLAST_PROGRAMS=blastn). Both of these global alignment tools incorporate the Needleman-Wunsch algorithm (Needleman, S. B. & Wunsch, C. D. [“A general method applicable to the search for similarities in the amino acid sequences of two proteins.” J. Mol. Biol. 48:443-453 (1970)]. A global alignment of nucleotide sequences using BLAST Global Alignment in this disclosure uses the following default parameters: match score=2; mismatch score=−3; Gap Cost Existence score=5; Gap Cost Extension Score=2. A global alignment of protein sequences in this disclosure using BLAST Global Alignment uses the following default parameters: Gap Cost Existence=11; Gap Cost Extension=1. A global alignment of nucleotide sequences using EMBOSS Needle in this disclosure uses the following default parameters: Output Format=pair; Matrix=DNAfull; Gap Open=10; Gap Extend=0.5; End Gap Penalty=false; End Gap Open=10; End Gap Extend=0.5. A global alignment of protein sequences using EMBOSS Needle in this disclosure uses the following default parameters: Output Format=pair; Matrix=BLOSUM62; Gap Open=10; Gap Extend=0.5; End Gap Penalty=false; End Gap Open=10; End Gap Extend=0.5.

Sequence similarity includes identical residues and nonidentical, e.g., biochemically related amino acids that share similar properties and may be interchangeable.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity [see, e.g., Watson et al., Molecular Biology of the Gene, The Benjamin/Cummings Pub. Co., p. 224 (4th Ed.; 1987)]. In addition, substitutions of structurally or functionally similar amino acids are less likely to disrupt biological activity. Exemplary conservative substitutions are set forth in Table A directly below.

TABLE A
EXEMPLARY CONSERVATIVE AMINO
ACID SUBSTITUTIONS

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala; Gly |
| Ser (S) | Thr |
| Thr (T) | Ser |
| Trp (W) | Tyr; Phe |
| Tyr (Y) | Trp; Phe |
| Val (V) | Ile; Leu |

Function-conservative variants of the antibodies of the invention are also contemplated by the present invention. “Function-conservative variants,” as used herein, refers to antibodies or fragments in which one or more amino acid residues have been changed without altering a desired property, such an antigen affinity and/or specificity. Such variants include, but are not limited to, replacement of an amino acid with one having similar properties, such as the conservative amino acid substitutions of Table A above.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

### Canine IgG's

In canine, there are four known IgG heavy chain subtypes, referred to as A, B, C, and D. These heavy chains represent four different subclasses of dog IgG, which are referred to as IgG-A (or IgGA), IgG-B (or IgGB), IgG-C (or IgGC) and IgG-D (or IgGD). The two known canine light chain subtypes are referred to as lambda (l) and kappa (k). The antibodies can be engineered to include modifications to the canine framework and/or the canine frame residues within the variable domains of a parental monoclonal antibody, e.g., to improve the properties of the antibody.

The nucleic acid and amino acid sequences of the four heavy chains were first identified by Tang et al. [Vet. Immunol. Immunopathol. 80: 259-270 (2001)]. The amino acid and nucleic sequences for these heavy chains are also available from the GenBank data bases. For example, the amino acid sequence of IgGA heavy chain has accession number AAL35301.1, IgGB has accession number AAL35302.1, IgGC has accession number AAL35303.1, and IgGD has accession number (AAL35304.1). Canine antibodies also contain two types of light chains, kappa and lambda. The DNA and amino acid sequence of these light chains can be obtained from GenBank Databases. For example, the kappa light chain amino acid sequence has accession number ABY 57289.1 and the lambda light chain has accession number ABY 55569.1.

In specific embodiments of the invention, besides binding cIL-4 or cIL-13, a canine antibody of the present invention can have one or two of the following attributes:
1 Lack of effector functions such as antibody-dependent cytotoxicity (ADCC) and complement-dependent cytotoxicity (CDC), and
2. be readily purified on a large scale using industry standard technologies such as that based on protein A chromatography. [See, U.S. Pat. No. 10,106,607 B2, the contents of which are hereby incorporated by reference in its entirety.]

None of the naturally occurring canine IgG isotypes satisfy both criteria. For example, IgG-B can be purified using protein A, but has high level of ADCC activity. On the other hand, IgG-A binds weakly to protein A, but also displays ADCC activity. Moreover, neither IgG-C nor IgG-D can be purified on protein A columns, although IgG-D displays no ADCC activity. (IgG-C has considerable ADCC activity). In certain embodiments, the present invention provides a modified canine IgG antibody that lacks the effector functions such as ADCC and can be easily purified using industry standard protein A chromatography.

The amino acid sequence for each of the four canine IgG Fc regions is based on the identified boundary of CH1 and CH2 domains as determined by Tang et al., supra. The present invention also provides antibodies of the present invention that comprise a canine fragment crystallizable region (cFc region) in which the cFc region has been genetically modified to augment, decrease, or eliminate one or more effector functions. In one aspect of the present invention, the genetically modified cFc region decreases or eliminates one or more effector functions. In another aspect of the invention the genetically modified cFc region augments one or more effector function. In certain embodiments, the genetically modified cFc region is a genetically modified canine IgGB Fc region. In another such embodiment, the genetically modified cFc region is a genetically modified canine IgGC Fc region. In a particular embodiment, the effector function is antibody-dependent cytotoxicity (ADCC) that is augmented, decreased, or eliminated. In another embodiment, the effector function is complement-dependent cytotoxicity (CDC) that is augmented, decreased, or eliminated. In yet another embodiment, the cFc region has been genetically modified to augment, decrease, or eliminate both the ADCC and the CDC.

In order to generate variants of canine IgG that lack effector functions, a number of mutant canine IgGB heavy chains were generated. These variants may include one or more of the following single or combined substitutions in the Fc portion of the heavy chain amino acid sequence: P4A, D31A, N63A, G64P, T65A, A93G, and P95A. Variant heavy chains (i.e., containing such amino acid substitutions) are cloned into expression plasmids and are transfected into suitable cells (e.g., HEK 293 cells) along with a plasmid containing the gene encoding a light chain. Intact antibodies are expressed and purified from the cells and then can be evaluated for binding to FcγRI and C1q to assess their potential for mediation of immune effector functions. [See, U.S. Pat. No. 10,106,607 B2, the contents of which are hereby incorporated by reference in its entirety.]

The known amino acid sequences of the four canine Fc regions: IgG-A, IgG-B, IgG-C and IgG-D are provided below. The corresponding four modified canine Fc regions having modified amino acid sequences, comprising the D31A and N63A substitutions, and which lack the effector functions such as ADCC are: IgG-Am, IgG-Bm, IgG-Cm, and IgG-Dm. These amino acid sequences also are provided below.

IgG-A
[SEQ ID NO: 99]
LGGPSVLIFPPKPKDILRITRTPEVTCVVLDLGREDPEVQISWFVDGKEVHTAKTQSREQ
QFNGTYRVVSVLPIEHQDWLTGKEFKCRVNHIDLPSPIERTISKARGRAHKPSVYVLPPSP
KELSSSDTVSITCLIKDFYPPDIDVEWQSNGQQEPERKHRMTPPQLDEDGSYFLYSKLSV
DKSRWQQGDPFTCAVMHETLQNHYTDLSLSHSPGK
IgG-Am
[SEQ ID NO: 100]
LGGPSVLIFPPKPKDILRITRTPEVTCVVLALGREDPEVQISWFVDGKEVHTAKTQSREQ
QFAGTYRVVSVLPIEHQDWLTGKEFKCRVNHIDLPSPIERTISKARGRAHKPSVYVLPPSP
KELSSSDTVSITCLIKDFYPPDIDVEWQSNGQQEPERKHRMTPPQLDEDGSYFLYSKLSV
DKSRWQQGDPFTCAVMHETLQNHYTDLSLSHSPGK
IgG-B
[SEQ ID NO: 101]
LGGPSVFIFPPKPKDTLLIARTPEVTCVVVDLDPEDPEVQISWFVDGKQMQTAKTQPREE
QFNGTYRVVSVLPIGHQDWLKGKQFTCKVNNKALPSPIERTISKARGQAHQPSVYVLPP
SREELSKNTVSLTCLIKDFFPPDIDVEWQSNGQQEPESKYRTTPPQLDEDGSYFLYSKLSV
DKSRWQRGDTFICAVMHEALHNHYTQESLSHSPGK
IgG-Bm
[SEQ ID NO: 102]
LGGPSVFIFPPKPKDTLLIARTPEVTCVVVALDPEDPEVQISWFVDGKQMQTAKTQPREE
QFAGTYRVVSVLPIGHQDWLKGKQFTCKVNNKALPSPIERTISKARGQAHQPSVYVLPP
SREELSKNTVSLTCLIKDFFPPDIDVEWQSNGQQEPESKYRTTPPQLDEDGSYFLYSKLSV
DKSRWQRGDTFICAVMHEALHNHYTQESLSHSPGK
IgG-C
[SEQ ID NO: 103]
LGGPSVFIFPPKPKDILVTARTPTVTCVVVDLDPENPEVQISWFVDSKQVQTANTQPREE
QSNGTYRVVSVLPIGHQDWLSGKQFKCKVNNKALPSPIEEIISKTPGQAHQPNVYVLPPS
RDEMSKNTVTLTCLVKDFFPPEIDVEWQSNGQQEPESKYRMTPPQLDEDGSYFLYSKLS
VDKSRWQRGDTFICAVMHEALHNHYTQISLSHSPGK
IgG-Cm
[SEQ ID NO: 104]
LGGPSVFIFPPKPKDILVTARTPTVTCVVVALDPENPEVQISWFVDSKQVQTANTQPREE
QSAGTYRVVSVLPIGHQDWLSGKQFKCKVNNKALPSPIEEIISKTPGQAHQPNVYVLPPS
RDEMSKNTVTLTCLVKDFFPPEIDVEWQSNGQQEPESKYRMTPPQLDEDGSYFLYSKLS
VDKSRWQRGDTFICAVMHEALHNHYTQISLSHSPGK
IgG-D
[SEQ ID NO: 105]
LGGPSVFIFPPKPKDILRITRTPEITCVVLDLGREDPEVQISWFVDGKEVHTAKTQPREQQ
FNSTYRVVSVLPIEHQDWLTGKEFKCRVNHIGLPSPIERTISKARGQAHQPSVYVLPPSPK
ELSSSDTVTLTCLIKDFFPPEIDVEWQSNGQPEPESKYHTTAPQLDEDGSYFLYSKLSVDK
SRWQQGDTFTCAVMHEALQNHYTDLSLSHSPGK
IgG-Dm
[SEQ ID NO: 106]
LGGPSVFIFPPKPKDILRITRTPEITCVVLALGREDPEVQISWFVDGKEVHTAKTQPREQQ
FASTYRVVSVLPIEHQDWLTGKEFKCRVNHIGLPSPIERTISKARGQAHQPSVYVLPPSPK
ELSSSDTVTLTCLIKDFFPPEIDVEWQSNGQPEPESKYHTTAPQLDEDGSYFLYSKLSVDK
SRWQQGDTFTCAVMHEALQNHYTDLSLSHSPGK
Human IgG1 Fc
[SEQ ID NO: 107]
PELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGK
Consensus Sequence:
[SEQ ID NO: 108]
LGGPSVFIFPPKPKDTLXIXRTPEVTCVVVDLGPEDPEVQISWFVDGKEVHTAKTQPREE
QFNSTYRVVSVLPILHQDWLKGKEFKCKVNNKALPSPIERTISKAXGQAHXPXVYVLPPS
REELSKNXTVSLTCLIKDFFPPDIDVEWQSNGQPEPEXKYRTTPPQLDEDGSYFLYSKLSV
DKSRWQRGDTFTCAVMHEALHNHYTQKSLSHSPGK
IgG-B-YD
[SEQ ID NO: 255]
LGGPSVFIFPPKPKDTLYIARDPEVTCVVVDLDPEDPEVQISWFVDGKQMQTAKTQPREE
QFNGTYRVVSVLPIGHQDWLKGKQFTCKVNNKALPSPIERTISKARGQAHQPSVYVLPP
SREELSKNTVSLTCLIKDFFPPDIDVEWQSNGQQEPESKYRTTPPQLDEDGSYFLYSKLSV
DKSRWQRGDTFICAVMHEALHNHYTQESLSHSPGK
IgG-Bm-YD
[SEQ ID NO: 256]
LGGPSVFIFPPKPKDTLYIARDPEVTCVVVALDPEDPEVQISWFVDGKQMQTAKTQPREE
QFAGTYRVVSVLPIGHQDWLKGKQFTCKVNNKALPSPIERTISKARGQAHQPSVYVLPP
SREELSKNTVSLTCLIKDFFPPDIDVEWQSNGQQEPESKYRTTPPQLDEDGSYFLYSKLSV
DKSRWQRGDTFICAVMHEALHNHYTQESLSHSPGK
IgG-B-NH
[SEQ ID NO: 257]
LGGPSVFIFPPKPKDTLLIARTPEVTCVVVDLDPEDPEVQISWFVDGKQMQTAKTQPREE
QFNGTYRVVSVLPIGHQDWLKGKQFTCKVNNKALPSPIERTISKARGQAHQPSVYVLPP
SREELSKNTVSLTCLIKDFFPPDIDVEWQSNGQQEPESKYRTTPPQLDEDGSYFLYSKLSV
DKSRWQRGDTFICAVMHEALHHHYTQESLSHSPGK
IgG-Bm-N
[SEQ ID NO: 258]
LGGPSVFIFPPKPKDTLLIARTPEVTCVVVALDPEDPEVQISWFVDGKQMQTAKTQPREE
QFAGTYRVVSVLPIGHQDWLKGKQFTCKVNNKALPSPIERTISKARGQAHQPSVYVLPP
SREELSKNTVSLTCLIKDFFPPDIDVEWQSNGQQEPESKYRTTPPQLDEDGSYFLYSKLSV
DKSRWQRGDTFICAVMHEALHHHYTQESLSHSPGK

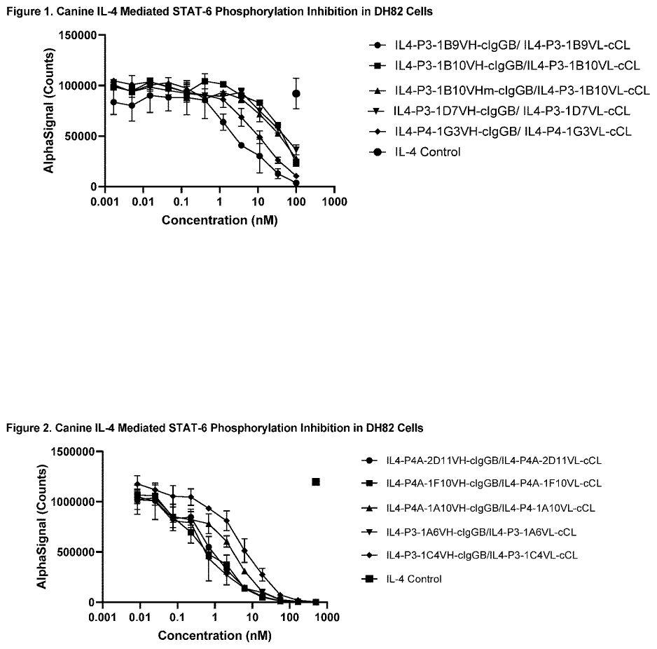

The present invention also provides canine antibodies with extended half-lives in vivo, comprising a canine IgG Fc region that comprises one or more amino acid substitutions, in which one or more amino acid residue substitutions is at an amino acid residue position of 252, 254, 256, 308, 433, 434, 436, or any combination thereof, (positions in bold in SEQ ID NOs: 99-108 above) in which the amino acid residue positions are numbered according to the EU index as in Kabat, as in FIG. 5 below. In particular embodiments the substitution at amino acid residue position 252 is with a tyrosine residue, and/or the substitution is at amino acid residue position 254, with a threonine residue, and/or the substitution is at amino acid residue position 256, with an aspartic acid residue or a glutamic acid residue, and/or the substitution is at amino acid residue position 308, with a proline residue, and/or the substitution is at amino acid residue position 433, with a lysine residue or a leucine residue, and/or the substitution is at amino acid residue position 434, with a phenylalanine residue, a histidine residue, or a tyrosine residue, and/or the substitution is at amino acid residue position 436, with a threonine residue. In more particular embodiments, the IgG Fc region has two substitutions: a substitution at amino acid residue position 252 with a tyrosine residue, and a substitution at amino acid residue position 256 with an aspartic acid residue (YD).

The corresponding canine hinge regions are:

cIgG-A hinge region
[SEQ ID NO: 109]
FNECRCTDTPPCPVPEP
cIgG-B hinge region
[SEQ ID NO: 110]
PKRENGRVPRPPDCPKCPT¹PEM
cIgG-C hinge region
[SEQ ID NO: 111]
AKECECKCNCNNCPCPGCGL
cIgG-D hinge region
[SEQ ID NO: 112]
PKESTCKCISPCPVPES
Modified cIgG-D hinge region
[SEQ ID NO: 113]
PKESTCKCIP²PCPVPES

The present invention also provides modified canine IgG-Ds, which in place of its natural IgG-D hinge region comprise a hinge region from IgG-A, IgG-B, IgG-C, or alternatively, the modified IgG-D hinge region can be genetically modified by replacing a serine residue with a proline residue, as indicated above. Such modifications can lead to a canine IgG-D lacking fab arm exchange. The modified canine IgG-Ds can be constructed using standard methods of recombinant DNA technology [e.g., Maniatis et al., Molecular Cloning, A Laboratory Manual (1982)]. In order to construct these variants, the nucleic acids encoding the amino acid sequence of canine IgG-D can be modified so that it encodes the modified IgG-Ds. The modified nucleic acid sequences are then cloned into expression plasmids for protein expression.

The present invention further provides full length canine heavy chains that can be matched with corresponding light chains to make a canine antibody. Accordingly, the present invention further provides antibodies to cIL-4 or cIL-13 (including isolated cIL-4 or cIL-13 antibodies) of the present invention and methods of use of the antibodies of the present invention in the treatment of a condition e.g., the treatment of atopic dermatitis in canines.

### Nucleic Acids

The present invention further comprises the nucleic acids encoding the antibodies of the present invention (see e.g., Examples below). In particular embodiments, a nucleic acid encoding the heavy chain of an antibody is provided along with the corresponding light chain of that antibody. Also included in the present invention are nucleic acids that encode immunoglobulin polypeptides comprising amino acid sequences that are at least about 70% identical (as defined above), preferably at least about 80% identical, more preferably at least about 90% identical and most preferably at least about 95% identical (e.g., 95% identical, 96% identical, 97% identical, 98% identical, 99% identical, or 100% identical) to the corresponding amino acid sequences of the canine antibodies described herein.

### Canine Antibodies

The construction of canine anti-IL-4 or IL-13 monoclonal antibodies can be performed using well-known genetic engineering methodology. For example, a nucleic acid sequence that encodes a canine heavy chain variable region and a nucleic acid sequence that encodes a canine light chain variable region can be determined for a given canine antibody, e.g., from a scFv as described in the Examples below. The nucleic acid sequence of the canine heavy chain variable region then can be combined with a nucleic acid sequence of a canine heavy constant region (including the corresponding hinge region), while the nucleic acid sequence of the canine light chain variable region can be combined with a nucleic acid sequence of a canine light constant region. Amino acid sequences for canine antibody constant regions are included herein and both the nucleic acid and amino acid sequences of the canine antibody constant regions are well known in the art and can be obtained by searching the NCBI gene and protein databases.

A canine anti-IL-4 or IL-13 antibody then can be produced recombinantly by a variety of methods that are known in the field. Mammalian cell lines available as hosts for expression of the antibodies or fragments disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. When recombinant expression vectors encoding the heavy chain or antigen-binding portion or fragment thereof, the light chain and/or antigen-binding fragment thereof are introduced into mammalian host cells, the antibodies are produced by culturing the host cells for a period of time sufficient to allow for expression of the antibody in the host cells or, more preferably, secretion of the antibody into the culture medium in which the host cells are grown.

Antibodies can be recovered from the culture medium using standard protein purification methods. Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

As noted above, in certain embodiments, the antibody or antigen binding fragment comprises a heavy chain constant region, e.g., a canine constant region, such as IgG-A, IgG-B, IgG-C and IgG-D□ canine heavy chain constant region or a variant thereof. In certain embodiments, the antibody or antigen binding fragment comprises a light chain constant region, e.g., a canine light chain constant region, such as lambda or kappa canine light chain constant region or variant thereof. By way of example, and not limitation, the canine heavy chain constant region can be from IgG-B and the canine light chain constant region can be from kappa.

### Pharmaceutical Compositions and Administration

To prepare pharmaceutical or sterile compositions comprising the antibodies of the present invention, these antibodies can be admixed with a pharmaceutically acceptable carrier or excipient. [See, e.g., Remington's Pharmaceutical Sciences and U.S. Pharmacopeia: National Formulary, Mack Publishing Company, Easton, PA (1984)].

Formulations of therapeutic and diagnostic agents may be prepared by mixing with acceptable carriers, excipients, or stabilizers in the form of, e.g., lyophilized powders, slurries, aqueous solutions or suspensions [see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY]. In one embodiment, the antibodies of the present invention are diluted to an appropriate concentration in a sodium acetate solution pH 5-6, and NaCl or sucrose is added for tonicity. Additional agents, such as polysorbate 20 or polysorbate 80, may be added to enhance stability.

Toxicity and therapeutic efficacy of the antibody compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, antibodies exhibiting high therapeutic indices are desirable. The data obtained from these cell culture assays and animal studies can be used in formulating a range of dosage for use in canines. The dosage of such compounds lies preferably within a range of circulating concentrations that include the ED₅₀ with little or no toxicity. The dosage may vary within this range depending upon the dosage form employed and the route of administration.

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial. In particular embodiments, the antibodies of the present invention can be administered by an invasive route such as by injection. In further embodiments of the invention, the antibodies of the present invention, or pharmaceutical composition thereof, is administered intravenously, subcutaneously, intramuscularly, intraarterially, or by inhalation, aerosol delivery. Administration by non-invasive routes (e.g., orally; for example, in a pill, capsule or tablet) is also within the scope of the present invention.

Compositions can be administered with medical devices known in the art. For example, a pharmaceutical composition of the invention can be administered by injection with a hypodermic needle, including, e.g., a prefilled syringe or autoinjector. The pharmaceutical compositions disclosed herein may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

The pharmaceutical compositions disclosed herein may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternatively, one may administer the antibodies of the present invention in a local rather than systemic manner, often in a depot or sustained release formulation.

The administration regimen depends on several factors, including the serum or tissue turnover rate of the therapeutic antibodies, the level of signs/symptoms, the immunogenicity of the therapeutic antibodies and the accessibility of the target cells in the biological matrix. Preferably, the administration regimen delivers sufficient therapeutic antibodies to effect improvement in the target disease/condition state, while simultaneously minimizing undesired side effects. Accordingly, the amount of biologic delivered depends in part on the particular therapeutic antibodies and the severity of the condition being treated. Guidance in selecting appropriate doses of therapeutic antibodies is available [see, e.g., Wawrzynczak Antibody Therapy, Bios Scientific Pub. Ltd, Oxfordshire, U K (1996); Kresina (ed.) Monoclonal Antibodies, Cytokines and Arthritis, Marcel Dekker, New York, NY (1991); Bach (ed.) Monoclonal Antibodies and Peptide Therapy in Autoimmune Diseases, Marcel Dekker, New York, NY (1993); Baert, et al. New Engl. J. Med. 348:601-608 (2003); Milgrom et al. New Engl. J. Med. 341:1966-1973 (1999); Slamon et al. New Engl. J. Med. 344:783-792 (2001); Beniaminovitz et al. New Engl. J. Med. 342:613-619 (2000); Ghosh et al. New Engl. J. Med. 348:24-32 (2003); Lipsky et al. New Engl. J. Med. 343:1594-1602 (2000)].

Determination of the appropriate dose is made by the veterinarian, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of the signs/symptoms.

Antibodies provided herein may be provided by continuous infusion, or by doses administered, e.g., daily, 1-7 times per week, weekly, bi-weekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. A total weekly dose is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more [see, e.g., Yang, et al. New Engl. J. Med. 349:427-434 (2003); Herold, et al. New Engl. J. Med. 346:1692-1698 (2002); Liu, et al. J. Neurol. Neurosurg. Psych. 67:451-456 (1999); Portielji, et al. Cancer Immunol. Immunother. 52:133-144 (2003)]. Doses may also be provided to achieve a pre-determined target concentration of antibodies of the present invention in the canine's serum, such as 0.1, 0.3, 1, 3, 10, 30, 100, 300 μg/ml or more. In other embodiments, antibodies of the present invention is administered subcutaneously or intravenously, on a weekly, biweekly, “every 4 weeks,” monthly, bimonthly, or quarterly basis at 10, 20, 50, 80, 100, 200, 500, 1000 or 2500 mg/subject.

As used herein, “inhibit” or “treat” or “treatment” includes a postponement of development of the signs/symptoms associated with a disorder and/or a reduction in the severity of the signs/symptoms of such disorder. The terms further include ameliorating existing uncontrolled or unwanted signs/symptoms, preventing additional signs/symptoms, and ameliorating or preventing the underlying causes of such signs/symptoms. Thus, the terms denote that a beneficial result has been conferred on a vertebrate subject (e.g., a canine) with a disorder, condition and/or sign/symptom, or with the potential to develop such a disorder, disease or sign/symptom.

As used herein, the terms “therapeutically effective amount”, “therapeutically effective dose” and “effective amount” refer to an amount of antibodies of the present invention that, when administered alone or in combination with an additional therapeutic agent to a cell, tissue, or subject, e.g., canine, is effective to cause a measurable improvement in one or more signs/symptoms of a disease or condition or the progression of such disease or condition. A therapeutically effective dose further refers to that amount of the antibodies sufficient to result in at least partial amelioration of signs/symptoms, e.g., treatment, healing, prevention or amelioration of the relevant medical condition, or an increase in rate of treatment, healing, prevention or amelioration of such conditions. When applied to a combination, a therapeutically effective dose refers to combined amounts of the active ingredients that result in the therapeutic effect, whether administered in combination, serially, or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess severity of the condition, e.g., atopic dermatitis.

The foregoing description of the specific embodiments will so fully reveal the general nature of the invention that others can, by applying knowledge within the skill of the art (including the contents of the references cited herein), readily modify and/or adapt for various applications, such as specific embodiments, without undue experimentation, without departing from the general concept of the present invention. Therefore, such adaptations and modifications are intended to be within the meaning and range of equivalents of the disclosed embodiments, based on the teaching and guidance presented herein.

All references cited herein, including journal articles or abstract, published, or corresponding patent applications, patents, or any other references, are incorporated by reference herein in its entirety, including all data, tables, figures, and text presented in the cited references. Additionally, the entire contents of the references cited within the references cited herein are also entirely incorporated by reference.

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Having now generally described the invention, the same will be more readily understood through reference to the following examples which are provided by way of illustration and are not intended to be limiting to the present invention. Further aspects and embodiments will be apparent to those skilled in the art.

### EXAMPLES

### Summary

### Canine Antibodies to IL-13

Recombinant tagged canine IL-13 proteins were used together with a comprehensive canine IgM/IgG/l/k scFv phage display library to obtain candidate scFvs for canine IL-13. A total of twelve initial candidate scFvs for canine IL-13 were obtained. All twelve (12) scFvs for IL-13 were sequenced and nucleic acids encoding recombinant canine antibodies to canine IL-13 were generated and assembled into vectors for recombinant protein expression. All of the recombinant scFvs were subjected to a cell-based blocking assay to demonstrate the blocking activity of these anti-IL13 scFv. Of these, only a total of five scFvs were found to sufficiently block the binding of canine IL-13 to its receptor, which is required for the activation of the STAT-6 signaling pathway. The DNA sequences of these scFvs were used to construct the full-length canine IgG antibody retaining the same variable regions as the scFvs. The DNA of the full-length antibodies were synthesized and assembled in an expression vector which were transfected into mammalian cells to produce the recombinant antibodies. The antigen binding and receptor blocking activities of these full-length antibodies were assessed. All five full-length antibodies were found to block the canine IL-13 activity in a cell-based pSTAT6 activation assay. These candidates were labeled as blockers. Among the five blockers, only three of them retain 10 nM or lower (stronger) IC50 blocking activity. The quality of all blockers was further assessed using a panel of analytical biochemical and biophysical tests using HPLC. Examples of these testing procedures include size exclusion chromatography to determine the purity of the antibody through monomer content, hydrophobic Interaction Chromatography to determine the potential residue oxidation, Ion Exchange Chromatography to test charge variants and Reverse Phase Chromatography for determination of conformational variants. The stability of these blockers also was assessed at 25° C. and 37° C. After these testing procedures were performed to eliminate unstable or heterogenous antibodies unstable or heterogenous antibodies, only one (1) candidate remained: i.e., IL13-P3-05VH-cIgGB/IL13-P3-05VLm-cCL. The light chain of IL13-P3-05VH-cIgGB/IL13-P3-05VLm-cCL has been specifically modified by replacing an asparagine (N) amino acid residue in its CDRL2 with a glutamine (Q) amino acid residue to remove a potential glycosylation site. The canine antibody IL13-P3-05VH-cIgGB/IL13-P3-05VLm-cCL binds CIL-13 and blocks the binding of cIL-13 to the canine Type II heterodimer interleukin-4 receptor (Type II cIL-4R), which consists of an IL-4Rα chain and an IL-13R alpha 1 chain; but it does not block the binding of cIL-13 to cIL-13R alpha 2.

### Canine Antibodies to IL-4

Recombinant tagged canine IL-4 proteins were used together with a comprehensive canine IgM/IgG/l/k scFv phage display library to obtain candidate scFvs for canine IL-4. A total of twenty-five (25) initial candidate scFvs for canine IL-4 were obtained. All twenty-five scFvs for IL-4 were sequenced and nucleic acids encoding recombinant canine antibodies to canine IL-4 were generated and assembled into vectors for recombinant protein expression. All of the recombinant scFvs were subjected to a cell-based blocking assay to demonstrate the blocking activity of these anti-IL-4 scFv. Of these, only a total of eleven (11) scFvs were found to sufficiently block the binding of canine IL-4 to its receptor, which is required for the activation of the STAT-6 signaling pathway. The DNA sequences of these scFvs were used to construct the full-length canine IgG antibody retaining the same variable regions as the scFvs. The DNA of the full-length antibodies were synthesized and assembled in an expression vector which were transfected into mammalian cells to produce the recombinant antibodies. The antigen binding and receptor blocking activities of these full-length antibodies were assessed. All eleven full-length antibodies were found to block the canine IL-4 activity in a cell-based pSTAT6 activation assay. These candidates were labeled as blockers. Among the eleven blockers, only seven of them retain 10 nM or lower (stronger) IC50 blocking activity. The quality of all blockers was further assessed using a panel of analytical biochemical and biophysical tests using HPLC. Examples of these testing procedures include size exclusion chromatography to determine the purity of the antibody through monomer content, hydrophobic Interaction Chromatography to determine the potential residue oxidation, Ion Exchange Chromatography to test charge variants and Reverse Phase Chromatography for determination of conformational variants. The stability of these blockers also was assessed at 25° C. and 37° C. After these testing procedures were performed to eliminate unstable or heterogenous antibodies unstable or heterogenous antibodies, only two (2) candidates remained: IL4-P4A-1F10VH-cIgGB/IL4-P4A-1F10VL-cCL and IL4-P4A-2D11VH-cIgGB/IL4-P4A-2D11VL-cCL.

### Example 1: Canine scFv Phage Display Library Panning

### Production of Canine IL-4 and IL-13 for Screening

In order to provide reagents for identification of antibodies specific to canine IL-4 or canine IL-13 respectively, and to provide reagents for immunological and functional assays, canine IL-4 and canine IL-13 proteins were produced recombinantly with various tags such HIS tags and Avi-Tags. The use of these tags was to facilitate screening and purification of proteins.

Canine IL-4 with C-terminal Avi-HIS tag:
[SEQ ID NO: 1]
HNFNITIKEIIKMLNILTARNDSCMELTVKDVFTAPKNTSDKEIFCRAATVLRQIYTHNCS
NRYLRGLYRNLSSMANKTCSMNEIKKSTLKDFLERLKVIMQKKYYRHGLNDIFEAQKIE
WHEHHHHHHHH
Canine IL-13 with C-terminal Avi-HIS tag
[SEQ ID NO: 2]
SPSPVTPSPTLKELIEELVNITQNQASLCNGSMVWSVNLTAGMYCAALESLINVSDCSAIQ
RTQRMLKALCSQKPAAGQISSERSRDTKIEVIQLVKNLLTYVRGVYRHGNFRGLNDIFEA
QKIEWHEHHHHHHHH

For isolation of canine scFv antibody fragments to canine IL-4 (cIL-4) and canine IL-13 (cIL-13) proteins, a comprehensive canine IgM/IgG/λ/κ scFv phage display library containing an estimated 40 billion canine scFv transformants was selected using a modification of conventional solid-phase panning as described in Barbas et al., [Phage Display: A Laboratory Manual, Cold Spring Harbor Press, (2001)]. For each round of panning, wells of a 96-well Costar 3490 ½ area plastic microplate were coated with 50 μl of 20 μg/ml streptavidin (SA) at 4° C. overnight (24 wells in panning 1, 16 wells in panning 2, 8 wells in panning 3, and 8 wells in panning 4). The plates were washed in PBS, the wells were blocked with 2% milk in PBS (MPBS), and the plates were incubated at 37° C. for 1 hour. Biotinylated avitag-cIL-4 or biotinylated avitag-cIL13 (36 pmoles) was added to each SA-coated well and captured by the plate-bound SA. Following a 1-hour incubation at 37° C., the plates were washed with PBS to remove any free biotinylated target. Equal aliquots of μκ, μλ, γκ, and γλ canine scFv phage display libraries were mixed together, blocked for 1 hour at room temperature (RT) in 2% milk/PBS, and 50 μl was added to each target-coated well. Panning was performed after a 2-hour incubation at 37° C., unbound phage were washed away 5 times during the first panning and 10 times during subsequent panning rounds with PBS supplemented with 0.5% Tween 20 (PBST). Each wash was performed with a 5-min incubation of wash buffer in wells to select for binders with longer off-rates. To avoid capturing scFv-phage specific for SA or the biotinylated avitag on the target protein, prior to each round of positive selection, phage were negatively selected against wells coated with SA and pre-loaded with an irrelevant biotinylated avitag protein. Using this 2-step negative/positive selection approach, the IgM/IgG/κ/λ canine scFv phage display library underwent 4 rounds of selection using acid elution of bound phage as described [Barbas et al., supra].

scFv phage ELISA. To assess the success of selection for IL-4 or IL-13-specific scFv-displayed phage particles, phage ELISAs were performed. To detect the binding of phage-displayed scFvs, microplates were coated with streptavidin and biotinylated avitag targets and control antigens as for the library panning as above. To the antigen-coated plate, samples were added of polyclonal phage from the PEG-precipitated initial library and libraries obtained after each round of panning (diluted 1:1000 in MPBS) or monoclonal phage prepared from randomly picked phage clones from output panning plates of the third round and fourth rounds of panning (non-PEG-precipitated phage diluted 1:100 in MPBS) to coated plates and incubated for 1 hour at 37° C. Plates were washed with PBST and a 1:5000 dilution of HRP-conjugated anti-M13 bacteriophage mAb (GE Healthcare, Chicago, IL) in MPBS was added. Plates were washed again, and bound HRP-conjugated secondary antibody was detected with the 2,2′-Azino-bis(3-ethylbenzthiazoline-6-sulfonic acid) substrate (ABTS). The OD was read at 405 nm after 30 min. using a Molecular Devices SpectraMax 340 spectrophotometer. Plates coated with no target antigen, streptavidin alone, and streptavidin with an irrelevant target antigen were used as negative controls.

### Example 2: Soluble scFv ELISA

In order to identify cohorts of target-specific scFv antibodies unlinked to phage, polyclonal phage preparations from selected rounds of panning were used to transfect a non-suppressor strain of E. coli (TOP10F′ cells) to create bacterial clones that express scFv unlinked to the pIII coat protein of M13 phage. Random bacterial colonies were selected, grown as described [Barbas et al., supra], and small-scale preparations of scFv were harvested from the periplasmic space of the bacteria. ELISAs to identify binders were performed as above for monoclonal scFv-phage except that an anti-hemagglutinin (HA) tag HRP-conjugated antibody was used for detection. Plasmid minipreps were prepared from each bacterial clone and the nucleotide sequences of the scFv heavy chain variable regions and light chain variable regions were determined. From both the ELISA analysis for positive binding clones and nucleotide sequencing of scFv-containing plasmid DNA, unique anti-cIL4 and anti-cIL13 antibody variable regions were identified. Glycerol stocks of bacterial clones were prepared and stored at −80° C. for subsequent use for large-scale scFv production.

### Example 3: Expression and Purification of scFv

Glycerol stocks of bacteria containing scFv constructs positive for antigen binding were used to seed cultures for the preparation of purified scFv used to confirm antigen binding and determine other properties including blockage of IL-4 and IL-13 to their respective receptors.

### Media, Buffers, Reagents, and Protocols

LB media
Difco LB
SB media
MOPS. Fisher: BP308-500 or equivalent.
Yeast extract. Fisher: BP1422-500 or equivalent.
Tryptone: Fisher: BP9726-500 or equivalent
IPTG. RPI: 156000-5.0 or equivalent.
Carbenicillin. Invitrogen: 10177-012 or equivalent.
Concentrated 40% glucose solution
Dextrose. Sigma: G-8270 or equivalent.

### Periplasmic Extraction Buffer I

100 mM Tris-HCl
20% sucrose
1 mM EDTA
adjust pH to 8.0

### Periplasmic Extraction Buffer II

5 mM MgCl₂

### High Salt Wash Buffer

50 mM Tris-HCl
300 mM NaCl
10 mM imidazole
adjust pH to 8.0

### Low Salt Wash Buffer

50 mM Tris-HCl
150 mM NaCl
10 mM imidazole
adjust pH to 8.0

### Elution Buffer

100 mM Tris-HCl
150 mM NaCl
300 mM imidazole
adjust pH to 8.0

### Concentrated Imidazole Solution

Make up 100 ml of a 2 M stock solution from solid in H₂O. Powder from Fisher: BP305-50 or equivalent.

### Concentrated NaCl Solution

5 M NaCl. Corning: 46-032-CV or equivalent.
EDTA 0.5 M solution. Gibco: 15575-038 or equivalent.
Sucrose. Fisher: S71203/S25590 or equivalent.
Ni-NTA Agarose. Qiagen: 1018244.
Concentrators: Amicon Ultra-4 centrifugal filters. 10 k cutoff. Millipore: UFC801096.
PBS —Mg, —Ca. Lonza: 17-516F or equivalent
   Purification of scFv Polypeptides from E. coli

### 1. Growth of Cultures.

E. coli from cultures used to generate mini-prep DNAs for sequence analysis are used to inoculate starter cultures. A glycerol stock scraping or 10 μl of culture to inoculate starter flasks was used.
Starter cultures are made up in small flasks (125 ml or 250 ml). Add 10 to 30 ml LB containing 100 μg/ml carbenicillin and 1% glucose per flask. Inoculate with scFv clone TOP10 E. coli (above). Shake overnight (O/N) at 37° C. at 250 rpm.
The following morning, add 1/20 volume of starter culture to SB media in 500 or 1000 ml flasks containing 100 μg/ml carbenicillin and 0.1% glucose. Shake at room temperature at 200 rpm. Cultures will start to get cloudy in 4-6 hours. Induce with 0.5 mM IPTG at around 5 hours and continue to shake at room temp O/N.
This protocol is written for 500 ml expression culture volumes in 1 liter flasks. This means that 25 ml of starter culture is used for inoculation. For 200 or 300 ml expression cultures, 10 or 15 mls of starter culture is used for inoculation.

### 2. Cell Harvest and Preparation of Periplasmic Extracts.

Begin harvest of cultures the following day at 22-26 hours post IPTG induction. Centrifuge at 4K rpm for 15′ at 4° C. Decant supernatants.
Extract pellets from 500 ml cultures in 20 ml periplasmic extraction buffer I. Use a 10 ml pipet to promote solubilization, getting rid of clumps. Transfer to 50 ml tubes. On ice ≥20′. Centrifuge at 4K rpm, 15′ at 4° C. Tip supernatants into new 50 ml tubes and store on ice.
Re-extract pellets with 20 ml periplasmic extraction buffer II. Use 10 ml pipet (one can use the same pipet from step I) to promote solubilization, getting rid of clumps. On ice ≥20′ Centrifuge 4K rpm, 15′ at 4° C. Combine buffer II supernatants with buffer I supernatants. Mix and keep on ice. The periplasmic extracts can be kept on ice O/N and up to several days if desired.
   3. Ni-NTA Binding, Washes and Elution of scFv Proteins
To 40 ml of periplasmic extract, add NaCl to 300 mM and imidazole to 10 mM final. Centrifuge 4K rpm, 15′ at 4° C.
Prepare washed Ni-NTA resin by pipetting 2 ml of 50% slurry into 50 ml tubes. Add 30 ml of high salt wash buffer. Mix briefly. Centrifuge at 2K rpm, 10′ at 4° C. Remove wash buffer taking care not to disturb the resin.
Then tip extract supernatants into tubes containing prewashed Ni-NTA agarose.
Rotate at 4° C. for 2 hours or overnight (O/N).
Centrifuge the resin at 2K rpm, 5′ at 4° C. Discard supernatants with pipet or aspirator. Add 30 ml high salt wash buffer. Mix until resin is resuspended. Re-spin 2K rpm, 5′ at 4° C. Remove supernatant.
Repeat above step 2× for a total of 3 high salt washes. Transfer the resin to 15 ml tubes using high salt buffer. Add high salt buffer to 12 ml final. Mix resin, re-centrifuge and aspirate.
After high salt supernatant removal, perform two more washes in 12 ml low salt wash buffer.
Add 5 ml of elution buffer per bead pellet. Mix briefly. Centrifuge resin at 2K rpm, 5′ at 4° C. Transfer supernatants to new tubes. Repeat with 2^{nd} 5 ml aliquot of elution buffer. Combine with the first elution.
   4. Concentration, Buffer Exchange and Analysis of scFv Preparations.
Prewash 4 ml Amicon 10 k cutoff ultrafiltration units with PBS. Add 3 ml PBS without Ca and Mg and centrifuge at 3.5K rpm for 15′ at 4° C. Discard PBS.
Concentrate periplasmic preparations to no less than 0.5 ml. This is 15-20′ at 3.5K rpm. Add 4.5 ml PBS. Re-concentrate to 0.5 ml. Add 2^{nd} 4.5 ml aliquot of PBS. Concentrate to 0.5 ml or less. Repeat a third time bringing the final volumes down to 0.5 ml.
Take 280 nm nanodrop readings with E1% of 16.0. Use flow through of Amicon units to blank the instrument. Make a table of concentrations and yields of scFvs. Any scFv prep with a concentration less than 0.2 mg/ml should be further concentrated to a smaller volume to obtain a higher protein value.
Determine volumes of scFv proteins to analyze 3 μg of each by SDS-PAGE. Run a BSA STD to quantify preparations by Coomassie stain and image analysis of gels.

### Example 4: Binding of scFv to Canine IL-4 and Canine IL-13

The in vitro binding of anti-canine IL-4 scFv and IL-13 scFv to canine IL-4 and canine IL-13, respectively, was determined by OCTET HTX using SA biosensors and Data Acquisition 12.0 software. First, biotin-labeled antigen (cIL-4 or cIL-13) was loaded onto the pre-rehydrated SA biosensors for 60 seconds (60s) at a concentration of 5 μg/mL. Second, the biosensors were placed into the OCTET Kinetics Buffer (PBS+0.02% Tween20, 0.1% BSA) for the blocking phase for 120s. Third, for the association phase, cIL-4 or cIL-13 antigen loaded biosensors were placed into 2-fold serial dilutions from 500 nM down to 7.8 nM of anti-cIL-4 or anti-cIL-13 scFvs, respectively, in OCTET Kinetics Buffer for 60s. The last well was buffer alone and that sensor was used for reference sensor subtraction. Finally, the biosensors were placed into OCTET Kinetics Buffer for the dissociation phase for 300s. The analysis was performed using Data Analysis 12.0 software and curves were fitted using a 1:1 binding model. Binding affinity measurement results indicate that the tested anti-cIL-4 scFvs have low nanomolar binding affinities ranging from 52 nM to 1.12 nM. Strong anti-cIL-4 scFv binders include P3-1A11, P3-1F7, and P3-1A6 with dissociation constants (KDs) of 1.12 nM, 1.20 nM, and 1.24 nM, respectively (see, Table 1 below). The anti-cIL-13 scFvs have binding affinities ranging from 157.7 nM to 8.24 nM. Strong anti-cIL-13 scFv binders include P2-3A10, P2-1H5, and P3-1B4 with KDs of 8.24 nM, 9.09 nM, and 13.07 nM, respectively (see, Table 2 below).

TABLE 1
BINDING KINETICS of ANTI-IL-4 scFv
DETERMINED by OCTET HTX
Anti-IL-4 scFv (1:1 Binding)

| Sample ID | KD (M) | ka (1/Ms) | kdis (1/s) |
|---|---|---|---|
| P3-06 | 1.43E−08 | 1.28E+06 | 1.82E−02 |
| P3-1 A11 | 1.12E−09 | 2.35E+05 | 2.63E−04 |
| P3-1 A6 | 1.24E−09 | 2.19E+05 | 2.72E−04 |
| P3-1 B10 | 4.57E−09 | 1.14E+06 | 5.22E−03 |
| P3-1 B4 | 6.83E−09 | 1.95E+05 | 1.33E−03 |
| P3-1 B9 | 4.50E−09 | 2.41E+05 | 1.08E−03 |
| P3-1 C4 | 4.13E−09 | 1.32E+05 | 5.47E−04 |
| P3-1 D11 | 5.29E−09 | 5.63E+05 | 2.98E−03 |
| P3-1 D7 | 1.75E−08 | 3.91E+05 | 7.29E−03 |
| P3-1 F4 | 5.08E−08 | 7.30E+05 | 3.67E−02 |
| P3-1 F6 | 2.89E−08 | 1.06E+06 | 3.06E−02 |
| P3-1 F7 | 1.20E−09 | 6.94E+05 | 8.31E−04 |
| P3-1 H3 | 2.00E−08 | 6.06E+05 | 1.21E−02 |
| P4-1 A10 | 9.92E−09 | 9.54E+05 | 1.10E−02 |
| P4-1 G3 | 3.94E−09 | 5.94E+05 | 2.47E−03 |
| P4-1 H1 | 4.87E−09 | 5.62E+05 | 2.63E−03 |
| P4-1 H8 | 2.30E−09 | 6.72E+05 | 1.55E−03 |
| P4A-1 A4 | 5.20E−08 | 7.04E+05 | 3.73E−02 |
| P4A-1 E10 | 1.03E−08 | 1.94E+05 | 1.73E−03 |
| P4A-1 A2 | 2.05E−08 | 4.43E+05 | 1.03E−02 |
| P4A-1 D9 | 3.60E−08 | 2.87E+05 | 1.17E−02 |
| P4A-1 F10 | 1.27E−08 | 3.11E+05 | 3.96E−03 |
| P4A-2 D11 | 4.38E−09 | 1.52E+06 | 7.75E−03 |
| P4A-2 G11 | 8.50E−09 | 1.37E+05 | 1.17E−03 |

TABLE 2
BINDING KINETICS of ANTI-IL-13 scFv
DETERMINED by OCTET HTX
Anti-IL-13 scFv (1:1 Binding)

| Sample ID | KD (M) | ka (1/Ms) | kdis (1/s) |
|---|---|---|---|
| P2-1H5 | 9.09E−09 | 3.21E+05 | 2.91E−03 |
| P2-3A10 | 8.24E−09 | 1.60E+06 | 1.32E−02 |
| P3-05 | 5.97E−08 | 1.76E+05 | 4.87E−03 |
| P3-1 B4 | 1.31E−08 | 3.20E+05 | 2.24E−03 |
| P3-1 C2 | 7.74E−08 | 2.96E+04 | 1.92E−03 |
| P3-1 C6 | 4.52E−08 | 3.04E+05 | 1.07E−02 |
| P3-1 G5 | 1.58E−07 | 1.04E+05 | 1.58E−02 |
| P4-1 E10 | 4.12E−08 | 1.86E+05 | 4.36E−03 |
| P4-1 E3 | 1.35E−07 | 3.00E+05 | 3.97E−02 |
| P4A-1B7 | 5.08E−08 | 1.02E+05 | 5.21E−03 |
| P4A-2E11 | 2.68E−08 | 1.74E+05 | 4.66E−03 |

### Example 5: Monovalent Binding of Full-Length Anti-Canine IL-4 mAbs and Anti-Canine IL-13 mAbs to Canine IL-4 and Canine IL-13, Respectively

Selected scFv specific to cIL-4 or cIL-13 were converted into full length canine IgG mAbs and tested for binding by OCTET. All kinetics measurements were performed by OCTET HTX using SA biosensors and Data Acquisition 12.0 software. First, each biotin-labeled full-length anti-canine IL-4 or anti-canine IL-13 mAb, at a concentration of 5 μg/mL, were loaded onto the pre-rehydrated SA biosensors until binding signal change ascended to a 1 nM threshold for all channels from the step start or for a maximum of 1200s to insure equal loading density. Second, the biosensors were placed into OCTET Kinetics Buffer (PBS+0.02% Tween20, 0.1% BSA) for the blocking phase for 60s. Third, for the association phase, full-length anti-canine IL-4 mAb or anti-canine IL-13 mAb loaded biosensors were placed into 2-fold serial dilutions from 1000 nM down to 15.6 nM of cIL-4 or cIL-13, respectively, in OCTET Kinetics Buffer for 60s. The last well was buffer alone and that sensor was used for reference sensor subtraction. Finally, the biosensors were placed into OCTET Kinetics Buffer for the dissociation phase for 180s. Analysis was performed using Data Analysis 12.0 software and curves were fitted using a 1:1 binding model. Binding affinity measurement results indicate that the tested full-length anti-cIL-4 mAbs have low nanomolar to low picomolar binding affinities ranging from 10.5 nM to 63.5 μM. Strong binders include IL4-P3-1B9VH-cIgGB/IL4-P3-1B9VL-cCL, IL4-P4A-1F10VH-cIgGB/IL4-P4A-1F10VL-cCL, and IL4-P3-1B10VHm-cIgGB/IL4-P3-1B10VL-cCL with KDs of 63.5 μM, 305 μM, and 622 μM, respectively (see, Table 3 below).

TABLE 3
BINDING KINETICS of ANTI-CIL-4 FULL LENGTH IgG DETERMINED by Octet HTX
Anti-cIL-4 Full-Length mAb 1:1 Binding

| Sample ID | KD (M) | ka (1/Ms) | kdis (1/s) |
|---|---|---|---|
| IL4-P3-1B10VHm-cIgGB/IL4-P3-1B10VL-cCL | 6.22E−10 | 1.30E+06 | 8.09E−04 |
| IL4-P3-1B9VH-cIgGB/IL4-P3-1B9VL-cCL | 6.35E−11 | 2.12E+05 | 2.11E−05 |
| IL4-P3-1C4VH-cIgGB/IL4-P3-1C4VL-cCL | 1.05E−08 | 1.80E+06 | 1.89E−02 |
| IL4-P3-1D7VH-cIgGB/IL4-P3-1D7VL-cCL | 2.01E−09 | 1.12E+06 | 2.26E−03 |
| IL4-P4-1G3VH-cIgGB/IL4-P4-1G3VL-cCL | 1.51E−09 | 1.61E+06 | 2.43E−03 |
| IL4-P4A-1A10VH-cIgGB/IL4-P4-1A10VL-cCL | 8.46E−10 | 1.24E+06 | 1.05E−03 |
| IL4-P4A-1F10VH-cIgGB/IL4-P4A-1F10VL-cCL | 3.05E−10 | 3.74E+05 | 1.14E−04 |

The tested full-length anti-cIL-13 mAbs have low nanomolar binding affinities ranging from 5.58 nM to 1.19 nM. Strong binders include IL13-P3-1G5VH-cIgGB/IL13-P3-1G5VL-cCL, IL13-P3-05VH-cIgGB/IL13-P3-05VLm-cCL, and IL13-P3-05VH-cIgGB/IL13-P3-05VL-cCL with KDs of 1.19 nM, 5.45 nM, and 5.58 nM, respectively. These results also are consistent with the modification of IL13-P3-05VH-cIgGB/IL13-P3-05VL-cCL to IL13-P3-05VH-cIgGB/IL13-P3-05VLm-cCL, by replacing the asparagine amino acid residue of the CDRL2 with a glutamine amino acid residue, having no significant measurable effect on the KD for the binding of this canine antibody to cIL-13 (see, Table 4 below).

TABLE 4
BINDING KINETICS of ANTI-CIL-13 FULL LENGTH IgG DETERMINED by OCTET HTX
Anti-cIL-13 Full-Length mAb (1:1 Binding)

| Sample ID | KD (M) | ka (1/Ms) | kdis (1/s) |
|---|---|---|---|
| IL13-P3-05VH-cIgGB/IL13-P3-05VL-cCL | 5.58E−09 | 2.52E+05 | 1.48E−03 |
| IL13-P3-05VH-cIgGB/IL13-P3-05VLm-cCL | 5.45E−09 | 1.62E+05 | 7.93E−04 |
| IL13-P3-1B4VH-cIgGB/IL13-P3-1B4VL-cCL | 8.12E−09 | 2.56E+05 | 2.08E−03 |
| IL13-P3-1G5VH-cIgGB/IL13-P3-1G5VL-cCL | 1.19E−09 | 1.30E+06 | 1.80E−03 |

### Example 6: Inhibition of STAT-6 Phosphorylation Inhibition by Antibodies

DH82 cells are a native canine macrophage-like cell capable of STAT-6 signaling upon activation with cIL-4 or cIL-13. DH82 cells can be used to measure the capacity of a canine antibody to cIL-4 to inhibit the cIL-4 mediated canine Type II heterodimer IL-4 receptor signaling pathway and the capacity of a canine antibody to cIL-13 to inhibit the cIL-13 mediated canine Type II heterodimer IL-4 receptor signaling pathway. As indicated above, the Type II heterodimer IL-4 receptor signaling pathway is involved in the inflammation associated with atopic dermatitis. The canine antibodies examined below specifically bind to either cIL-4 or cIL-13 respectively, and their capacity to inhibit cIL-4 or cIL-13 STAT-6 activation respectively, was measured using a DH82 cell-based assay.

### Methods

Actively growing DH82 cells (MAH NB reference 5028717-0077, passage 11) were prepared at a density of 2.5×10⁶ cells/mL in HBSS (1×: Corning, Catalog 21-022-CM) and 40 μL was added to each well of 96-well tissue culture plates to obtain 1×10⁵ cells/well. The plates were placed in a humidified, static incubator at 37° C. with 5% CO₂ for 1-2 hours. Antibodies were prepared at 4× concentrations diluted in HBSS and 20 μL/well was added to the wells in the cell plates. Recombinant canine IL-13 (R&D Systems, Catalog: 5894-CL/CF) or recombinant canine IL-4 (R&D Systems, Catalog: 752-CL/CF) were diluted separately in HBSS (final concentration 5 ng/ml cIL-13 and 2.5 ng/ml cIL-4 in the respective wells). Each well received appropriately 20 μL of either cIL-4 or cIL-13 and the plates were incubated for 15 minutes in a humidified, static incubator at 37° C. with 5% CO2. The cells were lysed with 20 μL of 5× Lysis Buffer (AlphaLISA p-STAT6 (Tyr641) Assay Kit: Perkin Elmer, Catalog: ALSU-PST6-A10K) and agitated on a plate shaker with 350 rpm for 10 minutes at room temperature. Measurement of phosphorylation on STAT-6 (Tyr641) was performed by transferring 30 μL of the lysates to 96-well ½ Area Plates with 15 μL/well of Acceptor Mix (AlphaLISA p-STAT6 (Tyr641) Assay Kit: Perkin Elmer, Catalog: ALSU-PST6-A10K), the plates were sealed and agitated for 2 minutes at 350 rpm and then incubated for 1-2 hours at room temperature. Following the incubation of cell lysate with Acceptor Mix, 15 L/well of Donor Mix from the same Perkin Elmer kit was added to the cell plates, which were sealed, covered with aluminum foil, agitated for 2 minutes at 350 rpm, and then incubated for 1-2 hours at room temperature. The AlphaSignal from each plate was read using AlphaScreen settings on Perkin Elmer EnVison.

### Results

The inhibition of canine IL-4 mediated STAT-6 phosphorylation by the antibodies specific to canine IL-4 (IC50) is shown in Table 5 below and in FIGS. 1, 2, and 4 (dose response curves). As can be seen, both IL4-P4A-1F10VH-cIgGB/IL4-P4A-1F10VL-cCL and IL4-P4A-2D11VH-cIgGB/IL4-P4A-2D11VL-cCL have low IC50 values, which is a desirable property for its use to bind cIL-4 and inhibit the cIL-4 mediated canine Type II heterodimer IL-4 receptor signaling pathway involved in atopic dermatitis.

The inhibition of canine IL-13 mediated STAT-6 phosphorylation by antibodies specific to canine IL-13 is shown in Table 6 (IC50) and FIG. 3 (dose response curve). The canine antibody IL13-P3-05VH-cIgGB/IL13-P3-05VL-cCL and its corresponding modified canine antibody IL13-P3-05VH-cIgGB/IL13-P3-05VLm-cCL proved to have the lowest IC50 values, which is a desirable property for their use to bind cIL-13 and inhibit the cIL-13 mediated canine Type II heterodimer IL-4 receptor signaling pathway involved in atopic dermatitis.

The results further indicate that either a canine antibody to cIL-13 or a canine antibody to cIL-4 can individually serve to significantly aid in the blocking of the inflammation associated with atopic dermatitis.

TABLE 5
INHIBITION of CANINE IL-4 MEDIATED STAT6 PHOSPHORYLATION
in DH82 CELLS by ANTI-IL-4 ANTIBODIES

| | IL-4 IC50 |
|---|---|
| Antibody | (nM) |
| IL4-P3-1B9VH-cIgGB/IL4-P3-1B9VL-cCL | 3.77 |
| IL4-P3-1B10VH-cIgGB/IL4-P3-1B10VL-cCL | 87.78 |
| IL4-P3-1B10VHm-cIgGB/IL4-P3-1B10VL-cCL | 29.96 |
| IL4-P3-1D7VH-cIgGB/IL4-P3-1D7VL-cCL | 37.61 |
| IL4-P4-1G3VH-cIgGB/IL4-P4-1G3VL-cCL | 8.31 |
| IL4-P4A-2D11VH-cIgGB/IL4-P4A-2D11VL-cCL | 0.91 |
| IL4-P4A-1F10VH-cIgGB/IL4-P4A-1F10VL-cCL | 0.58 |
| IL4-P4A-1A10VH-cIgGB/IL4-P4-1A10VL-cCL | 3.15 |
| IL4-P3-1A6VH-cIgGB/IL4-P3-1A6VL-cCL | 0.56 |
| IL4-P3-1C4VH-cIgGB/IL4-P3-1C4VL-cCL | 5.41 |
| IL4-P4A-1A4VH-cIgGB/IL4-P4A-1A4VL-cCL | 4.24 |
| IL4-P4A-1D9VH-cIgGB/IL4-P4A-1D9VL-cCL | 6.08 |

TABLE 6
INHIBITION of CANINE IL-13 MEDIATED STAT6 PHOSPHORYLATION
INHIBITION in DH82 CELLS by ANTIBODIES

| | IL-13 IC50 |
|---|---|
| Antibody | (nM) |
| IL13-P3-1G5VH-cIgGB/IL13-P3-1G5VL-cCL | 94.14 |
| IL13-P3-05VH-cIgGB/IL13-P3-05VL-cCL | 13.08 |
| IL13-P3-05VH-cIgGB/IL13-P3-05VLm-cCL | 4.44 |
| IL13-P3-1B4VH-cIgGB/IL13-P3-1B4VL-cCL | 97.45 |
| IL13-P3-1C6VH-cIgGB/IL13-P3-1C6VL-cCL | 47.93 |

### Example 7: Epitope Mapping

In order to identify potentially discontinuous epitopes recognized by the antibodies of the present invention to their respective antigens, e.g., canine IL-4 or canine IL-13, a method was employed that was based on chemical crosslinking using disuccinimidyl suberate (DSS) and mass spectrometry detection (XL-MS) at CovalX Inc. (CovalX® Instrument Incorporated; Covalx.com). With this method, the binding of a canine antibody to its corresponding canine antigen in the presence of a mass-labeled chemical crosslinker is performed. The cross-linking experiments allow the direct analysis of non-covalent interaction by High-Mass MALDI (matrix assisted laser desorption ionization) mass spectrometry. By mixing a protein sample containing non-covalent interactions with a specially developed cross-linking mixture [see, Bich et al. Anal. Chem., 82 (1), pp 172-179 (2010)], a non-covalent complex can be specifically detected with high-sensitivity. The covalent binding generated allows the interacting species to survive the sample preparation process and the MALDI ionization. A High-Mass detection system allows characterizing the interaction in the High-Mass range.

The presence of the antigen-antibody complex were confirmed using High Mass MALDI detection. Because the antibody/antigen (Ab/Ag) complexes were extremely stable after applying the crosslinking chemistry, five enzymes: (i) trypsin, (ii) chymotrypsin, (iii) ASP-N (a mass spectrometry (MS)-grade zinc metalloproteinase derived from a mutant strain of Pseudomonas fragi, (iv) elastase, and (v) thermolysin were used in parallel and digestion conditions were applied to the complex to provide many different overlapping peptides. Following the proteolysis an nLC Ultimate 3000-RSLC system in line with a Q-Exactive Plus MS/MS analysis was performed.

Employing chemical cross-linking, High-Mass MALDI mass spectrometry and nLC-Orbitrap mass spectrometry a molecular interface between canine IL-4 and either the canine antibody, IL4-2D11 (i.e., IL4-P4A-2D11VH-cIgGB/IL4-P4A-2D11VL-cCL) or the canine antibody IL4-1F10 (i.e., IL4-P4A-1F10VH-cIgGB/IL4-P4A-1F10VL-cCL) was characterized.

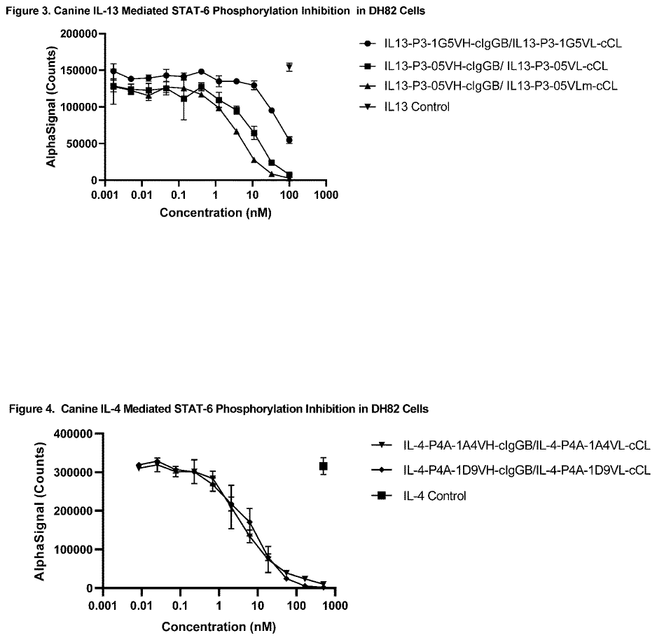

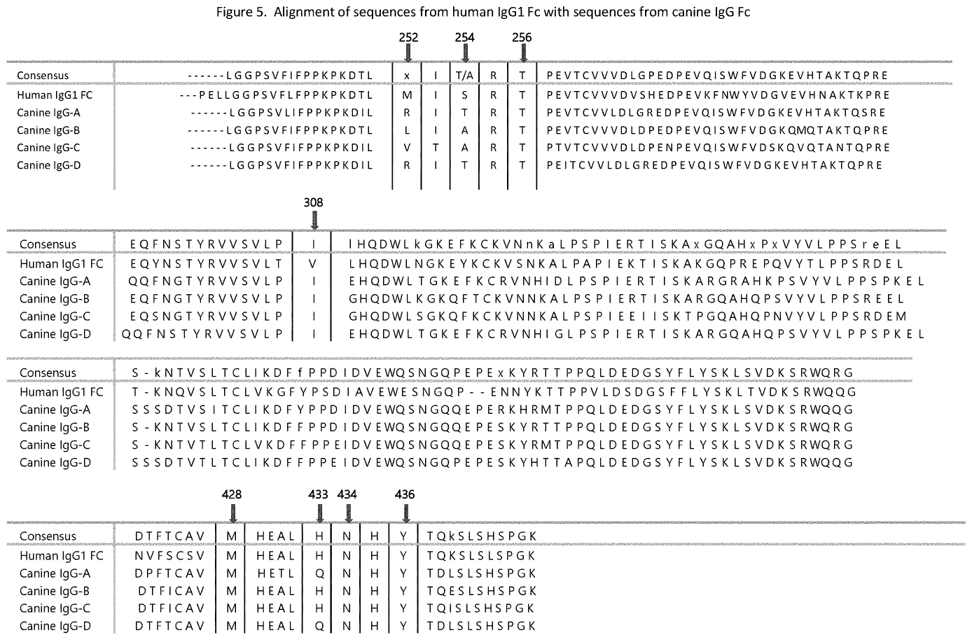

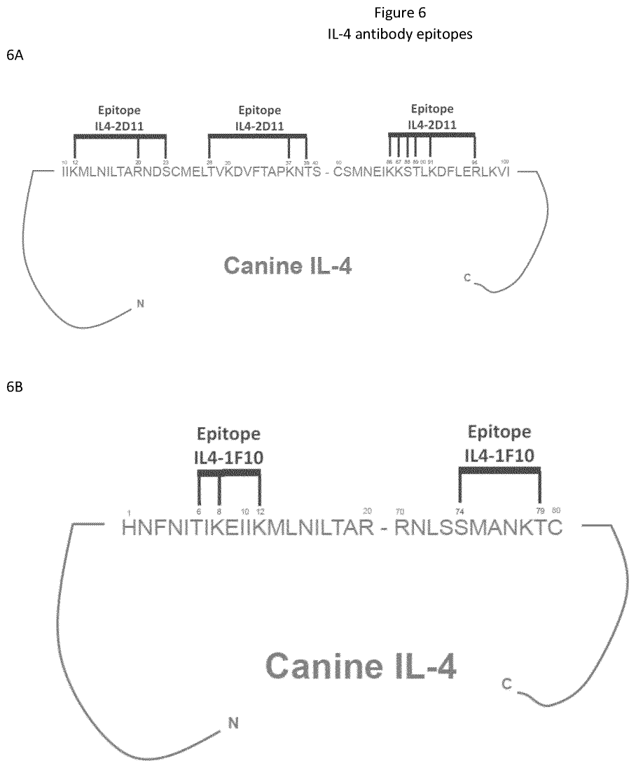

After trypsin, chymotrypsin, ASP-N, elastase and thermolysin proteolysis of the protein complex of canine IL-4/IL4-2D11 with deuterated d0d12, the nLC-Orbitrap MS/MS analysis detected 18 cross-linked peptides between canine IL-4 and IL4-2D11. The analysis indicated that the antibody binding included the following amino acid residues on canine IL-4 (SEQ ID NO: 1) at amino acid residue positions: K₁₂, R₂₀, S₂₃, T₂₈, K₃₇, T₃₉, K₈₆, K₈₇, S₈₈, T₈₉, K₉₁, and R₉₆. These results are illustrated in FIG. 6A.

After trypsin, chymotrypsin, ASP-N, elastase and thermolysin proteolysis of the protein complex of canine IL-4/IL4 1F10 with deuterated d0d12, the nLC-Orbitrap MS/MS analysis detected 18 cross-linked peptides between Canine IL-4 and IL4-1F10. The analysis indicates that the interaction includes the following amino acid residues on canine IL-4 (SEQ ID NO: 1) at amino acid residue positions: T₆, K₈, K₁₂, S₇₄, and T₇₉. These results are illustrated in FIG. 6B. It was noted that both canine antibodies to canine IL-4 specifically bind to K₁₂.

Using chemical cross-linking, High-Mass MALDI mass spectrometry and nLC-Orbitrap mass spectrometry a molecular interface between canine IL-13 and either canine IL13-P3-05Lm (IL13-P3-05VH-cIgGB/IL13-P3-05VLm-cCL), canine IL13-1G5 (IL13-P3-1G5VH-cIgGB/IL 13-P3-1G5VL-cCL), or IL13-1B4 (IL13-P3-1B4VH-cIgGB/IL13-P3-1B4VL-cCL) were characterized.

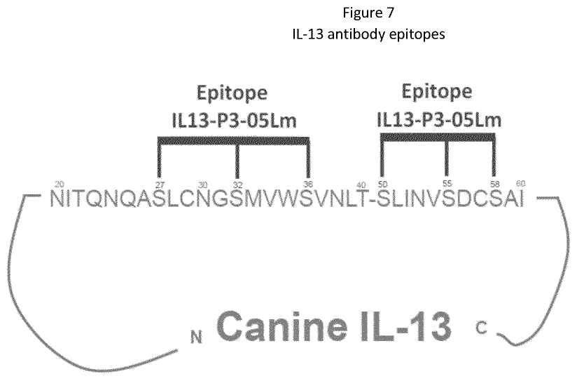

After trypsin, chymotrypsin, ASP-N, elastase and thermolysin proteolysis of the protein complex of canine IL-13/IL13-P3-05Lm with deuterated d0d12, the nLC-Orbitrap MS/MS analysis detected 14 cross-linked peptides between canine IL-13 and IL13-P3-05Lm. The analysis indicates that the interaction includes the following amino acid residues on canine IL-13 (SEQ ID NO: 2) at amino acid residue positions: S₂₇, S₃₂, S₃₆, S₅₀, S₅₅, and S₅₈. These results are illustrated in FIG. 7.

After trypsin, chymotrypsin, ASP-N, elastase and thermolysin proteolysis of the protein complex of canine IL-13/IL13-1G5 with deuterated d0d12, the nLC-Orbitrap MS/MS analysis detected 23 cross-linked peptides between canine IL-13 and IL13-1G5. The analysis indicates that the interaction includes the following amino acid residues on canine IL-13 (SEQ ID NO: 2) at amino acid residue positions: S₃₆, T₄₀, Y₄₄, S₈₂, R₈₄, S₈₅, R₈₆, T₈₈, K₈₉, K₉₇, R₁₀₄, Y₁₀₇, R₁₀₈, and H₁₀₉.

After trypsin, chymotrypsin, ASP-N, elastase and thermolysin proteolysis of the protein complex of canine IL-13/IL13-1B4 with deuterated d0d12, the nLC-Orbitrap MS/MS analysis detected 19 cross-linked peptides between canine IL-13 and IL13-1B4. The analysis indicates that the interaction includes the following amino acid residues on canine IL-13 (SEQ ID NO: 2) at amino acid residue positions: T₂₂, S₃₂, S₃₆, Y₄₄, S₅₀, S₅₅, S₅₈, R₈₆, T₈₈, and K₈₉.

It is notable that all three canine antibodies to canine IL-13 specifically bind to S₃₆. Even more notable is that both IL13-P3-05Lm and IL13-1B4 bind S₃₂, S₃₆, S₅₀, S₅₅, and S₅₈, which are five of the six binding sites identified with IL13-P3-05Lm. IL13-P3-05Lm was the best blocking antibody disclosed (see, Table 6 above). Aside from S36, both IL13-1B4 and IL13-1G5 also both bind to Y₄₄, R₈₆, T₈₈, and K₈₉. The respective epitopes recognized by the individual canine antibodies of the present invention were identified and are listed in Table 7 below.

TABLE 7
EPITOPES RECOGNIZED by CANINE ANTI-IL-4 and ANTI-IL-13 ANTIBODIES

| | | SEQ ID |
|---|---|---|
| ANTIBODY | EPITOPE | NO. |
| IL4-P4A-2D11VH-cIgGB/ | IIKMLNILTARNDSCMELTVKDVFTAPKNTS | 89 |
| IL4-P4A-2D11VL-cCL | CSMNEIKKSTLKDFLERLKVI | 90 |
| IL4-P4A-1F10VH-cIgGB/ | HNFNITIKEIIKMLNILTAR | 91 |
| IL4-P4A-1F10VL-cCL | RNLSSMANKTC | 92 |
| IL13-P3-1G5VH-cIgGB/ | NGSMVWSVNLTAGMYCAALES | 93 |
| IL13-P3-1G5VL-cCL | ISSERSRDTKIEVIQLVKNLLTYVRGVYRHG | 94 |
| IL13-P3-05VH-cIgGB/ | NITQNQASLCNGSMVWSVNLT | 95 |
| IL13-P3-05VLm-cCL | SLINVSDCSAI | 96 |
| IL13-P3-1B4VH-cIgGB/ | NITQNQASLCNGSMVWSVNLTAGMYCAALESLI | 97 |
| IL13-P3-1B4VL-cCL | ISSERSRDTKI | 98 |

Consensus sequence for the N-terminal epitope to Canine IL-4:
IIKMLNILTAR SEQ ID NO: 238
Consensus sequence for the epitope to Canine IL-13
QASLCNGSMVWSVNLT SEQ ID NO: 239
SLINVSDCSAI (SEQ ID NO: 96) or
ISSERSRDTKI (SEQ ID NO: 98)

### Example 8: Extension of Canine Antibody Half-Life

In order to extend the half-life of canine antibodies, specific amino acid substitutions were introduced into the Fc of two different canine monoclonal antibodies; one specific for IL-13 and one specific for IL-4. The IL-13 antibody with the unmodified Fc (IL-13 P3-05Lm) is represented by Heavy chain SEQ ID NO: 84 and light chain SEQ ID NO: 85. The IL-4 antibody with the unmodified Fc (IL-4-P4A-1F10) is represented by Heavy chain SEQ ID NO: 44 and Light chain SEQ ID NO: 45. The IL-13 antibody with the modified Fc (IL-13 P3-05-Lm-YD) is represented by heavy chain SEQ ID NO: 240 and light chain SEQ ID NO: 85 and includes a substitution of the amino acid at position 252 of the heavy chain (numbered according to the EU index as in Kabat) with Tyrosine (Y) and substitution of the amino acid at position 256 of the heavy chain (numbered according to the EU index as in Kabat) with Aspartic acid (D). The IL-4 antibody with the modified Fc (IL-4-P4A-1F10-YD) is represented by Heavy chain SEQ ID NO: 241 and Light chain SEQ ID NO: 45 and includes a substitution of the amino acid at position 252 of the heavy chain (numbered according to the EU index as in Kabat) with Tyrosine and a substitution of the amino acid at position 256 of the heavy chain (numbered according to the EU index as in Kabat) with Aspartic acid. The antibodies with the modified and unmodified Fc were administered to dogs.

One group of 6 dogs was administered both unmodified antibodies by SC route at about 3 mg/kg of each antibody. Another group of 6 dogs was administered both modified antibodies by SC route at about 3 mg/kg of each antibody. Animals were bled at various time points throughout the study and their sera collected and used to measure the serum concentration of each antibody by ELISA. The effect of amino acid substitutions on antibody half-life is shown in Table 8 below. The results demonstrate a clear extension in half-life as a result of the amino acid substitutions at position 252 and 256.

TABLE 8
EFFECT OF SPECIFIC AMINO ACID SUBSTITUTIONS
ON ANTIBODY HALF-LIFE

| | Mean half-life |
|---|---|
| Antibody | (days) |
| IL-4-P4A 1F10 | 9.9 |
| IL-4-P4A 1F10-YD | 14.7 |
| IL-13 P3-05-Lm | 9.1 |
| IL-13 P3-05-Lm-YD | 17 |

### Example 9: Assessment of Antibody Efficacy in Treatment of Atopic Dermatitis

The efficacy of the IL-13 and IL-4 antibodies of the present invention in treatment of atopic dermatitis is assessed in client-owned animals diagnosed with atopic dermatitis. For this purpose, groups of dogs are administered one or both antibodies of the invention by subcutaneous route for example at doses from 0.1 to 10 mg/kg. For comparison, a group of dogs is administered a placebo also via subcutaneous route. Animals are then monitored at regular intervals (for example weekly) for manifestation of key clinical signs of disease over 30 or 60-day period or longer. The success of treatment is typically determined by assessment of the impact of antibody treatment on extent of pruritus using so called Pruritus Visual Analog Scale (PVAS; a scale of 0-10 indicating the itch severity) and assessment of the impact of the antibody treatment on extent of skin lesion scores referred to as CADESI (canine atopic dermatitis extent and severity index) in treated dogs versus dogs treated with a placebo. Successful treatment outcome is usually indicated by a reduction of at least 20% in PAVS score and a reduction of at least 50% in CADESI scores; relative to baseline measurements.

Amino Acid Sequences of Canine IL-4 and Canine IL-13
Canine IL-4 with C-terminal Avi-HIS tag
[SEQ ID NO: 1]
HNFNITIKEIIKMLNILTARNDSCMELTVKDVFTAPKNTSDKEIFCRAATVLRQIYTHNCS
NRYLRGLYRNLSSMANKTCSMNEIKKSTLKDFLERLKVIMQKKYYRHGLNDIFEAQKIE
WHEHHHHHHHH
Canine IL-13 with C-terminal Avi-HIS tag [SEQ ID NO: 2]
SPSPVTPSPTLKELIEELVNITQNQASLCNGSMVWSVNLTAGMYCAALESLINVSDCSA
IQRTQRMLKALCSQKPAAGQISSERSRDTKIEVIQLVKNLLTYVRGVYRHGNFRGLNDIF
EAQKIEWHEHHHHHHHH
Amino Acid Sequences of scFvs Specific for Canine IL-4
IL4 P3-1 B9 [SEQ ID NO: 3]
QAVLTQPASVSAALGQRVTISCTGTATNVGGGYDVQWYQQFPGRPPKTIIYGNYNRPSG
VPDRFSASTSGTTATLTITGIQVADEADYYCQSFDTTFNGHVFGAGTQLTILGGGSSRSSS
SGGGGSGGGGELQLVELGGDLVKPGGSLRLSCVASGFTFSSYGMSWVRQSPGKGLQWV
ADISSSGSTYYADAVKGRFTISRDNAKSTLYLQMNSLRAEDTAVYYCAKAGYGSYYDF
GMDYWGHGTSVFVSSESPSPPNLTSGQAGQ
IL4 P3-1 B9 VH [SEQ ID NO: 4]
ELQLVELGGDLVKPGGSLRLSCVASGFTFSSYGMSWVRQSPGKGLQWVADISSSGSTYY
ADAVKGRFTISRDNAKSTLYLQMNSLRAEDTAVYYCAKAGYGSYYDFGMDYWGHGTS
VFVSSESPSPPNLTSGQAGQ
IL4 P3-1 B9 VL [SEQ ID NO: 5]
QAVLTQPASVSAALGQRVTISCTGTATNVGGGYDVQWYQQFPGRPPKTIIYGNYNRPSG
VPDRFSASTSGTTATLTITGIQVADEADYYCQSFDTTFNGHVFGAGTQLTIL
IL4 P3-1 B10 [SEQ ID NO: 6]
QSVLTQPTSVSAALGQRVTISCTGSNANIGSVYDVQWYQQLPGKSPKTIIYGNTHRPSGV
PVRFSGSKSGSTATLTITGIQAEDEADYYCQSYDATLHYVFGSGTQLTVLGGGSSRSSSS
GGGGSGGGGEVQLVESGGDLVKPGGSLRLSCVASGFTFSNYDMSWVRQAPGKGLQWV
STISYDGNNTYYSDAVKGRFTISRDNARNTLYLQMNSLRAEDTAVYYCAKEFDIPWDNF
DYWGQGTLVTVSSASTTAPSVTSGQAGQ
IL4 P3-1 B10 VH [SEQ ID NO: 7]
EVQLVESGGDLVKPGGSLRLSCVASGFTFSNYDMSWVRQAPGKGLQWVSTISYDGNNT
YYSDAVKGRFTISRDNARNTLYLQMNSLRAEDTAVYYCAKEFDIPWDNFDYWGQGTL
VTVSSASTTAPSVTSGQAGQ
IL4 P3-1 B10 VHm [SEQ ID NO: 236]
EVQLVESGGDLVKPGGSLRLSCVASGFTFSNYDMSWVRQAPGKGLQWVSTISYDGQNT
YYSDAVKGRFTISRDNARNTLYLQMNSLRAEDTAVYYCAKEFDIPWDNFDYWGQGTL
VTVSSASTTAPSVTSGQAGQ
IL4 P3-1 B10 VL [SEQ ID NO: 8]
QSVLTQPTSVSAALGQRVTISCTGSNANIGSVYDVQWYQQLPGKSPKTIIYGNTHRPSGV
PVRFSGSKSGSTATLTITGIQAEDEADYYCQSYDATLHYVFGSGTQLTVL
IL4 P3-1 D7 [SEQ ID NO: 9]
QSMLTQPASVSGSLGQRVAISCSGSTNNIGIFGATWYQHLPGKAPKFLLYSDGNRPSGVP
DRFSGSDSGNSVTLMITALQAEDEADYYCQSFDTTLGAYVFGSGTQLTILGGGSSRSSSS
GGGGSGGGGEVQLVQSGGDLVKPGGSLRLSCVASGFAFNGYDMDWVRLAPGKGLQW
LSEISSSGSPTYYADAVEGRFTISRDNAKNTLYLQMDSLRAEDTAVYYCARAGEYTYGY
EGYYFDYWGQGTLVTVSSASTTAPSVTSGQAGQ
IL4 P3-1 D7 VH [SEQ ID NO: 10]
EVQLVQSGGDLVKPGGSLRLSCVASGFAFNGYDMDWVRLAPGKGLQWLSEISSSGSPT
YYADAVEGRFTISRDNAKNTLYLQMDSLRAEDTAVYYCARAGEYTYGYEGYYFDYWG
QGTLVTVSSASTTAPSVTSGQAGQ
IL4 P3-1 D7 VL [SEQ ID NO: 11]
EVQLVQSGGDLVKPGGSLRLSCVASGFAFNGYDMDWVRLAPGKGLQWLSEISSSGSPT
YYADAVEGRFTISRDNAKNTLYLQMDSLRAEDTAVYYCARAGEYTYGYEGYYFDYWG
QGTLVTVSSASTTAPSVTSGQAGQ
IL4 P4-1 G3 [SEQ ID NO: 12]
QSVLNQLPSLSMNMGETATFTCEGDNIGHKYVNWYQQKPGQAPVLTIYEDNKRPSGIPE
RFSGSNSGNTATLTISGARAEDEADYYCESEVSTGIVFGGGTHLTVLGGGSSRSSSSGGG
GSGGGGEVQLVESGGDLVKPGGSLRLSCVASGFTFSSYDMDWVRQAPGKGLEWLSEIS
TNGDRTHYADAVKGRFAISRDNAKNTVYLQMNSLRAEDTAIYYCVRDGYEYLAFDYW
GQGTLVTVSSASTTAPSVTSGQAGQ
IL4 P4-1 G3 VH [SEQ ID NO: 13]
EVQLVESGGDLVKPGGSLRLSCVASGFTFSSYDMDWVRQAPGKGLEWLSEISTNGDRT
HYADAVKGRFAISRDNAKNTVYLQMNSLRAEDTAIYYCVRDGYEYLAFDYWGQGTLV
TVSSASTTAPSVTSGQAGQ
IL4 P4-1 G3 VL [SEQ ID NO: 14]
QSVLNQLPSLSMNMGETATFTCEGDNIGHKYVNWYQQKPGQAPVLTIYEDNKRPSGIPE
RFSGSNSGNTATLTISGARAEDEADYYCESEVSTGIVFGGGTHLTVL
IL4 P3-1 C4 5LC [SEQ ID NO: 15]
QAVLNQPASVSGSLGQRVTISCSGSTNNIGVVGASWYQQFPGKAPKLLVYSDGDRPSGV
PDRFSGSNSGYSDTLTITGLQAEDEADYYCQSFDTTLNAYVFGSGTQLTVLGGGSSRSSS
SGGGGSGGGGEVPLVESGGDLVKPGGSLRISCVASGITFNNDDMGWVRQAPGKGLQWL
AEISSSGTSTYYADAVKGRFTISRDNAKNTVYLQMNSLKAEDTGVYYCVGEDGYMALG
FWGQGTLVTVSSASTTAPSVTSGQAGQ
IL4 P3-1 C4 5LC VH [SEQ ID NO: 16]
EVPLVESGGDLVKPGGSLRISCVASGITFNNDDMGWVRQAPGKGLQWLAEISSSGTSTY
YADAVKGRFTISRDNAKNTVYLQMNSLKAEDTGVYYCVGEDGYMALGFWGQGTLVT
VSSASTTAPSVTSGQAGQ
IL4 P3-1 C4 5LC VL [SEQ ID NO: 17]
QAVLNQPASVSGSLGQRVTISCSGSTNNIGVVGASWYQQFPGKAPKLLVYSDGDRPSGV
PDRFSGSNSGYSDTLTITGLQAEDEADYYCQSFDTTLNAYVFGSGTQLTVL
IL4 P3-1 A6 [SEQ ID NO: 18]
QPVLTQPPSVSAALGQRVTISCNTNIGSPYDVQWYQQLPGKSPKTIIYGNSNRPSGVPVRF
SGSKSGSTATLTIAGIQAEDEADYYCQSYDDNVDGYVFGSGTQLTVLGGGSSRSSSSGG
GGSGGGGEVPLVESGGDLVKPGGSLRLSCVASGFTFHNYDLSWIRQLPGGGLQWVADI
DKSGLTFYADAVKGRFTISRDNAKNTLYLQMNSLRAEDTAVYYCATWPLGSWYTETFE
YWGQGTLVTVSSASTTAPSVTSGQAGQ
IL4 P3-1 A6 VH [SEQ ID NO: 19]
EVPLVESGGDLVKPGGSLRLSCVASGFTFHNYDLSWIRQLPGGGLQWVADIDKSGLTFY
ADAVKGRFTISRDNAKNTLYLQMNSLRAEDTAVYYCATWPLGSWYTETFEYWGQGTL
VTVSSASTTAPSVTSGQAGQ
IL4 P3-1 A6 VL [SEQ ID NO: 20]
QPVLTQPPSVSAALGQRVTISCNTNIGSPYDVQWYQQLPGKSPKTIIYGNSNRPSGVPVRF
SGSKSGSTATLTIAGIQAEDEADYYCQSYDDNVDGYVFGSGTQLTVL
IL4 P4-1 A10 [SEQ ID NO: 21]
QSVLNQLPSVSGSLGQKVTIRCSGNTDNIGMVGAAWYRQVPGEAPKLLLYSDGNRPSG
VPDRFSGSDSGRSATLTITGLQPEDEADYYCQSLDPTVPGNYLFGSGTQLTILGGGSSRSS
SSGGGGSGGGGEVQLVESGGDLVKPGGSLRLSCVASGFTFSDYDMTWVRQAPGKGLQ
WVADINNGGSSTTYADAVKGRFTISRDNAKNTLYLQMNSLRAEDTAMYYCAKEGDNT
WLSFDSWGQGTLVTVSSESPSPPNLTSGQAGQ
IL4 P4-1 A10 VH [SEQ ID NO: 22]
EVQLVESGGDLVKPGGSLRLSCVASGFTFSDYDMTWVRQAPGKGLQWVADINNGGSST
TYADAVKGRFTISRDNAKNTLYLQMNSLRAEDTAMYYCAKEGDNTWLSFDSWGQGTL
VTVSSESPSPPNLTSGQAGQ
IL4 P4-1 A10 VL [SEQ ID NO: 23]
QSVLNQLPSVSGSLGQKVTIRCSGNTDNIGMVGAAWYRQVPGEAPKLLLYSDGNRPSG
VPDRFSGSDSGRSATLTITGLQPEDEADYYCQSLDPTVPGNYLFGSGTQLTIL
IL4 P4A-1 F10 [SEQ ID NO: 24]
QTVVTQEPSLSVSPGGTVTLTCGLSSGSVSTGNYPGWYQQTQGRAPRSIIYDTNSRPSGV
PDRFSGSISGNKAVLTITGAQPEDEADYYCSLYTGIAYTDVVFGGGTHLTVLGGGSSRSS
SSGGGGSGGGGEVQLVQSGGDLVKPAGSLRLSCVASGFTFNSYAMSWVRQAPGKGLQ
WVAGINSGGSTTTYADAVKGRFTISRDNAKNTLYLQMNSLRAEDTAVYYCAKDQNSY
YPSDTFEYWGQGTLVTVSSESPSPPNLTSGQAGQ
IL4 P4A-1 F10 VH [SEQ ID NO: 25]
EVQLVQSGGDLVKPAGSLRLSCVASGFTFNSYAMSWVRQAPGKGLQWVAGINSGGSTT
TYADAVKGRFTISRDNAKNTLYLQMNSLRAEDTAVYYCAKDQNSYYPSDTFEYWGQG
TLVTVSSESPSPPNLTSGQAGQ
IL4 P4A-1 F10 VL [SEQ ID NO: 26]
QTVVTQEPSLSVSPGGTVTLTCGLSSGSVSTGNYPGWYQQTQGRAPRSIIYDTNSRPSGV
PDRFSGSISGNKAVLTITGAQPEDEADYYCSLYTGIAYTDVVFGGGTHLTVL
IL4 P4A-2 D11 [SEQ ID NO: 27]
QAVLTQPPSVSAALGQRVTISCSGSTDNVGTVGASWYQHLPGRAPKLLVYIVGDRPSGV
PDRFSGSKSGTSATLTITGLQAEDEADYYCSSWDDSLRGIVFGGGTHLTVLGGGSSRSSS
SGGGGSGGGGEEQLVEFGGDLVKPGESLRLSCVASGFTFNKYDMSWVRQAPGKGLQW
VATVADDESFAYYTDSVKGRFTISRDNAKNTLYLQMNSLRAEDTAMYYCVGKTGSYW
DIDFDLWGPGTLVTISSASTTAPSVTSGQAGQ
IL4 P4A-2 D11 VH [SEQ ID NO: 28]
EEQLVEFGGDLVKPGESLRLSCVASGFTFNKYDMSWVRQAPGKGLQWVATVADDESF
AYYTDSVKGRFTISRDNAKNTLYLQMNSLRAEDTAMYYCVGKTGSYWDIDFDLWGPG
TLVTISSASTTAPSVTSGQAGQ
IL4 P4A-2 D11 VL [SEQ ID NO: 29]
QAVLTQPPSVSAALGQRVTISCSGSTDNVGTVGASWYQHLPGRAPKLLVYIVGDRPSGV
PDRFSGSKSGTSATLTITGLQAEDEADYYCSSWDDSLRGIVFGGGTHLTVL
IL4 P4A-1 A4 [SEQ ID NO: 30]
SYVLSQPPSVSVTPSETAHITCGGNNIGSKYVQWIRQSAGQAPVTIIYGNTNRPTGIPERFS
GANSGNTATLTISGVLAEDEADYYCQVGDTDTTVFGGGTHLTVLGGGSSRSSSSGGGGS
GGGGEVQLVESGGDLVKPGGSLRLSCVASGFNFSRYSMSWVRQAPEKGLQLVAAVNSG
GSRTWYADAVQGRFTVSRDNAKNTLFLEMSSLKLEDTAMYYCAKDLWSTTIAEYDYW
GQGTLVTVSSASTTAPSVTSGQAGQ
IL4 P4A-1 A4 VH [SEQ ID NO: 31]
EVQLVESGGDLVKPGGSLRLSCVASGFNFSRYSMSWVRQAPEKGLQLVAAVNSGGSRT
WYADAVQGRFTVSRDNAKNTLFLEMSSLKLEDTAMYYCAKDLWSTTIAEYDYWGQGT
LVTVSSASTTAPSVTSGQAGQ
IL4 P4A-1 A4 VL [SEQ ID NO: 32]
SYVLSQPPSVSVTPSETAHITCGGNNIGSKYVQWIRQSAGQAPVTIIYGNTNRPTGIPERFS
GANSGNTATLTISGVLAEDEADYYCQVGDTDTTVFGGGTHLTVL
IL4 P4A-1 D9 [SEQ ID NO: 33]
QSVLNQPPSVSVNLGKTASITCEGDNIGDKYAFWYQQKPGQAPVLIIYDDSKRPSGIPER
LSGSNSVNTATLTISGARAEDEADYYCQVWDNSAKAIVFGGGTHLTVLGGGSSRSSSSG
GGGSGGGGEVQLVETGGDLVEPGGSLRLSCVASGFTFSGYYMSWIRQAPGKGLQWVA
DIRDSGGNTHYADAVRGRFTISRDNAKNTLYLQMDSLRAEDTAVYYCARDDWGLDYW
GQGTLVTVSSXSPSPPNLTSGQAGQ
IL4 P4A-1 D9 VH [SEQ ID NO: 34]
EVQLVETGGDLVEPGGSLRLSCVASGFTFSGYYMSWIRQAPGKGLQWVADIRDSGGNT
HYADAVRGRFTISRDNAKNTLYLQMDSLRAEDTAVYYCARDDWGLDYWGQGTLVTV
SSXSPSPPNLTSGQAGQ
IL4 P4A-1 D9 VL [SEQ ID NO: 35]
QSVLNQPPSVSVNLGKTASITCEGDNIGDKYAFWYQQKPGQAPVLIIYDDSKRPSGIPER
LSGSNSVNTATLTISGARAEDEADYYCQVWDNSAKAIVFGGGTHLTVL
IL4 P4A-1 E10 [SEQ ID NO: 36]
QSVLNQPPSVSAALGQRVTVSCTGTDTNIGSGYDVQWYQQVPGKSPTPIIYGNTNRPSG
VPERFSGSKSGNTATLTITGIQAEDEADYYCQSFDDNVDGYVFGSGTQLTILGGGSSRSSS
SGGGGSGGGGEVPLVESGGDLVKPGGSLRLSCVASGFTFSDYHMSWVRQAPGKGLQW
VADISSSGSTYYADAVKGRFTISRDNAKNTLYLQMNSLRAEDTAVYYCARQWGEFDYW
GQGTLVTVSSESPSPPNLTSGQAGQ
IL4 P4A-1 E10 VH [SEQ ID NO: 37]
EVPLVESGGDLVKPGGSLRLSCVASGFTFSDYHMSWVRQAPGKGLQWVADISSSGSTY
YADAVKGRFTISRDNAKNTLYLQMNSLRAEDTAVYYCARQWGEFDYWGQGTLVTVSS
ESPSPPNLTSGQAGQ
IL4 P4A-1 E10 VL [SEQ ID NO: 38]
QSVLNQPPSVSAALGQRVTVSCTGTDTNIGSGYDVQWYQQVPGKSPTPIIYGNTNRPSG
VPERFSGSKSGNTATLTITGIQAEDEADYYCQSFDDNVDGYVFGSGTQLTIL
IL4 P4A-2 G11 [SEQ ID NO: 39]
DIVMTQTPLSLSVSPGEPASISCKASQSLLYSDGNTYLYWFRQKPGQSPQRLISLVSNRDA
GVPDRFSGSGSGTDFTLRISRVEADDAGVYYCGQGIQDPITFGKGTHLEIKGGSSRSSSSG
GGGSGGGGEVPLVESGGDLVKPGGSLRLSCVASGFPFRSYGMNWVRQSPGGGLQWVA
GILFDGSATAYADAVRGRFTISRDNAKNTLYLQMNSLRAEDTAVYYCASDDGSYIEAYN
WFYYWGQGTLVTVSSASTTAPSVTSGQAGQ
IL4 P4A-2 G11 VH [SEQ ID NO: 40]
EVPLVESGGDLVKPGGSLRLSCVASGFPFRSYGMNWVRQSPGGGLQWVAGILFDGSAT
AYADAVRGRFTISRDNAKNTLYLQMNSLRAEDTAVYYCASDDGSYIEAYNWFYYWGQ
GTLVTVSSASTTAPSVTSGQAGQ
IL4 P4A-2 G11 VL [SEQ ID NO: 41]
DIVMTQTPLSLSVSPGEPASISCKASQSLLYSDGNTYLYWFRQKPGQSPQRLISLVSNRDA
GVPDRFSGSGSGTDFTLRISRVEADDAGVYYCGQGIQDPITFGKGTHLEIK

TABLE 9
SEQ ID NOS. FOR ANTIBODIES TO IL-4

| Antibody | scFv | VH | VHm | VL | HCDRs | LCDRs | HC | LC | ^{#}HC_{YD} |
|---|---|---|---|---|---|---|---|---|---|
| IL4 P3-1 B9 | 3 | 4 | | 5 | 156-158 | 159-161 | 52 | 53 | |
| IL4 P3-1 B10 | 6 | 7 | | 8 | 162-164 | 165-167 | 54 | 55 | |
| IL4 P3-1 B10m | — | 7 | 236 | — | 162, 237, 164 | 165-167 | 56 | 55 | |
| IL4 P3-1 D7 | 9 | 10 | | 11 | 168-170 | 171-173 | 57 | 58 | |
| IL4 P4-1 G3 | 12 | 13 | | 14 | 174-176 | 177-179 | 59 | 60 | |
| IL4 P3-1 C4 5LC | 15 | 16 | | 17 | 180-182 | 183-185 | 50 | 51 | |
| IL4 P3-1 A6 | 18 | 19 | | 20 | 186-188 | 189-191 | 48 | 49 | |
| IL4 P4-1 A10 | 21 | 22 | | 23 | 192-194 | 195-197 | 46 | 47 | |
| IL4 P4A-1 F10 | 24 | 25 | | 26 | 198-200 | 201-203 | 44 | 45 | |
| IL4 P4A-1 F10 | 24 | 25 | | 26 | 198-200 | 201-203 | — | 45 | 241 |
| IL4 P4A-2 D11 | 27 | 28 | | 29 | 204-206 | 207-209 | 42 | 43 | |
| IL4 P4A-1 A4 | 30 | 31 | | 32 | 210-212 | 213-215 | | | |
| IL4 P4A-1 D9 | 33 | 34 | | 35 | 216-218 | 219-221 | | | |
| IL4 P4A-1 E10 | 36 | 37 | | 38 | 222-224 | 225-227 | | | |
| IL4 P4A-2 G11 | 39 | 40 | | 41 | 228-230 | 231-233 | | | |

^{#}A heavy chain comprising a constant region that comprises a tyrosine (Y) residue at amino acid position 252 and an aspartic acid (D) residue at amino acid position 256, as numbered according to the EU index as in Kabat.

Amino Acid Sequences of scFvs Specific to Canine IL-13
IL13 P3-1 B4 [SEQ ID NO: 61]
SYVLTQSPSVSGSLGQTVTISCSGTTIGVVGANWYQQLPGKAPKLLVHSDGGRLSGVPD
RFSGSESGNSAALTITGLQAEDEADYYCQSFDTTLGYVFGSGTQLTILGGGSSRSSSSGGG
GSGGGGELTLQESGPGLVKPSQTLSLTCVVSGGSVTSNYYWNWIRQRPGRGLEWMGY
WTGSTNYNPAFQGRISITADTAKNHFSLQLTSMTTEDTAVYYCARASTWLHGMDFWGH
GTSLFVSSESPSPPNLTSGQAGQ
IL13 P3-1 B4 VH [SEQ ID NO: 62]
ELTLQESGPGLVKPSQTLSLTCVVSGGSVTSNYYWNWIRQRPGRGLEWMGYWTGSTNY
NPAFQGRISITADTAKNHFSLQLTSMTTEDTAVYYCARASTWLHGMDFWGHGTSLFVSS
ESPSPPNLTSGQAGQ
IL13 P3-1 B4 VL [SEQ ID NO: 63]
SYVLTQSPSVSGSLGQTVTISCSGTTIGVVGANWYQQLPGKAPKLLVHSDGGRLSGVPD
RFSGSESGNSAALTITGLQAEDEADYYCQSFDTTLGYVFGSGTQLTIL
IL13 P3-1 C2 [SEQ ID NO: 64]
QAVLNQPASVSGSLGQRVTISCSGSSSNIGRGFVGWYQQLPGTRPKTLIYGDTFRPSGVP
DRFFGSRSGSTATLTISGLQAEDEADYYCSTWDDSLAALLFGGGTHLTVLGGGSSRSSFS
GGGGSGGGGEVQLVESGGDLVKPGGSLRLSCAASGFTFTDYDMSWVRQAPGKGLQWV
ATTSNGGSGTYYSDAVKGRFTISRDNAKNTLYLEMNRLRAEDTAVYYCVKEIYPASIDF
WGLGTLVTVSSASTTAPSVTSGQAGQ
IL13 P3-1 C2 VH [SEQ ID NO: 65]
EVQLVESGGDLVKPGGSLRLSCAASGFTFTDYDMSWVRQAPGKGLQWVATTSNGGSG
TYYSDAVKGRFTISRDNAKNTLYLEMNRLRAEDTAVYYCVKEIYPASIDFWGLGTLVTV
SSASTTAPSVTSGQAGQ
IL13 P3-1 C2 VL [SEQ ID NO: 66]
QAVLNQPASVSGSLGQRVTISCSGSSSNIGRGFVGWYQQLPGTRPKTLIYGDTFRPSGVP
DRFFGSRSGSTATLTISGLQAEDEADYYCSTWDDSLAALLFGGGTHLTVL
IL13 P4A-2 E11 [SEQ ID NO: 67]
QSALTQTASVSGSLGQRVTISCTGATRNVGTVGVTWYQQVPGKAPKLLLFSNGDRPSW
VPDRFSGSTSGYSATLIINGVQAEDEAMYHCQSFDTMPDAHYMFGSGTQLTVLGGGSSR
SSSSGGGGSGGGGELTLQESGPGLVKPSQTLSLTCVVSGASVTSTNYWNWIRQRPGRGL
EWMGYWTGSTKYNPAFQGRISITADTAKNQFSLQLSSMTTEDTAVYYCARSSTWINGED
YWGQGTLVTVSSESPSPPNLTSGQAGQ
IL13 P4A-2 E11 VH [SEQ ID NO: 68]
ELTLQESGPGLVKPSQTLSLTCVVSGASVTSTNYWNWIRQRPGRGLEWMGYWTGSTKY
NPAFQGRISITADTAKNQFSLQLSSMTTEDTAVYYCARSSTWINGFDYWGQGTLVTVSS
ESPSPPNLTSGQAGQ
IL13 P4A-2 E11 VL [SEQ ID NO: 69]
QSALTQTASVSGSLGQRVTISCTGATRNVGTVGVTWYQQVPGKAPKLLLFSNGDRPSW
VPDRFSGSTSGYSATLIINGVQAEDEAMYHCQSFDTMPDAHYMFGSGTQLTVL
IL13 P2-1 H5 [SEQ ID NO: 70]
QSVLNQPPSVSAALGQRVTISCTGTNTNIGSGYSVQWYQQLPGESPKPIIYGSSNRPSGVP
ARFSGSKSGSTGTLTITGIQAEDEADYHCQSYDDSLDGHAVFGGGTQLTVLGGGSSRSSS
SGGGGSGGGGEVQLVQSGGDLVKPGGSLRLSCVASGFTFSGYDMSWVRQSPGKGLQW
VATVWSDGNTIYYADAVKGRFTISRDNAKNTVYLQMNSLRAEDTAMYYCAKSPYCTD
DYCPWGDYLDLWGQGTLVTVSSASTTAPSVTSGQAGQ
IL13 P2-1 H5 VH [SEQ ID NO: 71]
EVQLVQSGGDLVKPGGSLRLSCVASGFTFSGYDMSWVRQSPGKGLQWVATVWSDGNT
IYYADAVKGRFTISRDNAKNTVYLQMNSLRAEDTAMYYCAKSPYCTDDYCPWGDYLD
LWGQGTLVTVSSASTTAPSVTSGQAGQ
IL13 P2-1 H5 VL [SEQ ID NO: 72]
QSVLNQPPSVSAALGQRVTISCTGTNTNIGSGYSVQWYQQLPGESPKPIIYGSSNRPSGVP
ARFSGSKSGSTGTLTITGIQAEDEADYHCQSYDDSLDGHAVFGGGTQLTVL
IL13 P2-2 B10 [SEQ ID NO: 73]
QPVLTQPPSVSVSLGQTATISCSGESLNEYYAQWYQQKAGQAPVLIMYKDTERPSGIPDR
FSGSSSGNIHTLTISGTRAEDEADYYCGSLVSTYDYIFGSGTQLTVLGGGSSRSSSSGGGG
SGGGGEVQLVQSGGDLVKTGGSLRLSCVASGFTFSRYDMSWVRQAPGKGLQWVARISS
DGSTTYYADAVKGRFTISRDNAKNTLYLQMNSLRAEDTAMYYCAKDLVWSAWYGMD
YWGHGTSLFVSSASTTAPSVTSGQAGQ
IL13 P2-2 B10 VH [SEQ ID NO: 74]
EVQLVQSGGDLVKTGGSLRLSCVASGFTFSRYDMSWVRQAPGKGLQWVARISSDGSTT
YYADAVKGRFTISRDNAKNTLYLQMNSLRAEDTAMYYCAKDLVWSAWYGMDYWGH
GTSLFVSSASTTAPSVTSGQAGQ
IL13 P2-2 B10 VL [SEQ ID NO: 75]
QPVLTQPPSVSVSLGQTATISCSGESLNEYYAQWYQQKAGQAPVLIMYKDTERPSGIPDR
FSGSSSGNIHTLTISGTRAEDEADYYCGSLVSTYDYIFGSGTQLTVL
IL13 P3-1 G5 [SEQ ID NO: 76]
SYELTQPPSVSVSLGQTATISCSGESLSKYYAQWFQQKAGQAPVLVIYQDTERPSGIPDRF
SGSSSGNTHTLTISGARAEDEADYYCESAISPDTYVFGSGTQLTILGGGSSRSSSSGGGGS
GGGGEVQLVQSGGDLVKPGGTLRLSCVASGFTFSSYDMSWVRQSPGKGLQWVAAIWN
DGSNTEYADAVKGRFTVSRDNAKNTVYLEMNSLRAEDTAMYYCVKEGPSYGYFDYW
GQGTLVTVSSASTTAPSVTSGQAGQ
IL13 P3-1 G5 VH [SEQ ID NO: 77]
EVQLVQSGGDLVKPGGTLRLSCVASGFTFSSYDMSWVRQSPGKGLQWVAAIWNDGSN
TEYADAVKGRFTVSRDNAKNTVYLEMNSLRAEDTAMYYCVKEGPSYGYFDYWGQGT
LVTVSSASTTAPSVTSGQAGQ
IL13 P3-1 G5 VL [SEQ ID NO: 78]
SYELTQPPSVSVSLGQTATISCSGESLSKYYAQWFQQKAGQAPVLVIYQDTERPSGIPDRF
SGSSSGNTHTLTISGARAEDEADYYCESAISPDTYVFGSGTQLTIL
IL13 P3-05 [SEQ ID NO: 79]
SYVLTQPPSVTVPLRQTARISCGGDNIRSKNVHWYQQKPGQAPVLIIYNDSNRPTGIPERF
SGSNSGNMATLTIRGALAEDEADYYCQVWANDGYVFGSGTQLTVLGGGSSRSSSSGGG
GSGGGGEVQLVESGGDSVRPGGSLRLSCVASGFTFSRYNMGWVRQAPGKGLQWVAWI
YDGGSRTSYADAVKGRFTISRDNAKDTLYLQMDDLRVDDTAVYYCARDPSSWGSLFD
YWGQGTLVTVSSASTTAPSVTSGQAGQ
IL13 P3-05 VH [SEQ ID NO: 80]
EVQLVESGGDSVRPGGSLRLSCVASGFTFSRYNMGWVRQAPGKGLQWVAWIYDGGSR
TSYADAVKGRFTISRDNAKDTLYLQMDDLRVDDTAVYYCARDPSSWGSLFDYWGQGT
LVTVSSASTTAPSVTSGQAGQ
IL13 P3-05 VL [SEQ ID NO: 81]
SYVLTQPPSVTVPLRQTARISCGGDNIRSKNVHWYQQKPGQAPVLIIYNDSNRPTGIPERF
SGSNSGNMATLTIRGALAEDEADYYCQVWANDGYVFGSGTQLTVL
IL 13-05 VLm [SEQ ID NO: 234]
SYVLTQPPSVTVPLRQTARISCGGDNIRSKNVHWYQQKPGQAPVLIIYQDSNRPTGIPERF
SGSNSGNMATLTIRGALAEDEADYYCQVWANDGYVFGSGTQLTVL
IL13 P3-05m [SEQ ID NO: 254]
SYVLTQPPSVTVPLRQTARISCGGDNIRSKNVHWYQQKPGQAPVLIIYQDSNRPTGIPERF
SGSNSGNMATLTIRGALAEDEADYYCQVWANDGYVFGSGTQLTVLGGGSSRSSSSGGG
GSGGGGEVQLVESGGDSVRPGGSLRLSCVASGFTFSRYNMGWVRQAPGKGLQWVAWI
YDGGSRTSYADAVKGRFTISRDNAKDTLYLQMDDLRVDDTAVYYCARDPSSWGSLFD
YWGQGTLVTVSSASTTAPSVTSGQAGQ
IL 13 P3-1C6 [SEQ ID NO: 243]
QSVLTQPASVSGSLGQRVTISCSGRINNIGYFGANWYQQVPGKAPKLLVSSYGNRPSGV
PDRFSASNSANSATLTITGLQAEDEADYYCQSFDTTLGAVVFGGGTQLTVLGGGSSRSSS
SGGGGSGGGGELTLQESGPGLVKPSQTLSLTCVVSGGSVTNTYEWDWIRQRPGRGLEW
MGSWTDNTWYNPAFQGRISITADTAKNQLSLQLSSVTTGDTAVYFCARWTKRWGYDY
WGQGTLVTVSSASTTAPSVTSGQAGQ
IL 13 P3-1C6 VH [SEQ ID NO: 244]
ELTLQESGPGLVKPSQTLSLTCVVSGGSVTNTYEWDWIRQRPGRGLEWMGSWTDNTW
YNPAFQGRISITADTAKNQLSLQLSSVTTGDTAVYFCARWTKRWGYDYWGQGTLVTVS
S
IL 13 P3-1C6 VL [SEQ ID NO: 245]
QSVLTQPASVSGSLGQRVTISCSGRTNNIGYFGANWYQQVPGKAPKLLVSSYGNRPSGV
PDRFSASNSANSATLTITGLQAEDEADYYCQSFDTTLGAVVFGGGTQLTVL

TABLE 10
SEQ ID NOS. FOR ANTIBODIES TO IL-13

| Antibody | scFv | VH | VL | VLm | HCDRs | LCDRs | HC | LC | ^{#}HC_{YD} |
|---|---|---|---|---|---|---|---|---|---|
| IL13 P3-1 B4 | 61 | 62 | 63 | | 114-116 | 117-119 | 87 | 88 | |
| IL13 P3-1 C2 | 64 | 65 | 66 | | 120-122 | 123-125 | | | |
| IL13 P4A-2 E11 | 67 | 68 | 69 | | 126-128 | 129-131 | | | |
| IL13 P2-1 H5 | 70 | 71 | 72 | | 132-134 | 135-137 | | | |
| IL13 P2-2 B10 | 73 | 74 | 75 | | 138-140 | 141-143 | | | |
| IL13 P3-1 G5 | 76 | 77 | 78 | | 144-146 | 147-149 | 82 | 83 | |
| IL13 P3-05 | 79 | 80 | 81 | | 150-152 | 153-155 | 84 | 86 | |
| IL13 P3-05m | | 80 | — | 234 | 150-152 | 153, 235, 155 | 84 | 85 | |
| IL13 P3-05 | 79 | 80 | 81 | | 150-152 | 153-155 | — | 86 | 240 |
| IL13 P3-05m | 254 | 80 | — | 234 | 150-152 | 153, 235, 155 | — | 85 | 240 |
| IL13 1C6 | 243 | 244 | 245 | | 248-250 | 251-253 | 246 | 247 | |

^{#}A heavy chain comprising a constant region that comprises a tyrosine (Y) residue at amino acid position 252 and an aspartic acid (D) residue at amino acid position 256, as numbered according to the EU index as in Kabat.

TABLE 11
CDR SEQ ID NOs. FOR ANTIBODIES TO IL-13

| | CDR | SEQ ID |
|---|---|---|
| IL13 P3-1 B4 VH | HCDR1 | 114 |
| (SEQ ID NO: 62) | HCDR2 | 115 |
| | HCDR3 | 116 |
| IL13 P3-1 B4 VL | LCDR1 | 117 |
| (SEQ ID NO: 63) | LCDR2 | 118 |
| | LCDR3 | 119 |
| IL13 P3-1 C2 VH | HCDR1 | 120 |
| (SEQ ID NO: 65) | HCDR2 | 121 |
| | HCDR3 | 122 |
| IL13 P3-1 C2 VL | LCDR1 | 123 |
| (SEQ ID NO: 66) | LCDR2 | 124 |
| | LCDR3 | 125 |
| IL13 P4A-2 E11 VH | HCDR1 | 126 |
| (SEQ ID NO: 68) | HCDR2 | 127 |
| | HCDR3 | 128 |
| IL13 P4A-2 E11 VL | LCDR1 | 129 |
| (SEQ ID NO: 69) | LCDR2 | 130 |
| | LCDR3 | 131 |
| IL13 P2-1 H5 VH | HCDR1 | 132 |
| (SEQ ID NO: 71) | HCDR2 | 133 |
| | HCDR3 | 134 |
| IL13 P2-1 H5 VL | LCDR1 | 135 |
| (SEQ ID NO: 72) | LCDR2 | 136 |
| | LCDR3 | 137 |
| IL13 P2-2 B10 VH | HCDR1 | 138 |
| (SEQ ID NO: 74) | HCDR2 | 139 |
| | HCDR3 | 140 |
| IL13 P2-2 B10 VL | LCDR1 | 141 |
| (SEQ ID NO: 75) | LCDR2 | 142 |
| | LCDR3 | 143 |
| IL13 P3-1 G5 VH | HCDR1 | 144 |
| (SEQ ID NO: 77) | HCDR2 | 145 |
| | HCDR3 | 146 |
| IL13 P3-1 G5 VL | LCDR1 | 147 |
| (SEQ ID NO: 78) | LCDR2 | 148 |
| | LCDR3 | 149 |
| IL13 P3-05 VH | HCDR1 | 150 |
| (SEQ ID NO: 80) | HCDR2 | 151 |
| | HCDR3 | 152 |
| IL13 P3-05 VL | LCDR1 | 153 |
| (SEQ ID NO: 81) | LCDR2 | 154 |
| | LCDR3 | 155 |
| IL13 P3-05 VLm | LCDR1 | 153 |
| (SEQ ID NO: 234) | LCDR2 | 235 |
| | LCDR3 | 155 |
| IL-13-P3-1C6VH | HCDR1 | 248 |
| (SEQ ID NO: 244) | HCDR2 | 249 |
| | HCDR3 | 250 |
| IL-13-P3-1C6VL | LCDR1 | 251 |
| (SEQ ID NO: 245) | LCDR2 | 252 |
| | LCDR3 | 253 |

TABLE 12
CDRS SEQUENCES FOR ANTIBODIES TO IL-13

| IL13-P3-1B4 VH | | SEQ ID NO: |
|---|---|---|
| HCDR1 | SNYYWN | 114 |
| HCDR2 | YWTGSTNYNPAFQG | 115 |
| HCDR3 | ASTWLHGMDF | 116 |
| IL13-P3-1B4 VL | | |
| LCDR1 | SGTTIGVVGAN | 117 |
| LCDR2 | SDGGRLS | 118 |
| LCDR3 | QSFDTTLGYV | 119 |
| IL13 P3-1 C2 VH | | SEQ ID NO: |
| HCDR1 | DYDMS | 120 |
| HCDR2 | TTSNGGSGTYYSDAVKG | 121 |
| HCDR3 | EIYPASIDF | 122 |
| IL13 P3-1 C2 VL | | |
| HCDR1 | SGSSSNIGRGFVG | 123 |
| HCDR2 | GDTFRPS | 124 |
| HCDR3 | STWDDSLAALL | 125 |
| IL13-P4A-2 E11 VH | | SEQ ID NO: |
| HCDR1 | STNYWN | 126 |
| HCDR2 | YWTGSTKYNPAFQG | 127 |
| HCDR3 | SSTWINGFDY | 128 |
| IL13-P4A-2 E11 VL | | |
| LCDR1 | TGATRNVGTVGVT | 129 |
| LCDR2 | SNGDRPS | 130 |
| LCDR3 | QSFDTMPDAHYM | 131 |
| IL13 P2-1 H5 VH | | SEQ ID NO: |
| HCDR1 | GYDMS | 132 |
| HCDR2 | TVWSDGNTIYYADAVKG | 133 |
| HCDR3 | SPYCTDDYCPWGDYLDL | 134 |
| IL13 P2-1 H5 VL | | |
| LCDR1 | TGTNTNIGSGYSVQ | 135 |
| LCDR2 | GSSNRPS | 136 |
| LCDR3 | QSYDDSLDGHAV | 137 |
| IL13-P2-2 B10 VH | | |
| HCDR1 | RYDMS | 138 |
| HCDR2 | RISSDGSTTYYADAVKG | 139 |
| HCDR3 | DLVWSAWYGMDY | 140 |
| IL13-P2-2 B10 VL | | |
| LCDR1 | SGESLNEYYAQ | 141 |
| LCDR2 | KDTERPS | 142 |
| LCDR3 | GSLVSTYDYI | 143 |
| IL13 P3-1 G5 VH | | SEQ ID NO: |
| HCDR1 | SYDMS | 144 |
| HCDR2 | AIWNDGSNTEYADAVKG | 145 |
| HCDR3 | EGPSYGYFDY | 146 |
| IL13 P3-1 G5 VL | | |
| LCDR1 | SGESLSKYYAQ | 147 |
| LCDR2 | QDTERPS | 148 |
| LCDR3 | ESAISPDTYV | 149 |
| IL13 P3-05 VH | | SEQ ID NO: |
| HCDR1 | RYNMG | 150 |
| HCDR2 | WIYDGGSRTSYADAVKG | 151 |
| HCDR3 | DPSSWGSLFDY | 152 |
| IL13 P3-05 VL | | |
| LCDR1 | GGDNIRSKNVH | 153 |
| LCDR2 | NDSNRPT | 154 |
| LCDR3 | QVWANDGYV | 155 |
| IL13 P3-05 VLm | | |
| LCDR1 | GGDNIRSKNVH | 153 |
| LCDR2 | QDSNRPT | 235 |
| LCDR3 | QVWANDGYV | 155 |
| IL13 1C6 VH | | |
| HCDR1 | NTYEWD | 248 |
| HCDR2 | SWTDNTWYNPAFQG | 249 |
| HCDR3 | WTKRWGYDY | 250 |
| IL 13 1C6 VL | | |
| LCDR1 | SGRTNNIGYFGAN | 251 |
| LCDR2 | SYGNRPS | 252 |
| LCDR3 | QSFDTTLGAVV | 253 |

CDRS for IL-4 antibodies

| IL4 P3-1 B9 VH | | SEQ ID NO: |
|---|---|---|
| HCDR1 | SYGMS | 156 |
| HCDR2 | DISSSGSTYYADAVKG | 157 |
| HCDR3 | AGYGSYYDFGMDY | 158 |
| IL4 P3-1 B9 VL | | |
| LCDR1 | TGTATNVGGGYDVQ | 159 |
| LCDR2 | GNYNRPS | 160 |
| LCDR3 | QSFDTTFNGHV | 161 |
| IL4 P3-1 B10 VH | | |
| HCDR1 | NYDMS | 162 |
| HCDR2 | TISYDGNNTYYSDAVKG | 163 |
| HCDR3 | EFDIPWDNFDY | 164 |
| IL4 P3-1 B10 VHm | | |
| HCDR1 | NYDMS | 162 |
| HCDR2 | TISYDGQNTYYSDAVKG | 237 |
| HCDR3 | EFDIPWDNFDY | 164 |
| IL4 P3-1 B10 VL | | |
| LCDR1 | TGSNANIGSVYDVQ | 165 |
| LCDR2 | GNTHRPS | 166 |
| LCDR3 | QSYDATLHYV | 167 |
| IL4 P3-1 D7 VH | | SEQ ID NO: |
| HCDR1 | GYDMD | 168 |
| HCDR2 | EISSSGSPTYYADAVEG | 169 |
| HCDR3 | AGEYTYGYEGYYFDY | 170 |
| IL4 P3-1 D7 VL | | |
| LCDR1 | SGSTNNIGIFGAT | 171 |
| LCDR2 | SDGNRPS | 172 |
| LCDR3 | QSFDTTLGAYV | 173 |
| IL4 P4-1 G3 VH | | SEQ ID NO: |
| HCDR1 | SYDMD | 174 |
| HCDR2 | EISTNGDRTHYADAVKG | 175 |
| HCDR3 | DGYEYLAFDY | 176 |
| IL4 P4-1 G3 VL | | |
| LCDR1 | EGDNIGHKYVN | 177 |
| LCDR2 | EDNKRPS | 178 |
| LCDR3 | ESEVSTGIV | 179 |
| IL4 P3-1 C4 VH | | SEQ ID NO: |
| HCDR1 | NDDMG | 180 |
| HCDR2 | EISSSGTSTYYADAVKG | 181 |
| HCDR3 | EDGYMALGF | 182 |
| IL4 P3-1 C4 VL | | |
| LCDR1 | SGSTNNIGVVGAS | 183 |
| LCDR2 | SDGDRPS | 184 |
| LCDR3 | QSFDTTLNAYV | 185 |
| IL4 P3-1 A6 VH | | SEQ ID NO: |
| HCDR1 | NYDLS | 186 |
| HCDR2 | DIDKSGLTFYADAVKG | 187 |
| HCDR3 | WPLGSWYTETFEY | 188 |
| IL4 P3-1 A6 VL | | |
| LCDR1 | NTNIGSPYDVQ | 189 |
| LCDR2 | GNSNRPS | 190 |
| LCDR3 | QSYDDNVDGYV | 191 |
| IL4 P4-1 A10 VH | | |
| HCDR1 | DYDMT | 192 |
| HCDR2 | DINNGGSSTTYADAVKG | 193 |
| HCDR3 | EGDNTWLSFDS | 194 |
| IL4 P4-1 A10 VL | | |
| LCDR1 | SGNTDNIGMVGAA | 195 |
| LCDR2 | SDGNRPS | 196 |
| LCDR3 | QSLDPTVPGNYL | 197 |
| IL4 P4A-1 F10 VH | | SEQ ID NO: |
| HCDR1 | SYAMS | 198 |
| HCDR2 | GINSGGSTTTYADAVKG | 199 |
| HCDR3 | DQNSYYPSDTFEY | 200 |
| IL4 P4A-1 F10 VL | | |
| LCDR1 | GLSSGSVSTGNYPG | 201 |
| LCDR2 | DTNSRPS | 202 |
| LCDR3 | SLYTGIAYTDVV | 203 |
| IL4 P4A-2 D11 VH | | SEQ ID NO: |
| HCDR1 | KYDMS | 204 |
| HCDR2 | TVADDESFAYYTDSVKG | 205 |
| HCDR3 | KTGSYWDIDFD | 206 |
| IL4 P4A-2 D11 VL | | |
| LCDR1 | SGSTDNVGTVGAS | 207 |
| LCDR2 | IVGDRPS | 208 |
| LCDR3 | SSWDDSLRGIV | 209 |
| IL4 P4A-1 A4 VH | | |
| HCDR1 | RYSMS | 210 |
| HCDR2 | AVNSGGSRTWYADAVQG | 211 |
| HCDR3 | DLWSTTIAEYDY | 212 |
| IL4 P4A-1 A4 VL | | |
| LCDR1 | GGNNIGSKYVQ | 213 |
| LCDR2 | GNTNRPT | 214 |
| LCDR3 | QVGDTDTTV | 215 |
| IL4 P4A-1 D9 VH | | |
| HCDR1 | GYYMS | 216 |
| HCDR2 | DIRDSGGNTHYADAVRG | 217 |
| HCDR3 | DDWGLDY | 218 |
| IL4 P4A-1 D9 VL | | |
| LCDR1 | EGDNIGDKYAF | 219 |
| LCDR2 | DDSKRPS | 220 |
| LCDR3 | QVWDNSAKAIV | 221 |
| IL4 P4-A1 E10 VH | | SEQ ID NO: |
| HCDR1 | DYHMS | 222 |
| HCDR2 | DISSSGSTYYADAVKG | 223 |
| HCDR3 | QWGEFDY | 224 |
| IL4 P4-A1 E10 VL | | |
| LCDR1 | TGTDTNIGSGYDVQ | 225 |
| LCDR2 | GNTNRPS | 226 |
| LCDR3 | QSFDDNVDGYV | 227 |
| IL4 P4A-2 G11 VH | | |
| HCDR1 | SYGMN | 228 |
| HCDR2 | GILFDGSATAYADAVRG | 229 |
| HCDR3 | DDGSYIEAYNWFYY | 230 |
| IL4 P4A-2 G11 VL | | |
| LCDR1 | KASQSLLYSDGNTYLY | 231 |
| LCDR2 | LVSNRDA | 232 |
| LCDR3 | GQGIQDPIT | 233 |

TABLE 13
CDR SEQ ID NOS. FOR ANTIBODIES TO IL-4

| | CDR | SEQ ID |
|---|---|---|
| IL4 P3-1 B9 VH | HCDR1 | 156 |
| (SEQ ID NO: 4) | HCDR2 | 157 |
| | HCDR3 | 158 |
| IL4 P3-1 B9 VL | LCDR1 | 159 |
| (SEQ ID NO: 5) | LCDR2 | 160 |
| | LCDR3 | 161 |
| IL4 P3-1 B10 VH | HCDR1 | 162 |
| (SEQ ID NO: 7) | HCDR2 | 163 |
| | HCDR3 | 164 |
| IL4 P3-1 B10 VHm | HCDR1 | 162 |
| (SEQ ID NO: 236) | HCDR2 | 237 |
| | HCDR3 | 164 |
| IL4 P3-1 B10 VL | LCDR1 | 165 |
| (SEQ ID NO: 8) | LCDR2 | 166 |
| | LCDR3 | 167 |
| IL4 P3-1 D7 VH | HCDR1 | 168 |
| (SEQ ID NO: 10) | HCDR2 | 169 |
| | HCDR3 | 170 |
| IL4 P3-1 D7 VL | LCDR1 | 171 |
| (SEQ ID NO: 11) | LCDR2 | 172 |
| | LCDR3 | 173 |
| IL4 P4-1 G3 VH | HCDR1 | 174 |
| (SEQ ID NO: 13) | HCDR2 | 175 |
| | HCDR3 | 176 |
| IL4 P4-1 G3 VL | LCDR1 | 177 |
| (SEQ ID NO: 14) | LCDR2 | 178 |
| | LCDR3 | 179 |
| IL4 P3-1 C4 5LC VH | HCDR1 | 180 |
| (SEQ ID NO: 16) | HCDR2 | 181 |
| | HCDR3 | 182 |
| IL4 P3-1 C4 5LC VL | LCDR1 | 183 |
| (SEQ ID NO: 17) | LCDR2 | 184 |
| | LCDR3 | 185 |
| IL4 P3-1 A6 VH | HCDR1 | 186 |
| (SEQ ID NO: 19) | HCDR2 | 187 |
| | HCDR3 | 188 |
| IL4 P3-1 A6 VL | LCDR1 | 189 |
| (SEQ ID NO: 20) | LCDR2 | 190 |
| | LCDR3 | 191 |
| IL4 P4-1 A10 VH | HCDR1 | 192 |
| (SEQ ID NO: 22) | HCDR2 | 193 |
| | HCDR3 | 194 |
| IL4 P4-1 A10 VL | LCDR1 | 195 |
| (SEQ ID NO: 23) | LCDR2 | 196 |
| | LCDR3 | 197 |
| IL4 P4A-1 F10 VH | HCDR1 | 198 |
| (SEQ ID NO: 25) | HCDR2 | 199 |
| | HCDR3 | 200 |
| IL4 P4A-1 F10 VL | LCDR1 | 201 |
| (SEQ ID NO: 26) | LCDR2 | 202 |
| | LCDR3 | 203 |
| IL4 P4A-2 D11 VH | HCDR1 | 204 |
| (SEQ ID NO: 28) | HCDR2 | 205 |
| | HCDR3 | 206 |
| IL4 P4A-2 D11 VL | LCDR1 | 207 |
| (SEQ ID NO: 29) | LCDR2 | 208 |
| | LCDR3 | 209 |
| IL4 P4A-1 A4 VH | HCDR1 | 210 |
| (SEQ ID NO: 31) | HCDR2 | 211 |
| | HCDR3 | 212 |
| IL4 P4A-1 A4 VL | LCDR1 | 213 |
| (SEQ ID NO: 32) | LCDR2 | 214 |
| | LCDR3 | 215 |
| IL4 P4A-1 D9 VH | HCDR1 | 216 |
| (SEQ ID NO: 34) | HCDR2 | 217 |
| | HCDR3 | 218 |
| IL4 P4A-1 D9 VL | LCDR1 | 219 |
| (SEQ ID NO: 35) | LCDR2 | 220 |
| | LCDR3 | 221 |
| IL4 P4A-1 E10 VH | HCDR1 | 222 |
| (SEQ ID NO: 37) | HCDR2 | 223 |
| | HCDR3 | 224 |
| IL4 P4A-1 E10 VL | LCDR1 | 225 |
| (SEQ ID NO: 38) | LCDR2 | 226 |
| | LCDR3 | 227 |
| IL4 P4A-2 G11 VH | HCDR1 | 228 |
| (SEQ ID NO: 40) | HCDR2 | 229 |
| | HCDR3 | 230 |
| IL4 P4A-2 G11 VI | LCDR1 | 231 |
| (SEQ ID NO: 41) | LCDR2 | 232 |
| | LCDR3 | 233 |

TABLE 14
FULL LENGTH IgG ANTIBODIES SPECIFIC TO CANINE IL-4

| Antibody | AA sequence/SEQ ID NOs: for heavy and light chains |
|---|---|
| IL4-P4A-2D11VH- | IL4-P4A-2D11VH-clgGB [SEQ ID NO: 42] |
| cIgGB/ | EEQLVEFGGDLVKPGESLRLSCVASGFTFNKYDMSWVRQAP |
| IL4-P4A-2D11VL-cCL | GKGLQWVATVADDESFAYYTDSVKGRFTISRDNAKNTLYL |
| | QMNSLRAEDTAMYYCVGKTGSYWDIDFDLWGPGTLVTISS |
| | ASTTAPSVFPLAPSCGSTSGSTVALACLVSGYFPEPVTVSWNS |
| | GSLTSGVHTFPSVLQSSGLYSLSSMVTVPSSRWPSETFTCNVA |
| | HPASKTKVDKPVPKRENGRVPRPPDCPKCPAPEMLGGPSVFI |
| | FPPKPKDTLLIARTPEVTCVVVDLDPEDPEVQISWFVDGKQM |
| | QTAKTQPREEQFNGTYRVVSVLPIGHQDWLKGKQFTCKVNN |
| | KALPSPIERTISKARGQAHQPSVYVLPPSREELSKNTVSLTCLI |
| | KDFFPPDIDVEWQSNGQQEPESKYRTTPPQLDEDGSYFLYSK |
| | LSVDKSRWQRGDTFICAVMHEALHNHYTQESLSHSPGK |
| | IL4-P4A-2D11VL-cCL [SEQ ID NO: 43] |
| | QAVLTQPPSVSAALGQRVTISCSGSTDNVGTVGASWYQHLP |
| | GRAPKLLVYIVGDRPSGVPDRFSGSKSGTSATLTITGLQAEDE |
| | ADYYCSSWDDSLRGIVFGGGTHLTVLGQPKASPSVTLFPPSS |
| | EELGANKATLVCLISDFYPSGVTVAWKADGSPVTQGVETTK |
| | PSKQSNNKYAASSYLSLTPDKWKSHSSFSCLVTHEGSTVEKK |
| | VAPAECS |
| IL4-P4A-1F10VH- | IL4-P4A-1F10VH-cIgGB [SEQ ID NO: 44] |
| cIgGB/ | EVQLVQSGGDLVKPAGSLRLSCVASGFTFNSYAMSWVRQAP |
| IL4-P4A-1F10VL-cCL | GKGLQWVAGINSGGSTTTYADAVKGRFTISRDNAKNTLYLQ |
| | MNSLRAEDTAVYYCAKDQNSYYPSDTFEYWGQGTLVTVSS |
| | ASTTAPSVFPLAPSCGSTSGSTVALACLVSGYFPEPVTVSWNS |
| | GSLTSGVHTFPSVLQSSGLYSLSSMVTVPSSRWPSETFTCNVA |
| | HPASKTKVDKPVPKRENGRVPRPPDCPKCPAPEMLGGPSVFI |
| | FPPKPKDTLLIARTPEVTCVVVDLDPEDPEVQISWFVDGKQM |
| | QTAKTQPREEQFNGTYRVVSVLPIGHQDWLKGKQFTCKVNN |
| | KALPSPIERTISKARGQAHQPSVYVLPPSREELSKNTVSLTCLI |
| | KDFFPPDIDVEWQSNGQQEPESKYRTTPPQLDEDGSYFLYSK |
| | LSVDKSRWQRGDTFICAVMHEALHNHYTQESLSHSPGK |
| | IL4-P4A-1F10VL-cCL [SEQ ID NO: 45] |
| | QTVVTQEPSLSVSPGGTVTLTCGLSSGSVSTGNYPGWYQQT |
| | QGRAPRSIIYDTNSRPSGVPDRFSGSISGNKAVLTITGAQPEDE |
| | ADYYCSLYTGIAYTDVVFGGGTHLTVLGQPKASPSVTLFPPS |
| | SEELGANKATLVCLISDFYPSGVTVAWKADGSPVTQGVETT |
| | KPSKQSNNKYAASSYLSLTPDKWKSHSSFSCLVTHEGSTVEK |
| | KVAPAECS |
| IL4-P4A-1F10VH- | IL4-P4A-1F10VH-cIgGB-YD [SEQ ID NO: 241] |
| cIgGB-YD/IL4-P4A- | EVQLVQSGGDLVKPAGSLRLSCVASGFTFNSYAMSWVRQAP |
| 1F10VL-cCL | GKGLQWVAGINSGGSTTTYADAVKGRFTISRDNAKNTLYLQ |
| | MNSLRAEDTAVYYCAKDQNSYYPSDTFEYWGQGTLVTVSS |
| | ASTTAPSVFPLAPSCGSTSGSTVALACLVSGYFPEPVTVSWNS |
| | GSLTSGVHTFPSVLQSSGLYSLSSMVTVPSSRWPSETFTCNVA |
| | HPASKTKVDKPVPKRENGRVPRPPDCPKCPAPEMLGGPSVFI |
| | FPPKPKDTLYIARDPEVTCVVVDLDPEDPEVQISWFVDGKQM |
| | QTAKTQPREEQFNGTYRVVSVLPIGHQDWLKGKQFTCKVNN |
| | KALPSPIERTISKARGQAHQPSVYVLPPSREELSKNTVSLTCLI |
| | KDFFPPDIDVEWQSNGQQEPESKYRTTPPQLDEDGSYFLYSK |
| | LSVDKSRWQRGDTFICAVMHEALHNHYTQESLSHSPGK |
| | IL4-P4A-1F10VL-cCL [SEQ ID NO: 45] |
| | QTVVTQEPSLSVSPGGTVTLTCGLSSGSVSTGNYPGWYQQT |
| | QGRAPRSIIYDTNSRPSGVPDRFSGSISGNKAVLTITGAQPEDE |
| | ADYYCSLYTGIAYTDVVFGGGTHLTVLGQPKASPSVTLFPPS |
| | SEELGANKATLVCLISDFYPSGVTVAWKADGSPVTQGVETT |
| | KPSKQSNNKYAASSYLSLTPDKWKSHSSFSCLVTHEGSTVEK |
| | KVAPAECS |
| IL4-P4-1A10VH- | IL4-P4-1A10VH-cIgGB [SEQ ID NO: 46] |
| cIgGB/IL4-P4- | EVQLVESGGDLVKPGGSLRLSCVASGFTFSDYDMTWVRQAP |
| 1A10VL-cCL | GKGLQWVADINNGGSSTTYADAVKGRFTISRDNAKNTLYLQ |
| | MNSLRAEDTAMYYCAKEGDNTWLSFDSWGQGTLVTVSSAS |
| | TTAPSVFPLAPSCGSTSGSTVALACLVSGYFPEPVTVSWNSGS |
| | LTSGVHTFPSVLQSSGLYSLSSMVTVPSSRWPSETFTCNVAHP |
| | ASKTKVDKPVPKRENGRVPRPPDCPKCPAPEMLGGPSVFIFP |
| | PKPKDTLLIARTPEVTCVVVDLDPEDPEVQISWFVDGKQMQT |
| | AKTQPREEQFNGTYRVVSVLPIGHQDWLKGKQFTCKVNNK |
| | ALPSPIERTISKARGQAHQPSVYVLPPSREELSKNTVSLTCLIK |
| | DFFPPDIDVEWQSNGQQEPESKYRTTPPQLDEDGSYFLYSKL |
| | SVDKSRWQRGDTFICAVMHEALHNHYTQESLSHSPGK |
| | IL4-P4-1A10VL-cCL [SEQ ID NO: 47] |
| | QSVLNQLPSVSGSLGQKVTIRCSGNTDNIGMVGAAWYRQVP |
| | GEAPKLLLYSDGNRPSGVPDRFSGSDSGRSATLTITGLQPEDE |
| | ADYYCQSLDPTVPGNYLFGSGTQLTILGQPKASPSVTLFPPSS |
| | EELGANKATLVCLISDFYPSGVTVAWKADGSPVTQGVETTK |
| | PSKQSNNKYAASSYLSLTPDKWKSHSSFSCLVTHEGSTVEKK |
| | VAPAECS |
| IL4-P3-1A6VH- | IL4-P3-1A6VH-cIgGB [SEQ ID NO: 48] |
| cIgGB/ | EVPLVESGGDLVKPGGSLRLSCVASGFTFHNYDLSWIRQLPG |
| IL4-P3-1A6VL-cCL | GGLQWVADIDKSGLTFYADAVKGRFTISRDNAKNTLYLQM |
| | NSLRAEDTAVYYCATWPLGSWYTETFEYWGQGTLVTVSSA |
| | STTAPSVFPLAPSCGSTSGSTVALACLVSGYFPEPVTVSWNSG |
| | SLTSGVHTFPSVLQSSGLYSLSSMVTVPSSRWPSETFTCNVAH |
| | PASKTKVDKPVPKRENGRVPRPPDCPKCPAPEMLGGPSVFIF |
| | PPKPKDTLLIARTPEVTCVVVDLDPEDPEVQISWFVDGKQMQ |
| | TAKTQPREEQFNGTYRVVSVLPIGHQDWLKGKQFTCKVNNK |
| | ALPSPIERTISKARGQAHQPSVYVLPPSREELSKNTVSLTCLIK |
| | DFFPPDIDVEWQSNGQQEPESKYRTTPPQLDEDGSYFLYSKL |
| | SVDKSRWQRGDTFICAVMHEALHNHYTQESLSHSPGK |
| | IL4-P3-1A6VL-cCL [SEQ ID NO: 49] |
| | QPVLTQPPSVSAALGQRVTISCNTNIGSPYDVQWYQQLPGKS |
| | PKTIIYGNSNRPSGVPVRFSGSKSGSTATLTIAGIQAEDEADY |
| | YCQSYDDNVDGYVFGSGTQLTVLGQPKASPSVTLFPPSSEEL |
| | GANKATLVCLISDFYPSGVTVAWKADGSPVTQGVETTKPSK |
| | QSNNKYAASSYLSLTPDKWKSHSSFSCLVTHEGSTVEKKVA |
| | PAECS |
| IL4-P3-1C4VH-cIgGB/ | IL4-P3-1C4VH-cIgGB [SEQ ID NO: 50] |
| IL4-P3-1C4VL-cCL | EVPLVESGGDLVKPGGSLRISCVASGITFNNDDMGWVRQAP |
| | GKGLQWLAEISSSGTSTYYADAVKGRFTISRDNAKNTVYLQ |
| | MNSLKAEDTGVYYCVGEDGYMALGFWGQGTLVTVSSASTT |
| | APSVEPLAPSCGSTSGSTVALACLVSGYFPEPVTVSWNSGSLT |
| | SGVHTFPSVLQSSGLYSLSSMVTVPSSRWPSETFTCNVAHPA |
| | SKTKVDKPVPKRENGRVPRPPDCPKCPAPEMLGGPSVFIFPP |
| | KPKDTLLIARTPEVTCVVVDLDPEDPEVQISWFVDGKQMQT |
| | AKTQPREEQFNGTYRVVSVLPIGHQDWLKGKQFTCKVNNK |
| | ALPSPIERTISKARGQAHQPSVYVLPPSREELSKNTVSLTCLIK |
| | DFFPPDIDVEWQSNGQQEPESKYRTTPPQLDEDGSYFLYSKL |
| | SVDKSRWQRGDTFICAVMHEALHNHYTQESLSHSPGK |
| | IL4-P3-1C4VL-cCL [SEQ ID NO: 51] |
| | QAVLNQPASVSGSLGQRVTISCSGSTNNIGVVGASWYQQFPG |
| | KAPKLLVYSDGDRPSGVPDRFSGSNSGYSDTLTITGLQAEDE |
| | ADYYCQSFDTTLNAYVFGSGTQLTVLGQPKASPSVTLFPPSS |
| | EELGANKATLVCLISDFYPSGVTVAWKADGSPVTQGVETTK |
| | PSKQSNNKYAASSYLSLTPDKWKSHSSFSCLVTHEGSTVEKK |
| | VAPAECS |
| IL4-P3-1B9VH-cIgGB/ | IL4-P3-1B9VH-cIgGB [SEQ ID NO: 52] |
| IL4-P3-1B9VL-cCL | ELQLVELGGDLVKPGGSLRLSCVASGFTFSSYGMSWVRQSP |
| | GKGLQWVADISSSGSTYYADAVKGRFTISRDNAKSTLYLQM |
| | NSLRAEDTAVYYCAKAGYGSYYDFGMDYWGHGTSVFVSSA |
| | STTAPSVFPLAPSCGSTSGSTVALACLVSGYFPEPVTVSWNSG |
| | SLTSGVHTFPSVLQSSGLYSLSSMVTVPSSRWPSETFTCNVAH |
| | PASKTKVDKPVPKRENGRVPRPPDCPKCPAPEMLGGPSVFIF |
| | PPKPKDTLLIARTPEVTCVVVDLDPEDPEVQISWFVDGKQMQ |
| | TAKTQPREEQFNGTYRVVSVLPIGHQDWLKGKQFTCKVNNK |
| | ALPSPIERTISKARGQAHQPSVYVLPPSREELSKNTVSLTCLIK |
| | DFFPPDIDVEWQSNGQQEPESKYRTTPPQLDEDGSYFLYSKL |
| | SVDKSRWQRGDTFICAVMHEALHNHYTQESLSHSPGK |
| | IL4-P3-1B9VL-cCL [SEQ ID NO: 53] |
| | QAVLTQPASVSAALGQRVTISCTGTATNVGGGYDVQWYQQ |
| | FPGRPPKTIIYGNYNRPSGVPDRFSASTSGTTATLTITGIQVAD |
| | EADYYCQSFDTTFNGHVFGAGTQLTILGQPKASPSVTLFPPSS |
| | EELGANKATLVCLISDFYPSGVTVAWKADGSPVTQGVETTK |
| | PSKQSNNKYAASSYLSLTPDKWKSHSSFSCLVTHEGSTVEKK |
| | VAPAECS |
| IL4-P3-1B10VH- | IL4-P3-1B10VH-cIgGB [SEQ ID NO: 54] |
| cIgGB/ | EVQLVESGGDLVKPGGSLRLSCVASGFTFSNYDMSWVRQAP |
| IL4-P3-1B10VL-cCL | GKGLQWVSTISYDGNNTYYSDAVKGRFTISRDNARNTLYLQ |
| | MNSLRAEDTAVYYCAKEFDIPWDNFDYWGQGTLVTVSSAS |
| | TTAPSVFPLAPSCGSTSGSTVALACLVSGYFPEPVTVSWNSGS |
| | LTSGVHTFPSVLQSSGLYSLSSMVTVPSSRWPSETFTCNVAHP |
| | ASKTKVDKPVPKRENGRVPRPPDCPKCPAPEMLGGPSVFIFP |
| | PKPKDTLLIARTPEVTCVVVDLDPEDPEVQISWFVDGKQMQT |
| | AKTQPREEQFNGTYRVVSVLPIGHQDWLKGKQFTCKVNNK |
| | ALPSPIERTISKARGQAHQPSVYVLPPSREELSKNTVSLTCLIK |
| | DFFPPDIDVEWQSNGQQEPESKYRTTPPQLDEDGSYFLYSKL |
| | SVDKSRWQRGDTFICAVMHEALHNHYTQESLSHSPGK |
| | IL4-P3-1B10VL-cCL [SEQ ID NO: 55] |
| | QSVLTQPTSVSAALGQRVTISCTGSNANIGSVYDVQWYQQLP |
| | GKSPKTIIYGNTHRPSGVPVRFSGSKSGSTATLTITGIQAEDEA |
| | DYYCQSYDATLHYVFGSGTQLTVLGQPKASPSVTLFPPSSEE |
| | LGANKATLVCLISDFYPSGVTVAWKADGSPVTQGVETTKPS |
| | KQSNNKYAASSYLSLTPDKWKSHSSFSCLVTHEGSTVEKKV |
| | APAECS |
| IL4-P3-1B10VHm- | IL4-P3-1B10VHm-cIgGB [SEQ ID NO: 56] |
| cIgGB/ | EVQLVESGGDLVKPGGSLRLSCVASGFTFSNYDMSWVRQAP |
| IL4-P3-1B10VL-cCL | GKGLQWVSTISYDGQNTYYSDAVKGRFTISRDNARNTLYLQ |
| | MNSLRAEDTAVYYCAKEFDIPWDNFDYWGQGTLVTVSSAS |
| | TTAPSVFPLAPSCGSTSGSTVALACLVSGYFPEPVTVSWNSGS |
| | LTSGVHTFPSVLQSSGLYSLSSMVTVPSSRWPSETFTCNVAHP |
| | ASKTKVDKPVPKRENGRVPRPPDCPKCPAPEMLGGPSVFIFP |
| | PKPKDTLLIARTPEVTCVVVDLDPEDPEVQISWFVDGKQMQT |
| | AKTQPREEQFNGTYRVVSVLPIGHQDWLKGKQFTCKVNNK |
| | ALPSPIERTISKARGQAHQPSVYVLPPSREELSKNTVSLTCLIK |
| | DFFPPDIDVEWQSNGQQEPESKYRTTPPQLDEDGSYFLYSKL |
| | SVDKSRWQRGDTFICAVMHEALHNHYTQESLSHSPGK |
| | IL4-P3-1B10VL-cCL [SEQ ID NO: 55] |
| | QSVLTQPTSVSAALGQRVTISCTGSNANIGSVYDVQWYQQLP |
| | GKSPKTIIYGNTHRPSGVPVRFSGSKSGSTATLTITGIQAEDEA |
| | DYYCQSYDATLHYVFGSGTQLTVLGQPKASPSVTLFPPSSEE |
| | LGANKATLVCLISDFYPSGVTVAWKADGSPVTQGVETTKPS |
| | KQSNNKYAASSYLSLTPDKWKSHSSFSCLVTHEGSTVEKKV |
| | APAECS |
| IL4-P3-1D7VH- | IL4-P3-1D7VH-cIgGB [SEQ ID NO: 57] |
| cIgGB/IL4-P3- | EVQLVQSGGDLVKPGGSLRLSCVASGFAFNGYDMDWVRLA |
| 1D7VL-cCL | PGKGLQWLSEISSSGSPTYYADAVEGRFTISRDNAKNTLYLQ |
| | MDSLRAEDTAVYYCARAGEYTYGYEGYYFDYWGQGTL VT |
| | VSSASTTAPSVFPLAPSCGSTSGSTVALACLVSGYFPEPVTVS |
| | WNSGSLTSGVHTFPSVLQSSGLYSLSSMVTVPSSRWPSETFT |
| | CNVAHPASKTKVDKPVPKRENGRVPRPPDCPKCPAPEMLGG |
| | PSVFIFPPKPKDTLLIARTPEVTCVVVDLDPEDPEVQISWFVD |
| | GKQMQTAKTQPREEQFNGTYRVVSVLPIGHQDWLKGKQFT |
| | CKVNNKALPSPIERTISKARGQAHQPSVYVLPPSREELSKNTV |
| | SLTCLIKDFFPPDIDVEWQSNGQQEPESKYRTTPPQLDEDGSY |
| | FLYSKLSVDKSRWQRGDTFICAVMHEALHNHYTQESLSHSP |
| | GK |
| | IL4-P3-1D7VL-cCL [SEQ ID NO: 58] |
| | QSMLTQPASVSGSLGQRVAISCSGSTNNIGIFGATWYQHLPG |
| | KAPKFLLYSDGNRPSGVPDRFSGSDSGNSVTLMITALQAEDE |
| | ADYYCQSFDTTLGAYVFGSGTQLTILGQPKASPSVTLFPPSSE |
| | ELGANKATLVCLISDFYPSGVTVAWKADGSPVTQGVETTKP |
| | SKQSNNKYAASSYLSLTPDKWKSHSSFSCLVTHEGSTVEKK |
| | VAPAECS |
| IL4-P4-1G3VH- | IL4-P4-1G3VH-cIgGB [SEQ ID NO: 59] |
| cIgGB/ | EVQLVESGGDLVKPGGSLRLSCVASGFTFSSYDMDWVRQAP |
| IL4-P4-1G3VL-cCL | GKGLEWLSEISTNGDRTHYADAVKGRFAISRDNAKNTVYLQ |
| | MNSLRAEDTAIYYCVRDGYEYLAFDYWGQGTLVTVSSASTT |
| | APSVFPLAPSCGSTSGSTVALACLVSGYFPEPVTVSWNSGSLT |
| | SGVHTFPSVLQSSGLYSLSSMVTVPSSRWPSETFTCNVAHPA |
| | SKTKVDKPVPKRENGRVPRPPDCPKCPAPEMLGGPSVFIFPP |
| | KPKDTLLIARTPEVTCVVVDLDPEDPEVQISWFVDGKQMQT |
| | AKTQPREEQFNGTYRVVSVLPIGHQDWLKGKQFTCKVNNK |
| | ALPSPIERTISKARGQAHQPSVYVLPPSREELSKNTVSLTCLIK |
| | DFFPPDIDVEWQSNGQQEPESKYRTTPPQLDEDGSYFLYSKL |
| | SVDKSRWQRGDTFICAVMHEALHNHYTQESLSHSPGK |
| | IL4-P4-1G3VL-cCL [SEQ ID NO: 60] |
| | QSVLNQLPSLSMNMGETATFTCEGDNIGHKYVNWYQQKPG |
| | QAPVLTIYEDNKRPSGIPERFSGSNSGNTATLTISGARAEDEA |
| | DYYCESEVSTGIVFGGGTHLTVLGQPKASPSVTLFPPSSEELG |
| | ANKATLVCLISDFYPSGVTVAWKADGSPVTQGVETTKPSKQ |
| | SNNKYAASSYLSLTPDKWKSHSSFSCLVTHEGSTVEKKVAP |
| | AECS |

TABLE 15
FULL LENGTH IgGs SPECIFIC TO CANINE IL-13

| Antibody | Amino acid sequence/SEQ ID NOs: for heavy and light chains |
|---|---|
| IL13-P3-1G5VH- | IL13-P3-1G5VH-cIgGB [SEQ ID NO: 82] |
| cIgGB/IL13-P3-1G5VL- | EVQLVQSGGDLVKPGGTLRLSCVASGFTFSSYDMSWVR |
| cCL | QSPGKGLQWVAAIWNDGSNTEYADAVKGRFTVSRDNA |
| | KNTVYLEMNSLRAEDTAMYYCVKEGPSYGYFDYWGQG |
| | TLVTVSSASTTAPSVFPLAPSCGSTSGSTVALACLVSGYFP |
| | EPVTVSWNSGSLTSGVHTFPSVLQSSGLYSLSSMVTVPSS |
| | RWPSETFTCNVAHPASKTKVDKPVPKRENGRVPRPPDCP |
| | KCPAPEMLGGPSVFIFPPKPKDTLLIARTPEVTCVVVDLDP |
| | EDPEVQISWFVDGKQMQTAKTQPREEQFNGTYRVVSVLP |
| | IGHQDWLKGKQFTCKVNNKALPSPIERTISKARGQAHQPS |
| | VYVLPPSREELSKNTVSLTCLIKDFFPPDIDVEWQSNGQQ |
| | EPESKYRTTPPQLDEDGSYFLYSKLSVDKSRWQRGDTFIC |
| | AVMHEALHNHYTQESLSHSPGK |
| | IL13-P3-1G5VL-cCL [SEQ ID NO: 83] |
| | SYELTQPPSVSVSLGQTATISCSGESLSKYYAQWFQQKAG |
| | QAPVLVIYQDTERPSGIPDRFSGSSSGNTHTLTISGARAED |
| | EADYYCESAISPDTYVFGSGTQLTILGQPKASPSVTLFPPS |
| | SEELGANKATLVCLISDFYPSGVTVAWKADGSPVTQGVE |
| | TTKPSKQSNNKYAASSYLSLTPDKWKSHSSFSCLVTHEGS |
| | TVEKKVAPAECS |
| IL13-P3-05VH- | IL13-P3-05VH-cIgGB [SEQ ID NO: 84] |
| cIgGB/IL13-P3-05VLm- | EVQLVESGGDSVRPGGSLRLSCVASGFTFSRYNMGWVR |
| cCL | QAPGKGLQWVAWIYDGGSRTSYADAVKGRFTISRDNAK |
| | DTLYLQMDDLRVDDTAVYYCARDPSSWGSLFDYWGQG |
| | TLVTVSSASTTAPSVFPLAPSCGSTSGSTVALACLVSGYFP |
| | EPVTVSWNSGSLTSGVHTFPSVLQSSGLYSLSSMVTVPSS |
| | RWPSETFTCNVAHPASKTKVDKPVPKRENGRVPRPPDCP |
| | KCPAPEMLGGPSVFIFPPKPKDTLLIARTPEVTCVVVDLDP |
| | EDPEVQISWFVDGKQMQTAKTQPREEQFNGTYRVVSVLP |
| | IGHQDWLKGKQFTCKVNNKALPSPIERTISKARGQAHQPS |
| | VYVLPPSREELSKNTVSLTCLIKDFFPPDIDVEWQSNGQQ |
| | EPESKYRTTPPQLDEDGSYFLYSKLSVDKSRWQRGDTFIC |
| | AVMHEALHNHYTQESLSHSPGK |
| | IL13-P3-05VLm-cCL [SEQ ID NO: 85] |
| | SYVLTQPPSVTVPLRQTARISCGGDNIRSKNVHWYQQKP |
| | GQAPVLIIYQDSNRPTGIPERFSGSNSGNMATLTIRGALAE |
| | DEADYYCQVWANDGYVFGSGTQLTVLGQPKASPSVTLF |
| | PPSSEELGANKATLVCLISDFYPSGVTVAWKADGSPVTQ |
| | GVETTKPSKQSNNKYAASSYLSLTPDKWKSHSSFSCLVT |
| | HEGSTVEKKVAPAECS |
| IL13-P3-05VH- | IL13-P3-05VH-cIgGB [SEQ ID NO: 84] |
| cIgGB/IL13-P3-05VL-cCL | EVQLVESGGDSVRPGGSLRLSCVASGFTFSRYNMGWVR |
| | QAPGKGLQWVAWIYDGGSRTSYADAVKGRFTISRDNAK |
| | DTLYLQMDDLRVDDTAVYYCARDPSSWGSLFDYWGQG |
| | TLVTVSSASTTAPSVFPLAPSCGSTSGSTVALACLVSGYFP |
| | EPVTVSWNSGSLTSGVHTFPSVLQSSGLYSLSSMVTVPSS |
| | RWPSETFTCNVAHPASKTKVDKPVPKRENGRVPRPPDCP |
| | KCPAPEMLGGPSVFIFPPKPKDTLLIARTPEVTCVVVDLDP |
| | EDPEVQISWFVDGKQMQTAKTQPREEQFNGTYRVVSVLP |
| | IGHQDWLKGKQFTCKVNNKALPSPIERTISKARGQAHQPS |
| | VYVLPPSREELSKNTVSLTCLIKDFFPPDIDVEWQSNGQQ |
| | EPESKYRTTPPQLDEDGSYFLYSKLSVDKSRWQRGDTFIC |
| | AVMHEALHNHYTQESLSHSPGK |
| | IL13-P3-05VL-cCL [SEQ ID NO: 86] |
| | SYVLTQPPSVTVPLRQTARISCGGDNIRSKNVHWYQQKP |
| | GQAPVLIIYNDSNRPTGIPERFSGSNSGNMATLTIRGALAE |
| | DEADYYCQVWANDGYVFGSGTQLTVLGQPKASPSVTLF |
| | PPSSEELGANKATLVCLISDFYPSGVTVAWKADGSPVTQ |
| | GVETTKPSKQSNNKYAASSYLSLTPDKWKSHSSFSCLVT |
| | HEGSTVEKKVAPAECS |
| IL13-P3-1B4VH-cIgGB/ | IL13-1B4VH-cIgGB [SEQ ID NO: 87] |
| IL13-P3-1B4VL-cCL | ELTLQESGPGLVKPSQTLSLTCVVSGGSVTSNYYWNWIR |
| | QRPGRGLEWMGYWTGSTNYNPAFQGRISITADTAKNHFS |
| | LQLTSMTTEDTAVYYCARASTWLHGMDFWGHGTSLFVS |
| | SASTTAPSVFPLAPSCGSTSGSTVALACLVSGYFPEPVTVS |
| | WNSGSLTSGVHTFPSVLQSSGLYSLSSMVTVPSSRWPSET |
| | FTCNVAHPASKTKVDKPVPKRENGRVPRPPDCPKCPAPE |
| | MLGGPSVFIFPPKPKDTLLIARTPEVTCVVVDLDPEDPEV |
| | QISWFVDGKQMQTAKTQPREEQFNGTYRVVSVLPIGHQD |
| | WLKGKQFTCKVNNKALPSPIERTISKARGQAHQPSVYVL |
| | PPSREELSKNTVSLTCLIKDFFPPDIDVEWQSNGQQEPESK |
| | YRTTPPQLDEDGSYFLYSKLSVDKSRWQRGDTFICAVMH |
| | EALHNHYTQESLSHSPGK |
| | IL13-P3-1B4VL-cCL [SEQ ID NO: 88] |
| | SYVLTQSPSVSGSLGQTVTISCSGTTIGVVGANWYQQLPG |
| | KAPKLLVHSDGGRLSGVPDRFSGSESGNSAALTITGLQAE |
| | DEADYYCQSFDTTLGYVFGSGTQLTILGQPKASPSVTLFP |
| | PSSEELGANKATLVCLISDFYPSGVTVAWKADGSPVTQG |
| | VETTKPSKQSNNKYAASSYLSLTPDKWKSHSSFSCLVTHE |
| | GSTVEKKVAPAECS |
| IL13-P3-05VH-cIgGB- | IL13-P3-05VH-cIgGB-YD [SEQ ID NO: 240] |
| YD/IL13-P3-05VLm-cCL | EVQLVESGGDSVRPGGSLRLSCVASGFTFSRYNMGWVR |
| | QAPGKGLQWVAWIYDGGSRTSYADAVKGRFTISRDNAK |
| | DTLYLQMDDLRVDDTAVYYCARDPSSWGSLFDYWGQG |
| | TLVTVSSASTTAPSVFPLAPSCGSTSGSTVALACLVSGYFP |
| | EPVTVSWNSGSLTSGVHTFPSVLQSSGLYSLSSMVTVPSS |
| | RWPSETFTCNVAHPASKTKVDKPVPKRENGRVPRPPDCP |
| | KCPAPEMLGGPSVFIFPPKPKDTLYIARDPEVTCVVVDLD |
| | PEDPEVQISWFVDGKQMQTAKTQPREEQFNGTYRVVSVL |
| | PIGHQDWLKGKQFTCKVNNKALPSPIERTISKARGQAHQP |
| | SVYVLPPSREELSKNTVSLTCLIKDFFPPDIDVEWQSNGQ |
| | QEPESKYRTTPPQLDEDGSYFLYSKLSVDKSRWQRGDTFI |
| | CAVMHEALHNHYTQESLSHSPGK |
| | IL13-P3-05VLm-cCL [SEQ ID NO: 85] |
| | SYVLTQPPSVTVPLRQTARISCGGDNIRSKNVHWYQQKP |
| | GQAPVLIIYQDSNRPTGIPERFSGSNSGNMATLTIRGALAE |
| | DEADYYCQVWANDGYVFGSGTQLTVLGQPKASPSVTLF |
| | PPSSEELGANKATLVCLISDFYPSGVTVAWKADGSPVTQ |
| | GVETTKPSKQSNNKYAASSYLSLTPDKWKSHSSFSCLVT |
| | HEGSTVEKKVAPAECS |

| | |
|---|---|
| IL-13-P3-1C6VH-cIgGB/ | IL-13-P3-1C6VH-cIgGB [SEQ ID NO: 246] |
| IL-13-P3-1C6VL-cCL | ELTLQESGPGLVKPSQTLSLTCVVSGGSVTNTYEWDWIRQRPGRGLE |
| | WMGSWTDNTWYNPAFQGRISITADTAKNQLSLQLSSVTTGDTAVYF |
| | CARWTKRWGYDYWGQGTLVTVSSASTTAPSVFPLAPSCGSTSGSTVA |
| | LACLVSGYFPEPVTVSWNSGSLTSGVHTFPSVLQSSGLYSLSSMVTVPS |
| | SRWPSETFTCNVAHPASKTKVDKPVPKRENGRVPRPPDCPKCPAPEM |
| | LGGPSVFIFPPKPKDTLLIARTPEVTCVVVDLDPEDPEVQISWFVDGKQ |
| | MQTAKTQPREEQFNGTYRVVSVLPIGHQDWLKGKQFTCKVNNKALP |
| | SPIERTISKARGQAHQPSVYVLPPSREELSKNTVSLTCLIKDFFPPDIDVE |
| | WQSNGQQEPESKYRTTPPQLDEDGSYFLYSKLSVDKSRWQRGDTFIC |
| | AVMHEALHNHYTQESLSHSPGK |
| | IL-13-P3-1C6VL-cCL [SEQ ID NO: 247] |
| | QSVLTQPASVSGSLGQRVTISCSGRTNNIGYFGANWYQQVPGKAPKLL |
| | VSSYGNRPSGVPDRFSASNSANSATLTITGLQAEDEADYYCQSFDTTLG |
| | AVVFGGGTQLTVLGQPKASPSVTLFPPSSEELGANKATLVCLISDFYPSG |
| | VTVAWKADGSPVTQGVETTKPSKQSNNKYAASSYLSLTPDKWKSHSS |
| | FSCLVTHEGSTVEKKVAPAECS |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A canine antibody or antigen binding fragment thereof, that binds canine interleukin-13 (cIL-13), wherein the antibody comprises a heavy chain and a light chain that together comprise a set of six complementary determining regions (CDRs), three of which are heavy chain CDRs: CDR heavy 1 (HCDR1), CDR heavy 2 (HCDR2) and CDR heavy 3 (HCDR3) and three of which are light chain CDRs: CDR light 1 (LCDR1), CDR light 2 (LCDR2), and CDR light 3 (LCDR3); wherein each CDR comprises an amino acid sequence; and
wherein the set of six CDRs are selected from the group of sets consisting of (i), (ii), (iii), (iv), (v), (vi), (vii), (viii) and (ix);
wherein for set (i):
HCDR1 comprises the amino acid sequence of SEQ ID NO: 150;
HCDR2 comprises the amino acid sequence of SEQ ID NO: 151;
HCDR3 comprises the amino acid sequence of SEQ ID NO: 152;
LCDR1 comprises the amino acid sequence of SEQ ID NO: 153;
LCDR2 comprises the amino acid sequence of SEQ ID NO: 235; and
LCDR3 comprises the amino acid sequence of SEQ ID NO: 155;
wherein for set (ii):
HCDR1 comprises the amino acid sequence of SEQ ID NO: 150;
HCDR2 comprises the amino acid sequence of SEQ ID NO: 151;
HCDR3 comprises the amino acid sequence of SEQ ID NO: 152;
LCDR1 comprises the amino acid sequence of SEQ ID NO: 153;
LCDR2 comprises the amino acid sequence of SEQ ID NO: 154;
LCDR3 comprises the amino acid sequence of SEQ ID NO: 155;
wherein for set (iii)
HCDR1 comprises the amino acid sequence of SEQ ID NO: 126;
HCDR2 comprises the amino acid sequence of SEQ ID NO: 127;
HCDR3 comprises the amino acid sequence of SEQ ID NO: 128;
LCDR1 comprises the amino acid sequence of SEQ ID NO: 129;
LCDR2 comprises the amino acid sequence of SEQ ID NO: 130; and
LCDR3 comprises the amino acid sequence of SEQ ID NO: 131;
wherein for set (iv):
HCDR1 comprises the amino acid sequence of SEQ ID NO: 132;
HCDR2 comprises the amino acid sequence of SEQ ID NO: 133;
HCDR3 comprises the amino acid sequence of SEQ ID NO: 134;
LCDR1 comprises the amino acid sequence of SEQ ID NO: 135;
LCDR2 comprises the amino acid sequence of SEQ ID NO: 136; and
LCDR3 comprises the amino acid sequence of SEQ ID NO: 137;
wherein for set (v):
HCDR1 comprises the amino acid sequence of SEQ ID NO: 138;
HCDR2 comprises the amino acid sequence of SEQ ID NO: 139;
HCDR3 comprises the amino acid sequence of SEQ ID NO: 140;
LCDR1 comprises the amino acid sequence of SEQ ID NO: 141;
LCDR2 comprises the amino acid sequence of SEQ ID NO: 142; and
LCDR3 comprises the amino acid sequence of SEQ ID NO: 143;
wherein for set (vi)
HCDR1 comprises the amino acid sequence of SEQ ID NO: 144;
HCDR2 comprises the amino acid sequence of SEQ ID NO: 145;
HCDR3 comprises the amino acid sequence of SEQ ID NO: 146;
LCDR1 comprises the amino acid sequence of SEQ ID NO: 147;
LCDR2 comprises the amino acid sequence of SEQ ID NO: 148; and
LCDR3 comprises the amino acid sequence of SEQ ID NO: 149;
wherein for set (vii)
HCDR1 comprises the amino acid sequence of SEQ ID NO: 120;
HCDR2 comprises the amino acid sequence of SEQ ID NO: 121;
HCDR3 comprises the amino acid sequence of SEQ ID NO: 122;
LCDR1 comprises the amino acid sequence of SEQ ID NO: 123;
LCDR2 comprises the amino acid sequence of SEQ ID NO: 124; and
LCDR3 comprises the amino acid sequence of SEQ ID NO: 125; and
wherein for set (viii)
HCDR1 comprises the amino acid sequence of SEQ ID NO: 114;
HCDR2 comprises the amino acid sequence of SEQ ID NO: 115;
HCDR3 comprises the amino acid sequence of SEQ ID NO: 116;
LCDR1 comprises the amino acid sequence of SEQ ID NO: 117;
LCDR2 comprises the amino acid sequence of SEQ ID NO: 118; and
LCDR3 comprises the amino acid sequence of SEQ ID NO: 119;
wherein for set (ix)
HCDR1 comprises the amino acid sequence of SEQ ID NO: 248;
HCDR2 comprises the amino acid sequence of SEQ ID NO: 249;
HCDR3 comprises the amino acid sequence of SEQ ID NO: 250;
LCDR1 comprises the amino acid sequence of SEQ ID NO: 251;
LCDR2 comprises the amino acid sequence of SEQ ID NO: 252; and
LCDR3 comprises the amino acid sequence of SEQ ID NO: 253;
wherein the canine antibody or antigen binding fragment thereof bind to cIL-13.

2. The canine antibody of claim 1, or antigen binding fragment thereof, wherein the antibody comprises:
a heavy chain variable region comprising at least 90%, 95%, 98%, 99%, or 100% sequence identity with the amino acid sequence selected from the group consisting of SEQ ID NO: 80, SEQ ID NO: 62, SEQ ID NO: 65, SEQ ID NO: 68, SEQ ID NO: 71, SEQ ID NO: 74, SEQ ID NO: 77 and SEQ ID NO: 244, and
a light chain variable region comprising at least 90%, 95%, 98%, 99%, or 100% sequence identity with the amino acid sequence selected from the group consisting of SEQ ID NO: 234, SEQ ID NO: 81, SEQ ID NO: 63, SEQ ID NO: 66, SEQ ID NO: 69, SEQ ID NO: 72, SEQ ID NO: 75, SEQ ID NO: 78 and SEQ ID NO: 245.

3. (canceled)

4. The canine antibody of claim 1, or antigen binding fragment thereof wherein:
the heavy chain variable region comprises at least 90%, 95%, 98%, 99%, or 100% sequence identity with the amino acid sequence of SEQ ID NO: 62 and the light chain variable region comprises at least 90%, 95%, 98%, 99%, or 100% sequence identity with the amino acid sequence of SEQ ID NO: 63, or
the heavy chain variable region comprises at least 90%, 95%, 98%, 99%, or 100% sequence identity with the amino acid sequence of SEQ ID NO: 65 and the light chain variable region comprises at least 90%, 95%, 98%, 99%, or 100% sequence identity with the amino acid sequence of SEQ ID NO: 66 or
the heavy chain variable region comprises at least 90%, 95%, 98%, 99%, or 100% sequence identity with the amino acid sequence of SEQ ID NO: 68 and the light chain variable region comprises at least 90%, 95%, 98%, 99%, or 100% sequence identity with the amino acid sequence of SEQ ID NO: 69, or
the heavy chain variable region comprises at least 90%, 95%, 98%, 99%, or 100% sequence identity with the amino acid sequence of SEQ ID NO: 71 and the light chain variable region comprises at least 90%, 95%, 98%, 99%, or 100% sequence identity with the amino acid sequence of SEQ ID NO: 72,
the heavy chain variable region comprises at least 90%, 95%, 98%, 99%, or 100% sequence identity with the amino acid sequence of SEQ ID NO: 74 and the light chain variable region comprises at least 90%, 95%, 98%, 99%, or 100% sequence identity with the amino acid sequence of SEQ ID NO: 75, or
the heavy chain variable region comprises at least 90%, 95%, 98%, 99%, or 100% sequence identity with the amino acid sequence of SEQ ID NO: 77 and the light chain variable region comprises at least 90%, 95%, 98%, 99%, or 100% sequence identity with the amino acid sequence of SEQ ID NO: 78, or
the heavy chain variable region comprises at least 90%, 95%, 98%, 99%, or 100% sequence identity with the amino acid sequence of SEQ ID NO: 80 and the light chain variable region comprises at least 90%, 95%, 98%, 99%, or 100% sequence identity with the amino acid sequence of SEQ ID NO: 81, or
the heavy chain variable region comprises at least 90%, 95%, 98%, 99%, or 100% sequence identity with the amino acid sequence of SEQ ID NO: 80 and the light chain variable region comprises at least 90%, 95%, 98%, 99%, or 100% sequence identity with the amino acid sequence of SEQ ID NO: 234, or
the heavy chain variable region comprises at least 90%, 95%, 98%, 99%, or 100% sequence identity with the amino acid sequence of SEQ ID NO: 244 and the light chain variable region comprises at least 90%, 95%, 98%, 99%, or 100% sequence identity with the amino acid sequence of SEQ ID NO: 245

5. The canine antibody of claim 2, or antigen binding fragment thereof, which comprises a heavy chain variable region comprising at least 90%, 95%, 98%, 99%, or 100% sequence identity with the amino acid sequence of SEQ ID NO: 80.

6. The canine antibody of claim 2, or antigen binding fragment thereof, which comprises a light chain variable region comprising at least 90%, 95%, 98%, 99%, or 100% sequence identity with the amino acid sequence of SEQ ID NO: 234.

7. The canine antibody of claim 1, or antigen binding fragment thereof, wherein:
the HCDR1 comprises the amino acid sequence of SEQ ID NO: 150;
the HCDR2 comprises the amino acid sequence of SEQ ID NO: 151;
the HCDR3 comprises the amino acid sequence of SEQ ID NO: 152;
the LCDR1 comprises the amino acid sequence of SEQ ID NO: 153;
the LCDR2 comprises the amino acid sequence of SEQ ID NO: 235; and
the LCDR3 comprises the amino acid sequence of SEQ ID NO: 155.

8. The canine antibody claim 1, or antigen binding fragment thereof, that comprises a canine hinge region that comprises an amino acid sequence that comprises at least 90%, 95%, or 100% sequence identity with the amino acid sequence selected from the group consisting of SEQ ID NO: 109, SEQ ID NO: 110, SEQ ID NO: 111, SEQ ID NO: 112, and SEQ ID NO: 113.

9. The canine antibody of claim 1, or antigen binding fragment thereof, that comprises a canine fragment crystallizable region (cFc); wherein the cFc comprises an amino acid sequence that comprises at least 90%, 95%, 98%, 99%, or 100% sequence identity with the amino acid sequence selected from the group consisting of SEQ ID NO: 99, SEQ ID NO: 100, SEQ ID NO: 101, SEQ ID NO: 102, SEQ ID NO: 103, SEQ ID NO: 104, SEQ ID NO: 105, and SEQ ID NO: 106.

10. (canceled)

11. The canine antibody of claim 1, or antigen binding fragment thereof, wherein:
the heavy chain comprises at least 90%, 95%, 98%, 99%, or 100% sequence identity with the amino acid sequence of SEQ ID NO: 82 and the light chain comprises at least 90%, 95%, 98%, 99%, or 100% sequence identity with the amino acid sequence of SEQ ID NO: 83, or
the heavy chain comprises at least 90%, 95%, 98%, 99%, or 100% sequence identity with the amino acid sequence of SEQ ID NO: 84 and the light chain comprises at least 90%, 95%, 98%, 99%, or 100% sequence identity with the amino acid sequence of SEQ ID NO: 85, or
the heavy chain comprises at least 90%, 95%, 98%, 99%, or 100% sequence identity with the amino acid sequence of SEQ ID NO: 84 and the light chain comprises at least 90%, 95%, 98%, 99%, or 100% sequence identity with the amino acid sequence of SEQ ID NO: 86, or
the heavy chain comprises at least 90%, 95%, 98%, 99%, or 100% sequence identity with the amino acid sequence of SEQ ID NO: 240 and the light chain comprises at least 90%, 95%, 98%, 99%, or 100% sequence identity with the amino acid sequence of SEQ ID NO: 85, or
the heavy chain comprises at least 90%, 95%, 98%, 99%, or 100% sequence identity with the amino acid sequence of SEQ ID NO: 240 and the light chain comprises at least 90%, 95%, 98%, 99%, or 100% sequence identity with the amino acid sequence of SEQ ID NO: 86, or
the heavy chain comprises at least 90%, 95%, 98%, 99%, or 100% sequence identity with the amino acid sequence of SEQ ID NO: 87 and the light chain comprises at least 90%, 95%, 98%, 99%, or 100% sequence identity with the amino acid sequence of SEQ ID NO: 88, or
the heavy chain comprises at least 90%, 95%, 98%, 99%, or 100% sequence identity with the amino acid sequence of SEQ ID NO: 246 and the light chain comprises at least 90%, 95%, 98%, 99%, or 100% sequence identity with the amino acid sequence of SEQ ID NO: 247.

12. The canine antibody of claim 11, or antigen binding fragment thereof, wherein the heavy chain comprises at least 90%, 95%, 98%, 99%, or 100% sequence identity with the amino acid sequence of SEQ ID NO: 84 and the light chain comprises at least 90%, 95%, 98%, 99%, or 100% sequence identity with the amino acid sequence of SEQ ID NO: 85.

13. The canine antibody of claim 9, or antigen binding fragment thereof, wherein the cFc comprises one or more amino acid substitutions, wherein said one or more amino acid residue substitutions is at an amino acid residue position selected from the group consisting of 252, 254, 256, 308, 433, 434, 436, or any combination thereof, wherein the amino acid residue positions are numbered according to the EU index as in Kabat:
wherein when the substitution is at amino acid residue position 252, the substitution is with a tyrosine residue;
wherein when the substitution is at amino acid residue position 254, the substitution is with a threonine residue;
wherein when the substitution is at amino acid residue position 256, the substitution is with an aspartic acid residue or a glutamic acid residue;
wherein when the substitution is at amino acid residue position 308, the substitution is with a proline residue;
wherein when the substitution is at amino acid residue position 433, the substitution is with a lysine residue or a leucine residue;
wherein when the substitution is at amino acid residue position 434, the substitution is with a phenylalanine residue, a histidine residue, or a tyrosine residue; and
wherein when the substitution is at amino acid residue position 436, the substitution is with a threonine residue.

14. The canine antibody of claim 13, or antigen binding fragment thereof, wherein the cFc comprises two or more amino acid residue substitutions at the amino acid residue positions selected from the group consisting of:
(i) the substitution at amino acid residue position 252 with a tyrosine residue and the substitution at amino acid residue position 256 with an aspartic acid residue;
(ii) the substitution at amino acid residue position 256 with an aspartic acid residue and the substitution at amino acid residue position 434 with a tyrosine residue;
(iii) the substitution at amino acid residue position 308 with a proline residue and the substitution at amino acid residue position 434 with a tyrosine residue;
(iv) the substitution at amino acid residue position 433 with a lysine residue and the substitution at amino acid residue position 434 with a phenylalanine residue;
(v) the substitution at amino acid residue position 433 with a lysine residue and the substitution at amino acid residue position 434 with a tyrosine residue;
(vi) the substitution at amino acid residue position 433 with a leucine residue and the substitution at amino acid residue position 434 with a phenylalanine residue;
(vii) the substitution at amino acid residue position 433 with a leucine residue and the substitution at amino acid residue position 434 with a tyrosine residue;
(viii) the substitution at amino acid residue position 434 with a tyrosine residue and the substitution at amino acid residue position 436 with a threonine residue;
(ix) the substitution at amino acid residue position 252 with a tyrosine residue, the substitution at amino acid residue position 254 with a threonine residue and the substitution at amino acid residue position 256 with a glutamic acid residue;
(x) the substitution at amino acid residue position 256 with an aspartic acid residue, the substitution at amino acid residue position 308 with a proline residue, and the substitution at amino acid residue position 434 with a tyrosine residue;
(xi) the substitution at amino acid residue position 433 with a lysine residue, the substitution at amino acid residue position 434 with a phenylalanine residue, and the substitution at amino acid residue position 436 with a threonine residue;
(xii) the substitution at amino acid residue position 433 with a lysine residue, the substitution at amino acid residue position 434 with a tyrosine residue, and the substitution at amino acid residue position 436 with a threonine residue;
(xiii) the substitution at amino acid residue position 433 with a leucine residue, the substitution at amino acid residue position 434 with a phenylalanine residue, and the substitution at amino acid residue position 436 with a threonine residue; and
(xiv) the substitution at amino acid residue position 433 with a leucine residue, the substitution at amino acid residue position 434 with a tyrosine residue, and the substitution at amino acid residue position 436 with a threonine residue;
(XV) the substitution at amino acid residue position 434 with a histidine residue, preferably wherein the cFc comprises at least 90%, 95%, 98%, 99%, or 100% sequence identity with the amino acid sequence of SEQ ID NO: 257 or SEQ ID NO: 258.

15. The canine antibody of claim 14, or antigen binding fragment thereof, wherein the cFc comprises the substitution at amino acid residue position 252 with a tyrosine residue and the substitution at amino acid residue position 256 with an aspartic acid residue, preferably wherein the cFc comprises an amino acid sequence that comprises at least 90%, 95%, 98%, 99%, or 100% sequence identity with the amino acid sequence selected from the group consisting of SEQ ID NO: 255 or SEQ ID NO: 256.

16. The canine antibody of claim 15, or antigen binding fragment thereof, wherein the heavy chain comprises at least 90%, 95%, 98%, 99%, or 100% sequence identity with the amino acid sequence of SEQ ID NO: 240 and the light chain comprises at least 90%, 95%, 98%, 99%, or 100% sequence identity with the amino acid sequence of SEQ ID NO: 85 or SEQ ID NO: 86.

17. The canine antibody of claim 1, or antigen binding fragment thereof, having at least one property selected from the group consisting of:
binds canine interleukin-13 (cIL-13) and has an IC50 of lower than 50 nM for canine IL-13 mediated STAT-6 phosphorylation in DH82 cells,
binds canine interleukin-13 (cIL-13) and has an IC50 of lower than 50 nM for canine IL-13 mediated STAT-6 phosphorylation in DH82 cells,
binds to an epitope comprised by an amino acid sequence selected from the group consisting of SEQ ID NO: 239, SEQ ID NO: 95, SEQ ID NO: 96, and SEQ ID NO: 97, SEQ ID NO: 98, and any combination thereof, and
binds to one, two, three, four, five, or six serine residues on cIL-13 at amino acid residue positions: 27 (S₂₇), 32 (S₃₂), 36 (S₃₆), 50 (S₅₀), 55 (S₅₅), and 58 (S₅₈) of the amino acid sequence of SEQ ID NO: 2.

18.-20. (canceled)

21. A nucleic acid or a pair of nucleic acids, selected from the group consisting of:
a nucleic acid comprising a nucleotide sequence that encodes the heavy chain of the canine antibody or antigen binding fragment thereof of claim 1, wherein the nucleic acid encodes a heavy chain comprising at least 90%, 95%, 98%, 99%, or 100% sequence identity with the amino acid sequence selected from: SEQ ID NO: 84, SEQ ID NO: 240, SEQ ID NO: 82, SEQ ID NO: 87 or SEQ ID NO: 246,
a nucleic acid comprising a nucleotide sequence that encodes the light chain of the canine antibody or antigen binding fragment thereof of claim 1, wherein the nucleic acid encodes a light chain comprises at least 90%, 95%, 98%, 99%, or 100% sequence identity with the amino acid sequence selected from: SEQ ID NO: 85, SEQ ID NO: 86, SEQ ID NO: 83, SEQ ID NO: 88 or SEQ ID NO: 247 and
a pair of nucleic acids, wherein:
one of the pair of nucleic acids comprises a nucleotide sequence that encodes the light chain of a specific canine antibody of claim 1 and encodes the light chain of the canine antibody or antigen binding fragment thereof of claim 1, wherein the nucleic acid encodes a light chain comprises at least 90%, 95%, 98%, 99%, or 100% sequence identity with the amino acid sequence selected from: SEQ ID NO: 85, SEQ ID NO: 86, SEQ ID NO: 83, SEQ ID NO: 88 or SEQ ID NO: 247, and
the other of the pair of nucleic acids comprises a nucleotide sequence that encodes the heavy chain of said specific canine antibody, wherein the nucleic acid encodes a heavy chain comprising at least 90%, 95%, 98%, 99%, or 100% sequence identity with the amino acid sequence selected from: SEQ ID NO: 84, SEQ ID NO: 240, SEQ ID NO: 82, SEQ ID NO: 87 or SEQ ID NO: 246.

22.-23. (canceled)

24. An expression vector comprising a nucleic acid or the pair of nucleic acids of claim 21, and optionally, wherein the expression vector is present in a host cell.

25.-26. (canceled)

27. A pharmaceutical composition comprising the canine antibody of claim 1, or antigen binding fragment thereof, and a pharmaceutically acceptable carrier or diluent.

28.-29. (canceled)

30. A method of aiding in the blocking of the skin inflammation associated with atopic dermatitis, comprising administering to a subject in need thereof a therapeutically effective amount of the pharmaceutical composition of claim 27.

31. The method of claim 30, wherein the method further comprises aiding in the blocking of the skin inflammation and pruritus associated with atopic dermatitis.

32. (canceled)

33. A single-chain Fv (scFv) antibody comprising a set of six complementary determining regions (CDRs), three of which are heavy chain CDRs: CDR heavy 1 (HCDR1), CDR heavy 2 (HCDR2) and CDR heavy 3 (HCDR3) and three of which are light chain CDRs: CDR light 1 (LCDR1), CDR light 2 (LCDR2), and CDR light 3 (LCDR3); wherein each CDR comprises an amino acid sequence; and wherein the set of six CDRs is selected from the group of sets consisting of (i), (ii), (iii), (iv), (v), (vi), (vii), (viii) and set (ix);
wherein for set (i):
HCDR1 comprises the amino acid sequence of SEQ ID NO: 114;
HCDR2 comprises the amino acid sequence of SEQ ID NO: 115;
HCDR3 comprises the amino acid sequence of SEQ ID NO: 116;
LCDR1 comprises the amino acid sequence of SEQ ID NO: 117;
LCDR2 comprises the amino acid sequence of SEQ ID NO: 118; and
LCDR3 comprises the amino acid sequence of SEQ ID NO: 119;
wherein for set (ii):
HCDR1 comprises the amino acid sequence of SEQ ID NO: 120;
HCDR2 comprises the amino acid sequence of SEQ ID NO: 121;
HCDR3 comprises the amino acid sequence of SEQ ID NO: 122;
LCDR1 comprises the amino acid sequence of SEQ ID NO: 123;
LCDR2 comprises the amino acid sequence of SEQ ID NO: 124; and
LCDR3 comprises the amino acid sequence of SEQ ID NO: 125;
wherein for set (iii)
HCDR1 comprises the amino acid sequence of SEQ ID NO: 126;
HCDR2 comprises the amino acid sequence of SEQ ID NO: 127;
HCDR3 comprises the amino acid sequence of SEQ ID NO: 128;
LCDR1 comprises the amino acid sequence of SEQ ID NO: 129;
LCDR2 comprises the amino acid sequence of SEQ ID NO: 130; and
LCDR3 comprises the amino acid sequence of SEQ ID NO: 131;
wherein for set (iv):
HCDR1 comprises the amino acid sequence of SEQ ID NO: 132;
HCDR2 comprises the amino acid sequence of SEQ ID NO: 133;
HCDR3 comprises the amino acid sequence of SEQ ID NO: 134;
LCDR1 comprises the amino acid sequence of SEQ ID NO: 135;
LCDR2 comprises the amino acid sequence of SEQ ID NO: 136; and
LCDR3 comprises the amino acid sequence of SEQ ID NO: 137;
wherein for set (v):
HCDR1 comprises the amino acid sequence of SEQ ID NO: 138;
HCDR2 comprises the amino acid sequence of SEQ ID NO: 139;
HCDR3 comprises the amino acid sequence of SEQ ID NO: 140;
LCDR1 comprises the amino acid sequence of SEQ ID NO: 141;
LCDR2 comprises the amino acid sequence of SEQ ID NO: 142; and
LCDR3 comprises the amino acid sequence of SEQ ID NO: 143;
wherein for set (vi)
HCDR1 comprises the amino acid sequence of SEQ ID NO: 144;
HCDR2 comprises the amino acid sequence of SEQ ID NO: 145;
HCDR3 comprises the amino acid sequence of SEQ ID NO: 146;
LCDR1 comprises the amino acid sequence of SEQ ID NO: 147;
LCDR2 comprises the amino acid sequence of SEQ ID NO: 148; and
LCDR3 comprises the amino acid sequence of SEQ ID NO: 149;
wherein for set (vii)
HCDR1 comprises the amino acid sequence of SEQ ID NO: 150;
HCDR2 comprises the amino acid sequence of SEQ ID NO: 151;
HCDR3 comprises the amino acid sequence of SEQ ID NO: 152;
LCDR1 comprises the amino acid sequence of SEQ ID NO: 153;
LCDR2 comprises the amino acid sequence of SEQ ID NO: 235; and
LCDR3 comprises the amino acid sequence of SEQ ID NO: 155;
wherein for set (viii)
HCDR1 comprises the amino acid sequence of SEQ ID NO: 150;
HCDR2 comprises the amino acid sequence of SEQ ID NO: 151;
HCDR3 comprises the amino acid sequence of SEQ ID NO: 152;
LCDR1 comprises the amino acid sequence of SEQ ID NO: 153;
LCDR2 comprises the amino acid sequence of SEQ ID NO: 154; and
LCDR3 comprises the amino acid sequence of SEQ ID NO: 155;
wherein for set (ix)
HCDR1 comprises the amino acid sequence of SEQ ID NO: 248;
HCDR2 comprises the amino acid sequence of SEQ ID NO: 249;
HCDR3 comprises the amino acid sequence of SEQ ID NO: 250;
LCDR1 comprises the amino acid sequence of SEQ ID NO: 251;
LCDR2 comprises the amino acid sequence of SEQ ID NO: 252; and
LCDR3 comprises the amino acid sequence of SEQ ID NO: 253; and
wherein the scFv antibody binds cIL-13.

34.-35. (canceled)


SEQUENCE LISTING (258 sequences)

SEQ ID NO: 1 - protein, 131 aa, synthetic construct
HNFNITIKEIIKMLNILTARNDSCMELTVKDVFTAPKNTSDKEIFCRAATVLRQIYTHNCSNRYLRGLYRNLSSMANKTCSMNEIKKSTLKDFLERLKVIMQKKYYRHGLNDIFEAQKIEWHEHHHHHHHH

SEQ ID NO: 2 - protein, 136 aa, synthetic construct
SPSPVTPSPTLKELIEELVNITQNQASLCNGSMVWSVNLTAGMYCAALESLINVSDCSAIQRTQRMLKALCSQKPAAGQISSERSRDTKIEVIQLVKNLLTYVRGVYRHGNFRGLNDIFEAQKIEWHEHHHHHHHH

SEQ ID NO: 3 - protein, 266 aa, synthetic construct
QAVLTQPASVSAALGQRVTISCTGTATNVGGGYDVQWYQQFPGRPPKTIIYGNYNRPSGVPDRFSASTSGTTATLTITGIQVADEADYYCQSFDTTFNGHVFGAGTQLTILGGGSSRSSSSGGGGSGGGGELQLVELGGDLVKPGGSLRLSCVASGFTFSSYGMSWVRQSPGKGLQWVADISSSGSTYYADAVKGRFTISRDNAKSTLYLQMNSLRAEDTAVYYCAKAGYGSYYDFGMDYWGHGTSVFVSSESPSPPNLTSGQAGQ

SEQ ID NO: 4 - protein, 136 aa, synthetic construct
ELQLVELGGDLVKPGGSLRLSCVASGFTFSSYGMSWVRQSPGKGLQWVADISSSGSTYYADAVKGRFTISRDNAKSTLYLQMNSLRAEDTAVYYCAKAGYGSYYDFGMDYWGHGTSVFVSSESPSPPNLTSGQAGQ

SEQ ID NO: 5 - protein, 111 aa, synthetic construct
QAVLTQPASVSAALGQRVTISCTGTATNVGGGYDVQWYQQFPGRPPKTIIYGNYNRPSGVPDRFSASTSGTTATLTITGIQVADEADYYCQSFDTTFNGHVFGAGTQLTIL

SEQ ID NO: 6 - protein, 264 aa, synthetic construct
QSVLTQPTSVSAALGQRVTISCTGSNANIGSVYDVQWYQQLPGKSPKTIIYGNTHRPSGVPVRFSGSKSGSTATLTITGIQAEDEADYYCQSYDATLHYVFGSGTQLTVLGGGSSRSSSSGGGGSGGGGEVQLVESGGDLVKPGGSLRLSCVASGFTFSNYDMSWVRQAPGKGLQWVSTISYDGNNTYYSDAVKGRFTISRDNARNTLYLQMNSLRAEDTAVYYCAKEFDIPWDNFDYWGQGTLVTVSSASTTAPSVTSGQAGQ

SEQ ID NO: 7 - protein, 135 aa, synthetic construct
EVQLVESGGDLVKPGGSLRLSCVASGFTFSNYDMSWVRQAPGKGLQWVSTISYDGNNTYYSDAVKGRFTISRDNARNTLYLQMNSLRAEDTAVYYCAKEFDIPWDNFDYWGQGTLVTVSSASTTAPSVTSGQAGQ

SEQ ID NO: 8 - protein, 110 aa, synthetic construct
QSVLTQPTSVSAALGQRVTISCTGSNANIGSVYDVQWYQQLPGKSPKTIIYGNTHRPSGVPVRFSGSKSGSTATLTITGIQAEDEADYYCQSYDATLHYVFGSGTQLTVL

SEQ ID NO: 9 - protein, 268 aa, synthetic construct
QSMLTQPASVSGSLGQRVAISCSGSTNNIGIFGATWYQHLPGKAPKFLLYSDGNRPSGVPDRFSGSDSGNSVTLMITALQAEDEADYYCQSFDTTLGAYVFGSGTQLTILGGGSSRSSSSGGGGSGGGGEVQLVQSGGDLVKPGGSLRLSCVASGFAFNGYDMDWVRLAPGKGLQWLSEISSSGSPTYYADAVEGRFTISRDNAKNTLYLQMDSLRAEDTAVYYCARAGEYTYGYEGYYFDYWGQGTLVTVSSASTTAPSVTSGQAGQ

SEQ ID NO: 10 - protein, 139 aa, synthetic construct
EVQLVQSGGDLVKPGGSLRLSCVASGFAFNGYDMDWVRLAPGKGLQWLSEISSSGSPTYYADAVEGRFTISRDNAKNTLYLQMDSLRAEDTAVYYCARAGEYTYGYEGYYFDYWGQGTLVTVSSASTTAPSVTSGQAGQ

SEQ ID NO: 11 - protein, 139 aa, synthetic construct; identical to SEQ ID NO: 10

SEQ ID NO: 12 - protein, 259 aa, synthetic construct
QSVLNQLPSLSMNMGETATFTCEGDNIGHKYVNWYQQKPGQAPVLTIYEDNKRPSGIPERFSGSNSGNTATLTISGARAEDEADYYCESEVSTGIVFGGGTHLTVLGGGSSRSSSSGGGGSGGGGEVQLVESGGDLVKPGGSLRLSCVASGFTFSSYDMDWVRQAPGKGLEWLSEISTNGDRTHYADAVKGRFAISRDNAKNTVYLQMNSLRAEDTAIYYCVRDGYEYLAFDYWGQGTLVTVSSASTTAPSVTSGQAGQ

SEQ ID NO: 13 - protein, 134 aa, synthetic construct
EVQLVESGGDLVKPGGSLRLSCVASGFTFSSYDMDWVRQAPGKGLEWLSEISTNGDRTHYADAVKGRFAISRDNAKNTVYLQMNSLRAEDTAIYYCVRDGYEYLAFDYWGQGTLVTVSSASTTAPSVTSGQAGQ

SEQ ID NO: 14 - protein, 106 aa, synthetic construct
QSVLNQLPSLSMNMGETATFTCEGDNIGHKYVNWYQQKPGQAPVLTIYEDNKRPSGIPERFSGSNSGNTATLTISGARAEDEADYYCESEVSTGIVFGGGTHLTVL

SEQ ID NO: 15 - protein, 262 aa, synthetic construct
QAVLNQPASVSGSLGQRVTISCSGSTNNIGVVGASWYQQFPGKAPKLLVYSDGDRPSGVPDRFSGSNSGYSDTLTITGLQAEDEADYYCQSFDTTLNAYVFGSGTQLTVLGGGSSRSSSSGGGGSGGGGEVPLVESGGDLVKPGGSLRISCVASGITFNNDDMGWVRQAPGKGLQWLAEISSSGTSTYYADAVKGRFTISRDNAKNTVYLQMNSLKAEDTGVYYCVGEDGYMALGFWGQGTLVTVSSASTTAPSVTSGQAGQ

SEQ ID NO: 16 - protein, 133 aa, synthetic construct
EVPLVESGGDLVKPGGSLRISCVASGITFNNDDMGWVRQAPGKGLQWLAEISSSGTSTYYADAVKGRFTISRDNAKNTVYLQMNSLKAEDTGVYYCVGEDGYMALGFWGQGTLVTVSSASTTAPSVTSGQAGQ

SEQ ID NO: 17 - protein, 110 aa, synthetic construct
QAVLNQPASVSGSLGQRVTISCSGSTNNIGVVGASWYQQFPGKAPKLLVYSDGDRPSGVPDRFSGSNSGYSDTLTITGLQAEDEADYYCQSFDTTLNAYVFGSGTQLTVL

SEQ ID NO: 18 - protein, 263 aa, synthetic construct
QPVLTQPPSVSAALGQRVTISCNTNIGSPYDVQWYQQLPGKSPKTIIYGNSNRPSGVPVRFSGSKSGSTATLTIAGIQAEDEADYYCQSYDDNVDGYVFGSGTQLTVLGGGSSRSSSSGGGGSGGGGEVPLVESGGDLVKPGGSLRLSCVASGFTFHNYDLSWIRQLPGGGLQWVADIDKSGLTFYADAVKGRFTISRDNAKNTLYLQMNSLRAEDTAVYYCATWPLGSWYTETFEYWGQGTLVTVSSASTTAPSVTSGQAGQ

SEQ ID NO: 19 - protein, 136 aa, synthetic construct
EVPLVESGGDLVKPGGSLRLSCVASGFTFHNYDLSWIRQLPGGGLQWVADIDKSGLTFYADAVKGRFTISRDNAKNTLYLQMNSLRAEDTAVYYCATWPLGSWYTETFEYWGQGTLVTVSSASTTAPSVTSGQAGQ

SEQ ID NO: 20 - protein, 108 aa, synthetic construct
QPVLTQPPSVSAALGQRVTISCNTNIGSPYDVQWYQQLPGKSPKTIIYGNSNRPSGVPVRFSGSKSGSTATLTIAGIQAEDEADYYCQSYDDNVDGYVFGSGTQLTVL

SEQ ID NO: 21 - protein, 265 aa, synthetic construct
QSVLNQLPSVSGSLGQKVTIRCSGNTDNIGMVGAAWYRQVPGEAPKLLLYSDGNRPSGVPDRFSGSDSGRSATLTITGLQPEDEADYYCQSLDPTVPGNYLFGSGTQLTILGGGSSRSSSSGGGGSGGGGEVQLVESGGDLVKPGGSLRLSCVASGFTFSDYDMTWVRQAPGKGLQWVADINNGGSSTTYADAVKGRFTISRDNAKNTLYLQMNSLRAEDTAMYYCAKEGDNTWLSFDSWGQGTLVTVSSESPSPPNLTSGQAGQ

SEQ ID NO: 22 - protein, 135 aa, synthetic construct
EVQLVESGGDLVKPGGSLRLSCVASGFTFSDYDMTWVRQAPGKGLQWVADINNGGSSTTYADAVKGRFTISRDNAKNTLYLQMNSLRAEDTAMYYCAKEGDNTWLSFDSWGQGTLVTVSSESPSPPNLTSGQAGQ

SEQ ID NO: 23 - protein, 111 aa, synthetic construct
QSVLNQLPSVSGSLGQKVTIRCSGNTDNIGMVGAAWYRQVPGEAPKLLLYSDGNRPSGVPDRFSGSDSGRSATLTITGLQPEDEADYYCQSLDPTVPGNYLFGSGTQLTIL

SEQ ID NO: 24 - protein, 268 aa, synthetic construct
QTVVTQEPSLSVSPGGTVTLTCGLSSGSVSTGNYPGWYQQTQGRAPRSIIYDTNSRPSGVPDRFSGSISGNKAVLTITGAQPEDEADYYCSLYTGIAYTDVVFGGGTHLTVLGGGSSRSSSSGGGGSGGGGEVQLVQSGGDLVKPAGSLRLSCVASGFTFNSYAMSWVRQAPGKGLQWVAGINSGGSTTTYADAVKGRFTISRDNAKNTLYLQMNSLRAEDTAVYYCAKDQNSYYPSDTFEYWGQGTLVTVSSESPSPPNLTSGQAGQ

SEQ ID NO: 25 - protein, 137 aa, synthetic construct
EVQLVQSGGDLVKPAGSLRLSCVASGFTFNSYAMSWVRQAPGKGLQWVAGINSGGSTTTYADAVKGRFTISRDNAKNTLYLQMNSLRAEDTAVYYCAKDQNSYYPSDTFEYWGQGTLVTVSSESPSPPNLTSGQAGQ

SEQ ID NO: 26 - protein, 112 aa, synthetic construct
QTVVTQEPSLSVSPGGTVTLTCGLSSGSVSTGNYPGWYQQTQGRAPRSIIYDTNSRPSGVPDRFSGSISGNKAVLTITGAQPEDEADYYCSLYTGIAYTDVVFGGGTHLTVL

SEQ ID NO: 27 - protein, 265 aa, synthetic construct
QAVLTQPPSVSAALGQRVTISCSGSTDNVGTVGASWYQHLPGRAPKLLVYIVGDRPSGVPDRFSGSKSGTSATLTITGLQAEDEADYYCSSWDDSLRGIVFGGGTHLTVLGGGSSRSSSSGGGGSGGGGEEQLVEFGGDLVKPGESLRLSCVASGFTFNKYDMSWVRQAPGKGLQWVATVADDESFAYYTDSVKGRFTISRDNAKNTLYLQMNSLRAEDTAMYYCVGKTGSYWDIDFDLWGPGTLVTISSASTTAPSVTSGQAGQ

SEQ ID NO: 28 - protein, 136 aa, synthetic construct
EEQLVEFGGDLVKPGESLRLSCVASGFTFNKYDMSWVRQAPGKGLQWVATVADDESFAYYTDSVKGRFTISRDNAKNTLYLQMNSLRAEDTAMYYCVGKTGSYWDIDFDLWGPGTLVTISSASTTAPSVTSGQAGQ

SEQ ID NO: 29 - protein, 110 aa, synthetic construct
QAVLTQPPSVSAALGQRVTISCSGSTDNVGTVGASWYQHLPGRAPKLLVYIVGDRPSGVPDRFSGSKSGTSATLTITGLQAEDEADYYCSSWDDSLRGIVFGGGTHLTVL

SEQ ID NO: 30 - protein, 261 aa, synthetic construct
SYVLSQPPSVSVTPSETAHITCGGNNIGSKYVQWIRQSAGQAPVTIIYGNTNRPTGIPERFSGANSGNTATLTISGVLAEDEADYYCQVGDTDTTVFGGGTHLTVLGGGSSRSSSSGGGGSGGGGEVQLVESGGDLVKPGGSLRLSCVASGFNFSRYSMSWVRQAPEKGLQLVAAVNSGGSRTWYADAVQGRFTVSRDNAKNTLFLEMSSLKLEDTAMYYCAKDLWSTTIAEYDYWGQGTLVTVSSASTTAPSVTSGQAGQ

SEQ ID NO: 31 - protein, 136 aa, synthetic construct
EVQLVESGGDLVKPGGSLRLSCVASGFNFSRYSMSWVRQAPEKGLQLVAAVNSGGSRTWYADAVQGRFTVSRDNAKNTLFLEMSSLKLEDTAMYYCAKDLWSTTIAEYDYWGQGTLVTVSSASTTAPSVTSGQAGQ

SEQ ID NO: 32 - protein, 106 aa, synthetic construct
SYVLSQPPSVSVTPSETAHITCGGNNIGSKYVQWIRQSAGQAPVTIIYGNTNRPTGIPERFSGANSGNTATLTISGVLAEDEADYYCQVGDTDTTVFGGGTHLTVL

SEQ ID NO: 33 - protein, 258 aa, synthetic construct
  VARIANT 244: note = Any amino acid
QSVLNQPPSVSVNLGKTASITCEGDNIGDKYAFWYQQKPGQAPVLIIYDDSKRPSGIPERLSGSNSVNTATLTISGARAEDEADYYCQVWDNSAKAIVFGGGTHLTVLGGGSSRSSSSGGGGSGGGGEVQLVETGGDLVEPGGSLRLSCVASGFTFSGYYMSWIRQAPGKGLQWVADIRDSGGNTHYADAVRGRFTISRDNAKNTLYLQMDSLRAEDTAVYYCARDDWGLDYWGQGTLVTVSSXSPSPPNLTSGQAGQ

SEQ ID NO: 34 - protein, 131 aa, synthetic construct
  VARIANT 117: note = Any amino acid
EVQLVETGGDLVEPGGSLRLSCVASGFTFSGYYMSWIRQAPGKGLQWVADIRDSGGNTHYADAVRGRFTISRDNAKNTLYLQMDSLRAEDTAVYYCARDDWGLDYWGQGTLVTVSSXSPSPPNLTSGQAGQ

SEQ ID NO: 35 - protein, 108 aa, synthetic construct
QSVLNQPPSVSVNLGKTASITCEGDNIGDKYAFWYQQKPGQAPVLIIYDDSKRPSGIPERLSGSNSVNTATLTISGARAEDEADYYCQVWDNSAKAIVFGGGTHLTVL

SEQ ID NO: 36 - protein, 260 aa, synthetic construct
QSVLNQPPSVSAALGQRVTVSCTGTDTNIGSGYDVQWYQQVPGKSPTPIIYGNTNRPSGVPERFSGSKSGNTATLTITGIQAEDEADYYCQSFDDNVDGYVFGSGTQLTILGGGSSRSSSSGGGGSGGGGEVPLVESGGDLVKPGGSLRLSCVASGFTFSDYHMSWVRQAPGKGLQWVADISSSGSTYYADAVKGRFTISRDNAKNTLYLQMNSLRAEDTAVYYCARQWGEFDYWGQGTLVTVSSESPSPPNLTSGQAGQ

SEQ ID NO: 37 - protein, 130 aa, synthetic construct
EVPLVESGGDLVKPGGSLRLSCVASGFTFSDYHMSWVRQAPGKGLQWVADISSSGSTYYADAVKGRFTISRDNAKNTLYLQMNSLRAEDTAVYYCARQWGEFDYWGQGTLVTVSSESPSPPNLTSGQAGQ

SEQ ID NO: 38 - protein, 111 aa, synthetic construct
QSVLNQPPSVSAALGQRVTVSCTGTDTNIGSGYDVQWYQQVPGKSPTPIIYGNTNRPSGVPERFSGSKSGNTATLTITGIQAEDEADYYCQSFDDNVDGYVFGSGTQLTIL

SEQ ID NO: 39 - protein, 268 aa, synthetic construct
DIVMTQTPLSLSVSPGEPASISCKASQSLLYSDGNTYLYWFRQKPGQSPQRLISLVSNRDAGVPDRFSGSGSGTDFTLRISRVEADDAGVYYCGQGIQDPITFGKGTHLEIKGGSSRSSSSGGGGSGGGGEVPLVESGGDLVKPGGSLRLSCVASGFPFRSYGMNWVRQSPGGGLQWVAGILFDGSATAYADAVRGRFTISRDNAKNTLYLQMNSLRAEDTAVYYCASDDGSYIEAYNWFYYWGQGTLVTVSSASTTAPSVTSGQAGQ

SEQ ID NO: 40 - protein, 138 aa, synthetic construct
EVPLVESGGDLVKPGGSLRLSCVASGFPFRSYGMNWVRQSPGGGLQWVAGILFDGSATAYADAVRGRFTISRDNAKNTLYLQMNSLRAEDTAVYYCASDDGSYIEAYNWFYYWGQGTLVTVSSASTTAPSVTSGQAGQ

SEQ ID NO: 41 - protein, 112 aa, synthetic construct
DIVMTQTPLSLSVSPGEPASISCKASQSLLYSDGNTYLYWFRQKPGQSPQRLISLVSNRDAGVPDRFSGSGSGTDFTLRISRVEADDAGVYYCGQGIQDPITFGKGTHLEIK

SEQ ID NO: 42 - protein, 456 aa, synthetic construct
EEQLVEFGGDLVKPGESLRLSCVASGFTFNKYDMSWVRQAPGKGLQWVATVADDESFAYYTDSVKGRFTISRDNAKNTLYLQMNSLRAEDTAMYYCVGKTGSYWDIDFDLWGPGTLVTISSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 43 - protein, 216 aa, synthetic construct
QAVLTQPPSVSAALGQRVTISCSGSTDNVGTVGASWYQHLPGRAPKLLVYIVGDRPSGVPDRFSGSKSGTSATLTITGLQAEDEADYYCSSWDDSLRGIVFGGGTHLTVLGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 44 - protein, 457 aa, synthetic construct
EVQLVQSGGDLVKPAGSLRLSCVASGFTFNSYAMSWVRQAPGKGLQWVAGINSGGSTTTYADAVKGRFTISRDNAKNTLYLQMNSLRAEDTAVYYCAKDQNSYYPSDTFEYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 45 - protein, 218 aa, synthetic construct
QTVVTQEPSLSVSPGGTVTLTCGLSSGSVSTGNYPGWYQQTQGRAPRSIIYDTNSRPSGVPDRFSGSISGNKAVLTITGAQPEDEADYYCSLYTGIAYTDVVFGGGTHLTVLGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 46 - protein, 455 aa, synthetic construct
EVQLVESGGDLVKPGGSLRLSCVASGFTFSDYDMTWVRQAPGKGLQWVADINNGGSSTTYADAVKGRFTISRDNAKNTLYLQMNSLRAEDTAMYYCAKEGDNTWLSFDSWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 47 - protein, 217 aa, synthetic construct
QSVLNQLPSVSGSLGQKVTIRCSGNTDNIGMVGAAWYRQVPGEAPKLLLYSDGNRPSGVPDRFSGSDSGRSATLTITGLQPEDEADYYCQSLDPTVPGNYLFGSGTQLTILGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 48 - protein, 456 aa, synthetic construct
EVPLVESGGDLVKPGGSLRLSCVASGFTFHNYDLSWIRQLPGGGLQWVADIDKSGLTFYADAVKGRFTISRDNAKNTLYLQMNSLRAEDTAVYYCATWPLGSWYTETFEYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 49 - protein, 214 aa, synthetic construct
QPVLTQPPSVSAALGQRVTISCNTNIGSPYDVQWYQQLPGKSPKTIIYGNSNRPSGVPVRFSGSKSGSTATLTIAGIQAEDEADYYCQSYDDNVDGYVFGSGTQLTVLGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 50 - protein, 453 aa, synthetic construct
EVPLVESGGDLVKPGGSLRISCVASGITFNNDDMGWVRQAPGKGLQWLAEISSSGTSTYYADAVKGRFTISRDNAKNTVYLQMNSLKAEDTGVYYCVGEDGYMALGFWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 51 - protein, 216 aa, synthetic construct
QAVLNQPASVSGSLGQRVTISCSGSTNNIGVVGASWYQQFPGKAPKLLVYSDGDRPSGVPDRFSGSNSGYSDTLTITGLQAEDEADYYCQSFDTTLNAYVFGSGTQLTVLGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 52 - protein, 456 aa, synthetic construct
ELQLVELGGDLVKPGGSLRLSCVASGFTFSSYGMSWVRQSPGKGLQWVADISSSGSTYYADAVKGRFTISRDNAKSTLYLQMNSLRAEDTAVYYCAKAGYGSYYDFGMDYWGHGTSVFVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 53 - protein, 217 aa, synthetic construct
QAVLTQPASVSAALGQRVTISCTGTATNVGGGYDVQWYQQFPGRPPKTIIYGNYNRPSGVPDRFSASTSGTTATLTITGIQVADEADYYCQSFDTTFNGHVFGAGTQLTILGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 54 - protein, 455 aa, synthetic construct
EVQLVESGGDLVKPGGSLRLSCVASGFTFSNYDMSWVRQAPGKGLQWVSTISYDGNNTYYSDAVKGRFTISRDNARNTLYLQMNSLRAEDTAVYYCAKEFDIPWDNFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 55 - protein, 216 aa, synthetic construct
QSVLTQPTSVSAALGQRVTISCTGSNANIGSVYDVQWYQQLPGKSPKTIIYGNTHRPSGVPVRFSGSKSGSTATLTITGIQAEDEADYYCQSYDATLHYVFGSGTQLTVLGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 56 - protein, 455 aa, synthetic construct
EVQLVESGGDLVKPGGSLRLSCVASGFTFSNYDMSWVRQAPGKGLQWVSTISYDGQNTYYSDAVKGRFTISRDNARNTLYLQMNSLRAEDTAVYYCAKEFDIPWDNFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 57 - protein, 459 aa, synthetic construct
EVQLVQSGGDLVKPGGSLRLSCVASGFAFNGYDMDWVRLAPGKGLQWLSEISSSGSPTYYADAVEGRFTISRDNAKNTLYLQMDSLRAEDTAVYYCARAGEYTYGYEGYYFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 58 - protein, 216 aa, synthetic construct
QSMLTQPASVSGSLGQRVAISCSGSTNNIGIFGATWYQHLPGKAPKFLLYSDGNRPSGVPDRFSGSDSGNSVTLMITALQAEDEADYYCQSFDTTLGAYVFGSGTQLTILGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 59 - protein, 454 aa, synthetic construct
EVQLVESGGDLVKPGGSLRLSCVASGFTFSSYDMDWVRQAPGKGLEWLSEISTNGDRTHYADAVKGRFAISRDNAKNTVYLQMNSLRAEDTAIYYCVRDGYEYLAFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 60 - protein, 212 aa, synthetic construct
QSVLNQLPSLSMNMGETATFTCEGDNIGHKYVNWYQQKPGQAPVLTIYEDNKRPSGIPERFSGSNSGNTATLTISGARAEDEADYYCESEVSTGIVFGGGTHLTVLGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 61 - protein, 258 aa, synthetic construct
SYVLTQSPSVSGSLGQTVTISCSGTTIGVVGANWYQQLPGKAPKLLVHSDGGRLSGVPDRFSGSESGNSAALTITGLQAEDEADYYCQSFDTTLGYVFGSGTQLTILGGGSSRSSSSGGGGSGGGGELTLQESGPGLVKPSQTLSLTCVVSGGSVTSNYYWNWIRQRPGRGLEWMGYWTGSTNYNPAFQGRISITADTAKNHFSLQLTSMTTEDTAVYYCARASTWLHGMDFWGHGTSLFVSSESPSPPNLTSGQAGQ

SEQ ID NO: 62 - protein, 132 aa, synthetic construct
ELTLQESGPGLVKPSQTLSLTCVVSGGSVTSNYYWNWIRQRPGRGLEWMGYWTGSTNYNPAFQGRISITADTAKNHFSLQLTSMTTEDTAVYYCARASTWLHGMDFWGHGTSLFVSSESPSPPNLTSGQAGQ

SEQ ID NO: 63 - protein, 107 aa, synthetic construct
SYVLTQSPSVSGSLGQTVTISCSGTTIGVVGANWYQQLPGKAPKLLVHSDGGRLSGVPDRFSGSESGNSAALTITGLQAEDEADYYCQSFDTTLGYVFGSGTQLTIL

SEQ ID NO: 64 - protein, 262 aa, synthetic construct
QAVLNQPASVSGSLGQRVTISCSGSSSNIGRGFVGWYQQLPGTRPKTLIYGDTFRPSGVPDRFFGSRSGSTATLTISGLQAEDEADYYCSTWDDSLAALLFGGGTHLTVLGGGSSRSSFSGGGGSGGGGEVQLVESGGDLVKPGGSLRLSCAASGFTFTDYDMSWVRQAPGKGLQWVATTSNGGSGTYYSDAVKGRFTISRDNAKNTLYLEMNRLRAEDTAVYYCVKEIYPASIDFWGLGTLVTVSSASTTAPSVTSGQAGQ

SEQ ID NO: 65 - protein, 133 aa, synthetic construct
EVQLVESGGDLVKPGGSLRLSCAASGFTFTDYDMSWVRQAPGKGLQWVATTSNGGSGTYYSDAVKGRFTISRDNAKNTLYLEMNRLRAEDTAVYYCVKEIYPASIDFWGLGTLVTVSSASTTAPSVTSGQAGQ

SEQ ID NO: 66 - protein, 110 aa, synthetic construct
QAVLNQPASVSGSLGQRVTISCSGSSSNIGRGFVGWYQQLPGTRPKTLIYGDTFRPSGVPDRFFGSRSGSTATLTISGLQAEDEADYYCSTWDDSLAALLFGGGTHLTVL

SEQ ID NO: 67 - protein, 262 aa, synthetic construct
QSALTQTASVSGSLGQRVTISCTGATRNVGTVGVTWYQQVPGKAPKLLLFSNGDRPSWVPDRFSGSTSGYSATLIINGVQAEDEAMYHCQSFDTMPDAHYMFGSGTQLTVLGGGSSRSSSSGGGGSGGGGELTLQESGPGLVKPSQTLSLTCVVSGASVTSTNYWNWIRQRPGRGLEWMGYWTGSTKYNPAFQGRISITADTAKNQFSLQLSSMTTEDTAVYYCARSSTWINGFDYWGQGTLVTVSSESPSPPNLTSGQAGQ

SEQ ID NO: 68 - protein, 132 aa, synthetic construct
ELTLQESGPGLVKPSQTLSLTCVVSGASVTSTNYWNWIRQRPGRGLEWMGYWTGSTKYNPAFQGRISITADTAKNQFSLQLSSMTTEDTAVYYCARSSTWINGFDYWGQGTLVTVSSESPSPPNLTSGQAGQ

SEQ ID NO: 69 - protein, 111 aa, synthetic construct
QSALTQTASVSGSLGQRVTISCTGATRNVGTVGVTWYQQVPGKAPKLLLFSNGDRPSWVPDRFSGSTSGYSATLIINGVQAEDEAMYHCQSFDTMPDAHYMFGSGTQLTVL

SEQ ID NO: 70 - protein, 272 aa, synthetic construct
QSVLNQPPSVSAALGQRVTISCTGTNTNIGSGYSVQWYQQLPGESPKPIIYGSSNRPSGVPARFSGSKSGSTGTLTITGIQAEDEADYHCQSYDDSLDGHAVFGGGTQLTVLGGGSSRSSSSGGGGSGGGGEVQLVQSGGDLVKPGGSLRLSCVASGFTFSGYDMSWVRQSPGKGLQWVATVWSDGNTIYYADAVKGRFTISRDNAKNTVYLQMNSLRAEDTAMYYCAKSPYCTDDYCPWGDYLDLWGQGTLVTVSSASTTAPSVTSGQAGQ

SEQ ID NO: 71 - protein, 141 aa, synthetic construct
EVQLVQSGGDLVKPGGSLRLSCVASGFTFSGYDMSWVRQSPGKGLQWVATVWSDGNTIYYADAVKGRFTISRDNAKNTVYLQMNSLRAEDTAMYYCAKSPYCTDDYCPWGDYLDLWGQGTLVTVSSASTTAPSVTSGQAGQ

SEQ ID NO: 72 - protein, 112 aa, synthetic construct
QSVLNQPPSVSAALGQRVTISCTGTNTNIGSGYSVQWYQQLPGESPKPIIYGSSNRPSGVPARFSGSKSGSTGTLTITGIQAEDEADYHCQSYDDSLDGHAVFGGGTQLTVL

SEQ ID NO: 73 - protein, 262 aa, synthetic construct
QPVLTQPPSVSVSLGQTATISCSGESLNEYYAQWYQQKAGQAPVLIMYKDTERPSGIPDRFSGSSSGNIHTLTISGTRAEDEADYYCGSLVSTYDYIFGSGTQLTVLGGGSSRSSSSGGGGSGGGGEVQLVQSGGDLVKTGGSLRLSCVASGFTFSRYDMSWVRQAPGKGLQWVARISSDGSTTYYADAVKGRFTISRDNAKNTLYLQMNSLRAEDTAMYYCAKDLVWSAWYGMDYWGHGTSLFVSSASTTAPSVTSGQAGQ

SEQ ID NO: 74 - protein, 136 aa, synthetic construct
EVQLVQSGGDLVKTGGSLRLSCVASGFTFSRYDMSWVRQAPGKGLQWVARISSDGSTTYYADAVKGRFTISRDNAKNTLYLQMNSLRAEDTAMYYCAKDLVWSAWYGMDYWGHGTSLFVSSASTTAPSVTSGQAGQ

SEQ ID NO: 75 - protein, 107 aa, synthetic construct
QPVLTQPPSVSVSLGQTATISCSGESLNEYYAQWYQQKAGQAPVLIMYKDTERPSGIPDRFSGSSSGNIHTLTISGTRAEDEADYYCGSLVSTYDYIFGSGTQLTVL

SEQ ID NO: 76 - protein, 260 aa, synthetic construct
SYELTQPPSVSVSLGQTATISCSGESLSKYYAQWFQQKAGQAPVLVIYQDTERPSGIPDRFSGSSSGNTHTLTISGARAEDEADYYCESAISPDTYVFGSGTQLTILGGGSSRSSSSGGGGSGGGGEVQLVQSGGDLVKPGGTLRLSCVASGFTFSSYDMSWVRQSPGKGLQWVAAIWNDGSNTEYADAVKGRFTVSRDNAKNTVYLEMNSLRAEDTAMYYCVKEGPSYGYFDYWGQGTLVTVSSASTTAPSVTSGQAGQ

SEQ ID NO: 77 - protein, 134 aa, synthetic construct
EVQLVQSGGDLVKPGGTLRLSCVASGFTFSSYDMSWVRQSPGKGLQWVAAIWNDGSNTEYADAVKGRFTVSRDNAKNTVYLEMNSLRAEDTAMYYCVKEGPSYGYFDYWGQGTLVTVSSASTTAPSVTSGQAGQ

SEQ ID NO: 78 - protein, 107 aa, synthetic construct
SYELTQPPSVSVSLGQTATISCSGESLSKYYAQWFQQKAGQAPVLVIYQDTERPSGIPDRFSGSSSGNTHTLTISGARAEDEADYYCESAISPDTYVFGSGTQLTIL

SEQ ID NO: 79 - protein, 260 aa, synthetic construct
SYVLTQPPSVTVPLRQTARISCGGDNIRSKNVHWYQQKPGQAPVLIIYNDSNRPTGIPERFSGSNSGNMATLTIRGALAEDEADYYCQVWANDGYVFGSGTQLTVLGGGSSRSSSSGGGGSGGGGEVQLVESGGDSVRPGGSLRLSCVASGFTFSRYNMGWVRQAPGKGLQWVAWIYDGGSRTSYADAVKGRFTISRDNAKDTLYLQMDDLRVDDTAVYYCARDPSSWGSLFDYWGQGTLVTVSSASTTAPSVTSGQAGQ

SEQ ID NO: 80 - protein, 135 aa, synthetic construct
EVQLVESGGDSVRPGGSLRLSCVASGFTFSRYNMGWVRQAPGKGLQWVAWIYDGGSRTSYADAVKGRFTISRDNAKDTLYLQMDDLRVDDTAVYYCARDPSSWGSLFDYWGQGTLVTVSSASTTAPSVTSGQAGQ

SEQ ID NO: 81 - protein, 106 aa, synthetic construct
SYVLTQPPSVTVPLRQTARISCGGDNIRSKNVHWYQQKPGQAPVLIIYNDSNRPTGIPERFSGSNSGNMATLTIRGALAEDEADYYCQVWANDGYVFGSGTQLTVL

SEQ ID NO: 82 - protein, 454 aa, synthetic construct
EVQLVQSGGDLVKPGGTLRLSCVASGFTFSSYDMSWVRQSPGKGLQWVAAIWNDGSNTEYADAVKGRFTVSRDNAKNTVYLEMNSLRAEDTAMYYCVKEGPSYGYFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 83 - protein, 213 aa, synthetic construct
SYELTQPPSVSVSLGQTATISCSGESLSKYYAQWFQQKAGQAPVLVIYQDTERPSGIPDRFSGSSSGNTHTLTISGARAEDEADYYCESAISPDTYVFGSGTQLTILGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 84 - protein, 455 aa, synthetic construct
EVQLVESGGDSVRPGGSLRLSCVASGFTFSRYNMGWVRQAPGKGLQWVAWIYDGGSRTSYADAVKGRFTISRDNAKDTLYLQMDDLRVDDTAVYYCARDPSSWGSLFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 85 - protein, 212 aa, synthetic construct
SYVLTQPPSVTVPLRQTARISCGGDNIRSKNVHWYQQKPGQAPVLIIYQDSNRPTGIPERFSGSNSGNMATLTIRGALAEDEADYYCQVWANDGYVFGSGTQLTVLGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 86 - protein, 212 aa, synthetic construct
SYVLTQPPSVTVPLRQTARISCGGDNIRSKNVHWYQQKPGQAPVLIIYNDSNRPTGIPERFSGSNSGNMATLTIRGALAEDEADYYCQVWANDGYVFGSGTQLTVLGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 87 - protein, 452 aa, synthetic construct
ELTLQESGPGLVKPSQTLSLTCVVSGGSVTSNYYWNWIRQRPGRGLEWMGYWTGSTNYNPAFQGRISITADTAKNHFSLQLTSMTTEDTAVYYCARASTWLHGMDFWGHGTSLFVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 88 - protein, 213 aa, synthetic construct
SYVLTQSPSVSGSLGQTVTISCSGTTIGVVGANWYQQLPGKAPKLLVHSDGGRLSGVPDRFSGSESGNSAALTITGLQAEDEADYYCQSFDTTLGYVFGSGTQLTILGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 89 - protein, 31 aa, Canis lupus
IIKMLNILTARNDSCMELTVKDVFTAPKNTS

SEQ ID NO: 90 - protein, 21 aa, Canis lupus
CSMNEIKKSTLKDFLERLKVI

SEQ ID NO: 91 - protein, 20 aa, Canis lupus
HNFNITIKEIIKMLNILTAR

SEQ ID NO: 92 - protein, 11 aa, Canis lupus
RNLSSMANKTC

SEQ ID NO: 93 - protein, 21 aa, Canis lupus
NGSMVWSVNLTAGMYCAALES

SEQ ID NO: 94 - protein, 31 aa, Canis lupus
ISSERSRDTKIEVIQLVKNLLTYVRGVYRHG

SEQ ID NO: 95 - protein, 21 aa, Canis lupus
NITQNQASLCNGSMVWSVNLT

SEQ ID NO: 96 - protein, 11 aa, Canis lupus
SLINVSDCSAI

SEQ ID NO: 97 - protein, 41 aa, Canis lupus
NITQNQASLCNGSMVWSVNLTAGMYCAALESLINVSDCSAI

SEQ ID NO: 98 - protein, 11 aa, Canis lupus
ISSERSRDTKI

SEQ ID NO: 99 - protein, 216 aa, Canis lupus
LGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 100 - protein, 216 aa, synthetic construct
LGGPSVLIFPPKPKDILRITRTPEVTCVVLALGREDPEVQISWFVDGKEVHTAKTQSREQQFAGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 101 - protein, 215 aa, Canis lupus
LGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 102 - protein, 215 aa, synthetic construct
LGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 103 - protein, 215 aa, Canis lupus
LGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 104 - protein, 215 aa, synthetic construct
LGGPSVFIFPPKPKDILVTARTPTVTCVVVALDPENPEVQISWFVDSKQVQTANTQPREEQSAGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 105 - protein, 216 aa, Canis lupus
LGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 106 - protein, 216 aa, synthetic construct
LGGPSVFIFPPKPKDILRITRTPEITCVVLALGREDPEVQISWFVDGKEVHTAKTQPREQQFASTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 107 - protein, 216 aa, Homo sapiens
PELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 216 aa, synthetic construct
  VARIANT 18: note = Any amino acid
  VARIANT 20: note = Any amino acid
  VARIANT 106: note = Any amino acid
  VARIANT 111: note = Any amino acid
  VARIANT 113: note = Any amino acid
  VARIANT 128: note = Any amino acid
  VARIANT 158: note = Any amino acid
LGGPSVFIFPPKPKDTLXIXRTPEVTCVVVDLGPEDPEVQISWFVDGKEVHTAKTQPREEQFNSTYRVVSVLPILHQDWLKGKEFKCKVNNKALPSPIERTISKAXGQAHXPXVYVLPPSREELSKNXTVSLTCLIKDFFPPDIDVEWQSNGQPEPEXKYRTTPPQLDEDGSYFLYSKLSVDKSRWQRGDTFTCAVMHEALHNHYTQKSLSHSPGK

SEQ ID NO: 109 - protein, 17 aa, synthetic construct
FNECRCTDTPPCPVPEP

SEQ ID NO: 110 - protein, 22 aa, synthetic construct
  VARIANT 19: note = Threonine or Alanine
PKRENGRVPRPPDCPKCPXPEM

SEQ ID NO: 111 - protein, 20 aa, synthetic construct
AKECECKCNCNNCPCPGCGL

SEQ ID NO: 112 - protein, 17 aa, synthetic construct
PKESTCKCISPCPVPES

SEQ ID NO: 113 - protein, 17 aa, synthetic construct
PKESTCKCIPPCPVPES

SEQ ID NO: 114 - protein, 6 aa, synthetic construct
SNYYWN

SEQ ID NO: 115 - protein, 14 aa, synthetic construct
YWTGSTNYNPAFQG

SEQ ID NO: 116 - protein, 10 aa, synthetic construct
ASTWLHGMDF

SEQ ID NO: 117 - protein, 11 aa, synthetic construct
SGTTIGVVGAN

SEQ ID NO: 118 - protein, 7 aa, synthetic construct
SDGGRLS

SEQ ID NO: 119 - protein, 10 aa, synthetic construct
QSFDTTLGYV

SEQ ID NO: 120 - protein, 5 aa, synthetic construct
DYDMS

SEQ ID NO: 121 - protein, 17 aa, synthetic construct
TTSNGGSGTYYSDAVKG

SEQ ID NO: 122 - protein, 9 aa, synthetic construct
EIYPASIDF

SEQ ID NO: 123 - protein, 13 aa, synthetic construct
SGSSSNIGRGFVG

SEQ ID NO: 124 - protein, 7 aa, synthetic construct
GDTFRPS

SEQ ID NO: 125 - protein, 11 aa, synthetic construct
STWDDSLAALL

SEQ ID NO: 126 - protein, 6 aa, synthetic construct
STNYWN

SEQ ID NO: 127 - protein, 14 aa, synthetic construct
YWTGSTKYNPAFQG

SEQ ID NO: 128 - protein, 10 aa, synthetic construct
SSTWINGFDY

SEQ ID NO: 129 - protein, 13 aa, synthetic construct
TGATRNVGTVGVT

SEQ ID NO: 130 - protein, 7 aa, synthetic construct
SNGDRPS

SEQ ID NO: 131 - protein, 12 aa, synthetic construct
QSFDTMPDAHYM

SEQ ID NO: 132 - protein, 5 aa, synthetic construct
GYDMS

SEQ ID NO: 133 - protein, 17 aa, synthetic construct
TVWSDGNTIYYADAVKG

SEQ ID NO: 134 - protein, 17 aa, synthetic construct
SPYCTDDYCPWGDYLDL

SEQ ID NO: 135 - protein, 14 aa, synthetic construct
TGTNTNIGSGYSVQ

SEQ ID NO: 136 - protein, 7 aa, synthetic construct
GSSNRPS

SEQ ID NO: 137 - protein, 12 aa, synthetic construct
QSYDDSLDGHAV

SEQ ID NO: 138 - protein, 5 aa, synthetic construct
RYDMS

SEQ ID NO: 139 - protein, 17 aa, synthetic construct
RISSDGSTTYYADAVKG

SEQ ID NO: 140 - protein, 12 aa, synthetic construct
DLVWSAWYGMDY

SEQ ID NO: 141 - protein, 11 aa, synthetic construct
SGESLNEYYAQ

SEQ ID NO: 142 - protein, 7 aa, synthetic construct
KDTERPS

SEQ ID NO: 143 - protein, 10 aa, synthetic construct
GSLVSTYDYI

SEQ ID NO: 144 - protein, 5 aa, synthetic construct
SYDMS

SEQ ID NO: 145 - protein, 17 aa, synthetic construct
AIWNDGSNTEYADAVKG

SEQ ID NO: 146 - protein, 10 aa, synthetic construct
EGPSYGYFDY

SEQ ID NO: 147 - protein, 11 aa, synthetic construct
SGESLSKYYAQ

SEQ ID NO: 148 - protein, 7 aa, synthetic construct
QDTERPS

SEQ ID NO: 149 - protein, 10 aa, synthetic construct
ESAISPDTYV

SEQ ID NO: 150 - protein, 5 aa, synthetic construct
RYNMG

SEQ ID NO: 151 - protein, 17 aa, synthetic construct
WIYDGGSRTSYADAVKG

SEQ ID NO: 152 - protein, 11 aa, synthetic construct
DPSSWGSLFDY

SEQ ID NO: 153 - protein, 11 aa, synthetic construct
GGDNIRSKNVH

SEQ ID NO: 154 - protein, 7 aa, synthetic construct
NDSNRPT

SEQ ID NO: 155 - protein, 9 aa, synthetic construct
QVWANDGYV

SEQ ID NO: 156 - protein, 5 aa, synthetic construct
SYGMS

SEQ ID NO: 157 - protein, 16 aa, synthetic construct
DISSSGSTYYADAVKG

SEQ ID NO: 158 - protein, 13 aa, synthetic construct
AGYGSYYDFGMDY

SEQ ID NO: 159 - protein, 14 aa, synthetic construct
TGTATNVGGGYDVQ

SEQ ID NO: 160 - protein, 7 aa, synthetic construct
GNYNRPS

SEQ ID NO: 161 - protein, 11 aa, synthetic construct
QSFDTTFNGHV

SEQ ID NO: 162 - protein, 5 aa, synthetic construct
NYDMS

SEQ ID NO: 163 - protein, 17 aa, synthetic construct
TISYDGNNTYYSDAVKG

SEQ ID NO: 164 - protein, 11 aa, synthetic construct
EFDIPWDNFDY

SEQ ID NO: 165 - protein, 14 aa, synthetic construct
TGSNANIGSVYDVQ

SEQ ID NO: 166 - protein, 7 aa, synthetic construct
GNTHRPS

SEQ ID NO: 167 - protein, 10 aa, synthetic construct
QSYDATLHYV

SEQ ID NO: 168 - protein, 5 aa, synthetic construct
GYDMD

SEQ ID NO: 169 - protein, 17 aa, synthetic construct
EISSSGSPTYYADAVEG

SEQ ID NO: 170 - protein, 15 aa, synthetic construct
AGEYTYGYEGYYFDY

SEQ ID NO: 171 - protein, 13 aa, synthetic construct
SGSTNNIGIFGAT

SEQ ID NO: 172 - protein, 7 aa, synthetic construct
SDGNRPS

SEQ ID NO: 173 - protein, 11 aa, synthetic construct
QSFDTTLGAYV

SEQ ID NO: 174 - protein, 5 aa, synthetic construct
SYDMD

SEQ ID NO: 175 - protein, 17 aa, synthetic construct
EISTNGDRTHYADAVKG

SEQ ID NO: 176 - protein, 10 aa, synthetic construct
DGYEYLAFDY

SEQ ID NO: 177 - protein, 11 aa, synthetic construct
EGDNIGHKYVN

SEQ ID NO: 178 - protein, 7 aa, synthetic construct
EDNKRPS

SEQ ID NO: 179 - protein, 9 aa, synthetic construct
ESEVSTGIV

SEQ ID NO: 180 - protein, 5 aa, synthetic construct
NDDMG

SEQ ID NO: 181 - protein, 17 aa, synthetic construct
EISSSGTSTYYADAVKG

SEQ ID NO: 182 - protein, 9 aa, synthetic construct
EDGYMALGF

SEQ ID NO: 183 - protein, 13 aa, synthetic construct
SGSTNNIGVVGAS

SEQ ID NO: 184 - protein, 7 aa, synthetic construct
SDGDRPS

SEQ ID NO: 185 - protein, 11 aa, synthetic construct
QSFDTTLNAYV

SEQ ID NO: 186 - protein, 5 aa, synthetic construct
NYDLS

SEQ ID NO: 187 - protein, 16 aa, synthetic construct
DIDKSGLTFYADAVKG

SEQ ID NO: 188 - protein, 13 aa, synthetic construct
WPLGSWYTETFEY

SEQ ID NO: 189 - protein, 11 aa, synthetic construct
NTNIGSPYDVQ

SEQ ID NO: 190 - protein, 7 aa, synthetic construct
GNSNRPS

SEQ ID NO: 191 - protein, 11 aa, synthetic construct
QSYDDNVDGYV

SEQ ID NO: 192 - protein, 5 aa, synthetic construct
DYDMT

SEQ ID NO: 193 - protein, 17 aa, synthetic construct
DINNGGSSTTYADAVKG

SEQ ID NO: 194 - protein, 11 aa, synthetic construct
EGDNTWLSFDS

SEQ ID NO: 195 - protein, 13 aa, synthetic construct
SGNTDNIGMVGAA

SEQ ID NO: 196 - protein, 7 aa, synthetic construct
SDGNRPS

SEQ ID NO: 197 - protein, 12 aa, synthetic construct
QSLDPTVPGNYL

SEQ ID NO: 198 - protein, 5 aa, synthetic construct
SYAMS

SEQ ID NO: 199 - protein, 17 aa, synthetic construct
GINSGGSTTTYADAVKG

SEQ ID NO: 200 - protein, 13 aa, synthetic construct
DQNSYYPSDTFEY

SEQ ID NO: 201 - protein, 14 aa, synthetic construct
GLSSGSVSTGNYPG

SEQ ID NO: 202 - protein, 7 aa, synthetic construct
DTNSRPS

SEQ ID NO: 203 - protein, 12 aa, synthetic construct
SLYTGIAYTDVV

SEQ ID NO: 204 - protein, 5 aa, synthetic construct
KYDMS

SEQ ID NO: 205 - protein, 17 aa, synthetic construct
TVADDESFAYYTDSVKG

SEQ ID NO: 206 - protein, 11 aa, synthetic construct
KTGSYWDIDFD

SEQ ID NO: 207 - protein, 13 aa, synthetic construct
SGSTDNVGTVGAS

SEQ ID NO: 208 - protein, 7 aa, synthetic construct
IVGDRPS

SEQ ID NO: 209 - protein, 11 aa, synthetic construct
SSWDDSLRGIV

SEQ ID NO: 210 - protein, 5 aa, synthetic construct
RYSMS

SEQ ID NO: 211 - protein, 17 aa, synthetic construct
AVNSGGSRTWYADAVQG

SEQ ID NO: 212 - protein, 12 aa, synthetic construct
DLWSTTIAEYDY

SEQ ID NO: 213 - protein, 11 aa, synthetic construct
GGNNIGSKYVQ

SEQ ID NO: 214 - protein, 7 aa, synthetic construct
GNTNRPT

SEQ ID NO: 215 - protein, 9 aa, synthetic construct
QVGDTDTTV

SEQ ID NO: 216 - protein, 5 aa, synthetic construct
GYYMS

SEQ ID NO: 217 - protein, 17 aa, synthetic construct
DIRDSGGNTHYADAVRG

SEQ ID NO: 218 - protein, 7 aa, synthetic construct
DDWGLDY

SEQ ID NO: 219 - protein, 11 aa, synthetic construct
EGDNIGDKYAF

SEQ ID NO: 220 - protein, 7 aa, synthetic construct
DDSKRPS

SEQ ID NO: 221 - protein, 11 aa, synthetic construct
QVWDNSAKAIV

SEQ ID NO: 222 - protein, 5 aa, synthetic construct
DYHMS

SEQ ID NO: 223 - protein, 16 aa, synthetic construct
DISSSGSTYYADAVKG

SEQ ID NO: 224 - protein, 7 aa, synthetic construct
QWGEFDY

SEQ ID NO: 225 - protein, 14 aa, synthetic construct
TGTDTNIGSGYDVQ

SEQ ID NO: 226 - protein, 7 aa, synthetic construct
GNTNRPS

SEQ ID NO: 227 - protein, 11 aa, synthetic construct
QSFDDNVDGYV

SEQ ID NO: 228 - protein, 5 aa, synthetic construct
SYGMN

SEQ ID NO: 229 - protein, 17 aa, synthetic construct
GILFDGSATAYADAVRG

SEQ ID NO: 230 - protein, 14 aa, synthetic construct
DDGSYIEAYNWFYY

SEQ ID NO: 231 - protein, 16 aa, synthetic construct
KASQSLLYSDGNTYLY

SEQ ID NO: 232 - protein, 7 aa, synthetic construct
LVSNRDA

SEQ ID NO: 233 - protein, 9 aa, synthetic construct
GQGIQDPIT

SEQ ID NO: 234 - protein, 106 aa, synthetic construct
SYVLTQPPSVTVPLRQTARISCGGDNIRSKNVHWYQQKPGQAPVLIIYQDSNRPTGIPERFSGSNSGNMATLTIRGALAEDEADYYCQVWANDGYVFGSGTQLTVL

SEQ ID NO: 235 - protein, 7 aa, synthetic construct
QDSNRPT

SEQ ID NO: 236 - protein, 135 aa, synthetic construct
EVQLVESGGDLVKPGGSLRLSCVASGFTFSNYDMSWVRQAPGKGLQWVSTISYDGQNTYYSDAVKGRFTISRDNARNTLYLQMNSLRAEDTAVYYCAKEFDIPWDNFDYWGQGTLVTVSSASTTAPSVTSGQAGQ

SEQ ID NO: 237 - protein, 17 aa, synthetic construct
TISYDGQNTYYSDAVKG

SEQ ID NO: 238 - protein, 11 aa, synthetic construct
IIKMLNILTAR

SEQ ID NO: 239 - protein, 16 aa, synthetic construct
QASLCNGSMVWSVNLT

SEQ ID NO: 240 - protein, 455 aa, synthetic construct
EVQLVESGGDSVRPGGSLRLSCVASGFTFSRYNMGWVRQAPGKGLQWVAWIYDGGSRTSYADAVKGRFTISRDNAKDTLYLQMDDLRVDDTAVYYCARDPSSWGSLFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLYIARDPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 241 - protein, 457 aa, synthetic construct
EVQLVQSGGDLVKPAGSLRLSCVASGFTFNSYAMSWVRQAPGKGLQWVAGINSGGSTTTYADAVKGRFTISRDNAKNTLYLQMNSLRAEDTAVYYCAKDQNSYYPSDTFEYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLYIARDPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 242 - protein, 215 aa, synthetic construct
  VARIANT 18: note = Any amino acid
  VARIANT 20: note = Any amino acid
  VARIANT 106: note = Any amino acid
  VARIANT 111: note = Any amino acid
  VARIANT 113: note = Any amino acid
  VARIANT 157: note = Any amino acid
LGGPSVFIFPPKPKDTLXIXRTPEVTCVVVDLGPEDPEVQISWFVDGKEVHTAKTQPREEQFNSTYRVVSVLPILHQDWLKGKEFKCKVNNKALPSPIERTISKAXGQAHXPXVYVLPPSREELSKNTVSLTCLIKDFFPPDIDVEWQSNGQPEPEXKYRTTPPQLDEDGSYFLYSKLSVDKSRWQRGDTFTCAVMHEALHNHYTQKSLSHSPGK

SEQ ID NO: 243 - protein, 260 aa, synthetic construct
QSVLTQPASVSGSLGQRVTISCSGRTNNIGYFGANWYQQVPGKAPKLLVSSYGNRPSGVPDRFSASNSANSATLTITGLQAEDEADYYCQSFDTTLGAVVFGGGTQLTVLGGGSSRSSSSGGGGSGGGGELTLQESGPGLVKPSQTLSLTCVVSGGSVTNTYEWDWIRQRPGRGLEWMGSWTDNTWYNPAFQGRISITADTAKNQLSLQLSSVTTGDTAVYFCARWTKRWGYDYWGQGTLVTVSSASTTAPSVTSGQAGQ

SEQ ID NO: 244 - protein, 116 aa, synthetic construct
ELTLQESGPGLVKPSQTLSLTCVVSGGSVTNTYEWDWIRQRPGRGLEWMGSWTDNTWYNPAFQGRISITADTAKNQLSLQLSSVTTGDTAVYFCARWTKRWGYDYWGQGTLVTVSS

SEQ ID NO: 245 - protein, 110 aa, synthetic construct
QSVLTQPASVSGSLGQRVTISCSGRTNNIGYFGANWYQQVPGKAPKLLVSSYGNRPSGVPDRFSASNSANSATLTITGLQAEDEADYYCQSFDTTLGAVVFGGGTQLTVL

SEQ ID NO: 246 - protein, 451 aa, synthetic construct
ELTLQESGPGLVKPSQTLSLTCVVSGGSVTNTYEWDWIRQRPGRGLEWMGSWTDNTWYNPAFQGRISITADTAKNQLSLQLSSVTTGDTAVYFCARWTKRWGYDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 247 - protein, 216 aa, synthetic construct
QSVLTQPASVSGSLGQRVTISCSGRTNNIGYFGANWYQQVPGKAPKLLVSSYGNRPSGVPDRFSASNSANSATLTITGLQAEDEADYYCQSFDTTLGAVVFGGGTQLTVLGQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

SEQ ID NO: 248 - protein, 6 aa, synthetic construct
NTYEWD

SEQ ID NO: 249 - protein, 14 aa, synthetic construct
SWTDNTWYNPAFQG

SEQ ID NO: 250 - protein, 9 aa, synthetic construct
WTKRWGYDY

SEQ ID NO: 251 - protein, 13 aa, synthetic construct
SGRTNNIGYFGAN

SEQ ID NO: 252 - protein, 7 aa, synthetic construct
SYGNRPS

SEQ ID NO: 253 - protein, 11 aa, synthetic construct
QSFDTTLGAVV

SEQ ID NO: 254 - protein, 260 aa, synthetic construct
SYVLTQPPSVTVPLRQTARISCGGDNIRSKNVHWYQQKPGQAPVLIIYQDSNRPTGIPERFSGSNSGNMATLTIRGALAEDEADYYCQVWANDGYVFGSGTQLTVLGGGSSRSSSSGGGGSGGGGEVQLVESGGDSVRPGGSLRLSCVASGFTFSRYNMGWVRQAPGKGLQWVAWIYDGGSRTSYADAVKGRFTISRDNAKDTLYLQMDDLRVDDTAVYYCARDPSSWGSLFDYWGQGTLVTVSSASTTAPSVTSGQAGQ

SEQ ID NO: 255 - protein, 215 aa, synthetic construct
LGGPSVFIFPPKPKDTLYIARDPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 256 - protein, 215 aa, synthetic construct
LGGPSVFIFPPKPKDTLYIARDPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 257 - protein, 215 aa, synthetic construct
LGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHHHYTQESLSHSPGK

SEQ ID NO: 258 - protein, 215 aa, synthetic construct
LGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHHHYTQESLSHSPGK